SEQUENCE LISTING (104 sequences)

SEQ ID NO: 1 - DNA, 938 bp, Homo sapiens
  source 1..938: mol_type = unassigned DNA
gttgactaagtcaataatcagaatcagcaggtttgcagtcagattggcagggataagcagcctagctcaggagaagtgagtataaaagccccaggctgggagcagccatcacagaagtccactcattcttggcaggatggcttctcatcgtctgctcctcctctgccttgctggactggtatttgtgtctgaggctggccctacgggcaccggtgaatccaagtgtcctctgatggtcaaagttctagatgctgtccgaggcagtcctgccatcaatgtggccgtgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatgagggacttctcctccagtggacctgaaggacgagggatgggatttcatgtaaccaagagtattccatttttactaaagcagtgttttcacctcatatgctatgttagaagtccaggcagagacaataaaacattcctgtgaaaggcacttttcattccactttaacttgattttttaaattcccttattgtcccttccaaaaaaaagagaatcaaaattttacaaagaatcaaaggaattctagaaagtatctgggcagaacgctaggagagatccaaatttccattgtcttgcaagcaaagcacgtattaaatatgatctgcagccattaaaaagacacattctgtaaaaaaaaaaaa

SEQ ID NO: 2-3 - skipped (2 entries)

SEQ ID NO: 4 - DNA, 23 bp, Homo sapiens
  source 1..23: mol_type = unassigned DNA
gatgggatttcatgtaaccaaga

SEQ ID NO: 5 - DNA, 650 bp, Homo sapiens
  source 1..650: mol_type = unassigned DNA
acagaagtccactcattcttggcaggatggcttctcatcgtctgctcctcctctgccttgctggactggtatttgtgtctgaggctggccctacgggcaccggtgaatccaagtgtcctctgatggtcaaagttctagatgctgtccgaggcagtcctgccatcaatgtggccgtgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatgagggacttctcctccagtggacctgaaggacgagggatgggatttcatgtaaccaagagtattccatttttactaaagcagtgttttcacctcatatgctatgttagaagtccaggcagagacaataaaacattcctgtgaaaggcacttttcattccaaaaaaaaaaaaaaaaaaaaaaa

SEQ ID NO: 6 - skipped

SEQ ID NO: 7 - RNA, 23 nt, synthetic construct
  misc_feature 1..23: note = Description of Artificial Sequence: Synthetic oligonucleotide
  source 1..23: mol_type = other RNA
  modified_base 1: mod_base = OTHER; note = 2'-O-methyluridine-3'-phosphorothioate
  modified_base 2: mod_base = OTHER; note = 2'-fluorocytidine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methylguanosine-3'-phosphate
  modified_base 6: mod_base = OTHER; note = 2'-fluoroguanosine-3'-phosphate
  modified_base 7: mod_base = OTHER; note = 2'-O-methyluridine-3'-phosphate
  modified_base 8: mod_base = OTHER; note = 2'-O-methyluridine-3'-phosphate
  modified_base 9: mod_base = OTHER; note = 2'-fluoroadenosine-3'-phosphate
  modified_base 10: mod_base = OTHER; note = 2'-O-methylcytidine-3'-phosphate
  modified_base 11: mod_base = OTHER; note = 2'-O-methyladenosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-fluoroadenosine-3'-phosphate
  modified_base 15: mod_base = OTHER; note = 2'-O-methyladenosine-3'-phosphate
  modified_base 16: mod_base = OTHER; note = 2'-fluoroadenosine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methylcytidine-3'-phosphate
  modified_base 21: mod_base = OTHER; note = 2'-O-methyladenosine-3'-phosphorothioate
  modified_base 22: mod_base = OTHER; note = 2'-O-methyluridine-3'-phosphorothioate
  modified_base 23: mod_base = OTHER; note = 2'-O-methylcytidine-3'-phosphate
tcttggttacatgaaatcccatc

SEQ ID NO: 8-9 - skipped (2 entries)

SEQ ID NO: 10 - RNA, 21 nt, synthetic construct
  misc_feature 1..21: note = Description of Artificial Sequence: Synthetic oligonucleotide
  source 1..21: mol_type = other RNA
  modified_base 1: mod_base = OTHER; note = 2'-O-methyluridine-3'-phosphorothioate
  modified_base 2: mod_base = OTHER; note = 2'-O-methylguanosine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-O-methyluridine-3'-phosphate
  modified_base 7: mod_base = OTHER; note = 2'-fluorouridine-3'-phosphate
  modified_base 8: mod_base = OTHER; note = 2'-O-methyluridine-3'-phosphate
  modified_base 9: mod_base = OTHER; note = 2'-fluorocytidine-3'-phosphate
  modified_base 10: mod_base = OTHER; note = 2'-fluoroadenosine-3'-phosphate
  modified_base 11: mod_base = OTHER; note = 2'-fluorouridine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-O-methyladenosine-3'-phosphate
  modified_base 15: mod_base = OTHER; note = 2'-O-methyladenosine-3'-phosphate
  modified_base 16: mod_base = OTHER; note = 2'-O-methylcytidine-3'-phosphate
  modified_base 17: mod_base = OTHER; note = 2'-O-methylcytidine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methylguanosine-3'-phosphate
  modified_base 21: mod_base = OTHER; note = 2'-O-methyladenosine-3'-phosphate
tgggatttcatgtaaccaaga

SEQ ID NO: 11 - DNA, 21 bp, synthetic construct
  misc_feature 1..21: note = Description of Artificial Sequence: Synthetic oligonucleotide
  source 1..21: mol_type = other DNA
tgggatttcatgtaaccaaga

SEQ ID NO: 12 - RNA, 21 nt, synthetic construct
  misc_feature 1..21: note = Description of Artificial Sequence: Synthetic oligonucleotide
  source 1..21: mol_type = other RNA
tgggatttcatgtaaccaaga

SEQ ID NO: 13 - RNA, 23 nt, synthetic construct
  misc_feature 1..23: note = Description of Artificial Sequence: Synthetic oligonucleotide
  source 1..23: mol_type = other RNA
tcttggttacatgaaatcccatc

SEQ ID NO: 14 - protein, 8 aa, synthetic construct
  REGION 1..8: note = Description of Artificial Sequence: Synthetic peptide
GRGDSPKC

SEQ ID NO: 15 - RNA, 21 nt, synthetic construct
  misc_feature 1..21: note = Description of Artificial Sequence: Synthetic oligonucleotide
  source 1..21: mol_type = other RNA
  modified_base 1: mod_base = OTHER; note = 2'-O-methyluridine-3'-phosphorothioate
  modified_base 2: mod_base = OTHER; note = 2'-O-methylguanosine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-O-hexadecyl-uridine-3'-phosphate
  modified_base 7: mod_base = OTHER; note = 2'-fluorouridine-3'-phosphate
  modified_base 8: mod_base = OTHER; note = 2'-O-methyluridine-3'-phosphate
  modified_base 9: mod_base = OTHER; note = 2'-fluorocytidine-3'-phosphate
  modified_base 10: mod_base = OTHER; note = 2'-fluoroadenosine-3'-phosphate
  modified_base 11: mod_base = OTHER; note = 2'-fluorouridine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-O-methyladenosine-3'-phosphate
  modified_base 15: mod_base = OTHER; note = 2'-O-methyladenosine-3'-phosphate
  modified_base 16: mod_base = OTHER; note = 2'-O-methylcytidine-3'-phosphate
  modified_base 17: mod_base = OTHER; note = 2'-O-methylcytidine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphorothioate
  modified_base 20: mod_base = OTHER; note = 2'-O-methylguanosine-3'-phosphorothioate
  modified_base 21: mod_base = OTHER; note = 2'-O-methyladenosine-3'-phosphate
tgggatttcatgtaaccaaga

SEQ ID NO: 16 - RNA, 23 nt, synthetic construct
  misc_feature 1..23: note = Description of Artificial Sequence: Synthetic oligonucleotide
  source 1..23: mol_type = other RNA
  modified_base 1: mod_base = OTHER; note = 2'-O-methyluridine-3'-phosphorothioate
  modified_base 2: mod_base = OTHER; note = 2'-fluorocytidine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methylguanosine-3'-phosphate
  modified_base 6: mod_base = OTHER; note = 2'-fluoroguanosine-3'-phosphate
  modified_base 7: mod_base = OTHER; note = 2'-O-methyluridine-3'-phosphate
  modified_base 8: mod_base = OTHER; note = 2'-O-methyluridine-3'-phosphate
  modified_base 9: mod_base = OTHER; note = 2'-fluoroadenosine-3'-phosphate
  modified_base 10: mod_base = OTHER; note = 2'-O-methylcytidine-3'-phosphate
  modified_base 11: mod_base = OTHER; note = 2'-O-methyladenosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-fluoroadenosine-3'-phosphate
  modified_base 15: mod_base = OTHER; note = 2'-O-methyladenosine-3'-phosphate
  modified_base 16: mod_base = OTHER; note = 2'-fluoroadenosine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methylcytidine-3'-phosphate
  modified_base 21: mod_base = OTHER; note = 2'-O-methyladenosine-3'-phosphorothioate
  modified_base 22: mod_base = OTHER; note = 2'-O-methyluridine-3'-phosphorothioate
  modified_base 23: mod_base = OTHER; note = 2'-O-methylcytidine-3'-phosphate
tcttggttacatgaaatcccatc

SEQ ID NO: 17 - RNA, 23 nt, synthetic construct
  misc_feature 1..23: note = Description of Artificial Sequence: Synthetic oligonucleotide
  source 1..23: mol_type = other RNA
  modified_base 1: mod_base = OTHER; note = Vinyl-phosphonate 2'-O-methyluridine-3'-phosphorothioate
  modified_base 2: mod_base = OTHER; note = 2'-fluorocytidine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methylguanosine-3'-phosphate
  modified_base 6: mod_base = OTHER; note = 2'-fluoroguanosine-3'-phosphate
  modified_base 7: mod_base = OTHER; note = 2'-O-methyluridine-3'-phosphate
  modified_base 8: mod_base = OTHER; note = 2'-O-methyluridine-3'-phosphate
  modified_base 9: mod_base = OTHER; note = 2'-fluoroadenosine-3'-phosphate
  modified_base 10: mod_base = OTHER; note = 2'-O-methylcytidine-3'-phosphate
  modified_base 11: mod_base = OTHER; note = 2'-O-methyladenosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-fluoroadenosine-3'-phosphate
  modified_base 15: mod_base = OTHER; note = 2'-O-methyladenosine-3'-phosphate
  modified_base 16: mod_base = OTHER; note = 2'-fluoroadenosine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methylcytidine-3'-phosphate
  modified_base 21: mod_base = OTHER; note = 2'-O-methyladenosine-3'-phosphorothioate
  modified_base 22: mod_base = OTHER; note = 2'-O-methyluridine-3'-phosphorothioate
  modified_base 23: mod_base = OTHER; note = 2'-O-methylcytidine-3'-phosphate
tcttggttacatgaaatcccatc

SEQ ID NO: 18 - protein, 29 aa, unidentified
  REGION 1..29: note = Description of Unknown: GALA peptide
AALEALAEALEALAEALEALAEAAAAGGC

SEQ ID NO: 19 - protein, 30 aa, unidentified
  REGION 1..30: note = Description of Unknown: EALA peptide
AALAEALAEALAEALAEALAEALAAAAGGC

SEQ ID NO: 20 - protein, 15 aa, unidentified
  REGION 1..15: note = Description of Unknown: endosomolytic/fusogenic peptide
ALEALAEALEALAEA

SEQ ID NO: 21 - protein, 22 aa, unidentified
  REGION 1..22: note = Description of Unknown: INF-7 peptide
GLFEAIEGFIENGWEGMIWDYG

SEQ ID NO: 22 - protein, 23 aa, unidentified
  REGION 1..23: note = Description of Unknown: InfHA-2 peptide
GLFGAIAGFIENGWEGMIDGWYG

SEQ ID NO: 23 - protein, 48 aa, unidentified
  REGION 1..48: note = Description of Unknown: diINF-7 peptide
GLFEAIEGFIENGWEGMIDGWYGCGLFEAIEGFIENGWEGMIDGWYGC

SEQ ID NO: 24 - protein, 44 aa, unidentified
  REGION 1..44: note = Description of Unknown: diINF-3 peptide
GLFEAIEGFIENGWEGMIDGGCGLFEAIEGFIENGWEGMIDGGC

SEQ ID NO: 25 - protein, 35 aa, unidentified
  REGION 1..35: note = Description of Unknown: GLF peptide
GLFGALAEALAEALAEHLAEALAEALEALAAGGSC

SEQ ID NO: 26 - protein, 34 aa, unidentified
  REGION 1..34: note = Description of Unknown: GALA-INF3 peptide
GLFEAIEGFIENGWEGLAEALAEALEALAAGGSC

SEQ ID NO: 27 - protein, 41 aa, unidentified
  SITE 17: note = MOD_RES - Norleucine
  SITE 38: note = MOD_RES - Norleucine
  REGION 1..41: note = Description of Unknown: INF-5 peptide
GLFEAIEGFIENGWEGXIDGKGLFEAIEGFIENGWEGXIDG

SEQ ID NO: 28 - protein, 19 aa, unidentified
  REGION 1..19: note = Description of Unknown: JTS-1 peptide
LFEALLELLESLWELLLEA

SEQ ID NO: 29 - protein, 20 aa, unidentified
  REGION 1..20: note = Description of Unknown: ppTG1 peptide
GLFKALLKLLKSLWKLLLKA

SEQ ID NO: 30 - protein, 20 aa, unidentified
  REGION 1..20: note = Description of Unknown: ppTG20 peptide
GLFRALLRLLRSLWRLLLRA

SEQ ID NO: 31 - protein, 30 aa, unidentified
  REGION 1..30: note = Description of Unknown: KALA peptide
WEAKLAKALAKALAKHLAKALAKALKACEA

SEQ ID NO: 32 - protein, 22 aa, unidentified
  REGION 1..22: note = Description of Unknown: HA peptide
GLFFEAIAEFIEGGWEGLIEGC

SEQ ID NO: 33 - protein, 26 aa, unidentified
  REGION 1..26: note = Description of Unknown: Melittin peptide
GIGAVLKVLTTGLPALISWIKRKRQQ

SEQ ID NO: 34 - protein, 8 aa, unidentified
  REGION 1..8: note = Description of Unknown: endosomolytic/fusogenic peptide
HHHHHWYG

SEQ ID NO: 35 - protein, 10 aa, unidentified
  REGION 1..10: note = Description of Unknown: endosomolytic/fusogenic peptide
CHKKKKKKHC

SEQ ID NO: 36 - protein, 16 aa, Drosophila sp.
RQIKIWFQNRRMKWKK

SEQ ID NO: 37 - protein, 14 aa, Human immunodeficiency virus 1
GRKKRRQRRRPPQC

SEQ ID NO: 38 - protein, 27 aa, unidentified
  REGION 1..27: note = Description of Unknown: signal sequence based peptide
GALFLGWLGAAGSTMGAWSQPKKKRKV

SEQ ID NO: 39 - protein, 18 aa, Mus sp.
LLIILRRRIRKQAHAHSK

SEQ ID NO: 40 - protein, 26 aa, synthetic construct
  REGION 1..26: note = Description of Artificial Sequence: Synthetic peptide
GWTLNSAGYLLKINLKALAALAKKIL

SEQ ID NO: 41 - protein, 18 aa, synthetic construct
  REGION 1..18: note = Description of Artificial Sequence: Synthetic peptide
KLALKLALKALKAALKLA

SEQ ID NO: 42 - protein, 9 aa, synthetic construct
  REGION 1..9: note = Description of Artificial Sequence: Synthetic peptide
RRRRRRRRR

SEQ ID NO: 43 - protein, 10 aa, synthetic construct
  REGION 1..10: note = Description of Artificial Sequence: Synthetic peptide
KFFKFFKFFK

SEQ ID NO: 44 - protein, 37 aa, unidentified
  REGION 1..37: note = Description of Unknown: LL-37 peptide
LLGDFFRKSKEKIGKEFKRIVQRIKDFLRNLVPRTES

SEQ ID NO: 45 - protein, 31 aa, unidentified
  REGION 1..31: note = Description of Unknown: cecropin P1 peptide
SWLSKTAKKLENSAKKRISEGIAIAIQGGPR

SEQ ID NO: 46 - protein, 30 aa, unidentified
  REGION 1..30: note = Description of Unknown: alpha-defensin peptide
ACYCRIPACIAGERRYGTCIYQGRLWAFCC

SEQ ID NO: 47 - protein, 36 aa, unidentified
  REGION 1..36: note = Description of Unknown: beta-defensin peptide
DHYNCVSSGGQCLYSACPIFTKIQGTCYRGKAKCCK

SEQ ID NO: 48 - protein, 42 aa, unidentified
  REGION 1..42: note = Description of Unknown: PR-39 peptide
  SITE 42: note = Amidated residue
RRRPRPPYLPRPRPPPFFPPRLPPRIPPGFPPRFPPRFPGKR

SEQ ID NO: 49 - protein, 13 aa, unidentified
  REGION 1..13: note = Description of Unknown: indolicidin peptide
  SITE 13: note = Amidated residue
ILPWKWPWWPWRR

SEQ ID NO: 50 - protein, 16 aa, unidentified
  REGION 1..16: note = Description of Unknown: RFGF peptide
AAVALLPAVLLALLAP

SEQ ID NO: 51 - protein, 11 aa, unidentified
  REGION 1..11: note = Description of Unknown: RFGF analogue peptide
AALLPVLLAAP

SEQ ID NO: 52 - protein, 12 aa, unidentified
  REGION 1..12: note = Description of Unknown: bactenecin peptide
RKCRIVVIRVCR

SEQ ID NO: 53 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methylcytidine-3'-phosphate
  modified_base 4: mod_base = OTHER; note = 2'-O-methyluridine-3'-phosphate
  modified_base 6: mod_base = OTHER; note = 2'-O-methyluridine-3'-phosphate
  modified_base 7: mod_base = OTHER; note = 2'-O-methyluridine-3'-phosphate
  modified_base 8: mod_base = OTHER; note = 2'-O-methylcytidine-3'-phosphate
  modified_base 9: mod_base = OTHER; note = 2'-O-methyluridine-3'-phosphate
  modified_base 10: mod_base = OTHER; note = 2'-O-methyluridine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 17: mod_base = OTHER; note = 2'-O-methyluridine-3'-phosphate
  modified_base 21: mod_base = OTHER; note = 2'-deoxythymidine-3'-phosphorothioate N-(cholesterylcarboxamidocaproyl)-4-hydroxyprolinol (Hyp-C6-Chol)
  misc_feature 1..19: note = RNA
  misc_feature 20..21: note = DNA
cagtgttcttgctctataatt

SEQ ID NO: 54 - DNA, 21 bp, synthetic construct
  misc_feature 1..21: note = Description of Artificial Sequence: Synthetic oligonucleotide
  source 1..21: mol_type = other DNA
  modified_base 2: mod_base = OTHER; note = 2'-O-methyluridine-3'-phosphate
  modified_base 4: mod_base = OTHER; note = 2'-O-methyluridine-3'-phosphate
  modified_base 9: mod_base = OTHER; note = 2'-O-methylcytidine-3'-phosphate
  modified_base 15: mod_base = OTHER; note = 2'-O-methylcytidine-3'-phosphate
  misc_feature 1..19: note = RNA
  misc_feature 20..21: note = DNA
ttatagagcaagaacactgtt

SEQ ID NO: 55 - DNA, 23 bp, synthetic construct
  misc_feature 1..23: note = Description of Artificial Sequence: Synthetic oligonucleotide
  source 1..23: mol_type = other DNA
  modified_base 1: mod_base = OTHER; note = 2'-fluoroadenosine-3'-phosphorothioate
  modified_base 2: mod_base = OTHER; note = 2'-O-methyladenosine-3'-phosphorothioate
  modified_base 3: mod_base = OTHER; note = 2'-fluorocytidine-3'-phosphate
  modified_base 4: mod_base = OTHER; note = 2'-O-methyladenosine-3'-phosphate
  modified_base 5: mod_base = OTHER; note = 2'-fluoroguanosine-3'-phosphate
  modified_base 6: mod_base = OTHER; note = 2'-O-methyluridine-3'-phosphate
  modified_base 7: mod_base = OTHER; note = 2'-fluoroguanosine-3'-phosphate
  modified_base 8: mod_base = OTHER; note = 2'-O-methyluridine-3'-phosphate
  modified_base 9: mod_base = OTHER; note = 2'-fluorouridine-3'-phosphate
  modified_base 10: mod_base = OTHER; note = 2'-fluorocytidine-3'-phosphate
  modified_base 11: mod_base = OTHER; note = 2'-fluorouridine-3'-phosphate
  modified_base 12: mod_base = OTHER; note = 2'-O-methyluridine-3'-phosphate
  modified_base 13: mod_base = OTHER; note = 2'-fluoroguanosine-3'-phosphate
  modified_base 14: mod_base = OTHER; note = 2'-O-methylcytidine-3'-phosphate
  modified_base 15: mod_base = OTHER; note = 2'-fluorouridine-3'-phosphate
  modified_base 16: mod_base = OTHER; note = 2'-O-methylcytidine-3'-phosphate
  modified_base 17: mod_base = OTHER; note = 2'-fluorouridine-3'-phosphate
  modified_base 18: mod_base = OTHER; note = 2'-O-methyladenosine-3'-phosphate
  modified_base 19: mod_base = OTHER; note = 2'-fluorouridine-3'-phosphate
  modified_base 20: mod_base = OTHER; note = 2'-O-methyladenosine-3'-phosphorothioate
  modified_base 21: mod_base = OTHER; note = 2'-fluoroadenosine-3'-phosphate
  modified_base 23: mod_base = OTHER; note = 2'-deoxythymidine-3'-phosphate N-(cholesterylcarboxamidocaproyl)-4-hydroxyprolinol (Hyp-C6-Chol)
  misc_feature 1..21: note = RNA
  misc_feature 22..23: note = DNA
aacagtgttcttgctctataatt

SEQ ID NO: 56 - RNA, 23 nt, synthetic construct
  misc_feature 1..23: note = Description of Artificial Sequence: Synthetic oligonucleotide
  source 1..23: mol_type = other RNA
  modified_base 1: mod_base = OTHER; note = 2'-O-methyluridine-3'-phosphorothioate
  modified_base 2: mod_base = OTHER; note = 2'-fluorouridine-3'-phosphorothioate
  modified_base 3: mod_base = OTHER; note = 2'-O-methyladenosine-3'-phosphate
  modified_base 4: mod_base = OTHER; note = 2'-fluorouridine-3'-phosphate
  modified_base 5: mod_base = OTHER; note = 2'-O-methyladenosine-3'-phosphate
  modified_base 6: mod_base = OTHER; note = 2'-fluoroguanosine-3'-phosphate
  modified_base 7: mod_base = OTHER; note = 2'-O-methyladenosine-3'-phosphate
  modified_base 8: mod_base = OTHER; note = 2'-fluoroguanosine-3'-phosphate
  modified_base 9: mod_base = OTHER; note = 2'-O-methylcytidine-3'-phosphate
  modified_base 10: mod_base = OTHER; note = 2'-fluoroadenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-fluoroadenosine-3'-phosphate
  modified_base 15: mod_base = OTHER; note = 2'-O-methylcytidine-3'-phosphate
  modified_base 16: mod_base = OTHER; note = 2'-fluoroadenosine-3'-phosphate
  modified_base 17: mod_base = OTHER; note = 2'-O-methylcytidine-3'-phosphate
  modified_base 18: mod_base = OTHER; note = 2'-fluorouridine-3'-phosphate
  modified_base 19: mod_base = OTHER; note = 2'-O-methylguanosine-3'-phosphate
  modified_base 20: mod_base = OTHER; note = 2'-fluorouridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
ttatagagcaagaacactgtttt

SEQ ID NO: 57 - RNA, 23 nt, synthetic construct
  misc_feature 1..23: note = Description of Artificial Sequence: Synthetic oligonucleotide
  source 1..23: mol_type = other RNA
  modified_base 1: mod_base = OTHER; note = Vinyl-phosphonate 2'-O-methyluridine-3'-phosphorothioate
  modified_base 2: mod_base = OTHER; note = 2'-fluorouridine-3'-phosphorothioate
  modified_base 3: mod_base = OTHER; note = 2'-O-methyladenosine-3'-phosphate
  modified_base 4: mod_base = OTHER; note = 2'-fluorouridine-3'-phosphate
  modified_base 5: mod_base = OTHER; note = 2'-O-methyladenosine-3'-phosphate
  modified_base 6: mod_base = OTHER; note = 2'-fluoroguanosine-3'-phosphate
  modified_base 7: mod_base = OTHER; note = 2'-O-methyladenosine-3'-phosphate
  modified_base 8: mod_base = OTHER; note = 2'-fluoroguanosine-3'-phosphate
  modified_base 9: mod_base = OTHER; note = 2'-O-methylcytidine-3'-phosphate
  modified_base 10: mod_base = OTHER; note = 2'-fluoroadenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-fluoroadenosine-3'-phosphate
  modified_base 15: mod_base = OTHER; note = 2'-O-methylcytidine-3'-phosphate
  modified_base 16: mod_base = OTHER; note = 2'-fluoroadenosine-3'-phosphate
  modified_base 17: mod_base = OTHER; note = 2'-O-methylcytidine-3'-phosphate
  modified_base 18: mod_base = OTHER; note = 2'-fluorouridine-3'-phosphate
  modified_base 19: mod_base = OTHER; note = 2'-O-methylguanosine-3'-phosphate
  modified_base 20: mod_base = OTHER; note = 2'-fluorouridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
ttatagagcaagaacactgtttt

SEQ ID NO: 58 - RNA, 21 nt, synthetic construct
  misc_feature 1..21: note = Description of Artificial Sequence: Synthetic oligonucleotide
  source 1..21: mol_type = other RNA
  modified_base 1: mod_base = OTHER; note = 2'-fluoroadenosine-3'-phosphorothioate
  modified_base 2: mod_base = OTHER; note = 2'-O-methyladenosine-3'-phosphorothioate
  modified_base 3: mod_base = OTHER; note = 2'-fluorocytidine-3'-phosphate
  modified_base 4: mod_base = OTHER; note = 2'-O-methyladenosine-3'-phosphate
  modified_base 5: mod_base = OTHER; note = 2'-fluoroguanosine-3'-phosphate
  modified_base 6: mod_base = OTHER; note = 2'-O-methyluridine-3'-phosphate
  modified_base 7: mod_base = OTHER; note = 2'-fluoroguanosine-3'-phosphate
  modified_base 8: mod_base = OTHER; note = 2'-O-methyluridine-3'-phosphate
  modified_base 9: mod_base = OTHER; note = 2'-fluorouridine-3'-phosphate
  modified_base 10: mod_base = OTHER; note = 2'-fluorocytidine-3'-phosphate
  modified_base 11: mod_base = OTHER; note = 2'-fluorouridine-3'-phosphate
  modified_base 12: mod_base = OTHER; note = 2'-O-methyluridine-3'-phosphate
  modified_base 13: mod_base = OTHER; note = 2'-fluoroguanosine-3'-phosphate
  modified_base 14: mod_base = OTHER; note = 2'-O-methylcytidine-3'-phosphate
  modified_base 15: mod_base = OTHER; note = 2'-fluorouridine-3'-phosphate
  modified_base 16: mod_base = OTHER; note = 2'-O-methylcytidine-3'-phosphate
  modified_base 17: mod_base = OTHER; note = 2'-fluorouridine-3'-phosphate
  modified_base 18: mod_base = OTHER; note = 2'-O-methyladenosine-3'-phosphate
  modified_base 19: mod_base = OTHER; note = 2'-fluorouridine-3'-phosphorothioate
  modified_base 20: mod_base = OTHER; note = 2'-O-methyladenosine-3'-phosphorothioate
  modified_base 21: mod_base = OTHER; note = 2'-fluoroadenosine-3'-phosphate
aacagtgttcttgctctataa

SEQ ID NO: 59 - DNA, 23 bp, synthetic construct
  misc_feature 1..23: note = Description of Artificial Sequence: Synthetic oligonucleotide
  source 1..23: mol_type = other DNA
  modified_base 1: mod_base = OTHER; note = 2'-O-methyluridine-3'-phosphorothioate
  modified_base 2: mod_base = OTHER; note = 2'-O-methylguanosine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-O-methyluridine-3'-phosphate
  modified_base 7: mod_base = OTHER; note = 2'-fluorouridine-3'-phosphate
  modified_base 8: mod_base = OTHER; note = 2'-O-methyluridine-3'-phosphate
  modified_base 9: mod_base = OTHER; note = 2'-fluorocytidine-3'-phosphate
  modified_base 10: mod_base = OTHER; note = 2'-fluoroadenosine-3'-phosphate
  modified_base 11: mod_base = OTHER; note = 2'-fluorouridine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-O-methyladenosine-3'-phosphate
  modified_base 15: mod_base = OTHER; note = 2'-O-methyladenosine-3'-phosphate
  modified_base 16: mod_base = OTHER; note = 2'-O-methylcytidine-3'-phosphate
  modified_base 17: mod_base = OTHER; note = 2'-O-methylcytidine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methylguanosine-3'-phosphorothioate
  modified_base 21: mod_base = OTHER; note = 2'-O-methyladenosine-3'-phosphate
  modified_base 23: mod_base = OTHER; note = 2'-deoxythymidine-3'-phosphate N-(cholesterylcarboxamidocaproyl)-4-hydroxyprolinol (Hyp-C6-Chol)
  misc_feature 1..21: note = RNA
  misc_feature 22..23: note = DNA
tgggatttcatgtaaccaagatt

SEQ ID NO: 60 - RNA, 21 nt, synthetic construct
  misc_feature 1..21: note = Description of Artificial Sequence: Synthetic oligonucleotide
  source 1..21: mol_type = other RNA
  modified_base 1: mod_base = OTHER; note = 2'-O-methyluridine-3'-phosphorothioate
  modified_base 2: mod_base = OTHER; note = 2'-O-methylguanosine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-O-methyluridine-3'-phosphate
  modified_base 7: mod_base = OTHER; note = 2'-fluorouridine-3'-phosphate
  modified_base 8: mod_base = OTHER; note = 2'-O-methyluridine-3'-phosphate
  modified_base 9: mod_base = OTHER; note = 2'-fluorocytidine-3'-phosphate
  modified_base 10: mod_base = OTHER; note = 2'-fluoroadenosine-3'-phosphate
  modified_base 11: mod_base = OTHER; note = 2'-fluorouridine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-O-methyladenosine-3'-phosphate
  modified_base 15: mod_base = OTHER; note = 2'-O-methyladenosine-3'-phosphate
  modified_base 16: mod_base = OTHER; note = 2'-O-methylcytidine-3'-phosphate
  modified_base 17: mod_base = OTHER; note = 2'-O-methylcytidine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methylguanosine-3'-phosphate
  modified_base 21: mod_base = OTHER; note = 2'-O-methyladenosine-3'-phosphate N-(cholesterylcarboxamidocaproyl)-4-hydroxyprolinol (Hyp-C6-Chol)
tgggatttcatgtaaccaaga

SEQ ID NO: 61 - RNA, 21 nt, synthetic construct
  misc_feature 1..21: note = Description of Artificial Sequence: Synthetic oligonucleotide
  source 1..21: mol_type = other RNA
  modified_base 1: mod_base = OTHER; note = 2'-O-methyluridine-3'-phosphorothioate
  modified_base 2: mod_base = OTHER; note = 2'-O-methylguanosine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-O-methyluridine-3'-phosphate
  modified_base 7: mod_base = OTHER; note = 2'-fluorouridine-3'-phosphate
  modified_base 8: mod_base = OTHER; note = 2'-O-methyluridine-3'-phosphate
  modified_base 9: mod_base = OTHER; note = 2'-fluorocytidine-3'-phosphate
  modified_base 10: mod_base = OTHER; note = 2'-fluoroadenosine-3'-phosphate
  modified_base 11: mod_base = OTHER; note = 2'-fluorouridine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-O-methyladenosine-3'-phosphate
  modified_base 15: mod_base = OTHER; note = 2'-O-methyladenosine-3'-phosphate
  modified_base 16: mod_base = OTHER; note = 2'-O-methylcytidine-3'-phosphate
  modified_base 17: mod_base = OTHER; note = 2'-O-methylcytidine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methylguanosine-3'-phosphate
  modified_base 21: mod_base = OTHER; note = 2'-O-methyladenosine-3'-phosphate N-(stearylcarboxamidocaproyl)-4-hydroxyprolinol (Hyp-C6-C18)
tgggatttcatgtaaccaaga

SEQ ID NO: 62 - RNA, 23 nt, synthetic construct
  misc_feature 1..23: note = Description of Artificial Sequence: Synthetic oligonucleotide
  source 1..23: mol_type = other RNA
  modified_base 1: mod_base = OTHER; note = Vinyl-phosphonate 2'-O-methyluridine-3'-phosphate
  modified_base 2: mod_base = OTHER; note = 2'-fluorocytidine-3'-phosphate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methylguanosine-3'-phosphate
  modified_base 6: mod_base = OTHER; note = 2'-fluoroguanosine-3'-phosphate
  modified_base 7: mod_base = OTHER; note = 2'-O-methyluridine-3'-phosphate
  modified_base 8: mod_base = OTHER; note = 2'-O-methyluridine-3'-phosphate
  modified_base 9: mod_base = OTHER; note = 2'-fluoroadenosine-3'-phosphate
  modified_base 10: mod_base = OTHER; note = 2'-O-methylcytidine-3'-phosphate
  modified_base 11: mod_base = OTHER; note = 2'-O-methyladenosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-fluoroadenosine-3'-phosphate
  modified_base 15: mod_base = OTHER; note = 2'-O-methyladenosine-3'-phosphate
  modified_base 16: mod_base = OTHER; note = 2'-fluoroadenosine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methylcytidine-3'-phosphate
  modified_base 21: mod_base = OTHER; note = 2'-O-methyladenosine-3'-phosphorothioate
  modified_base 22: mod_base = OTHER; note = 2'-O-methyluridine-3'-phosphorothioate
  modified_base 23: mod_base = OTHER; note = 2'-O-methylcytidine-3'-phosphate
tcttggttacatgaaatcccatc

SEQ ID NO: 63 - RNA, 19 nt, synthetic construct
  misc_feature 1..19: note = Description of Artificial Sequence: Synthetic oligonucleotide
  source 1..19: mol_type = other RNA
cagtgttcttgctctataa

SEQ ID NO: 64 - RNA, 19 nt, synthetic construct
  misc_feature 1..19: note = Description of Artificial Sequence: Synthetic oligonucleotide
  source 1..19: mol_type = other RNA
ttatagagcaagaacactg

SEQ ID NO: 65 - RNA, 21 nt, synthetic construct
  misc_feature 1..21: note = Description of Artificial Sequence: Synthetic oligonucleotide
  source 1..21: mol_type = other RNA
aacagtgttcttgctctataa

SEQ ID NO: 66 - RNA, 23 nt, synthetic construct
  misc_feature 1..23: note = Description of Artificial Sequence: Synthetic oligonucleotide
  source 1..23: mol_type = other RNA
ttatagagcaagaacactgtttt

SEQ ID NO: 67 - RNA, 21 nt, synthetic construct
  misc_feature 1..21: note = Description of Artificial Sequence: Synthetic oligonucleotide
  source 1..21: mol_type = other RNA
atgggatttcatgtaaccaaa

SEQ ID NO: 68 - RNA, 21 nt, synthetic construct
  misc_feature 1..21: note = Description of Artificial Sequence: Synthetic oligonucleotide
  source 1..21: mol_type = other RNA
tttcatgtaaccaagagtatt

SEQ ID NO: 69 - RNA, 21 nt, synthetic construct
  misc_feature 1..21: note = Description of Artificial Sequence: Synthetic oligonucleotide
  source 1..21: mol_type = other RNA
tgtaaccaagagtattccatt

SEQ ID NO: 70 - RNA, 21 nt, synthetic construct
  misc_feature 1..21: note = Description of Artificial Sequence: Synthetic oligonucleotide
  source 1..21: mol_type = other RNA
aaccaagagtattccattttt

SEQ ID NO: 71 - RNA, 21 nt, synthetic construct
  misc_feature 1..21: note = Description of Artificial Sequence: Synthetic oligonucleotide
  source 1..21: mol_type = other RNA
tttttactaaagcagtgtttt

SEQ ID NO: 72 - RNA, 21 nt, synthetic construct
  misc_feature 1..21: note = Description of Artificial Sequence: Synthetic oligonucleotide
  source 1..21: mol_type = other RNA
ttactaaagcagtgttttcaa

SEQ ID NO: 73 - RNA, 21 nt, synthetic construct
  misc_feature 1..21: note = Description of Artificial Sequence: Synthetic oligonucleotide
  source 1..21: mol_type = other RNA
ctaaagcagtgttttcaccta

SEQ ID NO: 74 - RNA, 21 nt, synthetic construct
  misc_feature 1..21: note = Description of Artificial Sequence: Synthetic oligonucleotide
  source 1..21: mol_type = other RNA
ggcagagacaataaaacatta

SEQ ID NO: 75 - RNA, 21 nt, synthetic construct
  misc_feature 1..21: note = Description of Artificial Sequence: Synthetic oligonucleotide
  source 1..21: mol_type = other RNA
cagagacaataaaacattcct

SEQ ID NO: 76 - RNA, 21 nt, synthetic construct
  misc_feature 1..21: note = Description of Artificial Sequence: Synthetic oligonucleotide
  source 1..21: mol_type = other RNA
caataaaacattcctgtgaaa

SEQ ID NO: 77 - RNA, 23 nt, synthetic construct
  misc_feature 1..23: note = Description of Artificial Sequence: Synthetic oligonucleotide
  source 1..23: mol_type = other RNA
tttggttacatgaaatcccatcc

SEQ ID NO: 78 - RNA, 23 nt, synthetic construct
  misc_feature 1..23: note = Description of Artificial Sequence: Synthetic oligonucleotide
  source 1..23: mol_type = other RNA
aatactcttggttacatgaaatc

SEQ ID NO: 79 - RNA, 23 nt, synthetic construct
  misc_feature 1..23: note = Description of Artificial Sequence: Synthetic oligonucleotide
  source 1..23: mol_type = other RNA
aatggaatactcttggttacatg

SEQ ID NO: 80 - RNA, 23 nt, synthetic construct
  misc_feature 1..23: note = Description of Artificial Sequence: Synthetic oligonucleotide
  source 1..23: mol_type = other RNA
aaaaatggaatactcttggttac

SEQ ID NO: 81 - RNA, 23 nt, synthetic construct
  misc_feature 1..23: note = Description of Artificial Sequence: Synthetic oligonucleotide
  source 1..23: mol_type = other RNA
aaaacactgctttagtaaaaatg

SEQ ID NO: 82 - RNA, 23 nt, synthetic construct
  misc_feature 1..23: note = Description of Artificial Sequence: Synthetic oligonucleotide
  source 1..23: mol_type = other RNA
ttgaaaacactgctttagtaaaa

SEQ ID NO: 83 - RNA, 23 nt, synthetic construct
  misc_feature 1..23: note = Description of Artificial Sequence: Synthetic oligonucleotide
  source 1..23: mol_type = other RNA
taggtgaaaacactgctttagta

SEQ ID NO: 84 - RNA, 23 nt, synthetic construct
  misc_feature 1..23: note = Description of Artificial Sequence: Synthetic oligonucleotide
  source 1..23: mol_type = other RNA
taatgttttattgtctctgcctg

SEQ ID NO: 85 - RNA, 23 nt, synthetic construct
  misc_feature 1..23: note = Description of Artificial Sequence: Synthetic oligonucleotide
  source 1..23: mol_type = other RNA
aggaatgttttattgtctctgcc

SEQ ID NO: 86 - RNA, 23 nt, synthetic construct
  misc_feature 1..23: note = Description of Artificial Sequence: Synthetic oligonucleotide
  source 1..23: mol_type = other RNA
tttcacaggaatgttttattgtc

SEQ ID NO: 87 - RNA, 21 nt, synthetic construct
  misc_feature 1..21: note = Description of Artificial Sequence: Synthetic oligonucleotide
  source 1..21: mol_type = other RNA
  modified_base 1: mod_base = OTHER; note = 2'-O-hexadecyl-adenosine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-methyluridine-3'-phosphate
  modified_base 7: mod_base = OTHER; note = 2'-fluoroguanosine-3'-phosphate
  modified_base 8: mod_base = OTHER; note = 2'-O-methyluridine-3'-phosphate
  modified_base 9: mod_base = OTHER; note = 2'-fluorouridine-3'-phosphate
  modified_base 10: mod_base = OTHER; note = 2'-fluorocytidine-3'-phosphate
  modified_base 11: mod_base = OTHER; note = 2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
aacagtgttcttgctctataa

SEQ ID NO: 88 - RNA, 21 nt, synthetic construct
  misc_feature 1..21: note = Description of Artificial Sequence: Synthetic oligonucleotide
  source 1..21: mol_type = other RNA
  modified_base 1: mod_base = OTHER; note = 2'-O-methyladenosine-3'-phosphorothioate
  modified_base 2: mod_base = OTHER; note = 2'-O-hexadecyl-adenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-methyluridine-3'-phosphate
  modified_base 7: mod_base = OTHER; note = 2'-fluoroguanosine-3'-phosphate
  modified_base 8: mod_base = OTHER; note = 2'-O-methyluridine-3'-phosphate
  modified_base 9: mod_base = OTHER; note = 2'-fluorouridine-3'-phosphate
  modified_base 10: mod_base = OTHER; note = 2'-fluorocytidine-3'-phosphate
  modified_base 11: mod_base = OTHER; note = 2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
aacagtgttcttgctctataa

SEQ ID NO: 89 - RNA, 21 nt, synthetic construct
  misc_feature 1..21: note = Description of Artificial Sequence: Synthetic oligonucleotide
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-hexadecyl-guanosine-3'-phosphate
  modified_base 6: mod_base = OTHER; note = 2'-O-methyluridine-3'-phosphate
  modified_base 7: mod_base = OTHER; note = 2'-fluoroguanosine-3'-phosphate
  modified_base 8: mod_base = OTHER; note = 2'-O-methyluridine-3'-phosphate
  modified_base 9: mod_base = OTHER; note = 2'-fluorouridine-3'-phosphate
  modified_base 10: mod_base = OTHER; note = 2'-fluorocytidine-3'-phosphate
  modified_base 11: mod_base = OTHER; note = 2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
aacagtgttcttgctctataa

SEQ ID NO: 90 - RNA, 21 nt, synthetic construct
  misc_feature 1..21: note = Description of Artificial Sequence: Synthetic oligonucleotide
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-hexadecyl-uridine-3'-phosphate
  modified_base 7: mod_base = OTHER; note = 2'-fluoroguanosine-3'-phosphate
  modified_base 8: mod_base = OTHER; note = 2'-O-methyluridine-3'-phosphate
  modified_base 9: mod_base = OTHER; note = 2'-fluorouridine-3'-phosphate
  modified_base 10: mod_base = OTHER; note = 2'-fluorocytidine-3'-phosphate
  modified_base 11: mod_base = OTHER; note = 2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
aacagtgttcttgctctataa

SEQ ID NO: 91 - RNA, 21 nt, synthetic construct
  misc_feature 1..21: note = Description of Artificial Sequence: Synthetic oligonucleotide
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-methyluridine-3'-phosphate
  modified_base 7: mod_base = OTHER; note = 2'-O-hexadecyl-guanosine-3'-phosphate
  modified_base 8: mod_base = OTHER; note = 2'-O-methyluridine-3'-phosphate
  modified_base 9: mod_base = OTHER; note = 2'-fluorouridine-3'-phosphate
  modified_base 10: mod_base = OTHER; note = 2'-fluorocytidine-3'-phosphate
  modified_base 11: mod_base = OTHER; note = 2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
aacagtgttcttgctctataa

SEQ ID NO: 92 - RNA, 21 nt, synthetic construct
  misc_feature 1..21: note = Description of Artificial Sequence: Synthetic oligonucleotide
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-methyluridine-3'-phosphate
  modified_base 7: mod_base = OTHER; note = 2'-fluoroguanosine-3'-phosphate
  modified_base 8: mod_base = OTHER; note = 2'-O-methyluridine-3'-phosphate
  modified_base 9: mod_base = OTHER; note = 2'-fluorouridine-3'-phosphate
  modified_base 10: mod_base = OTHER; note = 2'-O-hexadecyl-cytidine-3'-phosphate
  modified_base 11: mod_base = OTHER; note = 2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
aacagtgttcttgctctataa

SEQ ID NO: 93 - RNA, 21 nt, synthetic construct
  misc_feature 1..21: note = Description of Artificial Sequence: Synthetic oligonucleotide
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-methyluridine-3'-phosphate
  modified_base 7: mod_base = OTHER; note = 2'-fluoroguanosine-3'-phosphate
  modified_base 8: mod_base = OTHER; note = 2'-O-methyluridine-3'-phosphate
  modified_base 9: mod_base = OTHER; note = 2'-fluorouridine-3'-phosphate
  modified_base 10: mod_base = OTHER; note = 2'-fluorocytidine-3'-phosphate
  modified_base 11: mod_base = OTHER; note = 2'-O-hexadecyl-uridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
aacagtgttcttgctctataa

SEQ ID NO: 94 - RNA, 21 nt, synthetic construct
  misc_feature 1..21: note = Description of Artificial Sequence: Synthetic oligonucleotide
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-methyluridine-3'-phosphate
  modified_base 7: mod_base = OTHER; note = 2'-fluoroguanosine-3'-phosphate
  modified_base 8: mod_base = OTHER; note = 2'-O-methyluridine-3'-phosphate
  modified_base 9: mod_base = OTHER; note = 2'-fluorouridine-3'-phosphate
  modified_base 10: mod_base = OTHER; note = 2'-fluorocytidine-3'-phosphate
  modified_base 11: mod_base = OTHER; note = 2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-hexadecyl-uridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
aacagtgttcttgctctataa

SEQ ID NO: 95 - RNA, 21 nt, synthetic construct
  misc_feature 1..21: note = Description of Artificial Sequence: Synthetic oligonucleotide
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-methyluridine-3'-phosphate
  modified_base 7: mod_base = OTHER; note = 2'-fluoroguanosine-3'-phosphate
  modified_base 8: mod_base = OTHER; note = 2'-O-methyluridine-3'-phosphate
  modified_base 9: mod_base = OTHER; note = 2'-fluorouridine-3'-phosphate
  modified_base 10: mod_base = OTHER; note = 2'-fluorocytidine-3'-phosphate
  modified_base 11: mod_base = OTHER; note = 2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphorothioate
  modified_base 20: mod_base = OTHER; note = 2'-O-hexadecyl-adenosine-3'-phosphorothioate
  modified_base 21: mod_base = OTHER; note = 2'-O-methyladenosine-3'-phosphate
aacagtgttcttgctctataa

SEQ ID NO: 96 - RNA, 21 nt, synthetic construct
  misc_feature 1..21: note = Description of Artificial Sequence: Synthetic oligonucleotide
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-methyluridine-3'-phosphate
  modified_base 7: mod_base = OTHER; note = 2'-fluoroguanosine-3'-phosphate
  modified_base 8: mod_base = OTHER; note = 2'-O-methyluridine-3'-phosphate
  modified_base 9: mod_base = OTHER; note = 2'-fluorouridine-3'-phosphate
  modified_base 10: mod_base = OTHER; note = 2'-fluorocytidine-3'-phosphate
  modified_base 11: mod_base = OTHER; note = 2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphorothioate
  modified_base 20: mod_base = OTHER; note = 2'-O-methyladenosine-3'-phosphorothioate
  modified_base 21: mod_base = OTHER; note = 2'-O-hexadecyl-adenosine-3'-phosphate
aacagtgttcttgctctataa

SEQ ID NO: 97 - RNA, 21 nt, synthetic construct
  misc_feature 1..21: note = Description of Artificial Sequence: Synthetic oligonucleotide
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-methyluridine-3'-phosphate
  modified_base 7: mod_base = OTHER; note = 2'-fluoroguanosine-3'-phosphate
  modified_base 8: mod_base = OTHER; note = 2'-O-methyluridine-3'-phosphate
  modified_base 9: mod_base = OTHER; note = 2'-fluorouridine-3'-phosphate
  modified_base 10: mod_base = OTHER; note = 2'-fluorocytidine-3'-phosphate
  modified_base 11: mod_base = OTHER; note = 2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
aacagtgttcttgctctataa

SEQ ID NO: 98 - RNA, 23 nt, synthetic construct
  misc_feature 1..23: note = Description of Artificial Sequence: Synthetic oligonucleotide
  source 1..23: mol_type = other RNA
  modified_base 1: mod_base = OTHER; note = Vinyl-phosphonate 2'-O-methyluridine-3'-phosphate
  modified_base 2: mod_base = OTHER; note = 2'-fluorouridine-3'-phosphate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-fluoroguanosine-3'-phosphate
  modified_base 7: mod_base = OTHER; note = 2'-O-methyladenosine-3'-phosphate
  modified_base 8: mod_base = OTHER; note = 2'-O-methylguanosine-3'-phosphate
  modified_base 9: mod_base = OTHER; note = 2'-O-methylcytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-fluoroadenosine-3'-phosphate
  modified_base 15: mod_base = OTHER; note = 2'-O-methylcytidine-3'-phosphate
  modified_base 16: mod_base = OTHER; note = 2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
ttatagagcaagaacactgtttt

SEQ ID NO: 99 - RNA, 23 nt, synthetic construct
  misc_feature 1..23: note = Description of Artificial Sequence: Synthetic oligonucleotide
  source 1..23: mol_type = other RNA
  modified_base 1: mod_base = OTHER; note = Vinyl-phosphonate 2'-O-methyluridine-3'-phosphate
  modified_base 2: mod_base = OTHER; note = 2'-fluorouridine-3'-phosphate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-hexadecyl-adenosine-3'-phosphate
  modified_base 6: mod_base = OTHER; note = 2'-fluoroguanosine-3'-phosphate
  modified_base 7: mod_base = OTHER; note = 2'-O-methyladenosine-3'-phosphate
  modified_base 8: mod_base = OTHER; note = 2'-O-methylguanosine-3'-phosphate
  modified_base 9: mod_base = OTHER; note = 2'-O-methylcytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-fluoroadenosine-3'-phosphate
  modified_base 15: mod_base = OTHER; note = 2'-O-methylcytidine-3'-phosphate
  modified_base 16: mod_base = OTHER; note = 2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
ttatagagcaagaacactgtttt

SEQ ID NO: 100 - RNA, 23 nt, synthetic construct
  misc_feature 1..23: note = Description of Artificial Sequence: Synthetic oligonucleotide
  source 1..23: mol_type = other RNA
  modified_base 1: mod_base = OTHER; note = Vinyl-phosphonate 2'-O-methyluridine-3'-phosphate
  modified_base 2: mod_base = OTHER; note = 2'-fluorouridine-3'-phosphate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-fluoroguanosine-3'-phosphate
  modified_base 7: mod_base = OTHER; note = 2'-O-methyladenosine-3'-phosphate
  modified_base 8: mod_base = OTHER; note = 2'-O-methylguanosine-3'-phosphate
  modified_base 9: mod_base = OTHER; note = 2'-O-methylcytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-fluoroadenosine-3'-phosphate
  modified_base 15: mod_base = OTHER; note = 2'-O-hexadecyl-cytidine-3'-phosphate
  modified_base 16: mod_base = OTHER; note = 2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
ttatagagcaagaacactgtttt

SEQ ID NO: 101 - RNA, 23 nt, synthetic construct
  misc_feature 1..23: note = Description of Artificial Sequence: Synthetic oligonucleotide
  source 1..23: mol_type = other RNA
  modified_base 1: mod_base = OTHER; note = Vinyl-phosphonate 2'-O-methyluridine-3'-phosphate
  modified_base 2: mod_base = OTHER; note = 2'-fluorouridine-3'-phosphate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-fluoroguanosine-3'-phosphate
  modified_base 7: mod_base = OTHER; note = 2'-O-methyladenosine-3'-phosphate
  modified_base 8: mod_base = OTHER; note = 2'-O-methylguanosine-3'-phosphate
  modified_base 9: mod_base = OTHER; note = 2'-O-methylcytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-fluoroadenosine-3'-phosphate
  modified_base 15: mod_base = OTHER; note = 2'-O-methylcytidine-3'-phosphate
  modified_base 16: mod_base = OTHER; note = 2'-O-hexadecyl-adenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
ttatagagcaagaacactgtttt

SEQ ID NO: 102 - DNA, 23 bp, synthetic construct
  misc_feature 1..23: note = Description of Artificial Sequence: Synthetic oligonucleotide
  source 1..23: mol_type = other DNA
  modified_base 1: mod_base = OTHER; note = Vinyl-phosphonate 2'-O-methyluridine-3'-phosphorothioate
  modified_base 2: mod_base = OTHER; note = 2'-fluorocytidine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methylguanosine-3'-phosphate
  modified_base 6: mod_base = OTHER; note = 2'-fluoroguanosine-3'-phosphate
  modified_base 7: mod_base = OTHER; note = Thymidine-glycol nucleic acid (GNA) S-Isomer
  modified_base 8: mod_base = OTHER; note = 2'-O-methyluridine-3'-phosphate
  modified_base 9: mod_base = OTHER; note = 2'-fluoroadenosine-3'-phosphate
  modified_base 10: mod_base = OTHER; note = 2'-O-methylcytidine-3'-phosphate
  modified_base 11: mod_base = OTHER; note = 2'-O-methyladenosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-fluoroadenosine-3'-phosphate
  modified_base 15: mod_base = OTHER; note = 2'-O-methyladenosine-3'-phosphate
  modified_base 16: mod_base = OTHER; note = 2'-fluoroadenosine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methylcytidine-3'-phosphate
  modified_base 21: mod_base = OTHER; note = 2'-O-methyladenosine-3'-phosphorothioate
  modified_base 22: mod_base = OTHER; note = 2'-O-methyluridine-3'-phosphorothioate
  modified_base 23: mod_base = OTHER; note = 2'-O-methylcytidine-3'-phosphate
  misc_feature 1..6: note = RNA
  misc_feature 7: note = DNA
  misc_feature 8..23: note = RNA
tcttggttacatgaaatcccatc

SEQ ID NO: 103 - RNA, 21 nt, synthetic construct
  misc_feature 1..21: note = Description of Artificial Sequence: Synthetic oligonucleotide
  source 1..21: mol_type = other RNA
  modified_base 1: mod_base = OTHER; note = 2'-O-methyluridine-3'-phosphorothioate
  modified_base 2: mod_base = OTHER; note = 2'-O-methylguanosine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-O-methyluridine-3'-phosphate
  modified_base 7: mod_base = OTHER; note = 2'-fluorouridine-3'-phosphate
  modified_base 8: mod_base = OTHER; note = 2'-O-methyluridine-3'-phosphate
  modified_base 9: mod_base = OTHER; note = 2'-fluorocytidine-3'-phosphate
  modified_base 10: mod_base = OTHER; note = 2'-fluoroadenosine-3'-phosphate
  modified_base 11: mod_base = OTHER; note = 2'-fluorouridine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-O-methyladenosine-3'-phosphate
  modified_base 15: mod_base = OTHER; note = 2'-O-methyladenosine-3'-phosphate
  modified_base 16: mod_base = OTHER; note = 2'-O-methylcytidine-3'-phosphate
  modified_base 17: mod_base = OTHER; note = 2'-O-methylcytidine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphorothioate
  modified_base 20: mod_base = OTHER; note = 2'-O-methylguanosine-3'-phosphorothioate
  modified_base 21: mod_base = OTHER; note = 2'-O-methyladenosine-3'-phosphate
tgggatttcatgtaaccaaga

SEQ ID NO: 104 - protein, 5 aa, synthetic construct
  SITE 4: note = D-amino acid
RGDFC

(19) United States
(12) Patent Application Publication
(10) Pub. No.: US 2026/0167961 A1
(43) Pub. Date: Jun. 18, 2026

(54) TRANSTHYRETIN (TTR) iRNA COMPOSITIONS AND METHODS OF USE THEREOF FOR TREATING OR PREVENTING TTR-ASSOCIATED OCULAR DISEASES

(71) Applicant: Alnylam Pharmaceuticals, Inc., Cambridge, MA (US)

(72) Inventors: Jayaprakash K. Nair, Cambridge, MA (US); Martin A. Maier, Belmont, MA (US); Vasant R. Jadhav, Sharon, MA (US); Mark Keating, Weston, MA (US); Kevin Fitzgerald, Brookline, MA (US); Stuart Milstein, Arlington, MA (US); Kirk Brown, Swampscott, MA (US); Muthiah Manoharan, Weston, MA (US)

(21) Appl. No.: 19/247,022

(22) Filed: Jun. 24, 2025

Related U.S. Application Data
(63) Continuation of application No. 18/225,919, filed on Jul. 25, 2023, now abandoned.
Application No. 18/225,919 is a continuation of application No. 17/213,294, filed on Mar. 26, 2021, now abandoned.
Application No. 17/213,294 is a continuation of application No. PCT/US2019/053050, filed on Sep. 26, 2019.
(60) Provisional application No. 62/738,256, filed on Sep. 28, 2018.
(60) Provisional application No. 62/844,174, filed on May 7, 2019.

(51) Int. Cl. C12N 15/113

(52) U.S. Cl. CPC C12N 15/113; C12N 2310/11; C12N 2310/14; C12N 2310/315; C12N 2310/321; C12N 2310/322; C12N 2310/3515; C12N 2320/34

(57) ABSTRACT

The present invention provides iRNA agents, e.g., double stranded iRNA agents, that target the transthyretin (TTR) gene and methods of using such iRNA agents for treating or preventing TTR-associated ocular diseases.

### RELATED APPLICATIONS

This application is a continuation of U.S. patent application Ser. No. 18/225,919, filed on Jul. 25, 2023, which is a continuation of U.S. patent application Ser. No. 17/213,294, filed on Mar. 26, 2021, now abandoned, which is a 35 § U.S.C. 111(a) continuation application of International Application No. PCT/US2019/053050, filed on Sep. 26, 2019, which in turn claims the benefit of priority to U.S. Provisional Application No. 62/738,256, filed on Sep. 28, 2018, and U.S. Provisional Application No. 62/844,174, filed on May 7, 2019. The entire contents of each of the foregoing applications are incorporated herein by reference.

This application is related to U.S. Provisional Application No. 62/668,072, filed on May 7, 2018, U.S. Provisional Application No. 62/738,747, filed on Sep. 28, 2018, U.S. Provisional Application No. 62/773,082, filed on Nov. 29, 2018, and International Application No. PCT/US2019/031170, filed on May 7, 2019. The entire contents of each of the foregoing applications are incorporated herein by reference.

This application is also related to U.S. Provisional Application No. 61/615,618, filed on Mar. 26, 2012, U.S. Provisional Application No. 61/680,098, filed on Aug. 6, 2012, U.S. application Ser. No. 14/358,972, filed on May 16, 2014, now U.S. Pat. No. 9,399,775, issued on Jul. 26, 2016, U.S. application Ser. No. 15/188,317, filed on Jun. 21, 2016, and International Application No. PCT/US2012/065691, filed on Nov. 16, 2012 and published as WO 2013/075035, on May 23, 2013. The entire contents of each of the foregoing applications are incorporated herein by reference.

In addition, this application is related to U.S. Provisional Application No. 62/199,563, filed on Jul. 31, 2015, U.S. Provisional Application No. 62/287,518, filed on Jan. 27, 2016, International Application No. PCT/US2016/044359, filed on Jul. 28, 2016 and published as WO 2017/023660 on Feb. 9, 2017, U.S. application Ser. No. 15/221,651, filed on Jul. 28, 2016, now U.S. Pat. No. 10,208,307, issued on Feb. 19, 2019, and U.S. application Ser. No. 16/223,362, filed on Dec. 18, 2018. The entire contents of each of the foregoing applications are incorporated herein by reference.

### SEQUENCE LISTING

The instant application contains a Sequence Listing which has been submitted electronically in XML file format and is hereby incorporated by reference in its entirety. Said XML copy, created on Jun. 11, 2025, is named 121301-09305_SL.xml and is 516,847 bytes in size.

### BACKGROUND OF THE INVENTION

Transthyretin (TTR) (also known as prealbumin) transports retinol-binding protein (RBP) and thyroxine (T4) and also acts as a carrier of retinol (vitamin A) through its association with RBP in the blood and the CSF. Transthyretin is named for its transport of thyroxine and retinol. TTR also functions as a protease and can cleave proteins including apoA-I (the major HDL apolipoprotein), amyloid β-peptide, and neuropeptide Y. See Liz, M. A. et al. (2010) IUBMB Life, 62(6):429-435.

TTR is a tetramer of four identical 127-amino acid subunits (monomers) that are rich in beta sheet structure. Each monomer has two 4-stranded beta sheets and the shape of a prolate ellipsoid. Antiparallel beta-sheet interactions link monomers into dimers. A short loop from each monomer forms the main dimer-dimer interaction. These two pairs of loops separate the opposed, convex beta-sheets of the dimers to form an internal channel.

The liver is the major site of TTR expression, however, TTR, is also expressed elsewhere, including the choroid plexus, retina (particularly retinal pigment epithelial cells (RPEs) and ciliary epilelial cells (CEs)) and pancreas.

Transthyretin is one of at least 27 distinct types of proteins that is a precursor protein in the formation of amyloid fibrils. See Guan, J. et al. (Nov. 4, 2011) Current perspectives on cardiac amyloidosis, Am J Physiol Heart Circ Physiol, doi:10.1152/ajpheart.00815.2011. Extracellular deposition of amyloid fibrils in organs and tissues is the hallmark of amyloidosis. Amyloid fibrils are composed of misfolded protein aggregates, which may result from either excess production of or specific mutations in precursor proteins. The amyloidogenic potential of TTR may be related to its extensive beta sheet structure; X-ray crystallographic studies indicate that certain amyloidogenic mutations destabilize the tetrameric structure of the protein. See, e.g., Saraiva M. J. M. (2002) Expert Reviews in Molecular Medicine, 4(12):1-11.

Amyloidosis is a general term for the group of amyloid diseases that are characterized by amyloid deposits. Amyloid diseases are classified based on their precursor protein; for example, the name starts with “A” for amyloid and is followed by an abbreviation of the precursor protein, e.g., ATTR for amyloidogenic transthyretin. Ibid.

There are numerous TTR-associated diseases, most of which are amyloid diseases. Normal-sequence TTR is associated with cardiac amyloidosis in people who are elderly and is termed senile systemic amyloidosis (SSA) (also called senile cardiac amyloidosis (SCA) or cardiac amyloidosis). SSA often is accompanied by microscopic deposits in many other organs. TTR amyloidosis manifests in various forms. When the peripheral nervous system is affected more prominently, the disease is termed familial amyloidotic polyneuropathy (FAP). When the heart is primarily involved but the nervous system is not, the disease is called familial amyloidotic cardiomyopathy (FAC). A third major type of TTR amyloidosis is leptomeningeal amyloidosis, also known as leptomeningeal or meningocerebrovascular amyloidosis, central nervous system (CNS) amyloidosis, or amyloidosis VII form. Mutations in TTR may also cause amyloidotic vitreous opacities, carpal tunnel syndrome, and euthyroid hyperthyroxinemia, which is a non-amyloidotic disease thought to be secondary to an increased association of thyroxine with TTR due to a mutant TTR molecule with increased affinity for thyroxine. See, e.g., Moses et al. (1982) J. Clin. Invest., 86, 2025-2033.

Abnormal amyloidogenic proteins may be either inherited or acquired through somatic mutations. Guan, J. et al. (Nov. 4, 2011) Current perspectives on cardiac amyloidosis, Am J Physiol Heart Circ Physiol, doi:10.1152/ajpheart.00815.2011. Transthyretin associated ATTR is the most frequent form of hereditary systemic amyloidosis. Lobato, L. (2003) J. Nephrol., 16:438-442. TTR mutations accelerate the process of TTR amyloid formation and are the most important risk factor for the development of ATTR. More than 85 amyloidogenic TTR variants are known to cause systemic familial amyloidosis. TTR mutations usually give rise to systemic amyloid deposition, with particular involvement of the peripheral nervous system, although some mutations are associated with cardiomyopathy or vitreous opacities. Ibid.

The V30M mutation is the most prevalent TTR mutation. See, e.g., Lobato, L. (2003) J Nephrol, 16:438-442. The V122I mutation is carried by 3.9% of the African American population and is the most common cause of FAC. Jacobson, D. R. et al. (1997) N. Engl. J. Med. 336 (7): 466-73. It is estimated that SSA affects more than 25% of the population over age 80. Westermark, P. et al. (1990) Proc. Natl. Acad. Sci. U.S.A. 87 (7): 2843-5.

Until the recent approval of the double stranded RNAi agent targeting hepatic TTR, Patisiran, liver transplantation was the only therapy for treatment of TTR-associated disease. Interestingly, however, liver transplantation does not inhibit ocular disease associated with TTR mutations (Hara, et al. (2010) Arch Opthamol 128:206-210). Therefore, Patisiran, which targets TTR produced in the liver, is will not inhibit TTR-associated ocular disease since efficient delivery of an iRNA agent to cells in vivo requires specific targeting and substantial protection from the extracellular environment, particularly serum protein. Thus, siRNA delivery into extra-hepatic tissues remains an obstacle, limiting the use of siRNA-based therapies.

As indicated above, one of the factors that limit the experimental and therapeutic application of iRNA agents in vivo is the ability to deliver intact siRNA efficiently. Particular difficulties have been associated with non-viral gene transfer into the retina in vivo. One of the challenges is to overcome the inner limiting membrane, which impedes the transfection of the retina. Additionally, negatively charged sugars of the vitreous have been shown to interact with positive DNA-transfection reagent complexes, promoting their aggregation, which impedes diffusion and cellular uptake.

Thus, there is a need for new and improved compositions and methods for delivering siRNA molecules into extra-hepatic tissues, such as ocular tissues, in vivo, for treatment of TTR-associated ocular diseases and disorders.

### SUMMARY OF THE INVENTION

The present invention provides RNAi agents, e.g., double stranded RNAi agents, and compositions targeting the Transthyretin (TTR) gene. The present invention also provides methods of inhibiting expression of TTR and methods of treating or preventing a TTR-associated ocular disease in a subject using the RNAi agents, e.g., double stranded RNAi agents, of the invention. The present invention is based, at least in part, on the discovery that conjugating a lipophilic moiety to one or more internal positions on at least one strand of a double-stranded iRNA agent targeting TTR, or to one or more positions on at least one strand within the double stranded region of a double-stranded iRNA agent targeting TTR, provides surprisingly good results for in vivo intraocular delivery of the double-stranded iRNAs, resulting in efficient entry into ocular tissues and efficient internalization into cells of the ocular system.

Accordingly, in one aspect, the present invention provides a double stranded RNAi agent comprising a sense strand complementary to an antisense strand, wherein the antisense strand comprises a region complementary to part of an mRNA encoding transthyretin (TTR), wherein each strand independently has 14 to 30 nucleotides, wherein the double stranded RNAi agent is represented by formula (III):

(III)
sense:
5′ np-Na-(X X X)i-Nb-Y Y Y-Nb-(Z Z Z)j-Na-nq 3′
antisense:
3′ np′-Na′-(X′X′X′)k-Nb′-Y′Y′Y′-Nb′-(Z′Z′Z′)l-
Na′-nq′ 5′

wherein i, j, k, and l are each independently 0 or 1, provided that at least one of i, j, k, and l is 1; p, p′, q, and q′ are each independently 0-6; each Na and Na′ independently represents an oligonucleotide sequence comprising 2-20 nucleotides which are modified, each sequence comprising at least two differently modified nucleotides; each Nb and Nb′ independently represents an oligonucleotide sequence comprising 1-10 nucleotides which are modified; each np, np′, nq, and nq′ independently represents an overhang nucleotide; XXX, YYY, ZZZ, X′X′X′, Y′Y′Y′, and Z′Z′Z′ each independently represent one motif of three identical modifications on three consecutive nucleotides; and wherein one or more lipophilic moieties are conjugated to one or more internal positions on at least one strand.

In certain embodiments, the lipophilic moiety is conjugated to position 20, position 15, position 7, position 6, or position 2 of the sense strand (counting from the 5′ end of the strand) or position 16 of the antisense strand (counting from the 5′ end of the strand). In certain embodiments, the lipophilic moiety is conjugated to position 20, position 15, or position 7 of the sense strand (counting from the 5′ end of the strand). In certain embodiments, the lipophilic moiety is conjugated to position 20 or position 15 of the sense strand (counting from the 5′ end of the strand). In certain embodiments, the lipophilic moiety is conjugated to position 16 of the antisense strand (counting from the 5′ end of the strand).

In certain embodiments, the antisense strand of the double stranded RNAi agent comprises a sequence that is complementary to 5′-TGGGATTTCATGTAACCAAGA-3′ (SEQ ID NO: 11).

In another aspect, the present invention provides a double stranded RNAi agent comprising a sense strand complementary to an antisense strand, wherein the antisense strand comprises a sequence that is complementary to nucleotides 504 to 526 of the transthyretin (TTR) gene (SEQ ID NO:1), wherein the sense strand is 21 nucleotides in length and the antisense strand is 23 nucleotides in length, wherein the double stranded RNAi agent is represented by formula (III):

(III)
sense:
5′ np-Na-(X X X)i-Nb-Y Y Y-Nb-(Z Z Z)j-Na-nq 3′
antisense:
3′ np′-Na′-(X′X′X′)k-Nb′-Y′Y′Y′-Nb′-(Z′Z′Z′)l-
Na′-nq′ 5′

wherein j=1; and i, k, and 1 are 0; p′ is 2; p, q, and q′ are 0; each Na and Na′ independently represents an oligonucleotide sequence comprising 2-10 nucleotides which are modified nucleotides; each Nb and Nb′ independently represents an oligonucleotide sequence comprising 0-7 nucleotides which are modified nucleotides; np′ represents an overhang nucleotide; YYY, ZZZ, and Y′Y′Y′, each independently represent one motif of three identical modifications on three consecutive nucleotides, wherein the Y nucleotides contain a 2′-fluoro modification, the Y′ nucleotides contain a 2′-O-methyl modification, and the Z nucleotides contain a 2′-O-methyl modification; and wherein one or more lipophilic moieties are conjugated to one or more internal positions on at least one strand.

In certain embodiments, the lipophilic moiety is conjugated to position 20, position 15, position 7, position 6, or position 2 of the sense strand (counting from the 5′ end of the strand) or position 16 of the antisense strand (counting from the 5′ end of the strand). In certain embodiments, the lipophilic moiety is conjugated to position 20, position 15, or position 7 of the sense strand (counting from the 5′ end of the strand). In certain embodiments, the lipophilic moiety is conjugated to position 20 or position 15 of the sense strand (counting from the 5′ end of the strand). In certain embodiments, the lipophilic moiety is conjugated to position 6 of the sense strand (counting from the 5′ end of the strand). In certain embodiments, the lipophilic moiety is conjugated to position 16 of the antisense strand (counting from the 5′ end of the strand).

In another aspect, the present invention provides a double stranded RNAi agent for inhibiting expression of TTR in a cell, wherein the double stranded RNAi agent comprises a sense strand and an antisense strand forming a double stranded region; wherein the sense strand comprises the nucleotide sequence 5′-UGGGAUUUCAUGUAACCAAGA-3′ (SEQ ID NO: 12) and the antisense strand comprises the nucleotide sequence 5′-UCUUGGUUACAUGAAAUCCCAUC-3′ (SEQ ID NO: 13); wherein substantially all of the nucleotides of the sense strand and substantially all of the nucleotides of the antisense strand comprise a modification; and wherein one or more lipophilic moieties are conjugated to one or more internal positions on at least one strand. In certain embodiments, the sense strand is 21 nucleotides in length, and the lipophilic moiety is conjugated to position 20, position 15, position 7, position 6, or position 2 of the sense strand (counting from the 5′ end of the strand) or position 16 of the antisense strand (counting from the 5′ end of the strand). In certain embodiments, the lipophilic moiety is conjugated to position 20, position 15, or position 7 of the sense strand (counting from the 5′ end of the strand). In certain embodiments, the lipophilic moiety is conjugated to position 20 or position 15 of the sense strand (counting from the 5′ end of the strand). In certain embodiments, the lipophilic moiety is conjugated to position 6 of the sense strand (counting from the 5′ end of the strand). In certain embodiments, the lipophilic moiety is conjugated to position 16 of the antisense strand (counting from the 5′ end of the strand). In certain embodiments, the double stranded RNAi agent further comprises a phosphate or phosphate mimic at the 5′-end of the antisense strand. In certain embodiments, the phosphate mimic is a 5′-vinyl phosphonate (VP).

In another aspect, the present invention provides a double stranded ribonucleic acid (RNAi) agent that inhibits expression of transthyretin (TTR) in a cell, comprising a sense strand differing by no more than 4 modified nucleotides from the nucleotide sequence of 5′-usgsggauUfuCfAfUfguaaccaaga-3′ (SEQ ID NO: 10) and an antisense strand differing by no more than 4 modified nucleotides from the nucleotide sequence 5′-usCfsuugGfuuAfcaugAfaAfucccasusc-3′ (SEQ ID NO: 7), wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively; and s is a phosphorothioate linkage; and wherein one or more lipophilic moieties are conjugated to one or more internal positions on at least one strand. In certain embodiments, the lipophilic moiety is conjugated to position 20, position 15, position 7, position 6, or position 2 of the sense strand (counting from the 5′ end of the strand) or position 16 of the antisense strand (counting from the 5′ end of the strand). In certain embodiments, the lipophilic moiety is conjugated to position 20, position 15, or position 7 of the sense strand (counting from the 5′ end of the strand). In certain embodiments, the lipophilic moiety is conjugated to position 20 or position 15 of the sense strand (counting from the 5′ end of the strand). In certain embodiments, the lipophilic moiety is conjugated to position 16 of the antisense strand (counting from the 5′ end of the strand). In certain embodiments, the double stranded RNAi agent further comprises a phosphate or phosphate mimic at the 5′-end of the antisense strand. In certain embodiments, the phosphate mimic is a 5′-vinyl phosphonate (VP).

In another aspect, the present invention provides a double stranded ribonucleic acid (RNAi) agent, comprising a sense strand and an antisense strand, wherein the sense strand comprises the nucleotide sequence 5′-usgsggauUfuCfAfUfguaaccaaga-3′ (SEQ ID NO: 10) and the antisense strand comprises the nucleotide sequence 5′-usCfsuugGfuuAfcaugAfaAfucccasusc-3′ (SEQ ID NO: 7), wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively; and s is a phosphorothioate linkage; and wherein one or more lipophilic moieties are conjugated to one or more internal positions on at least one strand. In certain embodiments, the lipophilic moiety is conjugated to position 20, position 15, position 7, position 6, or position 2 of the sense strand (counting from the 5′ end of the strand) or position 16 of the antisense strand (counting from the 5′ end of the strand). In certain embodiments, the lipophilic moiety is conjugated to position 20, position 15, or position 7 of the sense strand (counting from the 5′ end of the strand). In certain embodiments, the lipophilic moiety is conjugated to position 20 or position 15 of the sense strand (counting from the 5′ end of the strand). In certain embodiments, the lipophilic moiety is conjugated to position 16 of the antisense strand (counting from the 5′ end of the strand). In certain embodiments, the double stranded RNAi agent further comprises a phosphate or phosphate mimic at the 5′-end of the antisense strand. In certain embodiments, the phosphate mimic is a 5′-vinyl phosphonate (VP).

In another aspect, the present invention provides a double stranded RNAi agent for inhibiting expression of TTR in a cell, wherein the double stranded RNAi agent comprises a sense strand and an antisense strand forming a double stranded region; wherein the sense strand comprises the nucleotide sequence 5′-UGGGAUUUCAUGUAACCAAGA-3′ (SEQ ID NO: 12) and the antisense strand comprises the nucleotide sequence 5′-UCUUGGUUACAUGAAAUCCCAUC-3′ (SEQ ID NO: 13); wherein substantially all of the nucleotides of the sense strand and substantially all of the nucleotides of the antisense strand comprise a modification; and wherein one or more lipophilic moieties are conjugated to one or more positions on at least one strand within the double stranded region. In certain embodiments, the sense strand is 21 nucleotides in length, and the lipophilic moiety is conjugated to position 21, position 20, position 15, position 1, position 7, position 6, or position 2 of the sense strand (counting from the 5′ end of the strand) or position 16 of the antisense strand (counting from the 5′ end of the strand). In certain embodiments, the lipophilic moiety is conjugated to position 21, position 20, position 15, position 1, or position 7 of the sense strand (counting from the 5′ end of the strand). In certain embodiments, the lipophilic moiety is conjugated to position 21, position 20, or position 15 of the sense strand (counting from the 5′ end of the strand). In certain embodiments, the lipophilic moiety is conjugated to position 20 or position 15 of the sense strand (counting from the 5′ end of the strand). In certain embodiments, the lipophilic moiety is conjugated to position 6 of the sense strand (counting from the 5′ end of the strand). In certain embodiments, the antisense strand is 23 nucleotides in length and the lipophilic moiety is conjugated to position 16 of the antisense strand (counting from the 5′ end of the strand). In certain embodiments, the double stranded RNAi agent further comprises a phosphate or phosphate mimic at the 5′-end of the antisense strand. In certain embodiments, the phosphate mimic is a 5′-vinyl phosphonate (VP).

In another aspect, the present invention provides a double stranded ribonucleic acid (RNAi) agent that inhibits expression of transthyretin (TTR) in a cell, comprising a sense strand differing by no more than 4 modified nucleotides from the nucleotide sequence of 5′-usgsggauUfuCfAfUfguaaccaaga-3′ (SEQ ID NO: 10) and an antisense strand differing by no more than 4 modified nucleotides from the nucleotide sequence 5′-usCfsuugGfuuAfcaugAfaAfucccasusc-3′ (SEQ ID NO: 7), wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively; and s is a phosphorothioate linkage; and wherein one or more lipophilic moieties are conjugated to one or more positions on at least one strand within the double stranded region. In certain embodiments, the lipophilic moiety is conjugated to position 21, position 20, position 15, position 1, position 7, position 6, or position 2 of the sense strand (counting from the 5′ end of the strand) or position 16 of the antisense strand (counting from the 5′ end of the strand). In certain embodiments, the lipophilic moiety is conjugated to position 21, position 20, position 15, position 1, or position 7 of the sense strand (counting from the 5′ end of the strand). In certain embodiments, the lipophilic moiety is conjugated to position 21, position 20, or position 15 of the sense strand (counting from the 5′ end of the strand). In certain embodiments, the lipophilic moiety is conjugated to position 20 or position 15 of the sense strand (counting from the 5′ end of the strand). In certain embodiments, the lipophilic moiety is conjugated to position 6 of the sense strand (counting from the 5′ end of the strand). In certain embodiments, the lipophilic moiety is conjugated to position 16 of the antisense strand (counting from the 5′ end of the strand). In certain embodiments, the double stranded RNAi agent further comprises a phosphate or phosphate mimic at the 5′-end of the antisense strand. In certain embodiments, the phosphate mimic is a 5′-vinyl phosphonate (VP).

In another aspect, the present invention provides a double stranded ribonucleic acid (RNAi) agent, comprising a sense strand and an antisense strand, wherein the sense strand comprises the nucleotide sequence 5′-usgsggauUfuCfAfUfguaaccaaga-3′ (SEQ ID NO: 10) and the antisense strand comprises the nucleotide sequence 5′-usCfsuugGfuuAfcaugAfaAfucccasusc-3′ (SEQ ID NO: 7), wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively; and s is a phosphorothioate linkage; and wherein one or more lipophilic moieties are conjugated to one or more positions on at least one strand within the double stranded region. In certain embodiments, the lipophilic moiety is conjugated to position 21, position 20, position 15, position 1, position 7, position 6, or position 2 of the sense strand (counting from the 5′ end of the strand) or position 16 of the antisense strand (counting from the 5′ end of the strand). In certain embodiments, the lipophilic moiety is conjugated to position 21, position 20, position 15, position 1, or position 7 of the sense strand (counting from the 5′ end of the strand). In certain embodiments, the lipophilic moiety is conjugated to position 21, position 20, or position 15 of the sense strand (counting from the 5′ end of the strand). In certain embodiments, the lipophilic moiety is conjugated to position 20 or position 15 of the sense strand (counting from the 5′ end of the strand). In certain embodiments, the lipophilic moiety is conjugated to position 6 of the sense strand (counting from the 5′ end of the strand). In certain embodiments, the lipophilic moiety is conjugated to position 16 of the antisense strand (counting from the 5′ end of the strand). In certain embodiments, the double stranded RNAi agent further comprises a phosphate or phosphate mimic at the 5′-end of the antisense strand. In certain embodiments, the phosphate mimic is a 5′-vinyl phosphonate (VP).

In certain embodiments, the one or more lipophilic moieties are conjugated to one or more internal positions on at least one strand of the double stranded RNAi agent via a linker or carrier. In certain embodiments, the one or more lipophilic moieties are conjugated to one or more positions on at least one strand within the double stranded region via a linker or carrier.

In certain embodiments, the lipophilicity of the lipophilic moiety, measured by log Kow, exceeds 0.

In certain embodiments, the hydrophobicity of the double-stranded iRNA agent, measured by the unbound fraction in the plasma protein binding assay of the double-stranded iRNA agent, exceeds 0.2.

In certain embodiments, the plasma protein binding assay is an electrophoretic mobility shift assay using human serum albumin protein.

In certain embodiments, the internal positions include all positions except the terminal two positions from each end of the at least one strand of the double stranded RNAi agent.

In certain embodiments, the internal positions include all positions except the terminal three positions from each end of the at least one strand of the double stranded RNAi agent.

In certain embodiments, the internal positions exclude a cleavage site region of the sense strand of the double stranded RNAi agent. In certain embodiments, the positions within the double stranded region exclude a cleavage site region of the sense strand of the double stranded RNAi agent.

In certain embodiments, the internal positions include all positions except positions 9-12, counting from the 5′-end of the sense strand of the double stranded RNAi agent.

In certain embodiments, the internal positions include all positions except positions 11-13, counting from the 3′-end of the sense strand of the double stranded RNAi agent.

In certain embodiments, the internal positions exclude a cleavage site region of the antisense strand of the double stranded RNAi agent.

In certain embodiments, the internal positions include all positions except positions 12-14, counting from the 5′-end of the antisense strand of the double stranded RNAi agent.

In certain embodiments, the internal positions include all positions except positions 11-13 on the sense strand of the double stranded RNAi agent, counting from the 3′-end, and positions 12-14 on the antisense strand of the RNAi agent, counting from the 5′-end.

In certain embodiments, the one or more lipophilic moieties are conjugated to one or more of the internal positions selected from the group consisting of positions 4-8 and 13-18 on the sense strand, and positions 6-10 and 15-18 on the antisense strand, counting from the 5′end of each strand of the RNAi agent.

In certain embodiments, the one or more lipophilic moieties are conjugated to one or more of the internal positions selected from the group consisting of positions 5, 6, 7, 15, and 17 on the sense strand, and positions 15 and 17 on the antisense strand, counting from the 5′-end of each strand of the RNAi agent.

In certain embodiments, the lipophilic moiety is an aliphatic, alicyclic, or polyalicyclic compound.

In certain embodiments, the lipophilic moiety is selected from the group consisting of lipid, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine.

In certain embodiments, the lipophilic moiety contains a saturated or unsaturated C4-C30 hydrocarbon chain, and an optional functional group selected from the group consisting of hydroxyl, amine, carboxylic acid, sulfonate, phosphate, thiol, azide, and alkyne.

In certain embodiments, the lipophilic moiety contains a saturated or unsaturated C6-C18 hydrocarbon chain.

In certain embodiments, the lipophilic moiety contains a saturated or unsaturated C16 hydrocarbon chain.

In certain embodiments, the saturated or unsaturated C16 hydrocarbon chain is conjugated to position 6, counting from the 5′-end of the strand on the double stranded RNAi agent. In certain embodiments, the saturated or unsaturated C16 hydrocarbon chain is conjugated to position 6, counting from the 5′-end of the sense strand on the double stranded RNAi agent.

In certain embodiments, the lipophilic moiety is conjugated via a carrier that replaces one or more nucleotide(s) in the internal position(s) on the strand of the double stranded RNAi agent. In certain embodiments, the lipophilic moiety is conjugated via a carrier that replaces one or more nucleotide(s) in the double stranded region.

In certain embodiments, the carrier is a cyclic group selected from the group consisting of pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, and decalinyl; or is an acyclic moiety based on a serinol backbone or a diethanolamine backbone.

In certain embodiments, the lipophilic moiety is conjugated to the double-stranded iRNA agent via a linker containing an ether, thioether, urea, carbonate, amine, amide, maleimide-thioether, disulfide, phosphodiester, sulfonamide linkage, a product of a click reaction, or carbamate.

In certain embodiments, the lipophilic moiety is conjugated to a nucleobase, sugar moiety, or internucleosidic linkage.

In certain embodiments, the double stranded RNAi agent further comprises a ligand that mediates delivery to an ocular tissue.

In some embodiments, the ligand that mediates delivery to the ocular tissue is a targeting ligand that targets a receptor which mediates delivery to the ocular tissue.

In certain embodiments, the targeting ligand is selected from the group consisting of trans-retinol, RGD peptide, LDL receptor ligand, and carbohydrate based ligands.

In certain embodiments, the RGD peptide is H-Gly-Arg-Gly-Asp-Ser-Pro-Lys-Cys-OH (SEQ ID NO: 14) or Cyclo(-Arg-Gly-Asp-D-Phe-Cys) (SEQ ID NO: 104).

In certain embodiments, the double stranded RNAi agent further comprises a targeting ligand that targets a liver tissue.

In certain embodiments, the targeting ligand is a GalNAc conjugate.

In certain embodiments, the lipophilic moeity or targeting ligand is conjugated to the double stranded RNAi agent via a bio-cleavable linker selected from the group consisting of DNA, RNA, disulfide, amide, functionalized monosaccharides or oligosaccharides of galactosamine, glucosamine, glucose, galactose, mannose, and combinations thereof.

In certain embodiments, the 3′ end of the sense strand of the double stranded RNAi agent is protected via an end cap which is a cyclic group having an amine, the cyclic group being selected from the group consisting of pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, and decalinyl.

In certain embodiments, the RNAi agent comprises a terminal, chiral modification occurring at the first internucleotide linkage at the 3′ end of the antisense strand, having the linkage phosphorus atom in Sp configuration, a terminal, chiral modification occurring at the first internucleotide linkage at the 5′ end of the antisense strand, having the linkage phosphorus atom in Rp configuration, and a terminal, chiral modification occurring at the first internucleotide linkage at the 5′ end of the sense strand, having the linkage phosphorus atom in either Rp configuration or Sp configuration.

In certain embodiments, the RNAi agent comprises a terminal, chiral modification occurring at the first and second internucleotide linkages at the 3′ end of the antisense strand, having the linkage phosphorus atom in Sp configuration, a terminal, chiral modification occurring at the first internucleotide linkage at the 5′ end of the antisense strand, having the linkage phosphorus atom in Rp configuration, and a terminal, chiral modification occurring at the first internucleotide linkage at the 5′ end of the sense strand, having the linkage phosphorus atom in either Rp or Sp configuration.

In certain embodiments, the RNAi agent comprises a terminal, chiral modification occurring at the first, second and third internucleotide linkages at the 3′ end of the antisense strand, having the linkage phosphorus atom in Sp configuration, a terminal, chiral modification occurring at the first internucleotide linkage at the 5′ end of the antisense strand, having the linkage phosphorus atom in Rp configuration, and a terminal, chiral modification occurring at the first internucleotide linkage at the 5′ end of the sense strand, having the linkage phosphorus atom in either Rp or Sp configuration.

In certain embodiments, the RNAi agent comprises a terminal, chiral modification occurring at the first, and second internucleotide linkages at the 3′ end of the antisense strand, having the linkage phosphorus atom in Sp configuration, a terminal, chiral modification occurring at the third internucleotide linkages at the 3′ end of the antisense strand, having the linkage phosphorus atom in Rp configuration, a terminal, chiral modification occurring at the first internucleotide linkage at the 5′ end of the antisense strand, having the linkage phosphorus atom in Rp configuration, and a terminal, chiral modification occurring at the first internucleotide linkage at the 5′ end of the sense strand, having the linkage phosphorus atom in either Rp or Sp configuration.

In certain embodiments, the RNAi agent comprises a terminal, chiral modification occurring at the first, and second internucleotide linkages at the 3′ end of the antisense strand, having the linkage phosphorus atom in Sp configuration, a terminal, chiral modification occurring at the first, and second internucleotide linkages at the 5′ end of the antisense strand, having the linkage phosphorus atom in Rp configuration, and a terminal, chiral modification occurring at the first internucleotide linkage at the 5′ end of the sense strand, having the linkage phosphorus atom in either Rp or Sp configuration.

In certain embodiments, the double stranded RNAi agent is represented by formula (III):

(III)
sense:
5′ np-Na-(X X X)i-Nb-Y Y Y-Nb-(Z Z Z)j-Na-nq 3′
antisense:
3′ np′-Na′-(X′X′X′)k-Nb′-Y′Y′Y′-Nb′-(Z′Z′Z′)l-
Na′-nq′ 5′

wherein j is 1 or 2; or wherein l is 1; or wherein both j and l are 1.

In certain embodiments, the double stranded RNAi agent is represented by formula (III):

(III)
sense:
5′ np-Na-(X X X)i-Nb-Y Y Y-Nb-(Z Z Z)j-Na-nq 3′
antisense:
3′ np′-Na′-(X′X′X′)k-Nb′-Y′Y′Y′-Nb′-(Z′Z′Z′)l-
Na′-nq′ 5′

wherein XXX is complementary to X′X′X′, YYY is complementary to Y′Y′Y′, and ZZZ is complementary to Z′Z′Z′.

In certain embodiments, the YYY motif occurs at or near the cleavage site of the sense strand of the double stranded RNAi agent; or wherein the Y′Y′Y′ motif occurs at the 11, 12 and 13 positions of the antisense strand of the double stranded RNAi agent, from the 5-end.

In some embodiment, formula (III) is represented as formula (IIIa):

(IIIa)
sense:
5′ np-Na-Y Y Y-Nb-Z Z Z-Na-nq 3′
antisense:
3′ np′-Na′-Y′Y′Y′-Nb′-Z′Z′Z′-Na′nq′ 5′

wherein each Nb and Nb′ independently represents an oligonucleotide sequence comprising 1-5 modified nucleotides; or
formula (III) is represented as formula (IIIb):

(IIIb)
sense:
5′ np-Na-X X X-Nb-Y Y Y-Na-nq 3′
antisense:
3′ np′-Na′-X′X′X′-Nb′-Y′Y′Y′-Na′-nq′ 5′

wherein each Nb and Nb′ independently represents an oligonucleotide sequence comprising 1-5 modified nucleotides; or
formula (III) is represented as formula (IIIc):

(IIIc)
sense:
5′ np-Na-X X X-Nb-Y Y Y-Nb-Z Z Z-Na-nq 3′
antisense:
3′ np′-Na′-X′X′X′-Nb′-Y′Y′Y′-Nb′-Z′Z′Z′-Na′-nq′ 5′

wherein each Nb and Nb′ independently represents an oligonucleotide sequence comprising 1-5 modified nucleotides and each Na and Na′ independently represents an oligonucleotide sequence comprising 2-10 modified nucleotides.

In certain embodiments, the modifications on the nucleotides of the double stranded RNAi agent are selected from the group consisting of a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, and a nucleotide comprising a 5′-phosphate mimic, and combinations thereof.

In certain embodiments, the modifications on the nucleotides are 2′-O-methyl, 2′-fluoro or both.

In certain embodiments, the Y′ of formula (III) is 2′-O-methyl.

In certain embodiments, the Z nucleotides of formula (III) contain a 2′-O-methyl modification.

In certain embodiments, the modifications on the Na, Na′, Nb, and Nb′ nucleotides of formula (III) are 2′-O-methyl, 2′-fluoro or both.

In certain embodiments, the sense strand and the antisense strand of the RNAi agent form a duplex region which is 15-30 nucleotide pairs in length.

In certain embodiments, the duplex region is 17-25 nucleotide pairs in length.

In certain embodiments, the sense and antisense strands of the RNAi agent are each 15 to 30 nucleotides in length.

In certain embodiments, the sense and antisense strands of the RNAi agent are each 19 to 25 nucleotides in length.

In certain embodiments, each of the sense strand and the antisense strand of the RNAi agent independently have 21 to 23 nucleotides.

In certain embodiments, the sense strand of the RNAi agent has a total of 21 nucleotides and the antisense strand of the RNAi agent has a total of 23 nucleotides.

In certain embodiments, the RNAi agent further comprises at least one phosphorothioate or methylphosphonate internucleotide linkage.

In certain embodiments, the phosphorothioate or methylphosphonate internucleotide linkage is at the 3′-terminal of one strand.

In certain embodiments, the phosphorothioate or methylphosphonate internucleotide linkage is at the 3′-terminal of the antisense strand. In certain embodiments, the double stranded RNAi agent is represented by formula (III), wherein p′=2.

In certain embodiments, the double stranded RNAi agent is represented by formula (III), wherein at least one np′ is linked to a neighboring nucleotide via a phosphorothioate linkage.

In certain embodiments, the double stranded RNAi agent is represented by formula (III), wherein all np′ are linked to neighboring nucleotides via phosphorothioate linkages.

In certain embodiments, the double stranded RNAi agent further comprises a phosphate or phosphate mimic at the 5′-end of the antisense strand.

In certain embodiments, the phosphate mimic is a 5′-vinyl phosphonate (VP).

In certain embodiments, the base pair at the 1 position of the 5′-end of the antisense strand of the double stranded RNAi duplex is an AU base pair.

In certain embodiments, the sense strand of the double stranded RNAi agent comprises the nucleotide sequence 5′-UGGGAUUUCAUGUAACCAAGA-3′(SEQ ID NO: 12).

In certain embodiments, the sense strand of the RNAi agent comprises the nucleotide sequence 5′-UGGGAUUUCAUGUAACCAAGA-3′(SEQ ID NO: 12) and the antisense strand of the RNAi agent comprises the nucleotide sequence 5′-UCUUGGUUACAUGAAAUCCCAUC-3′ (SEQ ID NO: 13).

In certain embodiments, the sense and antisense strands of the double stranded RNAi agent comprise the nucleotide sequences 5′-usgsgga(Uhd)UfuCfAfUfguaaccaasgsa-3′ (SEQ ID NO: 15) and 5′-usCfsuugGfuuAfcaugAfaAfucccasusc-3′ (SEQ ID NO: 16), wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively; s is a phosphorothioate linkage; and (Uhd) is 2′-O-hexadecyl-uridine-3′-phosphate.

In certain embodiments, the sense and antisense strands of the double stranded RNAi agent comprise the nucleotide sequences 5′-usgsgga(Uhd)UfuCfAfUfguaaccaasgsa-3′ (SEQ ID NO: 15) and 5′-VPusCfsuugGfuuAfcaugAfaAfucccasusc-3′ (SEQ ID NO: 17), wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively; s is a phosphorothioate linkage; (Uhd) is 2′-O-hexadecyl-uridine-3′-phosphate; and VP is a vinyl phosphonate.

In another aspect, the present invention provides a double stranded ribonucleic acid (RNAi) agent that inhibits expression of transthyretin (TTR) in a cell, comprising a sense strand differing by no more than 4 modified nucleotides from the nucleotide sequence of 5′-usgsgga(Uhd)UfuCfAfUfguaaccaasgsa-3′ (SEQ ID NO: 15) and an antisense strand differing by no more than 4 modified nucleotides from the nucleotide sequence 5′-usCfsuugGfuuAfcaugAfaAfucccasusc-3′ (SEQ ID NO: 16), wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively; s is a phosphorothioate linkage; and (Uhd) is 2′-O-hexadecyl-uridine-3′-phosphate. In certain embodiments, the double stranded RNAi agent further comprises a phosphate or phosphate mimic at the 5′-end of the antisense strand. In certain embodiments, the phosphate mimic is a 5′-vinyl phosphonate (VP).

In certain embodiments, the sense strand of the double stranded RNAi agent differs by no more than 3 modified nucleotides from the nucleotide sequence of 5′-usgsgga(Uhd)UfuCfAfUfguaaccaasgsa-3′ (SEQ ID NO: 15) and the antisense strand differs by no more than 3 modified nucleotides from the nucleotide sequence 5′-usCfsuugGfuuAfcaugAfaAfucccasusc-3′ (SEQ ID NO: 16). In certain embodiments, the double stranded RNAi agent further comprises a phosphate or phosphate mimic at the 5′-end of the antisense strand. In certain embodiments, the phosphate mimic is a 5′-vinyl phosphonate (VP).

In certain embodiments, the sense strand of the double stranded RNAi agent differs by no more than 2 modified nucleotides from the nucleotide sequence of 5′-usgsgga(Uhd)UfuCfAfUfguaaccaasgsa-3′ (SEQ ID NO: 15) and the antisense strand differs by no more than 2 modified nucleotides from the nucleotide sequence 5′-usCfsuugGfuuAfcaugAfaAfucccasusc-3′ (SEQ ID NO: 16). In certain embodiments, the double stranded RNAi agent further comprises a phosphate or phosphate mimic at the 5′-end of the antisense strand. In certain embodiments, the phosphate mimic is a 5′-vinyl phosphonate (VP).

In certain embodiments, the sense strand of the double stranded RNAi agent differs by no more than 1 modified nucleotide from the nucleotide sequence of 5′-usgsgga(Uhd)UfuCfAfUfguaaccaasgsa-3′ (SEQ ID NO: 15) and the antisense strand differs by no more than 1 modified nucleotide from the nucleotide sequence 5′-usCfsuugGfuuAfcaugAfaAfucccasusc-3′ (SEQ ID NO: 16). In certain embodiments, the double stranded RNAi agent further comprises a phosphate or phosphate mimic at the 5′-end of the antisense strand. In certain embodiments, the phosphate mimic is a 5′-vinyl phosphonate (VP).

In certain embodiments, the sense strand of the double stranded RNAi agent comprises the nucleotide sequence 5′-usgsgga(Uhd)UfuCfAfUfguaaccaasgsa-3′ (SEQ ID NO: 15) and the antisense strand comprises the nucleotide sequence 5′-usCfsuugGfuuAfcaugAfaAfucccasusc-3 (SEQ ID NO: 16). In certain embodiments, the double stranded RNAi agent further comprises a phosphate or phosphate mimic at the 5′-end of the antisense strand. In certain embodiments, the phosphate mimic is a 5′-vinyl phosphonate (VP).

In certain embodiments, the double stranded RNAi agent comprises a sense strand and an antisense strand comprising sense strand and antisense strand nucleotide sequences selected from the group consisting of

(SEQ ID NO: 15)
5′-usgsgga(Uhd)UfuCfAfUfguaaccaasgsa-3′
and
(SEQ ID NO: 16)
5′-usCfsuugGfuuAfcaugAfaAfucccasusc-3′;
(AD-291845)
(SEQ ID NO: 59)
5′-usgsggauUfuCfAfUfguaaccaagsadTdTL10-3′
and
(SEQ ID NO: 17)
5′-VPusCfsuugGfuuAfcaugAfaAfucccasusc-3′;
(AD-70191)
(SEQ ID NO: 60)
5′-usgsggauUfuCfAfUfguaaccaagaL10-3′
and
(SEQ ID NO: 17)
5′-VPusCfsuugGfuuAfcaugAfaAfucccasusc-3′;
(AD70500)
(SEQ ID NO: 61)
5′-usgsggauUfuCfAfUfguaaccaagaL57-3′
and
(SEQ ID NO: 17)
5′-VPusCfsuugGfuuAfcaugAfaAfucccasusc-3′;
(AD-290674)
(SEQ ID NO: 96)
5′-asascaguGfuUfCfUfugcucuausas(Ahd)-3′
and
(SEQ ID NO: 98)
5′-VPuUfauaGfagcaagaAfcAfcuguususu-3′;
(AD-307586)
(SEQ ID NO: 95)
5′-asascaguGfuUfCfUfugcucuaus(Ahds)a-3′
and
(SEQ ID NO: 98)
5′-VPuUfauaGfagcaagaAfcAfcuguususu-3′;
(AD-307585)
(SEQ ID NO: 97)
5′-asascaguGfuUfCfUfugcucuausasa-3′
and
(SEQ ID NO: 101)
5′-VPuUfauaGfagcaagaAfc(Ahd)cuguususu-3′;
(AD-307601)
(SEQ ID NO: 94)
5′-asascaguGfuUfCfUfugc(Uhd)cuausasa-3′
and
(SEQ ID NO: 98)
5′-VPuUfauaGfagcaagaAfcAfcuguususu-3′;
(AD-307580)
(SEQ ID NO: 87)
5′-(Ahds)ascaguGfuUfCfUfugcucuausasa-3′
and
(SEQ ID NO: 98)
5′-VPuUfauaGfagcaagaAfcAfcuguususu-3′;
(AD-307566)
(SEQ ID NO: 91)
5′-asascagu(Ghd)uUfCfUfugcucuausasa-3′
and
(SEQ ID NO: 98)
5′-VPuUfauaGfagcaagaAfcAfcuguususu-3′;
(AD-307572)
(SEQ ID NO: 90)
5′-asascag(Uhd)GfuUfCfUfugcucuausasa-3′
and
(SEQ ID NO: 98)
5′-VPuUfauaGfagcaagaAfcAfcuguususu-3′;
( AD-307571)
5′-as(Ahds)caguGfuUfCfUfugcucuausasa-3′
(SEQ ID NO: 88)
and
(SEQ ID NO: 98)
5′-VPuUfauaGfagcaagaAfcAfcuguususu-3′;
(AD-307567)
(SEQ ID NO: 15)
5′-usgsgga(Uhd)UfuCfAfUfguaaccaasgsa-3′
and
(SEQ ID NO: 62)
5′-VPuCfuugGfuuAfcaugAfaAfucccasusc-3′;
(AD-291846)
(SEQ ID NO: 15)
5′-usgsgga(Uhd)UfuCfAfUfguaaccaasgsa-3′
and
(SEQ ID NO: 102)
5′-VPusCfsuugGf(Tgn)uAfcaugAfaAfucccasusc-3′;
(AD-592744)
(SEQ ID NO: 103)
5′-usgsggauUfuCfAfUfguaaccaasgsa-3′
and
(SEQ ID NO: 17)
5′-VPusCfsuugGfuuAfcaugAfaAfucccasusc-3′;
(AD-538697)
and
(SEQ ID NO: 15)
5′-usgsgga(Uhd)UfuCfAfUfguaaccaasgsa-3′
and
(SEQ ID NO: 16)
5′-usCfsuugGfuuAfcaugAfaAfucccasusc-3′,
(AD-597979)

wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively; (Ahd), (Ghd), and (Uhd) are 2′-O-hexadecyl-adenosine-3′-phosphate, 2′-O-hexadecyl-guanosine-3′-phosphate, and 2′-O-hexadecyl-uridine-3′-phosphate, respectively; s is a phosphorothioate linkage; VP is a vinyl phosphonate; L10 is and N-(cholesterylcarboxamidocaproyl)-4-hydroxyprolinol (Hyp-C6-Chol) conjugated to the 3′ end of the strand; and L57 is a N-(stearylcarboxamidocaproyl)-4-hydroxyprolinol (Hyp-C6-C18) conjugated to the 3′ end of the strand. In certain embodiments, the double stranded RNAi agent comprises a sense strand and an antisense strand comprising the nucleotide sequences of the duplex AD-291845.

In certain embodiments, the double stranded RNAi agent comprises a sense strand and an antisense strand consisting of sense strand and antisense strand nucleotide sequences selected from the group consisting of

(SEQ ID NO: 15)
5′-usgsgga(Uhd)UfuCfAfUfguaaccaasgsa-3′
and
(SEQ ID NO: 16)
5′-usCfsuugGfuuAfcaugAfaAfucccasusc-3′;
(AD-291845)
(SEQ ID NO: 59)
5′-usgsggauUfuCfAfUfguaaccaagsadTdTL10-3′
and
(SEQ ID NO: 17)
5′-VPusCfsuugGfuuAfcaugAfaAfucccasusc-3′;
(AD-70191)
(SEQ ID NO: 60)
5′-usgsggauUfuCfAfUfguaaccaagaL10-3′
and
(SEQ ID NO: 17)
5′-VPusCfsuugGfuuAfcaugAfaAfucccasusc-3′;
(AD70500)
(SEQ ID NO: 61)
5′-usgsggauUfuCfAfUfguaaccaagaL57-3′
and
(SEQ ID NO: 17)
5′-VPusCfsuugGfuuAfcaugAfaAfucccasusc-3′;
(AD-290674)
(SEQ ID NO: 96)
5′-asascaguGfuUfCfUfugcucuausas(Ahd)-3′
and
(SEQ ID NO: 98)
5′-VPuUfauaGfagcaagaAfcAfcuguususu-3′;
(AD-307586)
(SEQ ID NO: 95)
5′-asascaguGfuUfCfUfugcucuaus(Ahds)a-3′
and
(SEQ ID NO: 98)
5′-VPuUfauaGfagcaagaAfcAfcuguususu-3′;
(AD-307585)
(SEQ ID NO: 97)
5′-asascaguGfuUfCfUfugcucuausasa-3′
and
(SEQ ID NO: 101)
5′-VPuUfauaGfagcaagaAfc(Ahd)cuguususu-3′;
(AD-307601)
(SEQ ID NO: 94)
5′-asascaguGfuUfCfUfugc(Uhd)cuausasa-3′
and
(SEQ ID NO: 98)
5′-VPuUfauaGfagcaagaAfcAfcuguususu-3′;
(AD-307580)
(SEQ ID NO: 87)
5′-(Ahds)ascaguGfuUfCfUfugcucuausasa-3′
and
(SEQ ID NO: 98)
5′-VPuUfauaGfagcaagaAfcAfcuguususu-3′;
(AD-307566)
(SEQ ID NO: 91)
5′-asascagu(Ghd)uUfCfUfugcucuausasa-3′
and
(SEQ ID NO: 98)
5′-VPuUfauaGfagcaagaAfcAfcuguususu-3′;
(AD-307572)
(SEQ ID NO: 90)
5′-asascag(Uhd)GfuUfCfUfugcucuausasa-3′
and
(SEQ ID NO: 98)
5′-VPuUfauaGfagcaagaAfcAfcuguususu-3′;
(AD-307571)
(SEQ ID NO: 88)
5′-as(Ahds)caguGfuUfCfUfugcucuausasa-3′
and
(SEQ ID NO: 98)
5′-VPuUfauaGfagcaagaAfcAfcuguususu-3′;
(AD-307567)
(SEQ ID NO: 15)
5′-usgsgga(Uhd)UfuCfAfUfguaaccaasgsa-3′
and
(SEQ ID NO: 62)
5′-VPuCfuugGfuuAfcaugAfaAfucccasusc-3′;
(AD-291846)
(SEQ ID NO: 15)
5′-usgsgga(Uhd)UfuCfAfUfguaaccaasgsa-3′
and
(SEQ ID NO: 102)
5′-VPusCfsuugGf(Tgn)uAfcaugAfaAfucccasusc-3′;
(AD-592744)
(SEQ ID NO: 103)
5′-usgsggauUfuCfAfUfguaaccaasgsa-3′
and
(SEQ ID NO: 17)
5′-VPusCfsuugGfuuAfcaugAfaAfucccasusc-3′;
(AD-538697)
and
(SEQ ID NO: 15)
5′-usgsgga(Uhd)UfuCfAfUfguaaccaasgsa-3′
and
(SEQ ID NO: 16)
5′-usCfsuugGfuuAfcaugAfaAfucccasusc-3′,
(AD-597979)

wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively; (Ahd), (Ghd), and (Uhd) are 2′-O-hexadecyl-adenosine-3′-phosphate, 2′-O-hexadecyl-guanosine-3′-phosphate, and 2′-O-hexadecyl-uridine-3′-phosphate, respectively; s is a phosphorothioate linkage; VP is a vinyl phosphonate; L10 is and N-(cholesterylcarboxamidocaproyl)-4-hydroxyprolinol (Hyp-C6-Chol) conjugated to the 3′ end of the strand; and L57 is a N-(stearylcarboxamidocaproyl)-4-hydroxyprolinol (Hyp-C6-C18) conjugated to the 3′ end of the strand. In certain embodiments, the double stranded RNAi agent comprises a sense strand and an antisense strand consisting of the nucleotide sequences of the duplex AD-291845.

In another aspect, the present invention provides a double stranded ribonucleic acid (RNAi) agent that inhibits expression of transthyretin (TTR) in a cell, comprising a sense strand and an antisense strand, wherein the sense strand comprises a nucleotide sequence differing by no more than 4 modified nucleotides from the sense strand nucleotide sequence of a duplex selected from the group consisting of AD-291845, AD-70191, AD70500, AD-290674, AD-307586, AD-307585, AD-307601, AD-307580, AD-307566, AD-307572, AD-307571, AD-307567, AD-291846 AD-592744, AD-538697, and AD-597979, and wherein the antisense strand comprises a nucleotide sequence differing by no more than 4 modified nucleotides from the corresponding antisense strand nucleotide sequence of the duplex. In certain embodiments, the duplex is AD-291845.

In another aspect, the present invention provides a double stranded ribonucleic acid (RNAi) agent that inhibits expression of transthyretin (TTR) in a cell, comprising a sense strand and an antisense strand, wherein the sense strand comprises a nucleotide sequence differing by no more than 3 modified nucleotides from the sense strand nucleotide sequence of a duplex selected from the group consisting of AD-291845, AD-70191, AD70500, AD-290674, AD-307586, AD-307585, AD-307601, AD-307580, AD-307566, AD-307572, AD-307571, AD-307567, AD-291846 AD-592744, AD-538697, and AD-597979, and wherein the antisense strand comprises a nucleotide sequence differing by no more than 3 modified nucleotides from the corresponding antisense strand nucleotide sequence of the duplex. In certain embodiments, the duplex is AD-291845.

In another aspect, the present invention provides a double stranded ribonucleic acid (RNAi) agent that inhibits expression of transthyretin (TTR) in a cell, comprising a sense strand and an antisense strand, wherein the sense strand comprises a nucleotide sequence differing by no more than 2 modified nucleotides from the sense strand nucleotide sequence of a duplex selected from the group consisting of AD-291845, AD-70191, AD70500, AD-290674, AD-307586, AD-307585, AD-307601, AD-307580, AD-307566, AD-307572, AD-307571, AD-307567, AD-291846 AD-592744, AD-538697, and AD-597979, and wherein the antisense strand comprises a nucleotide sequence differing by no more than 2 modified nucleotides from the corresponding antisense strand nucleotide sequence of the duplex. In certain embodiments, the duplex is AD-291845.

In another aspect, the present invention provides a double stranded ribonucleic acid (RNAi) agent that inhibits expression of transthyretin (TTR) in a cell, comprising a sense strand and an antisense strand, wherein the sense strand comprises a nucleotide sequence differing by no more than 1 modified nucleotide from the sense strand nucleotide sequence of a duplex selected from the group consisting of AD-291845, AD-70191, AD70500, AD-290674, AD-307586, AD-307585, AD-307601, AD-307580, AD-307566, AD-307572, AD-307571, AD-307567, AD-291846 AD-592744, AD-538697, and AD-597979, and wherein the antisense strand comprises a nucleotide sequence differing by no more than 1 modified nucleotide from the corresponding antisense strand nucleotide sequence of the duplex. In certain embodiments, the duplex is AD-291845.

In certain embodiments, the sense strand of the double stranded RNAi agent consists of the nucleotide sequence 5′-usgsgga(Uhd)UfuCfAfUfguaaccaasgsa-3′ (SEQ ID NO: 15) and the antisense strand of the double stranded RNAi agent consists of the nucleotide sequence 5′-usCfsuugGfuuAfcaugAfaAfucccasusc-3′ (SEQ ID NO: 16). In certain embodiments, the double stranded RNAi agent further comprises a phosphate or phosphate mimic at the 5′-end of the antisense strand. In certain embodiments, the phosphate mimic is a 5′-vinyl phosphonate (VP).

In certain embodiments, the sense and antisense strands of the double stranded RNAi agent consist of the nucleotide sequences 5′-usgsgga(Uhd)UfuCfAfUfguaaccaasgsa-3′ (SEQ ID NO: 15) and 5′-VPusCfsuugGfuuAfcaugAfaAfucccasusc-3′ (SEQ ID NO: 17), wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively; s is a phosphorothioate linkage; (Uhd) is 2′-O-hexadecyl-uridine-3′-phosphate; and VP is a vinyl phosphonate.

In another aspect, the present invention provides a pharmaceutical composition comprising any of the double stranded RNAi agent of the invention.

In another aspect, the present invention provides a method of inhibiting transthyretin (TTR) expression in an ocular cell, the method comprising contacting the cell with the double stranded RNAi agent of the invention, thereby inhibiting expression of the TTR gene in the ocular cell.

In certain embodiments, the cell is within a subject.

In certain embodiments, the subject is a human.

In certain embodiments, the subject suffers from TTR-associated ocular disease.

In yet another aspect, the present invention provides a method of treating a subject suffering from a TTR-associated ocular disease, comprising administering to the subject a therapeutically effective amount of a double stranded RNAi agent of the invention.

In certain embodiments, the TTR-associated ocular disease or disorder is selected from the group consisting of TTR-associated glaucoma, TTR-associated vitreous opacities, TTR-associated retinal abnormalities, TTR-associated retinal amyloid deposit, TTR-associated retinal angiopathy, TTR-associated iris amyloid deposit, TTR-associated scalloped iris, and TTR-associated amyloid deposits on lens.

In certain embodiments, the subject carries a TTR gene mutation that is associated with the development of a TTR-associated disease.

In certain embodiments, the TTR-associated disease is selected from the group consisting of senile systemic amyloidosis (SSA), systemic familial amyloidosis, familial amyloidotic polyneuropathy (FAP), familial amyloidotic cardiomyopathy (FAC), leptomeningeal/Central Nervous System (CNS) amyloidosis, and hyperthyroxinemia.

In certain embodiments, the double stranded RNAi agent is administered to the subject via periocular, conjunctival, subtenon, intracameral, intravitreal, intraocular, anterior or posterior juxtascleral, subretinal, subconjunctival, retrobulbar, or intracanalicular administration.

In certain embodiments, the double stranded RNAi agent is chronically administered to the human subject.

In certain embodiments, the method further comprises administering to the subject an additional therapeutic agent.

In certain embodiments, the additional therapeutic agent is a TTR tetramer stabilizer and/or a non-steroidal anti-inflammatory agent.

In certain embodiments, the subject has received, or will receive a liver transplant.

In certain embodiments, the subject is administered a fixed dose of about 0.01 mg to about 1 mg of the double stranded RNAi agent. In certain embodiments, the subject is administered a fixed dose of about 0.001 mg to about 1 mg of the double stranded RNAi agent. In certain embodiments, the subject is administered a fixed dose of about 0.001 mg to about 0.1 mg of the double stranded RNAi agent.

In certain embodiments, the administration of the double stranded RNAi agent to the subject reduces transthyretin-mediated amyloidosis (ATTR amyloidosis) in the ciliary epithelium (CE) and retinal pigment epithelium (RPE) of subject's eye.

The present invention is further illustrated by the following detailed description and drawings.

### BRIEF DESCRIPTION OF THE DRAWINGS

FIG. 1 is a graph depicting the inhibition of ocular TTR expression in rat eyes following intravitreal administration of a single 50 μg dose of the indicated dsRNA agents.

FIG. 2A is a graph depicting the inhibition of TTR in the posterior ocular tissues of rats following intravitreal administration of a single 50 μg dose of the indicated dsRNA agents.

FIG. 2B is a graph depicting the inhibition of TTR expression in the anterior ocular tissues of rats following intravitreal administration of a single 50 μg dose of the indicated dsRNA agents.

FIG. 2C is an image of a histopathological analysis of ocular tissues in rat intravitreally administered PBS as a control.

FIG. 2D is an image of a histopathological analysis of ocular tissues in rat intravitreally administered a single 50 μg dose of the indicated dsRNA agent.

FIG. 3A is a graph depicting the inhibition of ocular human TTR expression in transgenic mouse eyes following intravitreal administration of a single 2.5 μg or 7.5 μg dose of AD-AD-70191.

FIG. 3B is a graph depicting the inhibition of ocular mouse TTR expression in transgenic mouse eyes following intravitreal administration of a single 2.5 μg or 7.5 μg dose of AD-70191.

FIG. 3C is a graph depicting the inhibition of ocular mouse cone-rod homeobox expression in transgenic mouse eyes following intravitreal administration of a single 2.5 μg or 7.5 μg dose of AD-70191.

FIG. 3D is a graph depicting the inhibition of ocular mouse rhodopsin expression in transgenic mouse eyes following intravitreal administration of a single 2.5 μg or 7.5 μg dose of AD-70191.

FIG. 4 is a graph depicting the inhibition of ocular TTR expression in the retinal pigmented epithelium (RPE) and ciliary epithelium (CE) of non-human primates following intravitreal administration of a single 3 mg dose of AD-291845 or AD-70500.

FIG. 5A is an image of an immunohistochemical (IHC) analysis of TTR protein expression in ocular tissues of non-human primates following intravitreal administration of PBS as a control. The RPE is at the bottom of the image and TTR staining is dark and medium gray.

FIG. 5B is an image of an immunohistochemical (IHC) analysis of TTR protein expression in ocular tissues of non-human primates following intravitreal administration of a single 3 mg dose of AD-291845. The RPE is at the bottom of the image and TTR staining is dark and medium gray.

FIG. 6A is a graph depicting the inhibition of ocular TTR mRNA expression in the ciliary body (CE) or retinal pigmented epithelium (RPE) of non-human primates following intravitreal administration of PBS or a single 0.1 mg, 0.3 mg, 1.0 mg, or 3.0 mg dose of AD-291845 at Day 28 post-administration.

FIG. 6B is a graph depicting the inhibition of ocular TTR protein expression in the vitreous humor of non-human primates following intravitreal administration of PBS or a single 0.1 mg, 0.3 mg, 1.0 mg, or 3.0 mg dose of AD-291845 at Day 28 post-administration.

FIG. 6C is a graph depicting the inhibition of ocular TTR protein expression in the aqueous humor of non-human primates following intravitreal administration of PBS or a single 0.1 mg, 0.3 mg, 1.0 mg, or 3.0 mg dose of AD-291845 at Day 28 post-administration.

FIG. 7A is a graph depicting the inhibition of ocular TTR mRNA expression in the retinal pigmented epithelium (RPE) of non-human primates following intravitreal administration of PBS or a single 1.0 mg or 3.0 mg dose of AD-291845 at Day 84 post-administration.

FIG. 7B is a graph depicting the inhibition of ocular TTR mRNA expression in the ciliary body (CE) of non-human primates following intravitreal administration of PBS or a single 1.0 mg or 3.0 mg dose of AD-291845 at Day 84 post-administration.

FIG. 7C is a graph depicting the inhibition of ocular TTR protein expression in the vitreous humor of non-human primates following intravitreal administration of PBS, a single 0.1 mg or 0.3 mg dose of AD-291845 at Day 28, or a single 1.0 mg or 3.0 mg dose of AD-291845 at Days 28, 56, and 84 post-administration.

FIG. 7D is a graph depicting the inhibition of ocular TTR protein expression in the aqueous humor of non-human primates following intravitreal administration of PBS, a single 0.1 mg or 0.3 mg dose of AD-291845 at Day 28, or a single 1.0 mg or 3.0 mg dose of AD-291845 at Days 28, 56, and 84 post-administration.

FIG. 8A is a graph depicting the inhibition of ocular TTR protein expression in the aqueous humor of non-human primates following intravitreal administration of PBS or a single 0.003 mg, 0.03 mg, 0.1 mg, or 0.3 mg dose of AD-291845 at Day 28 post-administration.

FIG. 8B is a graph depicting the inhibition of ocular TTR protein expression in the aqueous humor of non-human primates following intravitreal administration of PBS or a single 0.003 mg, 0.03 mg, 0.1 mg, or 0.3 mg dose of AD-291845 at the Day 28, day 84, and day 168 post-administration.

FIG. 8C is a graph depicting the inhibition of ocular TTR protein expression in the ciliary body of non-human primates following intravitreal administration of PBS or a single 0.003 mg, 0.03 mg, 0.1 mg, or 0.3 mg dose of AD-291845 at Day 168 post-administration.

FIG. 8D is a graph depicting the inhibition of ocular TTR protein expression in the retinal pigment epithelia (RPE) of non-human primates following intravitreal administration of PBS or a single 0.003 mg, 0.03 mg, 0.1 mg, or 0.3 mg dose of AD-291845 at Day 168 post-administration.

FIG. 9A is a graph depicting the inhibition of ocular TTR protein expression in the aqueous humor of non-human primates following intravitreal administration of PBS or a single 1.0 mg dose of AD-592744, AD-538697, or AD-597979 at the Day 28, Day 84, and Day 168 post-administration.

FIG. 9B is a graph depicting the inhibition of ocular TTR protein expression in the ciliary body of non-human primates following intravitreal administration of PBS or a single 1.0 mg dose of AD-592744, AD-538697, or AD-597979 at Day 168 post-administration.

FIG. 9C is a graph depicting the inhibition of ocular TTR protein expression in the retinal pigment epithelia (RPE) of non-human primates following intravitreal administration of PBS or a single 1.0 mg dose of AD-592744, AD-538697, or AD-597979 at Day 168 post-administration.

FIG. 10 is a graph depicting the inhibition of ocular TTR protein expression in the aqueous humor of non-human primates following intravitreal administration of PBS or a single dose of AD-538697, AD-579797, AD-291845, AD291846, or AD-592744 at the indicated dose.

### DETAILED DESCRIPTION OF THE INVENTION

The present invention provides RNAi agents, e.g., double stranded RNAi agents, and compositions targeting the Transthyretin (TTR) gene. The present invention also provides methods of inhibiting expression of TTR and methods of treating or preventing a TTR-associated ocular disease in a subject using the RNAi agents, e.g., double stranded RNAi agents, of the invention. The present invention is based, at least in part, on the discovery that conjugating a lipophilic moiety to one or more internal positions on at least one strand of the double-stranded iRNA agent targeting TTR, or one or more positions on at least one strand within the double stranded region of a double-stranded iRNA agent targeting TTR, provides surprisingly good results for in vivo intravitreal delivery of the double-stranded iRNAs, resulting in efficient entry of ocular tissues and efficient internalization into cells of the ocular system.

The following detailed description discloses how to make and use compositions containing iRNAs to selectively inhibit the expression of a TTR gene in an ocular cell, as well as compositions, uses, and methods for treating subjects having TTR-associated ocular diseases and disorders that would benefit from inhibition and/or reduction of the expression of a TTR gene in an ocular cell.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise.

The term “about” is used herein to mean within the typical ranges of tolerances in the art. For example, “about” can be understood as within about 2 standard deviations from the mean. In certain embodiments, about means +10%. In certain embodiments, about means +5%. When about is present before a series of numbers or a range, it is understood that “about” can modify each of the numbers in the series or range.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 19 nucleotides of a 21 nucleotide nucleic acid molecule” means that 19, 20, or 21 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, a duplex with an overhang of “no more than 2 nucleotides” has a 2, 1, or 0 nucleotide overhang. When “no more than” is present before a series of numbers or a range, it is understood that “no more than” can modify each of the numbers in the series or range. As used herein, ranges include both the upper and lower limit.

In the event of a conflict between a sequence and its indicated site on a transcript or other sequence, the nucleotide sequence recited in the specification takes precedence.

As used herein, a “transthyretin” (“TTR”) refers to the well known gene and protein. TTR is also known as prealbumin, HsT2651, PALB, and TBPA. TTR functions as a transporter of retinol-binding protein (RBP), thyroxine (T4) and retinol, and it also acts as a protease. The liver secretes TTR into the blood, and the choroid plexus secretes TTR into the cerebrospinal fluid. TTR is also expressed in the pancreas and the retinal pigment epithelium. The greatest clinical relevance of TTR is that both normal and mutant TTR protein can form amyloid fibrils that aggregate into extracellular deposits, causing amyloidosis. See, e.g., Saraiva M. J. M. (2002) Expert Reviews in Molecular Medicine, 4(12):1-11 for a review. The molecular cloning and nucleotide sequence of rat transthyretin, as well as the distribution of mRNA expression, was described by Dickson, P. W. et al. (1985) J. Biol. Chem. 260(13)8214-8219. The X-ray crystal structure of human TTR was described in Blake, C. C. et al. (1974) J Mol Biol 88, 1-12. The sequence of a human TTR mRNA transcript can be found at National Center for Biotechnology Information (NCBI) RefSeq accession number NM_000371 (e.g., SEQ ID NOs:1 and 5). The sequence of mouse TTR mRNA can be found at RefSeq accession number NM_013697.2, and the sequence of rat TTR mRNA can be found at RefSeq accession number NM_012681.1. Additional examples of TTR mRNA sequences are readily available using publicly available databases, e.g., GenBank, UniProt, and OMIM.

As used herein, “target sequence” refers to a contiguous portion of the nucleotide sequence of an mRNA molecule formed during the transcription of a TTR gene, including mRNA that is a product of RNA processing of a primary transcription product. In one embodiment, the target portion of the sequence will be at least long enough to serve as a substrate for iRNA-directed cleavage at or near that portion of the nucleotide sequence of an mRNA molecule formed during the transcription of a TTR gene. In one embodiment, the target sequence is within the protein coding region of the TTR gene. In another embodiment, the target sequence is within the 3′ UTR of the TTR gene.

The target sequence may be from about 9-36 nucleotides in length, e.g., about 15-30 nucleotides in length. For example, the target sequence can be from about 15-30 nucleotides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. In some embodiments, the target sequence is about 19 to about 30 nucleotides in length. In other embodiments, the target sequence is about 19 to about 25 nucleotides in length. In still other embodiments, the target sequence is about 19 to about 23 nucleotides in length. In some embodiments, the target sequence is about 21 to about 23 nucleotides in length. Ranges and lengths intermediate to the above recited ranges and lengths are also contemplated to be part of the invention.

In some embodiments of the invention, the target sequence of a TTR gene comprises nucleotides 615-637 of SEQ ID NO:1 or nucleotides 505-527 of SEQ ID NO:5 (i.e., 5′-GATGGGATTTCATGTAACCAAGA-3′; SEQ ID NO:4).

As used herein, the term “strand comprising a sequence” refers to an oligonucleotide comprising a chain of nucleotides that is described by the sequence referred to using the standard nucleotide nomenclature.

“G,” “C,” “A,” “T” and “U” each generally stand for a nucleotide that contains guanine, cytosine, adenine, thymidine and uracil as a base, respectively. However, it will be understood that the term “ribonucleotide” or “nucleotide” can also refer to a modified nucleotide, as further detailed below, or a surrogate replacement moiety (see, e.g., Table 3). The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention.

The terms “iRNA,” “RNAi agent,” “iRNA agent,”, “RNA interference agent” as used interchangeably herein, refer to an agent that contains RNA as that term is defined herein, and which mediates the targeted cleavage of an RNA transcript via an RNA-induced silencing complex (RISC) pathway. iRNA directs the sequence-specific degradation of mRNA through a process known as RNA interference (RNAi). The iRNA modulates, e.g., inhibits, the expression of a TTR gene in a cell, e.g., a cell within a subject, such as a mammalian subject.

In one embodiment, an RNAi agent of the invention includes a single stranded RNA that interacts with a target RNA sequence, e.g., a TTR target mRNA sequence, to direct the cleavage of the target RNA. Without wishing to be bound by theory it is believed that long double stranded RNA introduced into cells is broken down into double stranded short interfering RNAs (siRNAs) comprising a sense strand and an antisense strand by a Type III endonuclease known as Dicer (Sharp et al. (2001) Genes Dev. 15:485). Dicer, a ribonuclease-III-like enzyme, processes these dsRNA into 19-23 base pair short interfering RNAs with characteristic two base 3′ overhangs (Bernstein, et al., (2001) Nature 409:363). These siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect the invention relates to a single stranded siRNA (ssRNA) (the antisense strand of an siRNA duplex) generated within a cell and which promotes the formation of a RISC complex to effect silencing of the target gene, i.e., a TTR gene. Accordingly, the term “siRNA” is also used herein to refer to an RNAi as described above.

In another embodiment, the RNAi agent may be a single-stranded RNA that is introduced into a cell or organism to inhibit a target mRNA. Single-stranded RNAi agents bind to the RISC endonuclease, Argonaute 2, which then cleaves the target mRNA. The single-stranded siRNAs are generally 15-30 nucleotides and are chemically modified. The design and testing of single-stranded siRNAs are described in U.S. Pat. No. 8,101,348 and in Lima et al., (2012) Cell 150:883-894, the entire contents of each of which are hereby incorporated herein by reference. Any of the antisense nucleotide sequences described herein may be used as a single-stranded RNA as described herein or as chemically modified by the methods described in Lima et al., (2012) Cell 150:883-894.

In another embodiment, an “iRNA” for use in the compositions, uses, and methods of the invention is a double stranded RNA and is referred to herein as a “double stranded RNAi agent,” “double stranded RNA (dsRNA) molecule,” “dsRNA agent,” or “dsRNA”. The term “dsRNA” refers to a complex of ribonucleic acid molecules, having a duplex structure comprising two anti-parallel and substantially complementary nucleic acid strands, referred to as having “sense” and “antisense” orientations with respect to a target RNA, i.e., a TTR gene. In some embodiments of the invention, a double stranded RNA (dsRNA) triggers the degradation of a target RNA, e.g., an mRNA, through a post-transcriptional gene-silencing mechanism referred to herein as RNA interference or RNAi.

In general, the majority of nucleotides of each strand of a dsRNA molecule are ribonucleotides, but as described in detail herein, each or both strands can also include one or more non-ribonucleotides, e.g., a deoxyribonucleotide and/or a modified nucleotide. In addition, as used in this specification, an “RNAi agent” may include ribonucleotides with chemical modifications; an RNAi agent may include substantial modifications at multiple nucleotides.

As used herein, the term “modified nucleotide” refers to a nucleotide having, independently, a modified sugar moiety, a modified internucleotide linkage, and/or a modified nucleobase. Thus, the term modified nucleotide encompasses substitutions, additions or removal of, e.g., a functional group or atom, to internucleoside linkages, sugar moieties, or nucleobases. The modifications suitable for use in the agents of the invention include all types of modifications disclosed herein or known in the art. Any such modifications, as used in a siRNA type molecule, are encompassed by “RNAi agent” for the purposes of this specification and claims.

The duplex region may be of any length that permits specific degradation of a desired target RNA through a RISC pathway, and may range from about 9 to 36 base pairs in length, e.g., about 15-30 base pairs in length, for example, about 9, 10, 11, 12, 13, 14, 15, 16, 17, 18, 19, 20, 21, 22, 23, 24, 25, 26, 27, 28, 29, 30, 31, 32, 33, 34, 35, or 36 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated to be part of the invention.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” A hairpin loop can comprise at least one unpaired nucleotide. In some embodiments, the hairpin loop can comprise at least 2, at least 3, at least 4, at least 5, at least 6, at least 7, at least 8, at least 9, at least 10, at least 20, at least 23 or more unpaired nucleotides. In some embodiments, the hairpin loop can be 10 or fewer nucleotides. In some embodiments, the hairpin loop can be 8 or fewer unpaired nucleotides. In some embodiments, the hairpin loop can be 4-10 unpaired nucleotides. In some embodiments, the hairpin loop can be 4-8 nucleotides.

In certain embodiment, the two strands of double-stranded oligomeric compound can be linked together. The two strands can be linked to each other at both ends, or at one end only. By linking at one end is meant that 5′-end of first strand is linked to the 3′-end of the second strand or 3′-end of first strand is linked to 5′-end of the second strand. When the two strands are linked to each other at both ends, 5′-end of first strand is linked to 3′-end of second strand and 3′-end of first strand is linked to 5′-end of second strand. The two strands can be linked together by an oligonucleotide linker including, but not limited to, (N)n; wherein N is independently a modified or unmodified nucleotide and n is 3-23. In some embodiments, n is 3-10, e.g., 3, 4, 5, 6, 7, 8, 9, or 10. In some embodiments, the oligonucleotide linker is selected from the group consisting of GNRA, (G)4, (U)4, and (dT)4, wherein N is a modified or unmodified nucleotide and R is a modified or unmodified purine nucleotide. Some of the nucleotides in the linker can be involved in base-pair interactions with other nucleotides in the linker. The two strands can also be linked together by a non-nucleosidic linker, e.g. a linker described herein. It will be appreciated by one of skill in the art that any oligonucleotide chemical modifications or variations describe herein can be used in the oligonucleotide linker.

Hairpin and dumbbell type oligomeric compounds will have a duplex region equal to or at least 14, 15, 15, 16, 17, 18, 19, 29, 21, 22, 23, 24, or 25 nucleotide pairs. The duplex region can be equal to or less than 200, 100, or 50, in length. In some embodiments, ranges for the duplex region are 15-30, 17 to 23, 19 to 23, and 19 to 21 nucleotides pairs in length.

The hairpin oligomeric compounds can have a single strand overhang or terminal unpaired region, in some embodiments at the 3′, and in some embodiments on the antisense side of the hairpin. In some embodiments, the overhangs are 1-4, more generally 2-3 nucleotides in length. The hairpin oligomeric compounds that can induce RNA interference are also referred to as “shRNA” herein.

Where the two substantially complementary strands of a dsRNA are comprised by separate RNA molecules, those molecules need not, but can be covalently connected. Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, an RNAi may comprise one or more nucleotide overhangs.

In one embodiment, an RNAi agent of the invention is a dsRNA, each strand of which is 24-30 nucleotides in length, that interacts with a target RNA sequence, e.g., a TTR target mRNA sequence, to direct the cleavage of the target RNA. Without wishing to be bound by theory, long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188).

In one embodiment, an RNAi agent of the invention is a dsRNA agent, each strand of which comprises 19-23 nucleotides that interacts with a TTR RNA sequence to direct the cleavage of the target RNA. Without wishing to be bound by theory, long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). In one embodiment, an RNAi agent of the invention is a dsRNA of 24-30 nucleotides that interacts with a TTR RNA sequence to direct the cleavage of the target RNA.

As used herein, the term “nucleotide overhang” refers to at least one unpaired nucleotide that protrudes from the duplex structure of an iRNA, e.g., a dsRNA. For example, when a 3′-end of one strand of a dsRNA extends beyond the 5′-end of the other strand, or vice versa, there is a nucleotide overhang. A dsRNA can comprise an overhang of at least one nucleotide; alternatively, the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′-end, 3′-end or both ends of either an antisense or sense strand of a dsRNA. In one embodiment of the dsRNA, at least one strand comprises a 3′ overhang of at least 1 nucleotide. In another embodiment, at least one strand comprises a 3′ overhang of at least 2 nucleotides, e.g., 2, 3, 4, 5, 6, 7, 9, 10, 11, 12, 13, 14, or 15 nucleotides. In other embodiments, at least one strand of the RNAi agent comprises a 5′ overhang of at least 1 nucleotide. In certain embodiments, at least one strand comprises a 5′ overhang of at least 2 nucleotides, e.g., 2, 3, 4, 5, 6, 7, 9, 10, 11, 12, 13, 14, or 15 nucleotides. In still other embodiments, both the 3′ and the 5′ end of one strand of the RNAi agent comprise an overhang of at least 1 nucleotide.

In one embodiment, the antisense strand of a dsRNA has a 1-10 nucleotide, e.g., 0-3, 1-3, 2-4, 2-5, 4-10, 5-10, e.g., a 1, 2, 3, 4, 5, 6, 7, 8, 9, or 10 nucleotide, overhang at the 3′-end and/or the 5′-end. In one embodiment, the sense strand of a dsRNA has a 1-10 nucleotide, e.g., a 1, 2, 3, 4, 5, 6, 7, 8, 9, or 10 nucleotide, overhang at the 3′-end and/or the 5′-end. In another embodiment, one or more of the nucleotides in the overhang is replaced with a nucleoside thiophosphate.

In certain embodiments, the overhang on the sense strand or the antisense strand, or both, can include extended lengths longer than 10 nucleotides, e.g., 1-30 nucleotides, 2-30 nucleotides, 10-30 nucleotides, or 10-15 nucleotides in length. In certain embodiments, an extended overhang is on the sense strand of the duplex. In certain embodiments, an extended overhang is present on the 3′end of the sense strand of the duplex. In certain embodiments, an extended overhang is present on the 5′end of the sense strand of the duplex. In certain embodiments, an extended overhang is on the antisense strand of the duplex. In certain embodiments, an extended overhang is present on the 3′end of the antisense strand of the duplex. In certain embodiments, an extended overhang is present on the 5′end of the antisense strand of the duplex. In certain embodiments, one or more of the nucleotides in the overhang is replaced with a nucleoside thiophosphate. In certain embodiments, the overhang includes a self-complementary portion such that the overhang is capable of forming a hairpin structure that is stable under physiological conditions.

“Blunt” or “blunt end” means that there are no unpaired nucleotides at that end of the double stranded RNAi agent, i.e., no nucleotide overhang. A “blunt ended” RNAi agent is a dsRNA that is double stranded over its entire length, i.e., no nucleotide overhang at either end of the molecule. The RNAi agents of the invention include RNAi agents with nucleotide overhangs at one end (i.e., agents with one overhang and one blunt end) or with nucleotide overhangs at both ends.

The term “antisense strand” or “guide strand” refers to the strand of an iRNA, e.g., a dsRNA, which includes a region that is substantially complementary to a target sequence, e.g., a TTR mRNA. As used herein, the term “region of complementarity” refers to the region on the antisense strand that is substantially complementary to a sequence, for example a target sequence, e.g., a TTR nucleotide sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, 2, or 1 nucleotides of the 5′- and/or 3′-terminus of the iRNA. In one embodiment, a double stranded RNAi agent of the invention includes a nucleotide mismatch in the antisense strand. In another embodiment, a double stranded RNAi agent of the invention includes a nucleotide mismatch in the sense strand. In one embodiment, the nucleotide mismatch is, for example, within 5, 4, 3, 2, or 1 nucleotides from the 3′-terminus of the iRNA. In another embodiment, the nucleotide mismatch is, for example, in the 3′-terminal nucleotide of the iRNA.

The term “sense strand,” or “passenger strand” as used herein, refers to the strand of an iRNA that includes a region that is substantially complementary to a region of the antisense strand as that term is defined herein.

As used herein, the term “cleavage region” refers to a region that is located immediately adjacent to the cleavage site. The cleavage site is the site on the target at which cleavage occurs. In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides 10 and 11 of the antisense strand, and the cleavage region comprises nucleotides 11, 12 and 13.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an iRNA, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs and/or base pairs formed from non-natural and modified nucleotides, in so far as the above requirements with respect to their ability to hybridize are fulfilled. Such non-Watson-Crick base pairs include, but are not limited to, G:U Wobble or Hoogstein base pairing.

The terms “complementary,” “fully complementary” and “substantially complementary” herein can be used with respect to the base matching between the sense strand and the antisense strand of a dsRNA, or between the antisense strand of an iRNA agent and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding a TTR gene). For example, a polynucleotide is complementary to at least a part of a TTR mRNA if the sequence is substantially complementary to a non-interrupted portion of an mRNA encoding a TTR gene.

Accordingly, in some embodiments, the antisense polynucleotides disclosed herein are fully complementary to the target TTR sequence.

In other embodiments, the antisense polynucleotides disclosed herein are substantially complementary to the target TTR sequence and comprise a contiguous nucleotide sequence which is at least about 80% complementary over its entire length to the equivalent region of the nucleotide sequence of any one of SEQ ID NO:2, or a fragment of any one of SEQ ID NOs:1, 2, and 5, such as about 85%, about 86%, about 87%, about 88%, about 89%, about 90%, about % 91%, about 92%, about 93%, about 94%, about 95%, about 96%, about 97%, about 98%, or about 99% complementary.

In one embodiment, an RNAi agent of the invention includes a sense strand that is substantially complementary to an antisense polynucleotide which, in turn, is complementary to a target TTR sequence, and wherein the sense strand polynucleotide comprises a contiguous nucleotide sequence which is at least about 80% complementary over its entire length to the equivalent region of the nucleotide sequence of any one of the sequences in Table 4, such as about 85%, about 86%, about 87%, about 88%, about 89%, about 90%, about % 91%, about 92%, about 93%, about 94%, about 95%, about 96%, about 97%, about 98%, or about 99% complementary.

In another embodiment, an RNAi agent of the invention includes an antisense strand that is substantially complementary to the target TTR sequence and comprise a contiguous nucleotide sequence which is at least about 80% complementary over its entire length to the equivalent region of the nucleotide sequence of any one of the sequences in Table 4, such as about 85%, about 86%, about 87%, about 88%, about 89%, about 90%, about % 91%, about 92%, about 93%, about 94%, about 95%, about 96%, about 97%, about 98%, or about 99% complementary.

In some embodiments, the double-stranded region of a double-stranded iRNA agent is equal to or at least, 10, 11, 12, 13, 14, 15, 16, 17, 18, 19, 20, 21, 22, 23, 23, 24, 25, 26, 27, 28, 29, 30 or more nucleotide pairs in length.

In some embodiments, the antisense strand of a double-stranded iRNA agent is equal to or at least 14, 15, 16, 17, 18, 19, 20, 21, 22, 23, 23, 24, 25, 26, 27, 28, 29, or 30 nucleotides in length.

In some embodiments, the sense strand of a double-stranded iRNA agent is equal to or at least 10, 11, 12, 13, 14, 15, 16, 17, 18, 19, 20, 21, 22, 23, 23, 24, 25, 26, 27, 28, 29, or 30 nucleotides in length.

In one embodiment, the sense and antisense strands of the double-stranded iRNA agent are each 15 to 30 nucleotides in length.

In one embodiment, the sense and antisense strands of the double-stranded iRNA agent are each 19 to 25 nucleotides in length.

In one embodiment, the sense and antisense strands of the double-stranded iRNA agent are each 21 to 23 nucleotides in length.

In one embodiment, the sense strand of the iRNA agent is 21-nucleotides in length, and the antisense strand is 23-nucleotides in length, wherein the strands form a double-stranded region of 21 consecutive base pairs having a 2-nucleotide long single stranded overhangs at the 3-end.

In some embodiments, the majority of nucleotides of each strand are ribonucleotides, but as described in detail herein, each or both strands can also include one or more non-ribonucleotides, e.g., a deoxyribonucleotide and/or a modified nucleotide. In addition, an “iRNA” may include ribonucleotides with chemical modifications. Such modifications may include all types of modifications disclosed herein or known in the art. Any such modifications, as used in an iRNA molecule, are encompassed by “iRNA” for the purposes of this specification and claims.

In one aspect of the invention, an agent for use in the methods and compositions of the invention is a single-stranded antisense nucleic acid molecule that inhibits a target mRNA via an antisense inhibition mechanism. The single-stranded antisense RNA molecule is complementary to a sequence within the target mRNA. The single-stranded antisense oligonucleotides can inhibit translation in a stoichiometric manner by base pairing to the mRNA and physically obstructing the translation machinery, see Dias, N. et al., (2002) Mol Cancer Ther 1:347-355. The single-stranded antisense RNA molecule may be about 15 to about 30 nucleotides in length and have a sequence that is complementary to a target sequence. For example, the single-stranded antisense RNA molecule may comprise a sequence that is at least about 15, 16, 17, 18, 19, 20, or more contiguous nucleotides from any one of the antisense sequences described herein.

A “TTR-associated disease,” as used herein, is intended to include any disease associated with the TTR gene or protein. Such a disease may be caused, for example, by excess production of the TTR protein, by TTR gene mutations, by abnormal cleavage of the TTR protein, by abnormal interactions between TTR and other proteins or other endogenous or exogenous substances. A “TTR-associated disease” includes any type of TTR amyloidosis (ATTR) wherein TTR plays a role in the formation of abnormal extracellular aggregates or amyloid deposits. TTR-associated diseases include, but are not limited to, senile systemic amyloidosis (SSA), systemic familial amyloidosis, familial amyloidotic polyneuropathy (FAP), familial amyloidotic cardiomyopathy (FAC), leptomeningeal/Central Nervous System (CNS) amyloidosis, amyloidotic vitreous opacities, carpal tunnel syndrome, and hyperthyroxinemia. Symptoms of TTR amyloidosis include sensory neuropathy (e.g., paresthesia, hypesthesia in distal limbs), autonomic neuropathy (e.g., gastrointestinal dysfunction, such as gastric ulcer, or orthostatic hypotension), motor neuropathy, seizures, dementia, myelopathy, polyneuropathy, carpal tunnel syndrome, autonomic insufficiency, cardiomyopathy, vitreous opacities, renal insufficiency, nephropathy, substantially reduced mBMI (modified Body Mass Index), cranial nerve dysfunction, and corneal lattice dystrophy.

A “TTR-associated ocular disease or disorder” includes any disease or disorder associated with the TTR gene or protein in the eye. Such a disease may be caused, for example, by excess production of the TTR protein, by TTR gene mutations, by abnormal cleavage of the TTR protein, by abnormal interactions between TTR and other proteins or other endogenous or exogenous substances in the eye. A “TTR-associated ocular disease or disorder” includes any type of TTR amyloidosis (ATTR) wherein TTR plays a role in the formation of abnormal extracellular aggregates or amyloid deposits in the eye.

TTR-associated ocular diseases or disorders include, but are not limited to, TTR-associated glaucoma, TTR-associated vitreous opacities, TTR-associated retinal abnormalities, TTR-associated retinal amyloid deposit, TTR-associated retinal angiopathy, TTR-associated iris amyloid deposit, TTR-associated scalloped iris, and TTR-associated amyloid deposits on lens.

### II. Lipophilic Moieties

The present invention provides dsRNA agents comprising a sense strand and an antisense strand forming a double stranded region targeting a portion of a TTR gene, wherein one or more lipophilic moieties are conjugated to one or more internal positions on at least one strand, or one or more positions on at least one strand within the double stranded region of a double-stranded iRNA, optionally via a linker or carrier. The dsRNA agents of the invention comprising one or more lipophilic moieties conjugated to one or more internal nucleotides of at least one strand, or one or more positions on at least one strand within the double stranded region of a double-stranded iRNA, have optimal hydrophobicity for the enhanced in vivo delivery of the dsRNAs to an ocular cell.

The term “lipophile” or “lipophilic moiety” broadly refers to any compound or chemical moiety having an affinity for lipids. One way to characterize the lipophilicity of the lipophilic moiety is by the octanol-water partition coefficient, log K_{ow}, where K_{ow} is the ratio of a chemical's concentration in the octanol-phase to its concentration in the aqueous phase of a two-phase system at equilibrium. The octanol-water partition coefficient is a laboratory-measured property of a substance. However, it may also be predicted by using coefficients attributed to the structural components of a chemical which are calculated using first-principle or empirical methods (see, for example, Tetko et al., J. Chem. Inf Comput. Sci. 41:1407-21 (2001), the entire contents of which is incorporated herein by reference). It provides a thermodynamic measure of the tendency of the substance to prefer a non-aqueous or oily milieu rather than water (i.e. its hydrophilic/lipophilic balance). In principle, a chemical substance is lipophilic in character when its log K_{ow} exceeds 0. Typically, the lipophilic moiety possesses a log K_{ow} exceeding 1, exceeding 1.5, exceeding 2, exceeding 3, exceeding 4, exceeding 5, or exceeding 10. For instance, the log K_{ow} of 6-amino hexanol, for instance, is predicted to be approximately 0.7. Using the same method, the log K_{ow} of cholesteryl N-(hexan-6-ol) carbamate is predicted to be 10.7.

The lipophilicity of a molecule can change with respect to the functional group it carries. For instance, adding a hydroxyl group or amine group to the end of a lipophilic moiety can increase or decrease the partition coefficient (e.g., log K_{ow}) value of the lipophilic moiety.

Alternatively, the hydrophobicity of the double-stranded iRNA agent, conjugated to one or more lipophilic moieties, can be measured by its protein binding characteristics. For instance, the unbound fraction in the plasma protein binding assay of the double-stranded iRNA agent can be determined to positively correlate to the relative hydrophobicity of the double-stranded iRNA agent, which can positively correlate to the silencing activity of the double-stranded iRNA agent.

In one embodiment, the plasma protein binding assay determined is an electrophoretic mobility shift assay (EMSA) using human serum albumin protein. The hydrophobicity of the double-stranded iRNA agent, measured by fraction of unbound siRNA in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of siRNA.

Accordingly, conjugating the lipophilic moieties to the internal position(s) of the double-stranded iRNA agent, or position(s) within the double stranded portion of the RNAi agent, provides optimal hydrophobicity for the enhanced in vivo ocular delivery of siRNA.

In certain embodiments, the lipophilic moiety is an aliphatic, cyclic such as alicyclic, or polycyclic such as polyalicyclic compound, such as a steroid (e.g., sterol) or a linear or branched aliphatic hydrocarbon. The lipophilic moiety may generally comprise a hydrocarbon chain, which may be cyclic or acyclic. The hydrocarbon chain may comprise various substituents and/or one or more heteroatoms, such as an oxygen or nitrogen atom. Such lipophilic aliphatic moieties include, without limitation, saturated or unsaturated C₄-C₃₀ hydrocarbon (e.g., C₆-C₁₈ hydrocarbon), saturated or unsaturated fatty acids, waxes (e.g., monohydric alcohol esters of fatty acids and fatty diamides), terpenes (e.g., C₁₀ terpenes, C₁₅ sesquiterpenes, C₂₀ diterpenes, C₃₀ triterpenes, and C₄₀ tetraterpenes), and other polyalicyclic hydrocarbons. For instance, the lipophilic moiety may contain a C₄-C₃₀ hydrocarbon chain (e.g., C₄-C₃₀ alkyl or alkenyl). In some embodiment the lipophilic moiety contains a saturated or unsaturated C₆-C₁₈ hydrocarbon chain (e.g., a linear C₆-C₁₈ alkyl or alkenyl). In one embodiment, the lipophilic moiety contains a saturated or unsaturated C₁₆ hydrocarbon chain (e.g., a linear C₁₆ alkyl or alkenyl).

The lipophilic moiety may be attached to the iRNA agent by any method known in the art, including via a functional grouping already present in the lipophilic moiety or introduced into the iRNA agent, such as a hydroxy group (e.g., —CO—CH₂—OH). The functional groups already present in the lipophilic moiety or introduced into the iRNA agent include, but are not limited to, hydroxyl, amine, carboxylic acid, sulfonate, phosphate, thiol, azide, and alkyne.

Conjugation of the iRNA agent and the lipophilic moiety may occur, for example, through formation of an ether or a carboxylic or carbamoyl ester linkage between the hydroxy and an alkyl group R—, an alkanoyl group RCO— or a substituted carbamoyl group RNHCO—. The alkyl group R may be cyclic (e.g., cyclohexyl) or acyclic (e.g., straight-chained or branched; and saturated or unsaturated). Alkyl group R may be a butyl, pentyl, hexyl, heptyl, octyl, nonyl, decyl, undecyl, dodecyl, tridecyl, tetradecyl, pentadecyl, hexadecyl, heptadecyl or octadecyl group, or the like.

In some embodiments, the lipophilic moiety is conjugated to the double-stranded iRNA agent via a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

In another embodiment, the lipophilic moiety is a steroid, such as sterol. Steroids are polycyclic compounds containing a perhydro-1,2-cyclopentanophenanthrene ring system. Steroids include, without limitation, bile acids (e.g., cholic acid, deoxycholic acid and dehydrocholic acid), cortisone, digoxigenin, testosterone, cholesterol, and cationic steroids, such as cortisone. A “cholesterol derivative” refers to a compound derived from cholesterol, for example by substitution, addition or removal of substituents.

In another embodiment, the lipophilic moiety is an aromatic moiety. In this context, the term “aromatic” refers broadly to mono- and polyaromatic hydrocarbons. Aromatic groups include, without limitation, C₆-C₁₄ aryl moieties comprising one to three aromatic rings, which may be optionally substituted; “aralkyl” or “arylalkyl” groups comprising an aryl group covalently linked to an alkyl group, either of which may independently be optionally substituted or unsubstituted; and “heteroaryl” groups. As used herein, the term “heteroaryl” refers to groups having 5 to 14 ring atoms, preferably 5, 6, 9, or 10 ring atoms; having 6, 10, or 14π electrons shared in a cyclic array, and having, in addition to carbon atoms, between one and about three heteroatoms selected from the group consisting of nitrogen (N), oxygen (O), and sulfur (S).

As employed herein, a “substituted” alkyl, cycloalkyl, aryl, heteroaryl, or heterocyclic group is one having between one and about four, preferably between one and about three, more preferably one or two, non-hydrogen substituents. Suitable substituents include, without limitation, halo, hydroxy, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups.

In some embodiments, the lipophilic moiety is an aralkyl group, e.g., a 2-arylpropanoyl moiety. The structural features of the aralkyl group are selected so that the lipophilic moiety will bind to at least one protein in vivo. In certain embodiments, the structural features of the aralkyl group are selected so that the lipophilic moiety binds to serum, vascular, or cellular proteins. In certain embodiments, the structural features of the aralkyl group promote binding to albumin, an immunoglobulin, a lipoprotein, α-2-macroglobulin, or α-1-glycoprotein.

In certain embodiments, the ligand is naproxen or a structural derivative of naproxen. Procedures for the synthesis of naproxen can be found in U.S. Pat. Nos. 3,904,682 and 4,009,197, which are hereby incorporated by reference in their entirety. Naproxen has the chemical name (S)-6-Methoxy-α-methyl-2-naphthaleneacetic acid and the structure is

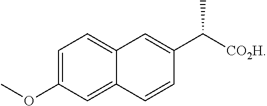 [SMILES: COc1ccc2cc([C@H](C)C(=O)O)ccc2c1]

In certain embodiments, the ligand is ibuprofen or a structural derivative of ibuprofen. Procedures for the synthesis of ibuprofen can be found in U.S. Pat. No. 3,228,831, which are hereby incorporated by reference in their entirety. The structure of ibuprofen is

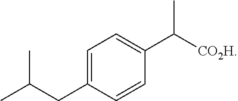

Additional exemplary aralkyl groups are illustrated in U.S. Pat. No. 7,626,014, which is incorporated herein by reference in its entirety.

In another embodiment, suitable lipophilic moieties include lipid, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, O3-(oleoyl)lithocholic acid, O3-(oleoyl)cholenic acid, ibuprofen, naproxen, dimethoxytrityl, or phenoxazine.

In certain embodiments, more than one lipophilic moieties can be incorporated into the double-strand iRNA agent, particularly when the lipophilic moiety has a low lipophilicity or hydrophobicity. In one embodiment, two or more lipophilic moieties are incorporated into the same strand of the double-strand iRNA agent. In one embodiment, each strand of the double-strand iRNA agent has one or more lipophilic moieties incorporated. In one embodiment, two or more lipophilic moieties are incorporated into the same position (i.e., the same nucleobase, same sugar moiety, or same internucleosidic linkage) of the double-strand iRNA agent. This can be achieved by, e.g., conjugating the two or more lipophilic moieties via a carrier, and/or conjugating the two or more lipophilic moieties via a branched linker, and/or conjugating the two or more lipophilic moieties via one or more linkers, with one or more linkers linking the lipophilic moieties consecutively.

The lipophilic moiety may be conjugated to the iRNA agent via a direct attachment to the ribosugar of the iRNA agent. Alternatively, the lipophilic moiety may be conjugated to the double-strand iRNA agent via a linker or a carrier.

In certain embodiments, the lipophilic moiety may be conjugated to the iRNA agent via one or more linkers (tethers).

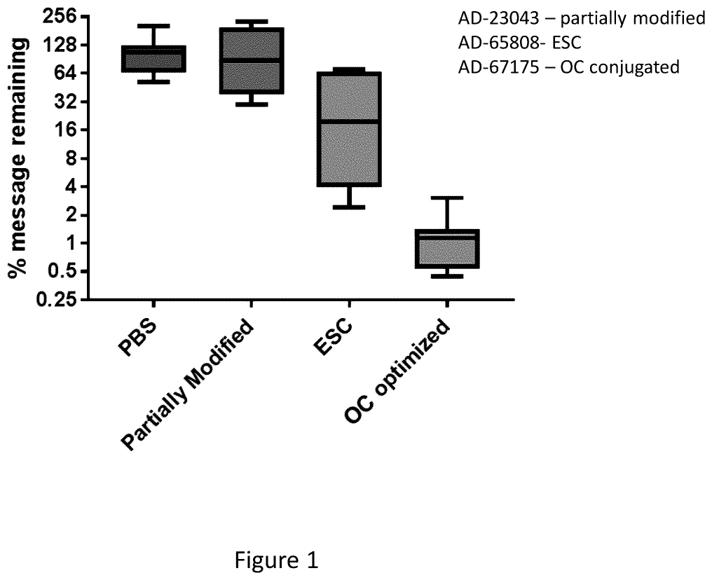

In one embodiment, the lipophilic moiety is conjugated to the double-stranded iRNA agent via a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate. Some exemplary linkages are illustrated in FIG. 1, Examples 2, 3, 5, 6, and 7.

### A. Linkers/Tethers

Linkers/Tethers are connected to the lipophilic moiety at a “tethering attachment point (TAP).” Linkers/Tethers may include any C₁-C₁₀₀ carbon-containing moiety, (e.g. C₁-C₇₅, C₁-C₅₀, C₁-C₂₀, C₁-C₁₀; C₁, C₂, C₃, C₄, C₅, C₆, C₇, C₈, C₉, or C₁₀), and may have at least one nitrogen atom. In certain embodiments, the nitrogen atom forms part of a terminal amino or amido (NHC(O)—) group on the linker/tether, which may serve as a connection point for the lipophilic moiety. Non-limited examples of linkers/tethers (underlined) include TAP-(CH₂)ₙNH—; TAP-C(O)(CH₂)ₙNH—; TAP-NR″″(CH₂)ₙNH—, TAP-C(O)—(CH₂)ₙ—C(O)—; TAP-C(O)—(CH₂)ₙ—C(O)O—; TAP-C(O)—O—; TAP-C(O)—(CH₂)ₙ—NH—C(O)—; TAP-C(O)—(CH₂)ₙ—; TAP-C(O)—NH—; TAP-C(O)—; TAP-(CH₂)ₙ—C(O)—; TAP-(CH₂)ₙ—C(O)O—; TAP-(CH₂)ₙ—; or TAP-(CH₂)ₙ—NH—C(O)—; in which n is 1-20 (e.g., 1, 2, 3, 4, 5, 6, 7, 8, 9, 10, 11, 12, 13, 14, 15, 16, 17, 18, 19, or 20) and R″″ is C₁-C₆ alkyl. Preferably, n is 5, 6, or 11. In other embodiments, the nitrogen may form part of a terminal oxyamino group, e.g., —ONH₂, or hydrazino group, —NHNH₂. The linker/tether may optionally be substituted, e.g., with hydroxy, alkoxy, perhaloalkyl, and/or optionally inserted with one or more additional heteroatoms, e.g., N, O, or S. Preferred tethered ligands may include, e.g., TAP-(CH₂)ₙNH(LIGAND); TAP-C(O)(CH₂)ₙNH(LIGAND); TAP-NR″″(CH₂)ₙNH(LIGAND); TAP-(CH₂)ₙONH(LIGAND); TAP-C(O)(CH₂)ₙONH(LIGAND); TAP-NR″″(CH₂)ₙONH(LIGAND); TAP-(CH₂)ₙNHNH₂(LIGAND), TAP-C(O)(CH₂)ₙNHNH₂(LIGAND); TAP-NR″″(CH₂)ₙNHNH₂(LIGAND); TAP-C(O)—(CH₂)ₙ—C(O)(LIGAND); TAP-C(O)—(CH₂)ₙ—C(O)O(LIGAND); TAP-C(O)—O(LIGAND); TAP-C(O)—(CH₂)ₙ—NH—C(O)(LIGAND); TAP-C(O)—(CH₂)ₙ(LIGAND); TAP-C(O)—NH(LIGAND); TAP-C(O)(LIGAND); TAP-(CH₂)ₙ—C(O) (LIGAND); TAP-(CH₂)ₙ—C(O)O(LIGAND); TAP-(CH₂)ₙ(LIGAND); or TAP-(CH₂)ₙ—NH—C(O)(LIGAND). In some embodiments, amino terminated linkers/tethers (e.g., NH₂, ONH₂, NH₂NH₂) can form an imino bond (i.e., C═N) with the ligand. In some embodiments, amino terminated linkers/tethers (e.g., NH₂, ONH₂, NH₂NH₂) can acylated, e.g., with C(O)CF₃.

In some embodiments, the linker/tether can terminate with a mercapto group (i.e., SH) or an olefin (e.g., CH═CH₂). For example, the tether can be TAP-(CH₂)ₙ—SH, TAP-C(O)(CH₂)ₙSH, TAP-(CH₂)ₙ—(CH═CH₂), or TAP-C(O)(CH₂)ₙ(CH═CH₂), in which n can be as described elsewhere. The tether may optionally be substituted, e.g., with hydroxy, alkoxy, perhaloalkyl, and/or optionally inserted with one or more additional heteroatoms, e.g., N, O, or S. The double bond can be cis or trans or E or Z.

In other embodiments, the linker/tether may include an electrophilic moiety, preferably at the terminal position of the linker/tether. Exemplary electrophilic moieties include, e.g., an aldehyde, alkyl halide, mesylate, tosylate, nosylate, or brosylate, or an activated carboxylic acid ester, e.g. an NHS ester, or a pentafluorophenyl ester. Preferred linkers/tethers (underlined) include TAP-(CH₂)ₙCHO; TAP-C(O)(CH₂)ₙCHO; or TAP-NR″″(CH₂)ₙCHO, in which n is 1-6 and R″″ is C₁-C₆ alkyl; or TAP-(CH₂)ₙC(O)ONHS; TAP-C(O)(CH₂)ₙC(O)ONHS; or TAP-NR″″(CH₂)ₙC(O)ONHS, in which n is 1-6 and R″″ is C₁-C₆ alkyl; TAP-(CH₂)ₙC(O)OC₆F₅; TAP-C(O)(CH₂)C(O)OC₆F₅; or TAP-NR″″(CH₂)C(O)OC₆F₅, in which n is 1-11 and R″″ is C₁-C₆ alkyl; or —(CH₂)ₙCH₂LG; TAP-C(O)(CH₂)ₙCH₂LG; or TAP-NR″″(CH₂)ₙCH₂LG, in which n can be as described elsewhere and R″″ is C₁-C₆ alkyl (LG can be a leaving group, e.g., halide, mesylate, tosylate, nosylate, brosylate). Tethering can be carried out by coupling a nucleophilic group of a ligand, e.g., a thiol or amino group with an electrophilic group on the tether.

In other embodiments, it can be desirable for the monomer to include a phthalimido group (K) at the terminal position of the linker/tether.

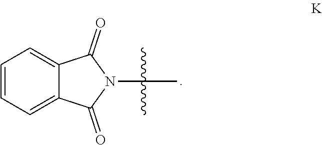 [SMILES: *N1C(=O)c2ccccc2C1=O]

In other embodiments, other protected amino groups can be at the terminal position of the linker/tether, e.g., alloc, monomethoxy trityl (MMT), trifluoroacetyl, Fmoc, or aryl sulfonyl (e.g., the aryl portion can be ortho-nitrophenyl or ortho, para-dinitrophenyl).

Any of the linkers/tethers described herein may further include one or more additional linking groups, e.g., —O—(CH₂)ₙ—, —(CH₂)ₙ—SS—, —(CH₂)ₙ—, or —(CH═CH)—.

### B. Cleavable Linkers/Tethers

In some embodiments, at least one of the linkers/tethers can be a redox cleavable linker, an acid cleavable linker, an esterase cleavable linker, a phosphatase cleavable linker, or a peptidase cleavable linker.

In one embodiment, at least one of the linkers/tethers can be a reductively cleavable linker (e.g., a disulfide group).

In one embodiment, at least one of the linkers/tethers can be an acid cleavable linker (e.g., a hydrazone group, an ester group, an acetal group, or a ketal group).

In one embodiment, at least one of the linkers/tethers can be an esterase cleavable linker (e.g., an ester group).

In one embodiment, at least one of the linkers/tethers can be a phosphatase cleavable linker (e.g., a phosphate group).

In one embodiment, at least one of the linkers/tethers can be an peptidase cleavable linker (e.g., a peptide bond).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some tethers will have a linkage group that is cleaved at a preferred pH, thereby releasing the iRNA agent from a ligand (e.g., a targeting or cell-permeable ligand, such as cholesterol) inside the cell, or into the desired compartment of the cell.

A chemical junction (e.g., a linking group) that links a ligand to an iRNA agent can include a disulfide bond. When the iRNA agent/ligand complex is taken up into the cell by endocytosis, the acidic environment of the endosome will cause the disulfide bond to be cleaved, thereby releasing the iRNA agent from the ligand (Quintana et al., Pharm Res. 19:1310-1316, 2002; Patri et al., Curr. Opin. Curr. Biol. 6:466-471, 2002). The ligand can be a targeting ligand or a second therapeutic agent that may complement the therapeutic effects of the iRNA agent.

A tether can include a linking group that is cleavable by a particular enzyme. The type of linking group incorporated into a tether can depend on the cell to be targeted by the iRNA agent. For example, an iRNA agent that targets an mRNA in liver cells can be conjugated to a tether that includes an ester group. Liver cells are rich in esterases, and therefore the tether will be cleaved more efficiently in liver cells than in cell types that are not esterase-rich. Cleavage of the tether releases the iRNA agent from a ligand that is attached to the distal end of the tether, thereby potentially enhancing silencing activity of the iRNA agent. Other cell-types rich in esterases include cells of the lung, renal cortex, and testis.

Tethers that contain peptide bonds can be conjugated to iRNA agents target to cell types rich in peptidases, such as liver cells and synoviocytes. For example, an iRNA agent targeted to synoviocytes, such as for the treatment of an inflammatory disease (e.g., rheumatoid arthritis), can be conjugated to a tether containing a peptide bond.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue, e.g., tissue the iRNA agent would be exposed to when administered to a subject. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

i. Redox Cleavable Linking Groups

One class of cleavable linking groups are redox cleavable linking groups that are cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

ii. Phosphate-Based Cleavable Linking Groups

Phosphate-based linking groups are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

iii. Acid Cleavable Linking Groups

Acid cleavable linking groups are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, ketals, acetals, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

iv. Ester-Based Linking Groups

Ester-based linking groups are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

v. Peptide-Based Cleaving Groups

Peptide-based linking groups are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide cleavable linking groups have the general formula —NHCHR¹C(O)NHCHR²C(O)—, where R¹ and R² are the R groups of the two adjacent amino acids. These candidates can be evaluated using methods analogous to those described above.

vi. Biocleavable Linkers/Tethers

The linkers can also include biocleavable linkers that are nucleotide and non-nucleotide linkers or combinations thereof that connect two parts of a molecule, for example, one or both strands of two individual siRNA molecule to generate a bis(siRNA). In some embodiments, mere electrostatic or stacking interaction between two individual siRNAs can represent a linker. The non-nucleotide linkers include tethers or linkers derived from monosaccharides, disaccharides, oligosaccharides, and derivatives thereof, aliphatic, alicyclic, heterocyclic, and combinations thereof.

In some embodiments, at least one of the linkers (tethers) is a bio-cleavable linker selected from the group consisting of DNA, RNA, disulfide, amide, functionalized monosaccharides or oligosaccharides of galactosamine, glucosamine, glucose, galactose, and mannose, and combinations thereof.

In one embodiment, the bio-cleavable carbohydrate linker may have 1 to 10 saccharide units, which have at least one anomeric linkage capable of connecting two siRNA units. When two or more saccharides are present, these units can be linked via 1-3, 1-4, or 1-6 sugar linkages, or via alkyl chains.

Exemplary bio-cleavable linkers include:

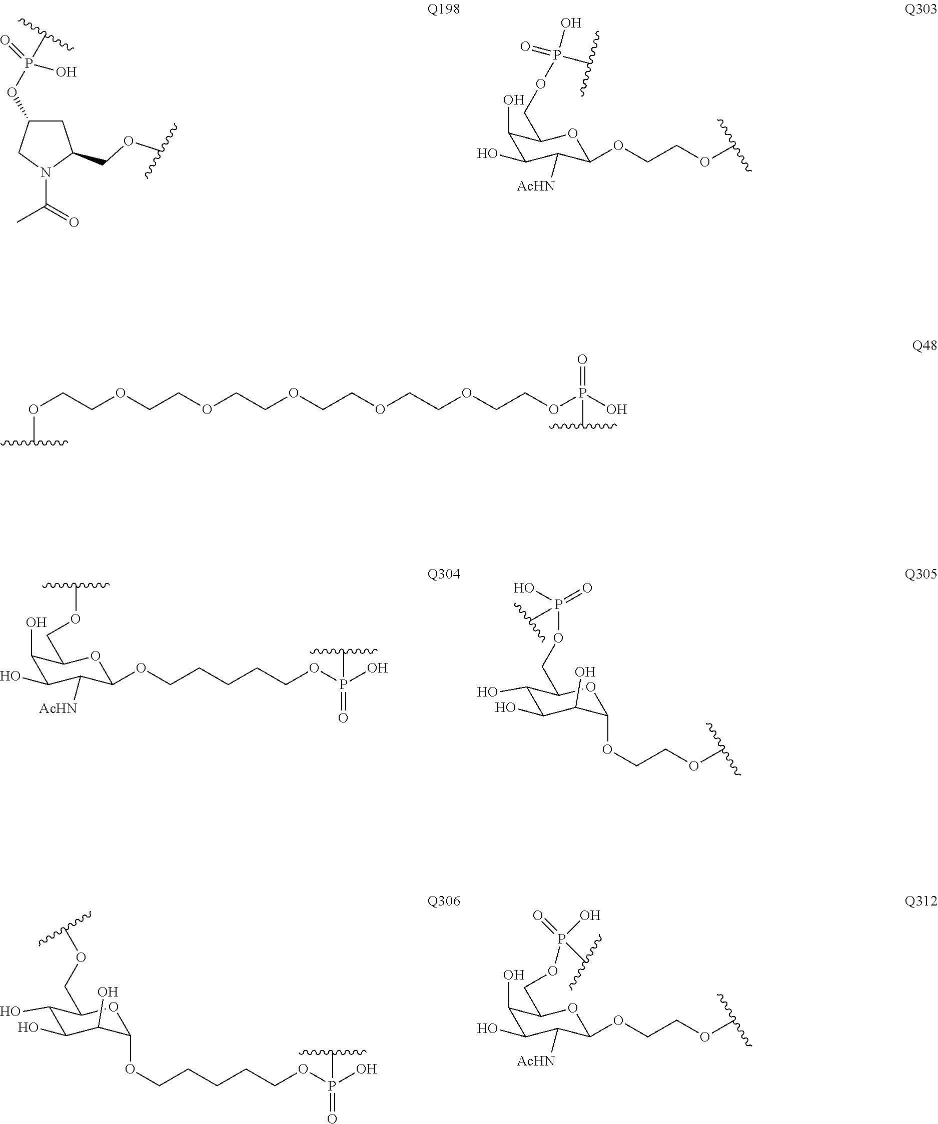

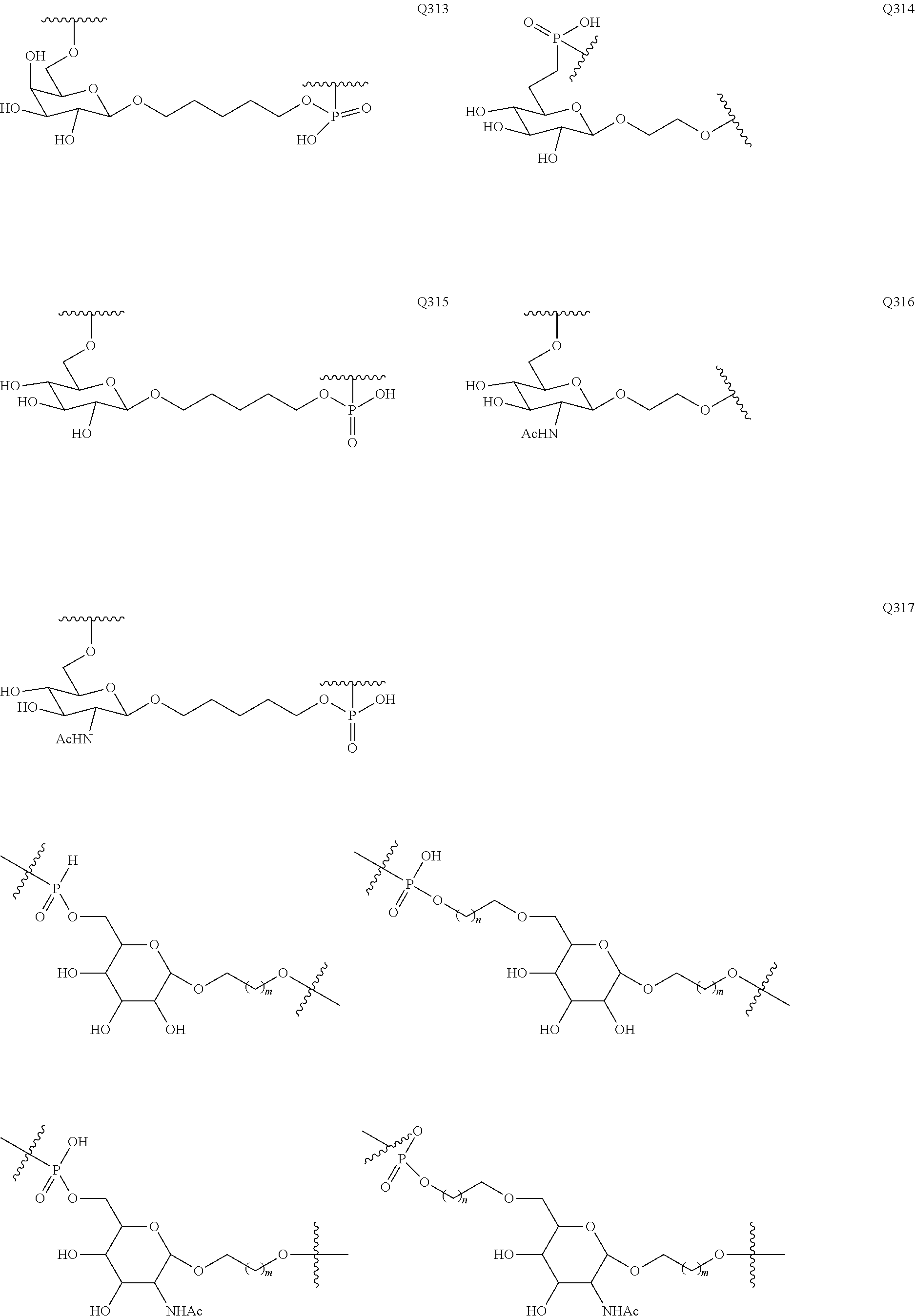

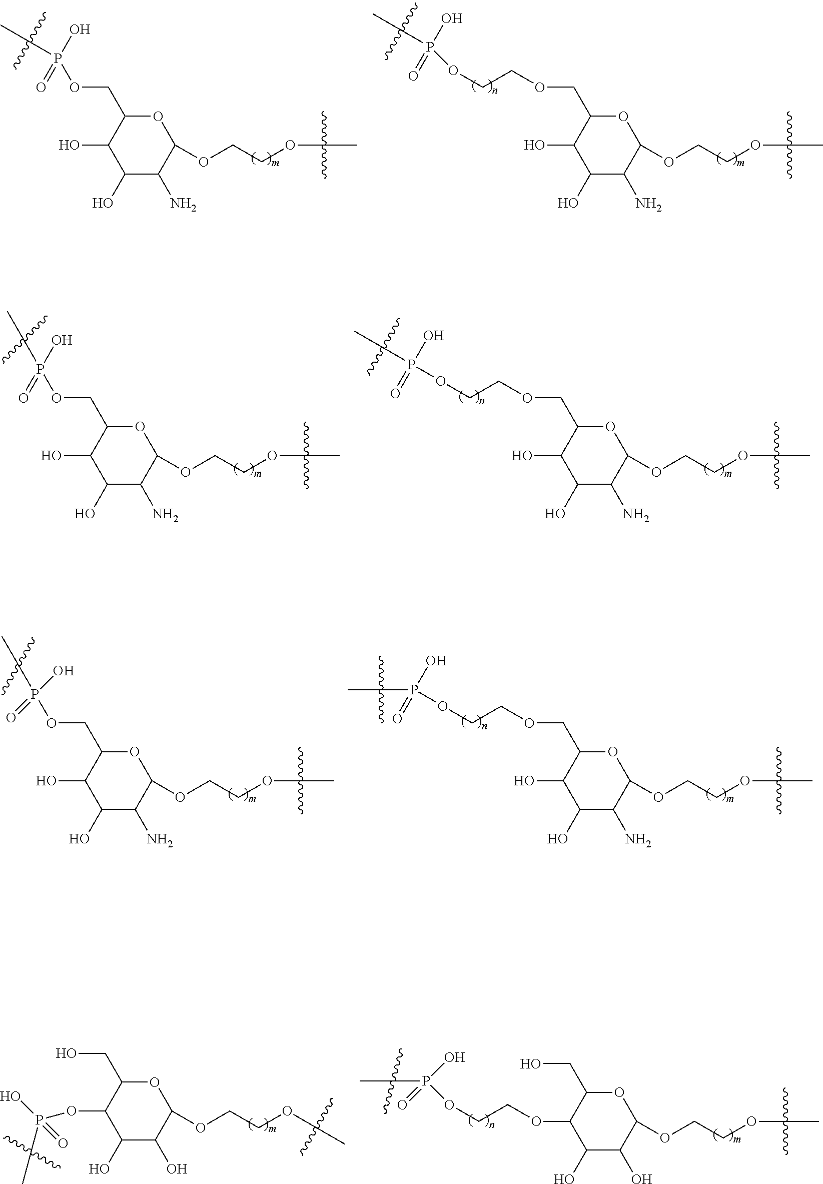

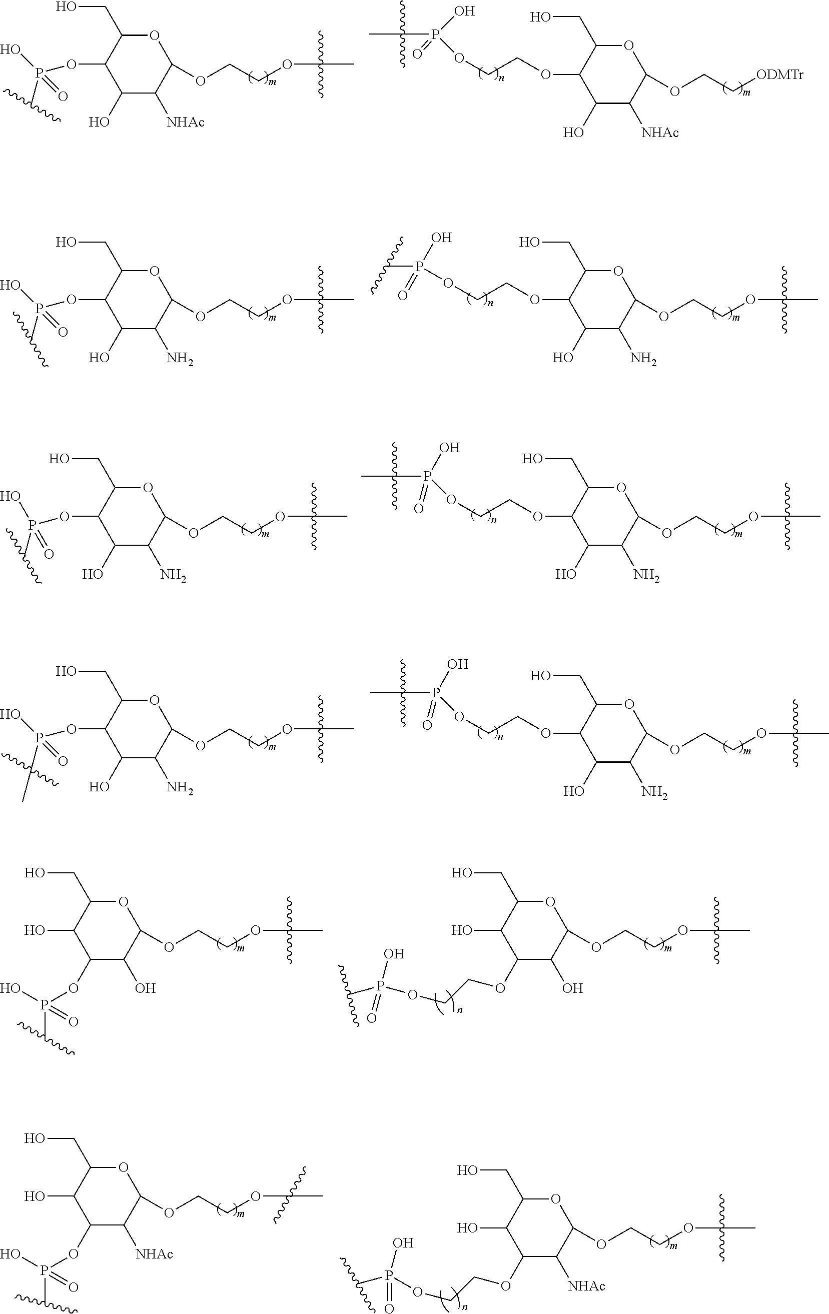

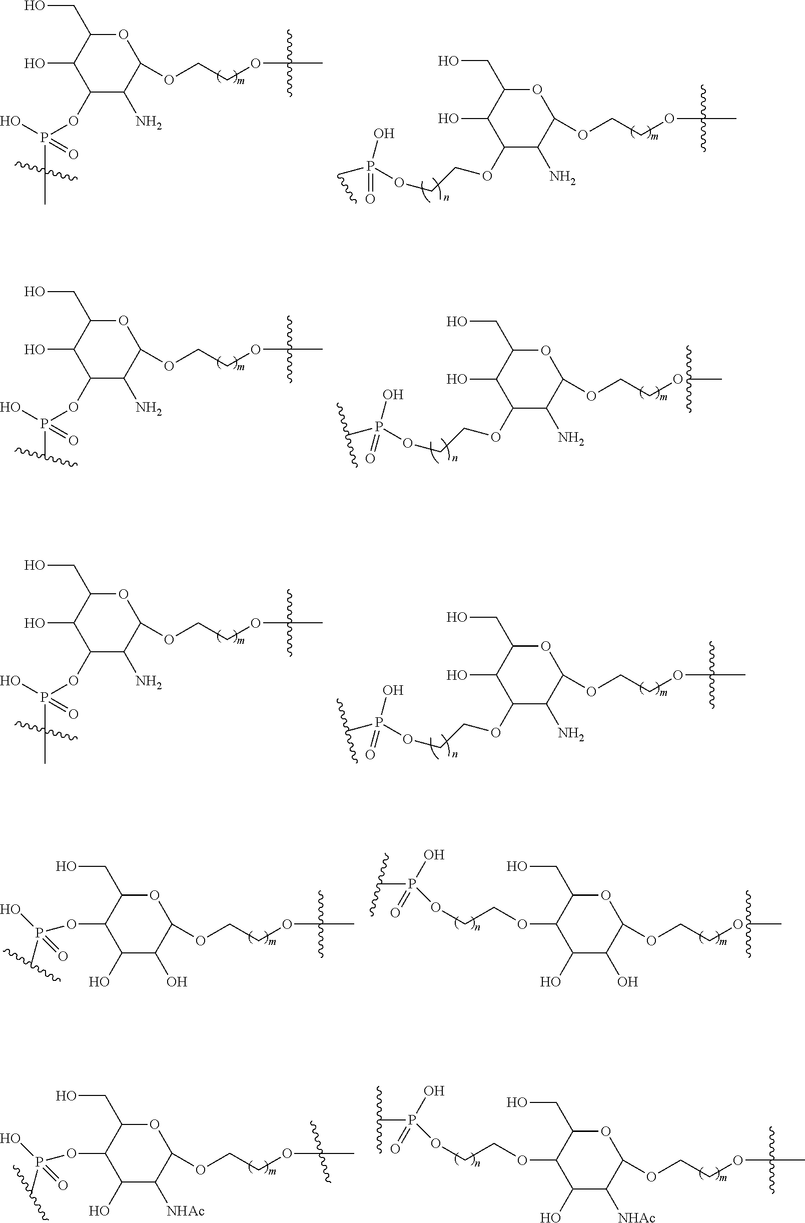

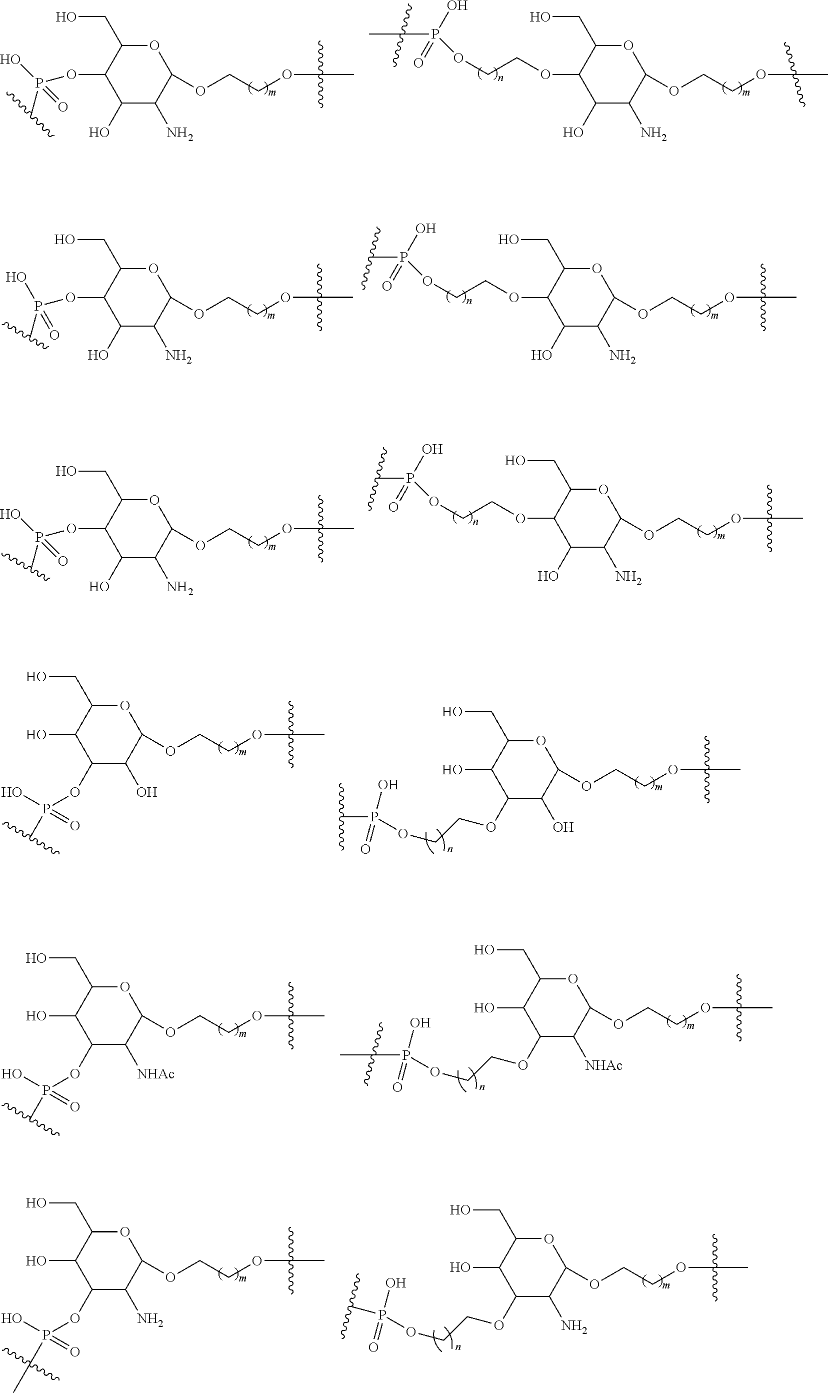

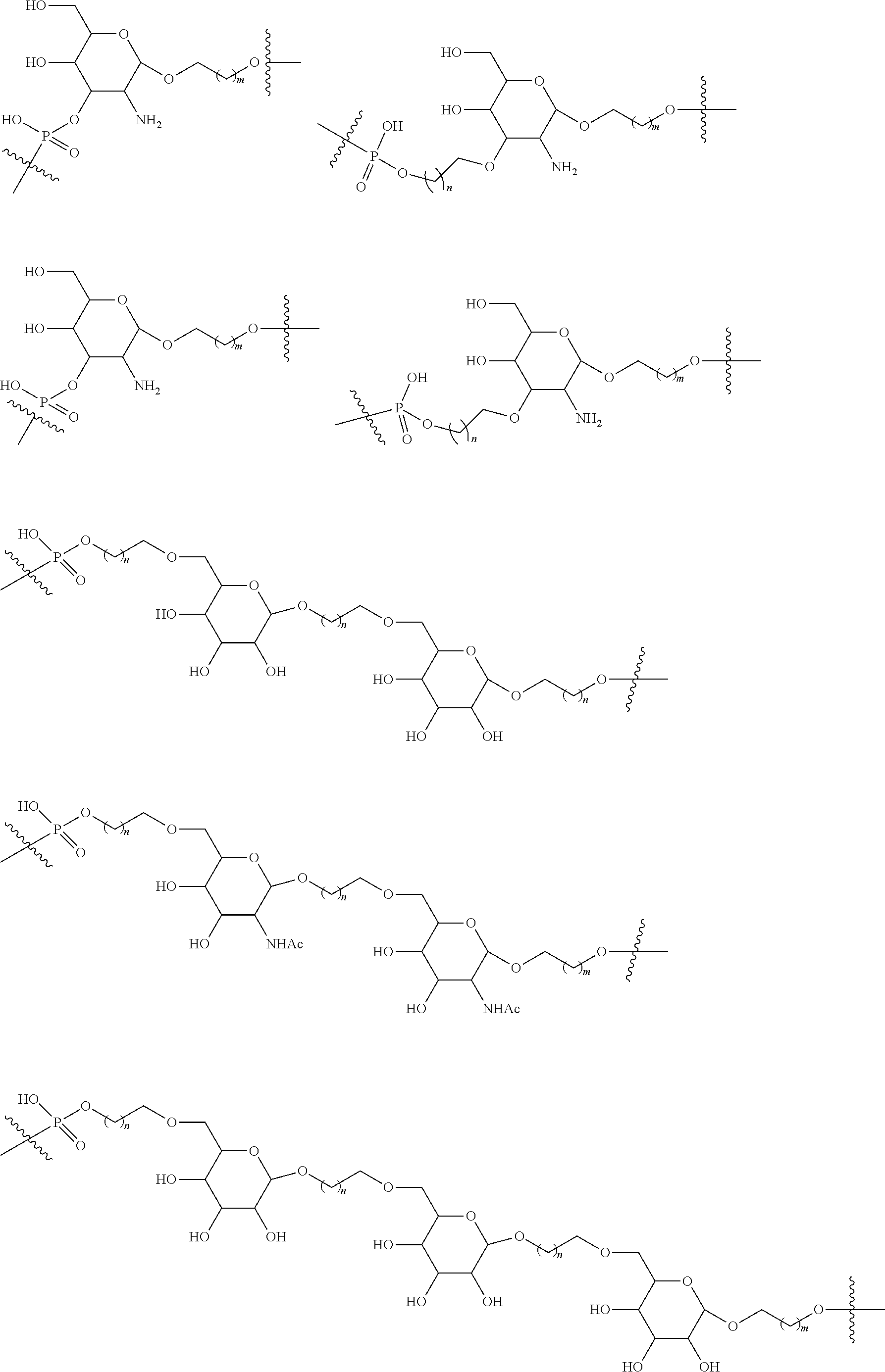

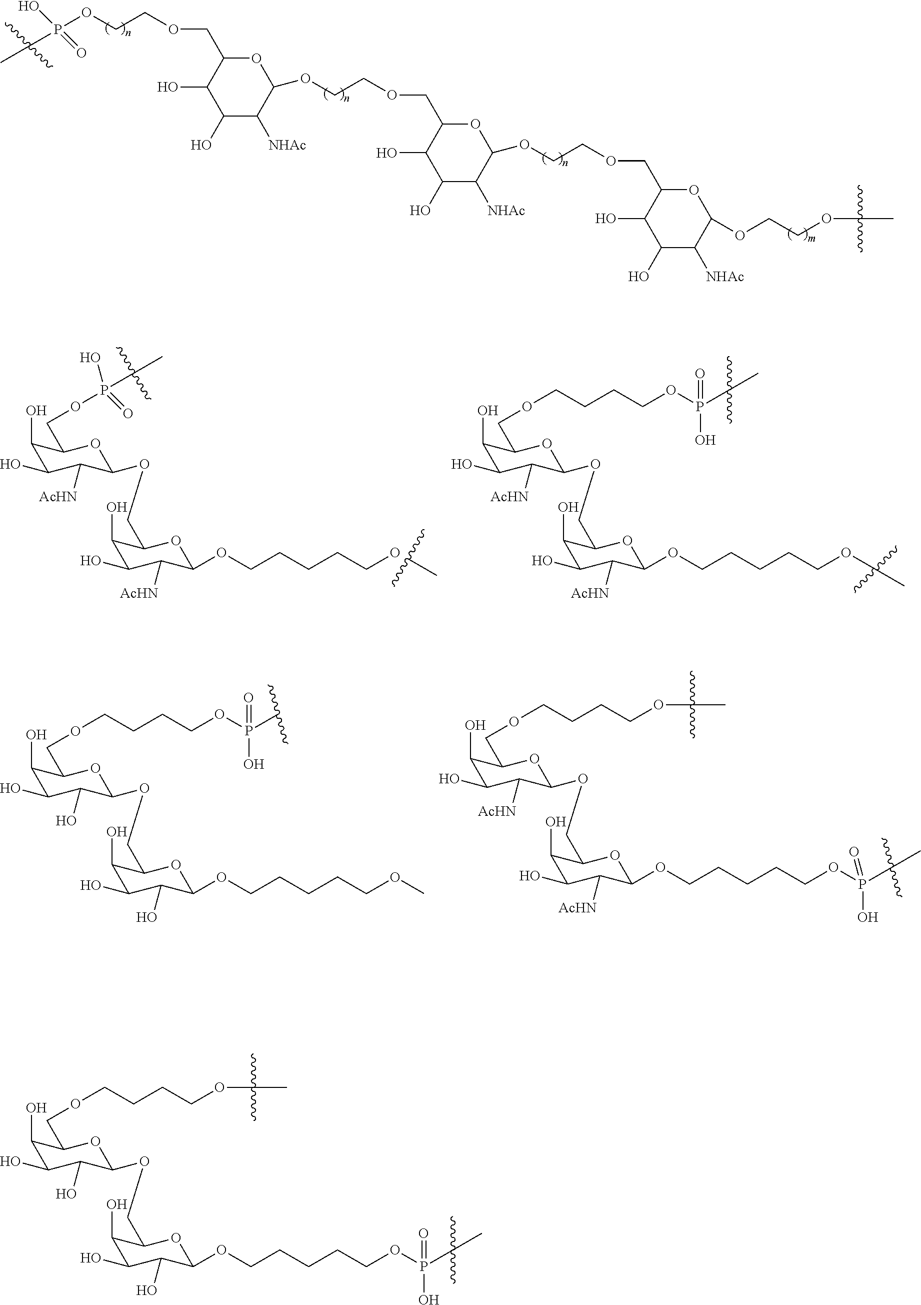

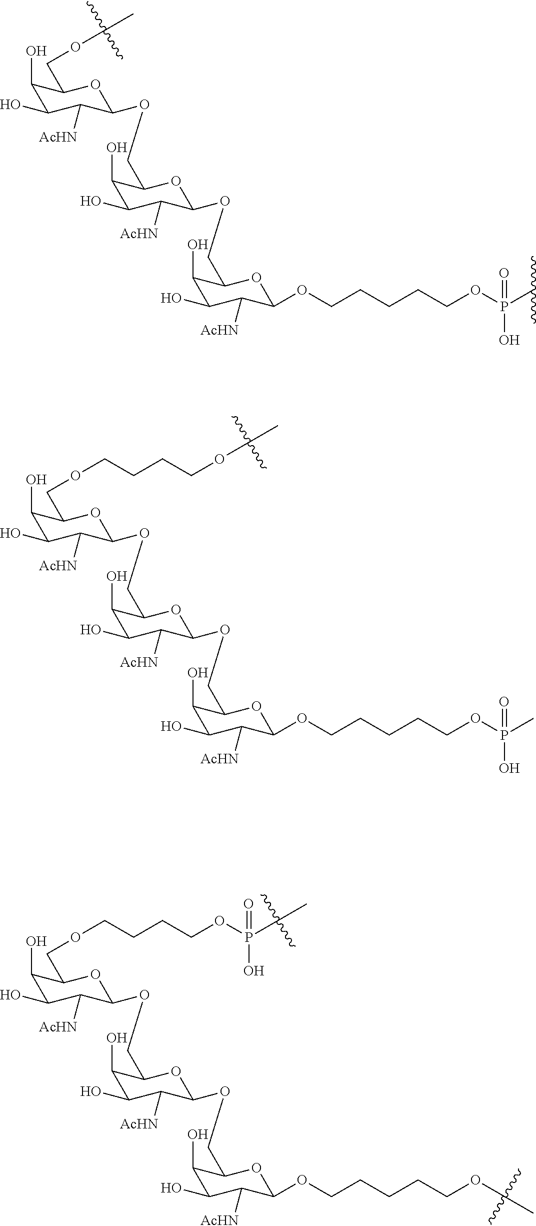 [SMILES: *OCC1OC(OCC2OC(OCC3OC(OCCCCCOP(*)(=O)O)C(NC(C)=O)C(O)C3O)C(NC(C)=O)C(O)C2O)C(NC(C)=O)C(O)C1O ; *OCCCCOCC1OC(OCC2OC(OCC3OC(OCCCCCOP(C)(=O)O)C(NC(C)=O)C(O)C3O)C(NC(C)=O)C(O)C2O)C(NC(C)=O)C(O)C1O ; *OCCCCCOC1OC(COC2OC(COC3OC(COCCCCOP(*)(=O)O)C(O)C(O)C3NC(C)=O)C(O)C(O)C2NC(C)=O)C(O)C(O)C1NC(C)=O]

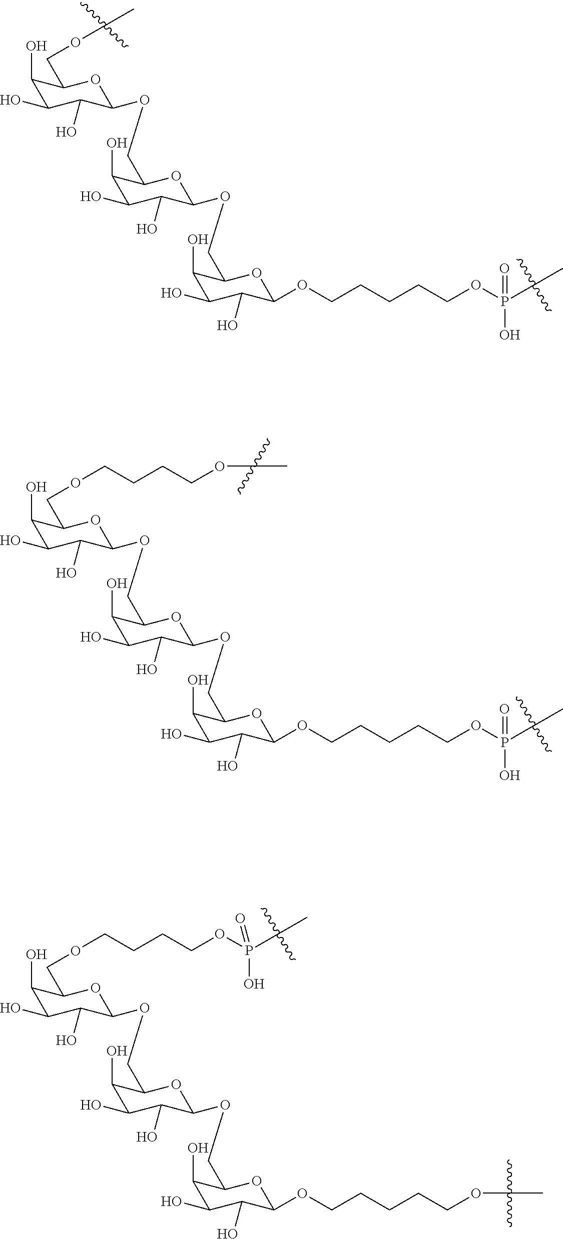 [SMILES: *OCC1OC(OCC2OC(OCC3OC(OCCCCCOP(*)(=O)O)C(O)C(O)C3O)C(O)C(O)C2O)C(O)C(O)C1O ; *OCCCCOCC1OC(OCC2OC(OCC3OC(OCCCCCOP(*)(=O)O)C(O)C(O)C3O)C(O)C(O)C2O)C(O)C(O)C1O ; *OCCCCCOC1OC(COC2OC(COC3OC(COCCCCOP(*)(=O)O)C(O)C(O)C3O)C(O)C(O)C2O)C(O)C(O)C1O]

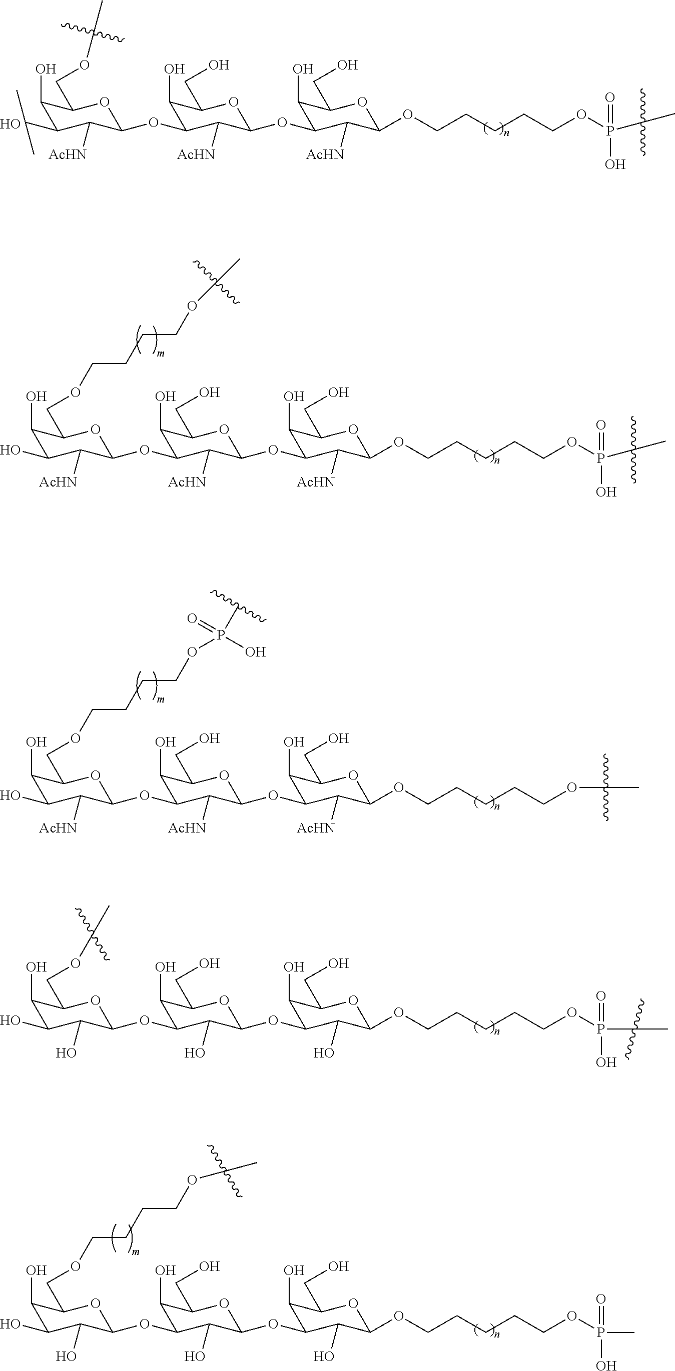

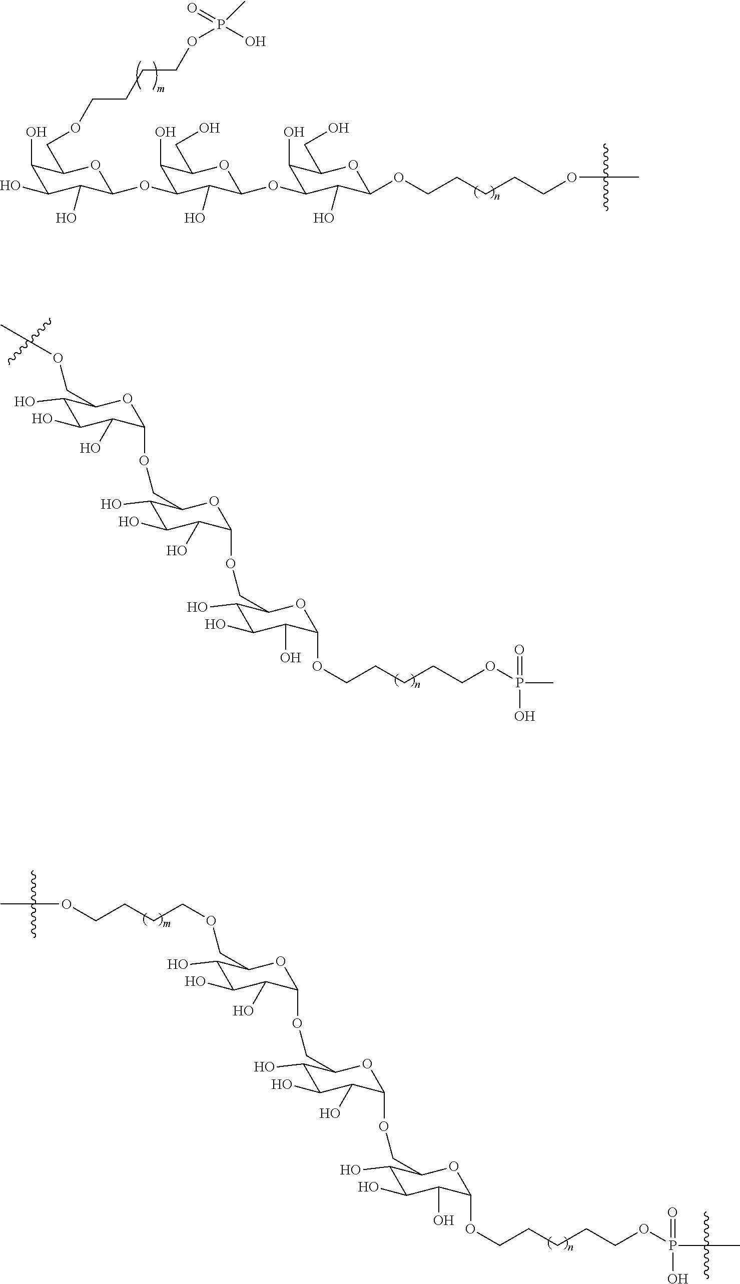

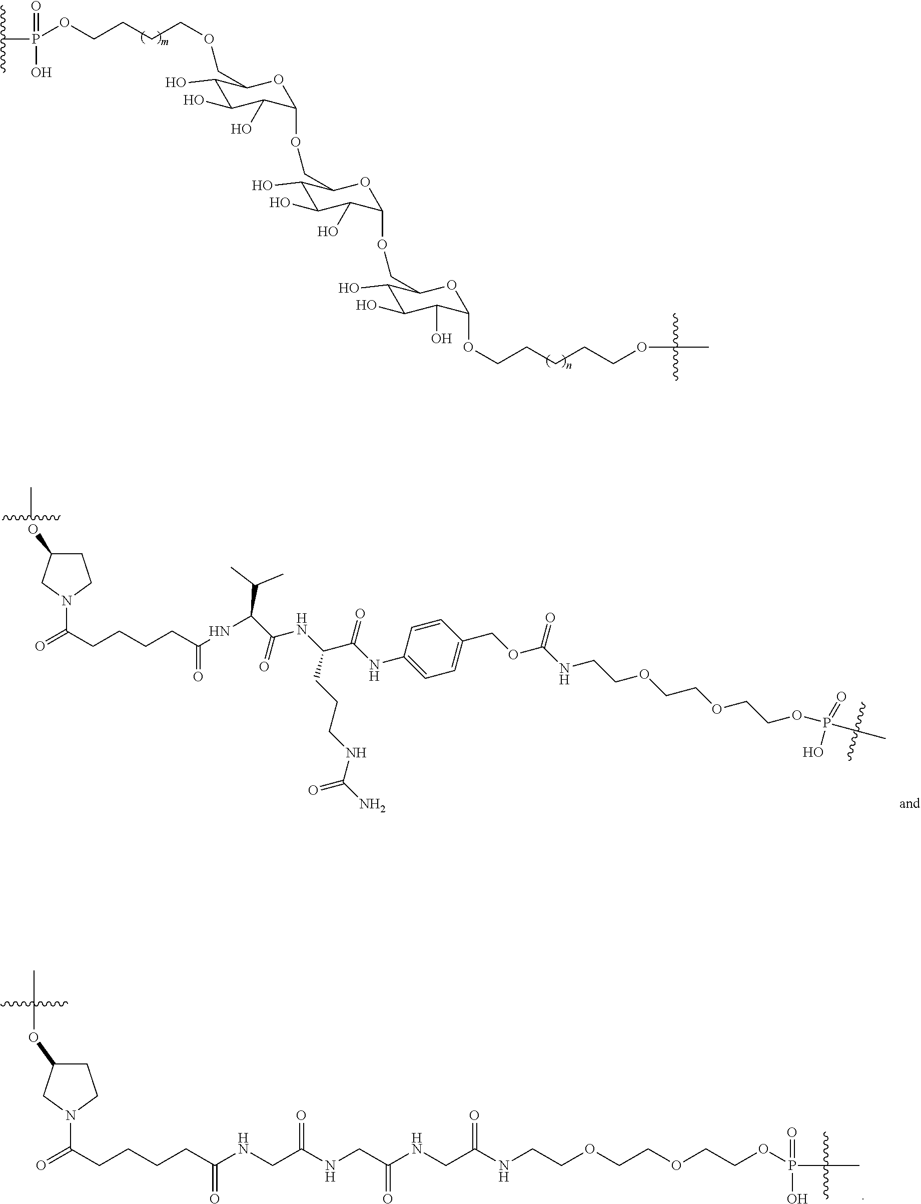

Additional exemplary bio-cleavable linkers are illustrated in Schemes 28-30.

More discussion about the biocleavable linkers may be found in PCT application No. PCT/US18/14213, entitled “Endosomal Cleavable Linkers,” filed on Jan. 18, 2018, the content of which is incorporated herein by reference in its entirety.

### C. Carriers

In certain embodiments, the lipophilic moiety is conjugated to the iRNA agent via a carrier that replaces one or more nucleotide(s).

The carrier can be a cyclic group or an acyclic group. In one embodiment, the cyclic group is selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalin. In one embodiment, the acyclic group is a moiety based on a serinol backbone or a diethanolamine backbone.

In some embodiments, the carrier replaces one or more nucleotide(s) in the internal position(s) of the double-stranded iRNA agent. In some embodiments, the carrier replaces one or more nucleotide(s) within the double stranded portion of the double-stranded iRNA agent.

In other embodiments, the carrier replaces the nucleotides at the terminal end of the sense strand or antisense strand. In one embodiment, the carrier replaces the terminal nucleotide on the 3′ end of the sense strand, thereby functioning as an end cap protecting the 3′ end of the sense strand. In one embodiment, the carrier is a cyclic group having an amine, for instance, the carrier may be pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, or decalinyl.

A ribonucleotide subunit in which the ribose sugar of the subunit has been so replaced is referred to herein as a ribose replacement modification subunit (RRMS). The carrier can be a cyclic or acyclic moiety and include two “backbone attachment points” (e.g., hydroxyl groups) and a ligand (e.g., the lipophilic moiety). The lipophilic moiety can be directly attached to the carrier or indirectly attached to the carrier by an intervening linker/tether, as described above.

The ligand-conjugated monomer subunit may be the 5′ or 3′ terminal subunit of the iRNA molecule, i.e., one of the two “W” groups may be a hydroxyl group, and the other “W” group may be a chain of two or more unmodified or modified ribonucleotides. Alternatively, the ligand-conjugated monomer subunit may occupy an internal position, or a position within the double stranded region, and both “W” groups may be one or more unmodified or modified ribonucleotides. More than one ligand-conjugated monomer subunit may be present in an iRNA agent.

i. Sugar Replacement-Based Monomers, e.g., Ligand-Conjugated Monomers (Cyclic)

Cyclic sugar replacement-based monomers, e.g., sugar replacement-based ligand-conjugated monomers, are also referred to herein as RRMS monomer compounds. The carriers may have the general formula (LCM-2) provided below (In that structure preferred backbone attachment points can be chosen from R¹ or R²; R³ or R⁴; or R⁹ and R¹⁰ if Y is CR⁹R¹⁰ (two positions are chosen to give two backbone attachment points, e.g., R¹ and R⁴, or R⁴ and R⁹)). Preferred tethering attachment points include R⁷; R⁵ or R⁶ when X is CH₂. The carriers are described below as an entity, which can be incorporated into a strand. Thus, it is understood that the structures also encompass the situations wherein one (in the case of a terminal position) or two (in the case of an internal position) of the attachment points, e.g., R³ or R²; R³ or R⁴; or R⁹ or R¹⁰ (when Y is CR⁹R¹⁰), is connected to the phosphate, or modified phosphate, e.g., sulfur containing, backbone. E.g., one of the above-named R groups can be —CH₂—, wherein one bond is connected to the carrier and one to a backbone atom, e.g., a linking oxygen or a central phosphorus atom.

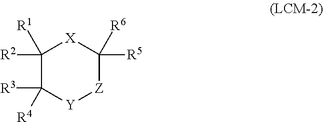

wherein:
X is N(CO)R⁷, NR⁷ or CH₂;
Y is NR⁸, O, S, CR⁹R¹⁰;
Z is CR¹¹R¹² or absent;
Each of R¹, R², R³, R⁴, R⁹, and R¹⁰ is, independently, H, OR^{a}, or (CH₂)ₙOR^{b}, provided that at least two of R¹, R², R³, R⁴, R⁹, and R¹⁰ are OR^{a} and/or (CH₂)ₙOR^{b};

Each of R⁵, R⁶, R¹¹, and R¹² is, independently, a ligand, H, C₁-C₆ alkyl optionally substituted with 1-3 R¹³, or C(O)NHR⁷; or R⁵ and R¹¹ together are C₃-C₈ cycloalkyl optionally substituted with
R⁷ can be a ligand, e.g., R⁷ can be R^{d}, or R⁷ can be a ligand tethered indirectly to the carrier, e.g., through a tethering moiety, e.g., C₁-C₂₀ alkyl substituted with NR^{c}R^{d}; or C₁-C₂₀ alkyl substituted with NHC(O)R^{d};
R⁸ is H or C₁-C₆ alkyl;
R¹³ is hydroxy, C₁-C₄ alkoxy, or halo;
R¹⁴ is NR^{c}R⁷;
R¹⁵ is C₁-C₆ alkyl optionally substituted with cyano, or C₂-C₆ alkenyl;
R¹⁶ is C₁-C₁₀ alkyl;
R¹⁷ is a liquid or solid phase support reagent;
L is —C(O)(CH₂)_{q}C(O)—, or —C(O)(CH₂)_{q}S—;
R^{a} is a protecting group, e.g., CAr₃; (e.g., a dimethoxytrityl group) or Si(X^{5′})(X^{5″})(X^{5′″}) in which (X^{5′}), (X^{5″}), and (X^{5′″}) are as described elsewhere.
R^{b} is P(O)(O—)H, P(OR¹⁵)N(R¹⁶)₂ or L-R¹⁷;
R^{c} is H or C₁-C₆ alkyl;
R^{d} is H or a ligand;

Each Ar is, independently, C₆-C₁₀ aryl optionally substituted with C₁-C₄ alkoxy;
n is 1-4; and q is 0-4.

Exemplary carriers include those in which, e.g., X is N(CO)R⁷ or NR⁷, Y is CR⁹R¹⁰, and Z is absent; or X is N(CO)R⁷ or NR⁷, Y is CR⁹R¹⁰, and Z is CR¹¹R¹²; or X is N(CO)R⁷ or NR⁷, Y is NR⁸, and Z is CR¹¹R¹²; or X is N(CO)R⁷ or NR⁷, Y is O, and Z is CR¹¹R¹²; or X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

In certain embodiments, the carrier may be based on the pyrroline ring system or the 4-hydroxyproline ring system, e.g., X is N(CO)R⁷ or NR⁷, Y is CR⁹R¹⁰, and Z is absent (D).

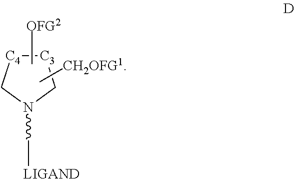

OFG¹ is preferably attached to a primary carbon, e.g., an exocyclic alkylene group, e.g., a methylene group, connected to one of the carbons in the five-membered ring (—CH₂OFG¹ in D). OFG² is preferably attached directly to one of the carbons in the five-membered ring (—OFG² in D). For the pyrroline-based carriers, —CH₂OFG¹ may be attached to C-2 and OFG² may be attached to C-3; or —CH₂OFG¹ may be attached to C-3 and OFG² may be attached to C-4. In certain embodiments, CH₂OFG¹ and OFG² may be geminally substituted to one of the above-referenced carbons. For the 3-hydroxyproline-based carriers, —CH₂OFG¹ may be attached to C-2 and OFG² may be attached to C-4. The pyrroline- and 4-hydroxyproline-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen. Preferred examples of carrier D include the following:

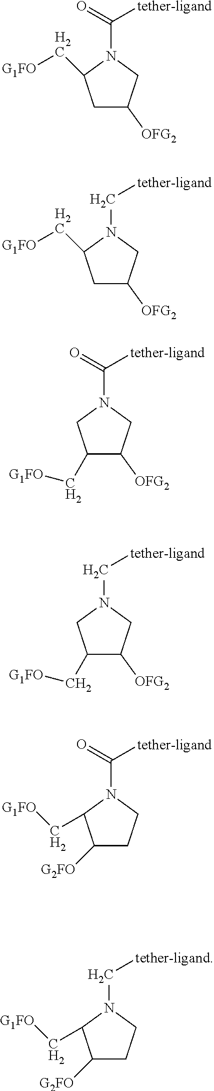

In certain embodiments, the carrier may be based on the piperidine ring system (E), e.g., X is N(CO)R⁷ or NR⁷, Y is CR⁹R¹⁰, and Z is CR¹¹R¹².

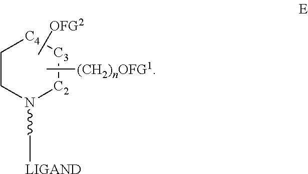

OFG¹ is preferably attached to a primary carbon, e.g., an exocyclic alkylene group, e.g., a methylene group (n=1) or ethylene group (n=2), connected to one of the carbons in the six-membered ring [—(CH₂)ₙOFG¹ in E]. OFG² is preferably attached directly to one of the carbons in the six-membered ring (—OFG² in E). —(CH₂)ₙOFG¹ and OFG² may be disposed in a geminal manner on the ring, i.e., both groups may be attached to the same carbon, e.g., at C-2, C-3, or C-4. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3. The piperidine-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

In certain embodiments, the carrier may be based on the piperazine ring system (F), e.g., X is N(CO)R⁷ or NR⁷, Y is NR⁸, and Z is CR¹¹R¹², or the morpholine ring system (G), e.g., X is N(CO)R⁷ or NR⁷, Y is O, and Z is CR¹¹R¹².

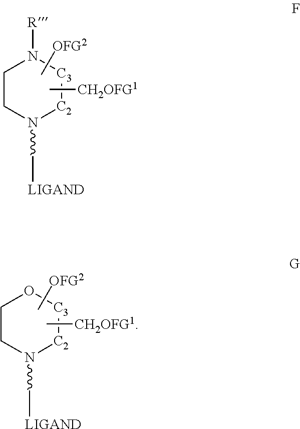

OFG¹ is preferably attached to a primary carbon, e.g., an exocyclic alkylene group, e.g., a methylene group, connected to one of the carbons in the six-membered ring (—CH₂OFG¹ in F or G). OFG² is preferably attached directly to one of the carbons in the six-membered rings (—OFG² in F or G). For both F and G, —CH₂OFG¹ may be attached to C-2 and OFG² may be attached to C-3; or vice versa. In certain embodiments, CH₂OFG¹ and OFG² may be geminally substituted to one of the above-referenced carbons. The piperazine- and morpholine-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). R′″ can be, e.g., C₁-C₆ alkyl, preferably CH₃. The tethering attachment point is preferably nitrogen in both F and G. In certain embodiments, the carrier may be based on the decalin ring system, e.g., X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

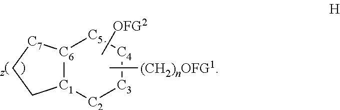

OFG¹ is preferably attached to a primary carbon, e.g., an exocyclic methylene group (n=1) or ethylene group (n=2) connected to one of C-2, C-3, C-4, or C-5 [—(CH₂)ₙOFG¹ in H]. OFG² is preferably attached directly to one of C-2, C-3, C-4, or C-5 (—OFG² in H). —(CH₂)ₙOFG¹ and OFG² may be disposed in a geminal manner on the ring, i.e., both groups may be attached to the same carbon, e.g., at C-2, C-3, C-4, or C-5. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3; —(CH₂)ₙOFG¹ may be attached to C-4 and OFG² may be attached to C-5; or —(CH₂)ₙOFG¹ may be attached to C-5 and OFG² may be attached to C-4. The decalin or indane-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). In a preferred embodiment, the substituents at C-1 and C-6 are trans with respect to one another. The tethering attachment point is preferably C-6 or C-7.

Other carriers may include those based on 3-hydroxyproline (J).

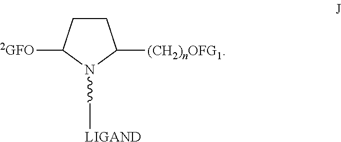

Thus, —(CH₂)ₙOFG¹ and OFG² may be cis or trans with respect to one another. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

Details about more representative cyclic, sugar replacement-based carriers can be found in U.S. Pat. Nos. 7,745,608 and 8,017,762, which are herein incorporated by reference in their entireties.

ii. Sugar Replacement-Based Monomers (Acyclic) Acyclic sugar replacement-based monomers, e.g., sugar replacement-based ligand-conjugated monomers, are also referred to herein as ribose replacement monomer subunit (RRMS) monomer compounds. Preferred acyclic carriers can have formula LCM-3 or LCM-4:

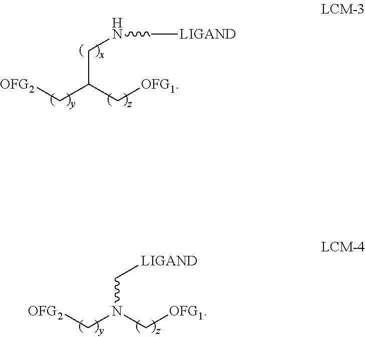

In some embodiments, each of x, y, and z can be, independently of one another, 0, 1, 2, or 3. In formula LCM-3, when y and z are different, then the tertiary carbon can have either the R or S configuration. In preferred embodiments, x is zero and y and z are each 1 in formula LCM-3 (e.g., based on serinol), and y and z are each 1 in formula LCM-3. Each of formula LCM-3 or LCM-4 below can optionally be substituted, e.g., with hydroxy, alkoxy, perhaloalkyl.

Details about more representative acyclic, sugar replacement-based carriers can be found in U.S. Pat. Nos. 7,745,608 and 8,017,762, which are herein incorporated by reference in their entireties.

In some embodiments, the double stranded iRNA agent comprises one or more lipophilic moieties conjugated to the 5′ end of the sense strand or the 5′ end of the antisense strand.

In certain embodiments, the lipophilic moiety is conjugated to the 5′-end of a strand via a carrier and/or linker. In one embodiment, the lipophilic moiety is conjugated to the 5′-end of a strand via a carrier of a formula:

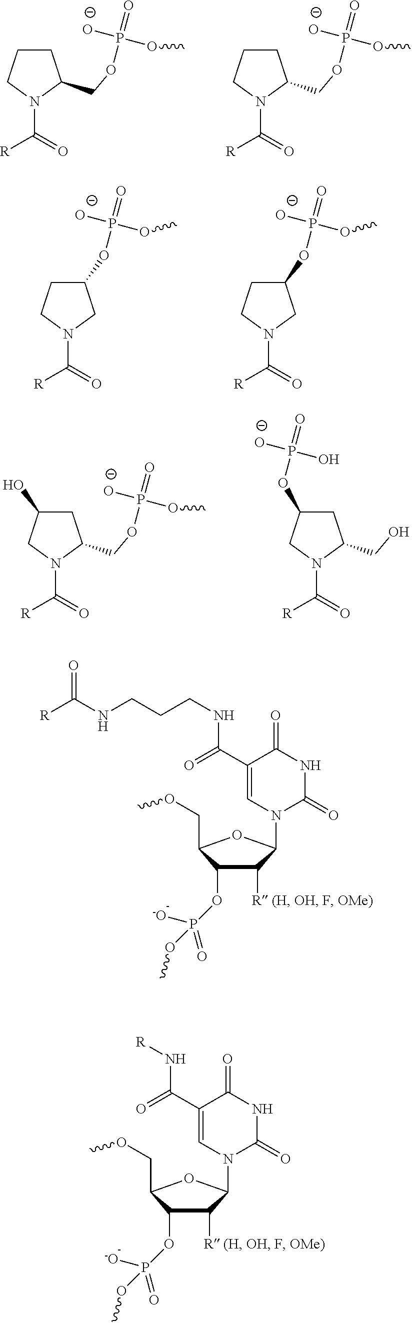

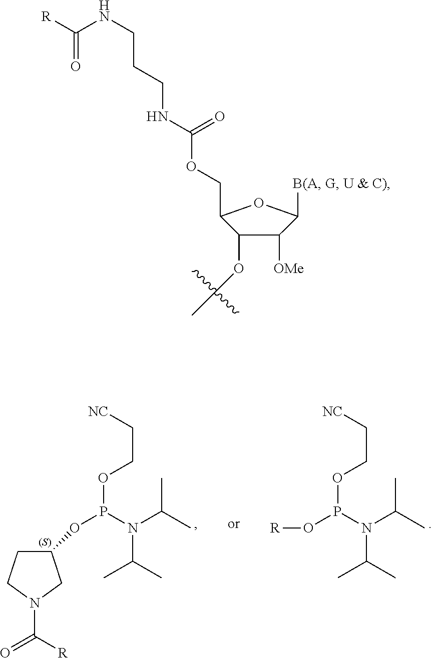

R is a ligand such as the lipophilic moiety.

In some embodiments, the double stranded iRNA agent comprises one or more lipophilic moieties conjugated to the 3′ end of the sense strand or the 3′ end of the antisense strand.

In certain embodiments, the lipophilic moiety is conjugated to the 3′-end of a strand via a carrier and/or linker. In one embodiment, the lipophilic moiety is conjugated to the 3′-end of a strand via a carrier of a formula:

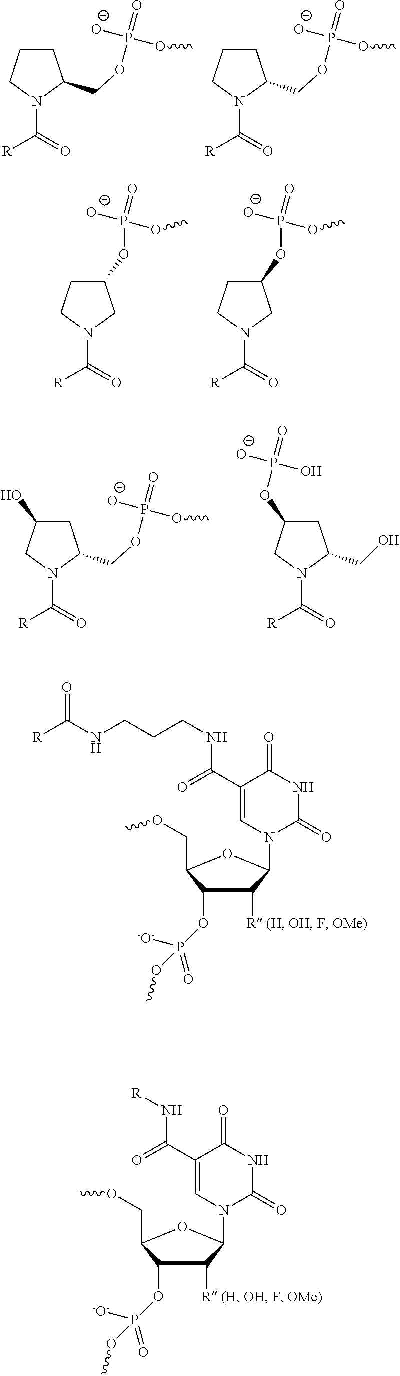

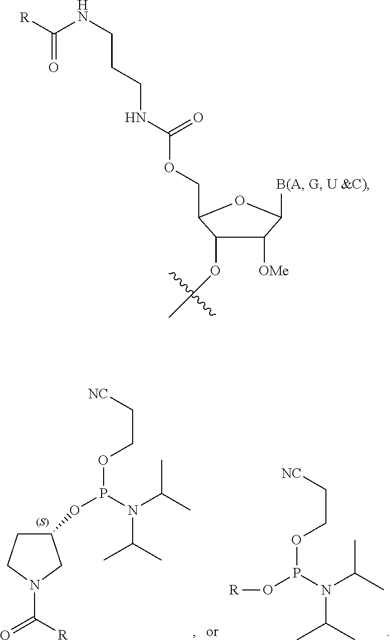

R is a ligand such as the lipophilic moiety.

In some embodiments, the double stranded iRNA agent comprises one or more lipophilic moieties conjugated to both ends of the sense strand.

In some embodiments, the double stranded iRNA agent comprises one or more lipophilic moieties conjugated to both ends of the antisense strand.

In some embodiments, the double stranded iRNA agent comprises one or more lipophilic moieties conjugated to the 5′ end or 3′ end of the sense strand, and one or more lipophilic moieties conjugated to the 5′ end or 3′ end of the antisense strand,

In some embodiments, the lipophilic moiety is conjugated to the terminal end of a strand via one or more linkers (tethers) and/or a carrier.

In one embodiment, the lipophilic moiety is conjugated to the terminal end of a strand via one or more linkers (tethers).

In one embodiment, the lipophilic moiety is conjugated to the 5′ end of the sense strand or antisense strand via a cyclic carrier, optionally via one or more intervening linkers (tethers).

In some embodiments, the lipophilic moiety is conjugated to one or more internal positions on at least one strand. Internal positions of a strand refers to the nucleotide on any position of the strand, except the terminal position from the 3′ end and 5′ end of the strand (e.g., excluding 2 positions: position 1 counting from the 3′ end and position 1 counting from the 5′ end).

In one embodiment, the lipophilic moiety is conjugated to one or more internal positions on at least one strand, which include all positions except the terminal two positions from each end of the strand (e.g., excluding 4 positions: positions 1 and 2 counting from the 3′ end and positions 1 and 2 counting from the 5′ end). In one embodiment, the lipophilic moiety is conjugated to one or more internal positions on at least one strand, which include all positions except the terminal three positions from each end of the strand (e.g., excluding 6 positions: positions 1, 2, and 3 counting from the 3′ end and positions 1, 2, and 3 counting from the 5′ end).

In one embodiment, the lipophilic moiety is conjugated to one or more internal positions on at least one strand, except the cleavage site region of the sense strand, for instance, the lipophilic moiety is not conjugated to positions 9-12 counting from the 5′-end of the sense strand. Alternatively, the internal positions exclude positions 11-13 counting from the 3′-end of the sense strand.

In one embodiment, the lipophilic moiety is conjugated to one or more internal positions on at least one strand, which exclude the cleavage site region of the antisense strand. For instance, the internal positions exclude positions 12-14 counting from the 5′-end of the antisense strand.

In one embodiment, the lipophilic moiety is conjugated to one or more internal positions on at least one strand, which exclude positions 11-13 on the sense strand, counting from the 3′-end, and positions 12-14 on the antisense strand, counting from the 5′-end.

In one embodiment, one or more lipophilic moieties are conjugated to one or more of the following internal positions: positions 4-8 and 13-18 on the sense strand, and positions 6-10 and 15-18 on the antisense strand, counting from the 5′end of each strand.

In one embodiment, one or more lipophilic moieties are conjugated to one or more of the following internal positions: positions 5, 6, 7, 15, and 17 on the sense strand, and positions 15 and 17 on the antisense strand, counting from the 5′end of each strand.

In some embodiments, the lipophilic moiety is conjugated to one or more positions in the double stranded region on at least one strand. The double stranded region does not include single stranded overhang or hairpin loop regions.

In some embodiments, the lipophilic moiety is conjugated to a nucleobase, sugar moiety, or internucleosidic linkage of the double-stranded iRNA agent.

Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a lipophilic moiety is conjugated to a nucleobase, the preferred position is one that does not interfere with hybridization, i.e., does not interfere with the hydrogen bonding interactions needed for base pairing. In one embodiment, the lipophilic moieties may be conjugated to a nucleobase via a linker containing an alkyl, alkenyl or amide linkage. Exemplary conjugations of the lipophilic moieties to the nucleobase are illustrated in FIG. 1 and Example 7.

Conjugation to sugar moieties of nucleosides can occur at any carbon atom. Exemplary carbon atoms of a sugar moiety that a lipophilic moiety can be attached to include the 2′, 3′, and 5′ carbon atoms. A lipophilic moiety can also be attached to the 1′ position, such as in an abasic residue. In one embodiment, the lipophilic moieties may be conjugated to a sugar moiety, via a 2′-0 modification, with or without a linker. Exemplary conjugations of the lipophilic moieties to the sugar moiety (via a 2′-0 modification) are illustrated in FIG. 1 and Examples 1, 2, 3, and 6.

Internucleosidic linkages can also bear lipophilic moieties. For phosphorus-containing linkages (e.g., phosphodiester, phosphorothioate, phosphorodithioate, phosphoroamidate, and the like), the lipophilic moiety can be attached directly to the phosphorus atom or to an O, N, or S atom bound to the phosphorus atom. For amine- or amide-containing internucleosidic linkages (e.g., PNA), the lipophilic moiety can be attached to the nitrogen atom of the amine or amide or to an adjacent carbon atom.

There are numerous methods for preparing conjugates of oligonuclotides. Generally, an oligonucleotide is attached to a conjugate moiety by contacting a reactive group (e.g., OH, SH, amine, carboxyl, aldehyde, and the like) on the oligonucleotide with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

In one embodiment, a first (complementary) RNA strand and a second (sense) RNA strand can be synthesized separately, wherein one of the RNA strands comprises a pendant lipophilic moiety, and the first and second RNA strands can be mixed to form a dsRNA. The step of synthesizing the RNA strand preferably involves solid-phase synthesis, wherein individual nucleotides are joined end to end through the formation of internucleotide 3′-5′ phosphodiester bonds in consecutive synthesis cycles.

In one embodiment, a lipophilic molecule having a phosphoramidite group is coupled to the 3′-end or 5′-end of either the first (complementary) or second (sense) RNA strand in the last synthesis cycle. In the solid-phase synthesis of an RNA, the nucleotides are initially in the form of nucleoside phosphoramidites. In each synthesis cycle, a further nucleoside phosphoramidite is linked to the —OH group of the previously incorporated nucleotide. If the lipophilic molecule has a phosphoramidite group, it can be coupled in a manner similar to a nucleoside phosphoramidite to the free OH end of the RNA synthesized previously in the solid-phase synthesis. The synthesis can take place in an automated and standardized manner using a conventional RNA synthesizer. Synthesis of the lipophilic molecule having the phosphoramidite group may include phosphitylation of a free hydroxyl to generate the phosphoramidite group.

Synthesis procedures of lipophilic moiety-conjugated phosphoramidites are exemplified in Examples 1, 2, 4, 5, 6, and 7. Examples of procedures of post-synthesis conjugation of lipophilic moieties or other ligands are illustrated in Example 3.

In general, the oligonucleotides can be synthesized using protocols known in the art, for example, as described in Caruthers et al., Methods in Enzymology (1992) 211:3-19; WO 99/54459; Wincott et al., Nucl. Acids Res. (1995) 23:2677-2684; Wincott et al., Methods Mol. Bio., (1997) 74:59; Brennan et al., Biotechnol. Bioeng. (1998) 61:33-45; and U.S. Pat. No. 6,001,311; each of which is hereby incorporated by reference in its entirety. In general, the synthesis of oligonucleotides involves conventional nucleic acid protecting and coupling groups, such as dimethoxytrityl at the 5′-end, and phosphoramidites at the 3′-end. In a non-limiting example, small scale syntheses are conducted on an Expedite 8909 RNA synthesizer sold by Applied Biosystems, Inc. (Weiterstadt, Germany), using ribonucleoside phosphoramidites sold by ChemGenes Corporation (Ashland, Mass.). Alternatively, syntheses can be performed on a 96-well plate synthesizer, such as the instrument produced by Protogene (Palo Alto, Calif.), or by methods such as those described in Usman et al., J. Am. Chem. Soc. (1987) 109:7845; Scaringe, et al., Nucl. Acids Res. (1990) 18:5433; Wincott, et al., Nucl. Acids Res. (1990) 23:2677-2684; and Wincott, et al., Methods Mol. Bio. (1997) 74:59, each of which is hereby incorporated by reference in its entirety.

The nucleic acid molecules of the present invention may be synthesized separately and joined together post-synthetically, for example, by ligation (Moore et al., Science (1992) 256:9923; WO 93/23569; Shabarova et al., Nucl. Acids Res. (1991) 19:4247; Bellon et al., Nucleosides & Nucleotides (1997) 16:951; Bellon et al., Bioconjugate Chem. (1997) 8:204; or by hybridization following synthesis and/or deprotection. The nucleic acid molecules can be purified by gel electrophoresis using conventional methods or can be purified by high pressure liquid chromatography (HPLC; see Wincott et al., supra, the totality of which is hereby incorporated herein by reference) and re-suspended in water.

### III. iRNAs of the Invention

The present invention provides iRNAs which selectively inhibit the expression of one or more TTR genes. In one embodiment, the iRNA agent includes double stranded ribonucleic acid (dsRNA) molecules for inhibiting the expression of a TTR gene in an ocular cell, such as an ocular cell within a subject, e.g., a mammal, such as a human having a TTR-associated ocular disease. The dsRNA includes an antisense strand having a region of complementarity which is complementary to at least a part of an mRNA formed in the expression of a TTR gene. The region of complementarity is about 30 nucleotides or less in length (e.g., about 30, 29, 28, 27, 26, 25, 24, 23, 22, 21, 20, 19, or 18 nucleotides or less in length). Upon contact with an ocular cell expressing the TTR gene, the iRNA selectively inhibits the expression of the TTR gene (e.g., a human, a primate, a non-primate, or a bird TTR gene) by at least about 10% as assayed by, for example, a PCR or branched DNA (bDNA)-based method, or by a protein-based method, such as by immunofluorescence analysis, using, for example, Western Blotting or flowcytometric techniques.

A dsRNA includes two RNA strands that are complementary and hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of a TTR gene. The other strand (the sense strand) includes a region that is complementary to the antisense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides.

Generally, the duplex structure is between 15 and 30 base pairs in length, e.g., between, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated to be part of the invention.

Similarly, the region of complementarity to the target sequence is between 15 and 30 nucleotides in length, e.g., between 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. Ranges and lengths intermediate to the above recited ranges and lengths are also contemplated to be part of the invention.

In some embodiments, the dsRNA is about 15 to about 20 nucleotides in length, or about 25 to about 30 nucleotides in length. In general, the dsRNA is long enough to serve as a substrate for the Dicer enzyme. For example, it is well-known in the art that dsRNAs longer than about 21-23 nucleotides in length may serve as substrates for Dicer. As the ordinarily skilled person will also recognize, the region of an RNA targeted for cleavage will most often be part of a larger RNA molecule, often an mRNA molecule. Where relevant, a “part” of an mRNA target is a contiguous sequence of an mRNA target of sufficient length to allow it to be a substrate for RNAi-directed cleavage (i.e., cleavage through a RISC pathway).

One of skill in the art will also recognize that the duplex region is a primary functional portion of a dsRNA, e.g., a duplex region of about 9 to 36 base pairs, e.g., about 10-36, 11-36, 12-36, 13-36, 14-36, 15-36, 9-35, 10-35, 11-35, 12-35, 13-35, 14-35, 15-35, 9-34, 10-34, 11-34, 12-34, 13-34, 14-34, 15-34, 9-33, 10-33, 11-33, 12-33, 13-33, 14-33, 15-33, 9-32, 10-32, 11-32, 12-32, 13-32, 14-32, 15-32, 9-31, 10-31, 11-31, 12-31, 13-32, 14-31, 15-31, 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs. Thus, in one embodiment, to the extent that it becomes processed to a functional duplex, of e.g., 15-30 base pairs, that targets a desired RNA for cleavage, an RNA molecule or complex of RNA molecules having a duplex region greater than 30 base pairs is a dsRNA. Thus, an ordinarily skilled artisan will recognize that in one embodiment, a miRNA is a dsRNA. In another embodiment, a dsRNA is not a naturally occurring miRNA. In another embodiment, an iRNA agent useful to target TTR gene expression is not generated in the target cell by cleavage of a larger dsRNA.

A dsRNA as described herein can further include one or more single-stranded nucleotide overhangs e.g., 1, 2, 3, or 4 nucleotides. dsRNAs having at least one nucleotide overhang can have unexpectedly superior inhibitory properties relative to their blunt-ended counterparts. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′-end, 3′-end or both ends of either an antisense or sense strand of a dsRNA. In certain embodiments, longer, extended overhangs are possible.

A dsRNA can be synthesized by standard methods known in the art as further discussed below, e.g., by use of an automated DNA synthesizer, such as are commercially available from, for example, Biosearch, Applied Biosystems, Inc.

iRNA compounds of the invention may be prepared using a two-step procedure. First, the individual strands of the double stranded RNA molecule are prepared separately. Then, the component strands are annealed. The individual strands of the siRNA compound can be prepared using solution-phase or solid-phase organic synthesis or both. Organic synthesis offers the advantage that the oligonucleotide strands comprising unnatural or modified nucleotides can be easily prepared. Single-stranded oligonucleotides of the invention can be prepared using solution-phase or solid-phase organic synthesis or both.

An siRNA can be produced, e.g., in bulk, by a variety of methods. Exemplary methods include: organic synthesis and RNA cleavage, e.g., in vitro cleavage.

An siRNA can be made by separately synthesizing a single stranded RNA molecule, or each respective strand of a double-stranded RNA molecule, after which the component strands can then be annealed.

A large bioreactor, e.g., the OligoPilot II from Pharmacia Biotec AB (Uppsala Sweden), can be used to produce a large amount of a particular RNA strand for a given siRNA. The OligoPilotII reactor can efficiently couple a nucleotide using only a 1.5 molar excess of a phosphoramidite nucleotide. To make an RNA strand, ribonucleotides amidites are used. Standard cycles of monomer addition can be used to synthesize the 21 to 23 nucleotide strand for the siRNA. Typically, the two complementary strands are produced separately and then annealed, e.g., after release from the solid support and deprotection.

Organic synthesis can be used to produce a discrete siRNA species. The complementary of the species to a TTR gene can be precisely specified. For example, the species may be complementary to a region that includes a polymorphism, e.g., a single nucleotide polymorphism. Further the location of the polymorphism can be precisely defined. In some embodiments, the polymorphism is located in an internal region, e.g., at least 4, 5, 7, or 9 nucleotides from one or both of the termini.

In one embodiment, RNA generated is carefully purified to remove ends iRNA is cleaved in vitro into siRNAs, for example, using a Dicer or comparable RNAse III-based activity. For example, the dsiRNA can be incubated in an in vitro extract from Drosophila or using purified components, e.g., a purified RNAse or RISC complex (RNA-induced silencing complex). See, e.g., Ketting et al. Genes Dev 2001 Oct. 15; 15(20):2654-9 and Hammond Science 2001 Aug. 10; 293(5532):1146-50.

dsiRNA cleavage generally produces a plurality of siRNA species, each being a particular 21 to 23 nt fragment of a source dsiRNA molecule. For example, siRNAs that include sequences complementary to overlapping regions and adjacent regions of a source dsiRNA molecule may be present.

Regardless of the method of synthesis, the siRNA preparation can be prepared in a solution (e.g., an aqueous and/or organic solution) that is appropriate for formulation. For example, the siRNA preparation can be precipitated and redissolved in pure double-distilled water, and lyophilized. The dried siRNA can then be resuspended in a solution appropriate for the intended formulation process.

In one aspect, a dsRNA of the invention includes at least two nucleotide sequences, a sense sequence and an anti-sense sequence. The sense strand is selected from the group of sequences provided in Table 4, and the corresponding antisense strand of the sense strand is selected from the group of sequences in Table 4. In this aspect, one of the two sequences is complementary to the other of the two sequences, with one of the sequences being substantially complementary to a sequence of an mRNA generated in the expression of a TTR gene. As such, in this aspect, a dsRNA will include two oligonucleotides, where one oligonucleotide is described as the sense strand in Table 4, and the second oligonucleotide is described as the corresponding antisense strand of the sense strand in Table 4. In one embodiment, the substantially complementary sequences of the dsRNA are contained on separate oligonucleotides. In another embodiment, the substantially complementary sequences of the dsRNA are contained on a single oligonucleotide.

It will be understood that, although some of the sequences provided herein are described as modified and/or conjugated sequences, the RNA of the iRNA of the invention e.g., a dsRNA of the invention, may comprise any one of the sequences provides herein that is un-modified, un-conjugated, and/or modified and/or conjugated differently than described therein.

The skilled person is well aware that dsRNAs having a duplex structure of between about 20 and 23 base pairs, e.g., 21, base pairs have been hailed as particularly effective in inducing RNA interference (Elbashir et al., EMBO 2001, 20:6877-6888). However, others have found that shorter or longer RNA duplex structures can also be effective (Chu and Rana (2007) RNA 14:1714-1719; Kim et al. (2005) Nat Biotech 23:222-226). In the embodiments described above, by virtue of the nature of the oligonucleotide sequences provided in Table 4, dsRNAs described herein can include at least one strand of a length of minimally 21 nucleotides. It can be reasonably expected that shorter duplexes having one of the sequences of Table 4 minus only a few nucleotides on one or both ends can be similarly effective as compared to the dsRNAs described above. Hence, dsRNAs having a sequence of at least 15, 16, 17, 18, 19, 20, or more contiguous nucleotides derived from one of the sequences of Table 4, and differing in their ability to inhibit the expression of a TTR gene by not more than about 5, 10, 15, 20, 25, or 30% inhibition from a dsRNA comprising the full sequence, are contemplated to be within the scope of the present invention.

In addition, the RNAs provided in Table 4 identify a site(s) in a TTR transcript that is susceptible to RISC-mediated cleavage. As such, the present invention further features iRNAs that target within one of these sites. As used herein, an iRNA is said to target within a particular site of an RNA transcript if the iRNA promotes cleavage of the transcript anywhere within that particular site. Such an iRNA will generally include at least about 15 contiguous nucleotides from one of the sequences provided in Table 4 coupled to additional nucleotide sequences taken from the region contiguous to the selected sequence in a TTR gene.

While a target sequence is generally about 15-30 nucleotides in length, there is wide variation in the suitability of particular sequences in this range for directing cleavage of any given target RNA. Various software packages and the guidelines set out herein provide guidance for the identification of optimal target sequences for any given gene target, but an empirical approach can also be taken in which a “window” or “mask” of a given size (as a non-limiting example, 21 nucleotides) is literally or figuratively (including, e.g., in silico) placed on the target RNA sequence to identify sequences in the size range that can serve as target sequences. By moving the sequence “window” progressively one nucleotide upstream or downstream of an initial target sequence location, the next potential target sequence can be identified, until the complete set of possible sequences is identified for any given target size selected. This process, coupled with systematic synthesis and testing of the identified sequences (using assays as described herein or as known in the art) to identify those sequences that perform optimally can identify those RNA sequences that, when targeted with an iRNA agent, mediate the best inhibition of target gene expression. Thus, while the sequences identified, for example, in Table 4 represent effective target sequences, it is contemplated that further optimization of inhibition efficiency can be achieved by progressively “walking the window” one nucleotide upstream or downstream of the given sequences to identify sequences with equal or better inhibition characteristics.

Further, it is contemplated that for any sequence identified, e.g., in Table 4, further optimization could be achieved by systematically either adding or removing nucleotides to generate longer or shorter sequences and testing those sequences generated by walking a window of the longer or shorter size up or down the target RNA from that point. Again, coupling this approach to generating new candidate targets with testing for effectiveness of iRNAs based on those target sequences in an inhibition assay as known in the art and/or as described herein can lead to further improvements in the efficiency of inhibition. Further still, such optimized sequences can be adjusted by, e.g., the introduction of modified nucleotides as described herein or as known in the art, addition or changes in overhang, or other modifications as known in the art and/or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes) as an expression inhibitor.

An iRNA as described herein can contain one or more mismatches to the target sequence. In one embodiment, an iRNA as described herein contains no more than 3 mismatches. If the antisense strand of the iRNA contains mismatches to a target sequence, it is preferable that the area of mismatch is not located in the center of the region of complementarity. If the antisense strand of the iRNA contains mismatches to the target sequence, it is preferable that the mismatch be restricted to be within the last 5 nucleotides from either the 5′- or 3′-end of the region of complementarity. For example, for a 23 nucleotide iRNA agent the strand which is complementary to a region of a TTR gene, generally does not contain any mismatch within the central 13 nucleotides. The methods described herein or methods known in the art can be used to determine whether an iRNA containing a mismatch to a target sequence is effective in inhibiting the expression of a TTR gene. Consideration of the efficacy of iRNAs with mismatches in inhibiting expression of a TTR gene is important, especially if the particular region of complementarity in a TTR gene is known to have polymorphic sequence variation within the population.

A. iRNAs of the Invention Comprising Modified Nucleotides

In some embodiments, the double-stranded iRNA agent of the invention comprises at least one nucleic acid modification described herein. For example, at least one modification selected from the group consisting of modified internucleoside linkage, modified nucleobase, modified sugar, and any combinations thereof. Without limitations, such a modification can be present anywhere in the double-stranded iRNA agent of the invention. For example, the modification can be present in one of the RNA molecules.

The naturally occurring base portion of a nucleoside is typically a heterocyclic base. The two most common classes of such heterocyclic bases are the purines and the pyrimidines. For those nucleosides that include a pentofuranosyl sugar, a phosphate group can be linked to the 2′, 3′ or 5′ hydroxyl moiety of the sugar. In forming oligonucleotides, those phosphate groups covalently link adjacent nucleosides to one another to form a linear polymeric compound. Within oligonucleotides, the phosphate groups are commonly referred to as forming the internucleoside backbone of the oligonucleotide. The naturally occurring linkage or backbone of RNA and of DNA is a 3′ to 5′ phosphodiester linkage.

In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable with the compounds described herein. The unmodified or natural nucleobases can be modified or replaced to provide iRNAs having improved properties. For example, nuclease resistant oligonucleotides can be prepared with these bases or with synthetic and natural nucleobases (e.g., inosine, xanthine, hypoxanthine, nubularine, isoguanisine, or tubercidine) and any one of the oligomer modifications described herein. Alternatively, substituted or modified analogs of any of the above bases and “universal bases” can be employed. When a natural base is replaced by a non-natural and/or universal base, the nucleotide is said to comprise a modified nucleobase and/or a nucleobase modification herein. Modified nucleobase and/or nucleobase modifications also include natural, non-natural and universal bases, which comprise conjugated moieties, e.g. a ligand described herein. Preferred conjugate moieties for conjugation with nucleobases include cationic amino groups which can be conjugated to the nucleobase via an appropriate alkyl, alkenyl or a linker with an amide linkage.

An oligomeric compound described herein can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Exemplary modified nucleobases include, but are not limited to, other synthetic and natural nucleobases such as inosine, xanthine, hypoxanthine, nubularine, isoguanisine, tubercidine, 2-(halo)adenine, 2-(alkyl)adenine, 2-(propyl)adenine, 2 (amino)adenine, 2-(aminoalkyll)adenine, 2 (aminopropyl)adenine, 2 (methylthio) N6 (isopentenyl)adenine, 6 (alkyl)adenine, 6 (methyl)adenine, 7 (deaza)adenine, 8 (alkenyl)adenine, 8-(alkyl)adenine, 8 (alkynyl)adenine, 8 (amino)adenine, 8-(halo)adenine, 8-(hydroxyl)adenine, 8 (thioalkyl)adenine, 8-(thiol)adenine, N6-(isopentyl)adenine, N6 (methyl)adenine, N6, N6 (dimethyl)adenine, 2-(alkyl)guanine, 2 (propyl)guanine, 6-(alkyl)guanine, 6 (methyl)guanine, 7 (alkyl)guanine, 7 (methyl)guanine, 7 (deaza)guanine, 8 (alkyl)guanine, 8-(alkenyl)guanine, 8 (alkynyl)guanine, 8-(amino)guanine, 8 (halo)guanine, 8-(hydroxyl)guanine, 8 (thioalkyl)guanine, 8-(thiol)guanine, N (methyl)guanine, 2-(thio)cytosine, 3 (deaza) 5 (aza)cytosine, 3-(alkyl)cytosine, 3 (methyl)cytosine, 5-(alkyl)cytosine, 5-(alkynyl)cytosine, 5 (halo)cytosine, 5 (methyl)cytosine, 5 (propynyl)cytosine, 5 (propynyl)cytosine, 5 (trifluoromethyl)cytosine, 6-(azo)cytosine, N4 (acetyl)cytosine, 3 (3 amino-3 carboxypropyl)uracil, 2-(thio)uracil, 5 (methyl) 2 (thio)uracil, 5 (methylaminomethyl)-2 (thio)uracil, 4-(thio)uracil, 5 (methyl) 4 (thio)uracil, 5 (methylaminomethyl)-4 (thio)uracil, 5 (methyl) 2,4 (dithio)uracil, 5 (methylaminomethyl)-2,4 (dithio)uracil, 5 (2-aminopropyl)uracil, 5-(alkyl)uracil, 5-(alkynyl)uracil, 5-(allylamino)uracil, 5 (aminoallyl)uracil, 5 (aminoalkyl)uracil, 5 (guanidiniumalkyl)uracil, 5 (1,3-diazole-1-alkyl)uracil, 5-(cyanoalkyl)uracil, 5-(dialkylaminoalkyl)uracil, 5 (dimethylaminoalkyl)uracil, 5-(halo)uracil, 5-(methoxy)uracil, uracil-5 oxyacetic acid, 5 (methoxycarbonylmethyl)-2-(thio)uracil, 5 (methoxycarbonyl-methyl)uracil, 5 (propynyl)uracil, 5 (propynyl)uracil, 5 (trifluoromethyl)uracil, 6 (azo)uracil, dihydrouracil, N3 (methyl)uracil, 5-uracil (i.e., pseudouracil), 2 (thio)pseudouracil, 4 (thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4 (thio)pseudouracil, 5-(methyl)-4 (thio)pseudouracil, 5-(alkyl)-2,4 (dithio)pseudouracil, 5-(methyl)-2,4 (dithio)pseudouracil, 1 substituted pseudouracil, 1 substituted 2(thio)-pseudouracil, 1 substituted 4 (thio)pseudouracil, 1 substituted 2,4-(dithio)pseudouracil, 1 (aminocarbonylethylenyl)-pseudouracil, 1 (aminocarbonylethylenyl)-2(thio)-pseudouracil, 1 (aminocarbonylethylenyl)-4 (thio)pseudouracil, 1 (aminocarbonylethylenyl)-2,4-(dithio)pseudouracil, 1 (aminoalkylaminocarbonylethylenyl)-pseudouracil, 1 (aminoalkylamino-carbonylethylenyl)-2(thio)-pseudouracil, 1 (aminoalkylaminocarbonylethylenyl)-4 (thio)pseudouracil, 1 (aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5 nitroindole, 3 nitropyrrole, 6-(aza)pyrimidine, 2 (amino)purine, 2,6-(diamino)purine, 5 substituted pyrimidines, N2-substituted purines, N6-substituted purines, 06-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Alternatively, substituted or modified analogs of any of the above bases and “universal bases” can be employed.

As used herein, a universal nucleobase is any nucleobase that can base pair with all of the four naturally occurring nucleobases without substantially affecting the melting behavior, recognition by intracellular enzymes or activity of the iRNA duplex. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof (see for example, Loakes, 2001, Nucleic Acids Research, 29, 2437-2447).

Further nucleobases include those disclosed in U.S. Pat. No. 3,687,808; those disclosed in International Application No. PCT/US09/038425, filed Mar. 26, 2009; those disclosed in the “Concise Encyclopedia Of Polymer Science And Engineering,” pages 858-859, Kroschwitz, J. I., ed. John Wiley & Sons, 1990; those disclosed by English et al., “Angewandte Chemie, International Edition,” 1991, 30, 613; those disclosed in “Modified Nucleosides in Biochemistry, Biotechnology and Medicine,” Herdewijin, P. Ed. Wiley-VCH, 2008; and those disclosed by Sanghvi, Y. S., Chapter 15, dsRNA Research and Applications, pages 289-302, Crooke, S. T. and Lebleu, B., Eds., CRC Press, 1993. Contents of all of the above are herein incorporated by reference.

In certain embodiments, a modified nucleobase is a nucleobase that is fairly similar in structure to the parent nucleobase, such as for example a 7-deaza purine, a 5-methyl cytosine, or a G-clamp. In certain embodiments, nucleobase mimetic include more complicated structures, such as for example a tricyclic phenoxazine nucleobase mimetic. Methods for preparation of the above noted modified nucleobases are well known to those skilled in the art.

Double-stranded iRNA agent of the inventions provided herein can comprise one or more (e.g., 1, 2, 3, 4, 5, 6, 7, 8, 9, 10, 11, 12, 13, 14, 15 or more) monomer, including a nucleoside or nucleotide, having a modified sugar moiety. For example, the furanosyl sugar ring of a nucleoside can be modified in a number of ways including, but not limited to, addition of a substituent group, bridging of two non-geminal ring atoms to form a locked nucleic acid or bicyclic nucleic acid. In certain embodiments, oligomeric compounds comprise one or more (e.g., 1, 2, 3, 4, 5, 6, 7, 8, 9, 10, 11, 12, 13, 14, 15 or more) monomers that are LNA.

In some embodiments of a locked nucleic acid, the 2′ position of furanosyl is connected to the 4′ position by a linker selected independently from —[C(R1)(R2)]ₙ-, —[C(R1)(R2)]ₙ-O—, —[C(R1)(R2)]ₙ-N(R1)-, —[C(R1)(R2)]ₙ-N(R1)-O—, —[C(R1R2)]ₙ-O—N(R1)-, —C(R1)=C(R2)-O—, —C(R1)=N—, —C(R1)=N—O—, —C(═NR1)-, —C(═NR1)-O—, —C(═O)—, —C(═O)O—, —C(═S)—, —C(═S)O—, —C(═S)S—, —O—, —Si(R1)2-, —S(═O)ₓ— and —N(R1)-;
wherein:
x is 0, 1, or 2;
n is 1, 2, 3, or 4;
each R1 and R2 is, independently, H, a protecting group, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJ1, NJ1J2, SJ1, N3, COOJ1, acyl (C(═O)—H), substituted acyl, CN, sulfonyl (S(═O)2-J1), or sulfoxyl (S(═O)-J1); and
each J1 and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted C1-C12 aminoalkyl or a protecting group.

In some embodiments, each of the linkers of the LNA compounds is, independently, —[C(R1)(R2)]n-, —[C(R1)(R2)]n-O—, —C(R1R2)-N(R1)-O— or —C(R1R2)-O—N(R1)-. In another embodiment, each of said linkers is, independently, 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R1)-2′ and 4′-CH₂—N(R1)-O-2′- wherein each R1 is, independently, H, a protecting group or C1-C12 alkyl.

Certain LNA's have been prepared and disclosed in the patent literature as well as in scientific literature (Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; WO 94/14226; WO 2005/021570; Singh et al., J. Org. Chem., 1998, 63, 10035-10039; Examples of issued US patents and published applications that disclose LNA s include, for example, U.S. Pat. Nos. 7,053,207; 6,268,490; 6,770,748; 6,794,499; 7,034,133; and 6,525,191; and U.S. Pre-Grant Publication Nos. 2004-0171570; 2004-0219565; 2004-0014959; 2003-0207841; 2004-0143114; and 20030082807.

Also provided herein are LNAs in which the 2′-hydroxyl group of the ribosyl sugar ring is linked to the 4′ carbon atom of the sugar ring thereby forming a methyleneoxy (4′-CH₂—O-2′) linkage to form the bicyclic sugar moiety (reviewed in Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; see also U.S. Pat. Nos. 6,268,490 and 6,670,461). The linkage can be a methylene (—CH₂—) group bridging the 2′ oxygen atom and the 4′ carbon atom, for which the term methyleneoxy (4′-CH₂-0-2′) LNA is used for the bicyclic moiety; in the case of an ethylene group in this position, the term ethyleneoxy (4′-CH₂CH₂—O-2′) LNA is used (Singh et al., Chem. Commun., 1998, 4, 455-456: Morita et al., Bioorganic Medicinal Chemistry, 2003, 11, 2211-2226). Methyleneoxy (4′-CH₂—O-2′) LNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides comprising BNAs have been described (Wahlestedt et al., Proc. Natl. Acad. Sci. U.S.A., 2000, 97, 5633-5638).

An isomer of methyleneoxy (4′-CH₂—O-2′) LNA that has also been discussed is alpha-L-methyleneoxy (4′-CH₂—O-2′) LNA which has been shown to have superior stability against a 3′-exonuclease. The alpha-L-methyleneoxy (4′-CH₂—O-2′) LNA's were incorporated into antisense gapmers and chimeras that showed potent antisense activity (Frieden et al., Nucleic Acids Research, 2003, 21, 6365-6372).

The synthesis and preparation of the methyleneoxy (4′-CH₂—O-2′) LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) LNA, phosphorothioate-methyleneoxy (4′-CH₂—O-2′) LNA and 2′-thio-LNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-LNA, a novel conformationally restricted high-affinity oligonucleotide analog has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported.

Modified sugar moieties are well known and can be used to alter, typically increase, the affinity of the antisense compound for its target and/or increase nuclease resistance. A representative list of preferred modified sugars includes but is not limited to bicyclic modified sugars, including methyleneoxy (4′-CH₂—O-2′) LNA and ethyleneoxy (4′-(CH₂)₂—O-2′ bridge) ENA; substituted sugars, especially 2′-substituted sugars having a 2′-F, 2′-OCH₃ or a 2′-O(CH₂)₂—OCH₃ substituent group; and 4′-thio modified sugars. Sugars can also be replaced with sugar mimetic groups among others.

Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; and 6,600,032; and WO 2005/121371.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR, n=1-50; “locked” nucleic acids (LNA) in which the furanose portion of the nucleoside includes a bridge connecting two carbon atoms on the furanose ring, thereby forming a bicyclic ring system; O-AMINE or O—(CH₂)ₙAMINE (n=1-10, AMINE=NH₂; alkylamino, dialkylamino, heterocyclyl, arylamino, diaryl amino, heteroaryl amino, diheteroaryl amino, ethylene diamine or polyamino); and O—CH₂CH₂(NCH₂CH₂NMe₂)₂.

“Deoxy” modifications include hydrogen (i.e. deoxyribose sugars, which are of particular relevance to the single-strand overhangs); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino); —NHC(O)R (R=alkyl, cycloalkyl, aryl, aralkyl, heteroaryl or sugar); cyano; mercapto; alkyl-thio-alkyl; thioalkoxy; thioalkyl; alkyl; cycloalkyl; aryl; alkenyl and alkynyl, which can be optionally substituted with e.g., an amino functionality.

Other suitable 2′-modifications, e.g., modified MOE, are described in U.S. Patent Application Publication No. 20130130378, contents of which are herein incorporated by reference.

A modification at the 2′ position can be present in the arabinose configuration The term “arabinose configuration” refers to the placement of a substituent on the C2′ of ribose in the same configuration as the 2′-OH is in the arabinose.

The sugar can comprise two different modifications at the same carbon in the sugar, e.g., gem modification. The sugar group can also contain one or more carbons that possess the opposite stereochemical configuration than that of the corresponding carbon in ribose. Thus, an oligomeric compound can include one or more monomers containing e.g., arabinose, as the sugar. The monomer can have an alpha linkage at the 1′ position on the sugar, e.g., alpha-nucleosides. The monomer can also have the opposite configuration at the 4′-position, e.g., C5′ and H4′ or substituents replacing them are interchanged with each other. When the C5′ and H4′ or substituents replacing them are interchanged with each other, the sugar is said to be modified at the 4′ position.

Double-stranded iRNA agent of the inventions disclosed herein can also include abasic sugars, i.e., a sugar which lack a nucleobase at C-1′ or has other chemical groups in place of a nucleobase at C1′. See for example U.S. Pat. No. 5,998,203, content of which is herein incorporated in its entirety. These abasic sugars can also be further containing modifications at one or more of the constituent sugar atoms. Double-stranded iRNA agent of the inventions can also contain one or more sugars that are the L isomer, e.g. L-nucleosides. Modification to the sugar group can also include replacement of the 4′-O with a sulfur, optionally substituted nitrogen or CH₂ group. In some embodiments, linkage between C1′ and nucleobase is in a configuration.

Sugar modifications can also include acyclic nucleotides, wherein a C—C bonds between ribose carbons (e.g., C1′-C2′, C2′-C3′, C3′-C4′, C4′-O4′, C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

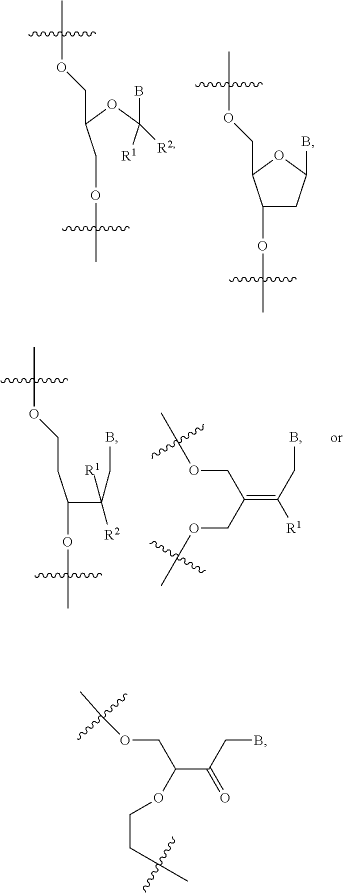

wherein B is a modified or unmodified nucleobase, R₁ and R₂ independently are H, halogen, OR₃, or alkyl; and R₃ is H, alkyl, cycloalkyl, aryl, aralkyl, heteroaryl or sugar).

In some embodiments, sugar modifications are selected from the group consisting of 2′-H, 2′-O-Me (2′-O-methyl), 2′-O-MOE (2′-O-methoxyethyl), 2′-F, 2′-O—[2-(methylamino)-2-oxoethyl](2′-O-NMA), 2′-S-methyl, 2′-O—CH₂-(4′-C) (LNA), 2′-O—CH₂CH₂-(4′-C) (ENA), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE) and gem 2′-OMe/2′F with 2′-O-Me in the arabinose configuration.

It is to be understood that when a particular nucleotide is linked through its 2′-position to the next nucleotide, the sugar modifications described herein can be placed at the 3′-position of the sugar for that particular nucleotide, e.g., the nucleotide that is linked through its 2′-position. A modification at the 3′ position can be present in the xylose configuration The term “xylose configuration” refers to the placement of a substituent on the C3′ of ribose in the same configuration as the 3′-OH is in the xylose sugar.

The hydrogen attached to C4′ and/or C1′ can be replaced by a straight- or branched-optionally substituted alkyl, optionally substituted alkenyl, optionally substituted alkynyl, wherein backbone of the alkyl, alkenyl and alkynyl can contain one or more of 0, S, S(O), SO₂, N(R′), C(O), N(R′)C(O)O, OC(O)N(R′), CH(Z′), phosphorous containing linkage, optionally substituted aryl, optionally substituted heteroaryl, optionally substituted heterocyclic or optionally substituted cycloalkyl, where R′ is hydrogen, acyl or optionally substituted aliphatic, Z′ is selected from the group consisting of OR₁₁, COR₁₁, CO₂R₁₁,

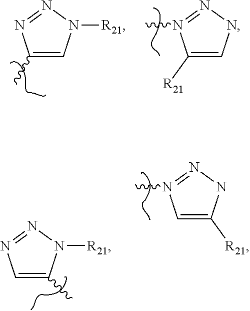

NR₂₁R₃₁, CONR₂₁R₃₁, CON(H)NR₂₁R₃₁, ONR₂₁R₃₁, CON(H)N═CR₄₁R₅₁, N(R₂₁)C(═NR₃₁)NR₂₁R₃₁, N(R₂₁)C(O)NR₂₁R₃₁, N(R₂₁)C(S)NR₂₁R₃₁, OC(O)NR₂₁R₃₁, SC(O)NR₂₁R₃₁, N(R₂₁)C(S)OR₁₁, N(R₂₁)C(O)OR₁₁, N(R₂₁)C(O)SR₁₁, N(R₂₁)N═CR₄₁R₅₁, ON═CR₄₁R₅₁, SO₂R₁₁, SOR₁₁, SR₁₁, and substituted or unsubstituted heterocyclic; R₂₁ and R₃₁ for each occurrence are independently hydrogen, acyl, unsubstituted or substituted aliphatic, aryl, heteroaryl, heterocyclic, OR₁₁, COR₁₁, CO₂R₁₁, or NR₁₁R₁₁′; or R₂₁ and R₃₁, taken together with the atoms to which they are attached, form a heterocyclic ring; R₄₁ and R₅₁ for each occurrence are independently hydrogen, acyl, unsubstituted or substituted aliphatic, aryl, heteroaryl, heterocyclic, OR₁₁, COR₁, or CO₂R₁₁, or NR₁₁R₁₁′; and R₁₁ and R₁₁′ are independently hydrogen, aliphatic, substituted aliphatic, aryl, heteroaryl, or heterocyclic. In some embodiments, the hydrogen attached to the C4′ of the 5′ terminal nucleotide is replaced.

In some embodiments, C4′ and C5′ together form an optionally substituted heterocyclic, preferably comprising at least one —PX(Y)—, wherein X is H, OH, OM, SH, optionally substituted alkyl, optionally substituted alkoxy, optionally substituted alkylthio, optionally substituted alkylamino or optionally substituted dialkylamino, where M is independently for each occurrence an alki metal or transition metal with an overall charge of +1; and Y is O, S, or NR′, where R′ is hydrogen, optionally substituted aliphatic. Preferably this modification is at the 5′ terminal of the iRNA.

In certain embodiments, LNA's include bicyclic nucleoside having the formula:

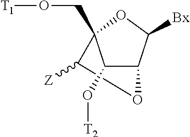

wherein:
Bx is a heterocyclic base moiety;
T₁ is H or a hydroxyl protecting group;
T₂ is H, a hydroxyl protecting group or a reactive phosphorus group;
Z is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, or substituted amide.

In some embodiments, each of the substituted groups, is, independently, mono or poly substituted with optionally protected substituent groups independently selected from halogen, oxo, hydroxyl, OJ1, NJ1J2, SJ1, N3, OC(═X)J1, OC(═X)NJ1J2, NJ3C(═X)NJ1J2 and CN, wherein each J1, J2 and J3 is, independently, H or C₁-C₆ alkyl, and X is O, S or NJ1.

In certain such embodiments, each of the substituted groups, is, independently, mono or poly substituted with substituent groups independently selected from halogen, oxo, hydroxyl, OJ1, NJ1J2, SJ1, N3, OC(═X)J1, and NJ3C(═X)NJ1J2, wherein each J1, J2 and J3 is, independently, H, C₁-C₆ alkyl, or substituted C₁-C₆ alkyl and X is O or NJ1.

In certain embodiments, the Z group is C₁-C₆ alkyl substituted with one or more Xx, wherein each Xx is independently OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is O, S or NJ1. In another embodiment, the Z group is C₁-C₆ alkyl substituted with one or more Xx, wherein each Xx is independently halo (e.g., fluoro), hydroxyl, alkoxy (e.g., CH₃O—), substituted alkoxy or azido.

In certain embodiments, the Z group is —CH₂Xx, wherein Xx is OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is O, S or NJ1. In another embodiment, the Z group is —CH₂Xx, wherein Xx is halo (e.g., fluoro), hydroxyl, alkoxy (e.g., CH₃O—) or azido.

In certain such embodiments, the Z group is in the (R)-configuration:

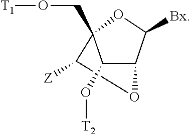

In certain such embodiments, the Z group is in the S)-configuration:

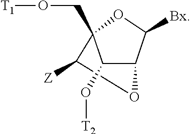

In certain embodiments, each T₁ and T₂ is a hydroxyl protecting group. A preferred list of hydroxyl protecting groups includes benzyl, benzoyl, 2,6-dichlorobenzyl, t-butyldimethylsilyl, t-butyldiphenylsilyl, mesylate, tosylate, dimethoxytrityl (DMT), 9-phenylxanthine-9-yl (Pixyl) and 9-(p-methoxyphenyl)xanthine-9-yl (MOX). In certain embodiments, T₁ is a hydroxyl protecting group selected from acetyl, benzyl, t-butyldimethylsilyl, t-butyldiphenylsilyl and dimethoxytrityl wherein a more preferred hydroxyl protecting group is T₁ is 4,4′-dimethoxytrityl.

In certain embodiments, T₂ is a reactive phosphorus group wherein preferred reactive phosphorus groups include diisopropylcyanoethoxy phosphoramidite and H-phosphonate. In certain embodiments T₁ is 4,4′-dimethoxytrityl and T₂ is diisopropylcyanoethoxy phosphoramidite.

In certain embodiments, the compounds of the invention comprise at least one monomer of the formula:

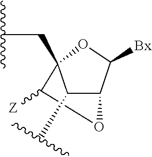

or of the formula:

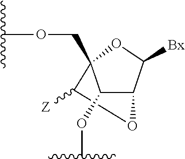

or of the formula:

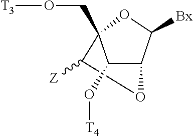

wherein
Bx is a heterocyclic base moiety;
T₃ is H, a hydroxyl protecting group, a linked conjugate group or an internucleoside linking group attached to a nucleoside, a nucleotide, an oligonucleoside, an oligonucleotide, a monomeric subunit or an oligomeric compound;
T₄ is H, a hydroxyl protecting group, a linked conjugate group or an internucleoside linking group attached to a nucleoside, a nucleotide, an oligonucleoside, an oligonucleotide, a monomeric subunit or an oligomeric compound;
wherein at least one of T₃ and T₄ is an internucleoside linking group attached to a nucleoside, a nucleotide, an oligonucleoside, an oligonucleotide, a monomeric subunit or an oligomeric compound; and
Z is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, or substituted amide.

In some embodiments, each of the substituted groups, is, independently, mono or poly substituted with optionally protected substituent groups independently selected from halogen, oxo, hydroxyl, OJ1, NJ1J2, SJ1, N3, OC(═X)J1, OC(═X)NJ1J2, NJ3C(═X)NJ1J2 and CN, wherein each J1, J2 and J3 is, independently, H or C₁-C₆ alkyl, and X is O, S or NJ1.

In some embodiments, each of the substituted groups, is, independently, mono or poly substituted with substituent groups independently selected from halogen, oxo, hydroxyl, OJ1, NJ1J2, SJ1, N3, OC(═X)J1, and NJ3C(═X)NJ1J2, wherein each J1, J2 and J3 is, independently, H or C₁-C₆ alkyl, and X is O or NJ1.

In certain such embodiments, at least one Z is C₁-C₆ alkyl or substituted C₁-C₆ alkyl. In certain embodiments, each Z is, independently, C₁-C₆ alkyl or substituted C₁-C₆ alkyl. In certain embodiments, at least one Z is C₁-C₆ alkyl. In certain embodiments, each Z is, independently, C₁-C₆ alkyl. In certain embodiments, at least one Z is methyl. In certain embodiments, each Z is methyl. In certain embodiments, at least one Z is ethyl. In certain embodiments, each Z is ethyl. In certain embodiments, at least one Z is substituted C₁-C₆ alkyl. In certain embodiments, each Z is, independently, substituted C₁-C₆ alkyl. In certain embodiments, at least one Z is substituted methyl. In certain embodiments, each Z is substituted methyl. In certain embodiments, at least one Z is substituted ethyl. In certain embodiments, each Z is substituted ethyl.

In certain embodiments, at least one substituent group is C₁-C₆ alkoxy (e.g., at least one Z is C₁-C₆ alkyl substituted with one or more C₁-C₆ alkoxy). In another embodiment, each substituent group is, independently, C₁-C₆ alkoxy (e.g., each Z is, independently, C₁-C₆ alkyl substituted with one or more C₁-C₆ alkoxy).

In certain embodiments, at least one C₁-C₆ alkoxy substituent group is CH₃O— (e.g., at least one Z is CH₃OCH₂—). In another embodiment, each C₁-C₆ alkoxy substituent group is CH₃O— (e.g., each Z is CH₃OCH₂—).

In certain embodiments, at least one substituent group is halogen (e.g., at least one Z is C₁-C₆ alkyl substituted with one or more halogen). In certain embodiments, each substituent group is, independently, halogen (e.g., each Z is, independently, C₁-C₆ alkyl substituted with one or more halogen). In certain embodiments, at least one halogen substituent group is fluoro (e.g., at least one Z is CH₂FCH₂—, CHF₂CH₂— or CF₃CH₂—). In certain embodiments, each halo substituent group is fluoro (e.g., each Z is, independently, CH₂FCH₂—, CHF₂CH₂— or CF₃CH₂—).

In certain embodiments, at least one substituent group is hydroxyl (e.g., at least one Z is C1-C6 alkyl substituted with one or more hydroxyl). In certain embodiments, each substituent group is, independently, hydroxyl (e.g., each Z is, independently, C₁-C₆ alkyl substituted with one or more hydroxyl). In certain embodiments, at least one Z is HOCH₂—. In another embodiment, each Z is HOCH₂—.

In certain embodiments, at least one Z is CH₃—, CH₃CH₂—, CH₂OCH₃—, CH₂F— or HOCH₂—. In certain embodiments, each Z is, independently, CH₃—, CH₃CH₂—, CH₂OCH₃—, CH₂F— or HOCH₂—.

In certain embodiments, at least one Z group is C₁-C₆ alkyl substituted with one or more Xx, wherein each Xx is, independently, OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is O, S or NJ1. In another embodiment, at least one Z group is C₁-C₆ alkyl substituted with one or more Xx, wherein each Xx is, independently, halo (e.g., fluoro), hydroxyl, alkoxy (e.g., CH₃O—) or azido.

In certain embodiments, each Z group is, independently, C₁-C₆ alkyl substituted with one or more Xx, wherein each Xx is independently OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is O, S or NJ1. In another embodiment, each Z group is, independently, C₁-C₆ alkyl substituted with one or more Xx, wherein each Xx is independently halo (e.g., fluoro), hydroxyl, alkoxy (e.g., CH₃O—) or azido.

In certain embodiments, at least one Z group is —CH₂Xx, wherein Xx is OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is O, S or NJ1 In certain embodiments, at least one Z group is —CH₂Xx, wherein Xx is halo (e.g., fluoro), hydroxyl, alkoxy (e.g., CH₃O—) or azido.

In certain embodiments, each Z group is, independently, —CH₂Xx, wherein each Xx is, independently, OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is O, S or NJ1. In another embodiment, each Z group is, independently, —CH₂Xx, wherein each Xx is, independently, halo (e.g., fluoro), hydroxyl, alkoxy (e.g., CH₃O—) or azido.

In certain embodiments, at least one Z is CH₃—. In another embodiment, each Z is, CH₃—.

In certain embodiments, the Z group of at least one monomer is in the (R)-configuration represented by the formula:

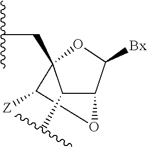

or the formula:

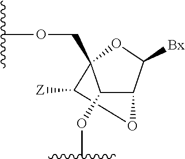

or the formula:

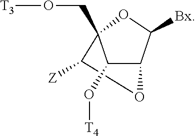

In certain embodiments, the Z group of each monomer of the formula is in the (R)-configuration.

In certain embodiments, the Z group of at least one monomer is in the (S)-configuration represented by the formula:

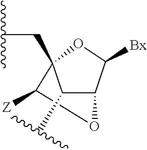

or the formula:

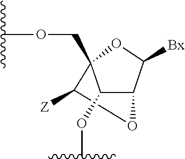

or the formula:

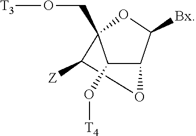

In certain embodiments, the Z group of each monomer of the formula is in the (S)-configuration.

In certain embodiments, T₃ is H or a hydroxyl protecting group. In certain embodiments, T₄ is H or a hydroxyl protecting group. In a further embodiment T₃ is an internucleoside linking group attached to a nucleoside, a nucleotide or a monomeric subunit. In certain embodiments, T₄ is an internucleoside linking group attached to a nucleoside, a nucleotide or a monomeric subunit. In certain embodiments, T₃ is an internucleoside linking group attached to an oligonucleoside or an oligonucleotide. In certain embodiments, T₄ is an internucleoside linking group attached to an oligonucleoside or an oligonucleotide. In certain embodiments, T₃ is an internucleoside linking group attached to an oligomeric compound. In certain embodiments, T₄ is an internucleoside linking group attached to an oligomeric compound. In certain embodiments, at least one of T₃ and T₄ comprises an internucleoside linking group selected from phosphodiester or phosphorothioate.

In certain embodiments, double-stranded iRNA agent of the invention comprise at least one region of at least two contiguous monomers of the formula:

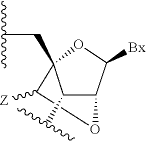

or of the formula:

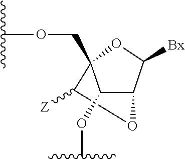

or of the formula:

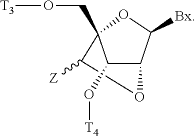

In certain such embodiments, LNAs include, but are not limited to, (A) α-L-Methyleneoxy (4′-CH₂—O-2′) LNA, (B) β-D-Methyleneoxy (4′-CH₂—O-2′) LNA, (C) Ethyleneoxy (4′-(CH₂)₂—O-2′) LNA, (D) Aminooxy (4′-CH₂—O—N(R)-2′) LNA and (E) Oxyamino (4′-CH₂—N(R)—O-2′) LNA, as depicted below:

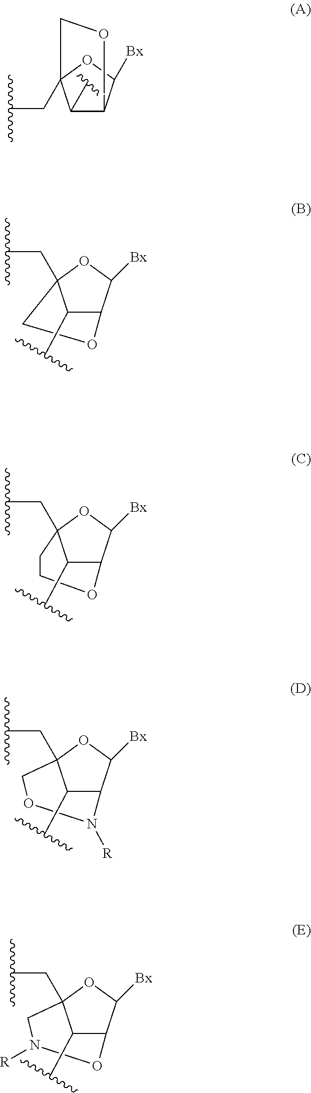

In certain embodiments, the double-stranded iRNA agent of the invention comprises at least two regions of at least two contiguous monomers of the above formula. In certain embodiments, the double-stranded iRNA agent of the invention comprises a gapped motif. In certain embodiments, the double-stranded iRNA agent of the invention comprises at least one region of from about 8 to about 14 contiguous β-D-2′-deoxyribofuranosyl nucleosides. In certain embodiments, the Double-stranded iRNA agent of the invention comprises at least one region of from about 9 to about 12 contiguous β-D-2′-deoxyribofuranosyl nucleosides.

In certain embodiments, the double-stranded iRNA agent of the invention comprises at least one (e.g., 1, 2, 3, 4, 5, 6, 7, 8, 9, 10, 11, 12, 13, 14, 15 or more) comprises at least one (S)-cEt monomer of the formula:

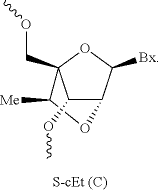

wherein Bx IS heterocyclic base moiety.

In certain embodiments, monomers include sugar mimetics. In certain such embodiments, a mimetic is used in place of the sugar or sugar-internucleoside linkage combination, and the nucleobase is maintained for hybridization to a selected target. Representative examples of a sugar mimetics include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside and nucleobase mimetics are well known to those skilled in the art.

Described herein are linking groups that link monomers (including, but not limited to, modified and unmodified nucleosides and nucleotides) together, thereby forming an oligomeric compound, e.g., an oligonucleotide. Such linking groups are also referred to as intersugar linkage. The two main classes of linking groups are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S).

Representative non-phosphorus containing linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligonucleotides. In certain embodiments, linkages having a chiral atom can be prepared as racemic mixtures, as separate enantiomers. Representative chiral linkages include, but are not limited to, alkylphosphonates and phosphorothioates. Methods of preparation of phosphorous-containing and non-phosphorous-containing linkages are well known to those skilled in the art.

The phosphate group in the linking group can be modified by replacing one of the oxygens with a different substituent. One result of this modification can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the linkage can be replaced by any of the following: S, Se, BR₃ (R is hydrogen, alkyl, aryl), C (i.e. an alkyl group, an aryl group, etc. . . . ), H, NR₂ (R is hydrogen, optionally substituted alkyl, aryl), or OR (R is optionally substituted alkyl or aryl). The phosphorous atom in an unmodified phosphate group is achiral. However, replacement of one of the non-bridging oxygens with one of the above atoms or groups of atoms renders the phosphorous atom chiral; in other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. Thus, the non-bridging oxygens can be independently any one of O, S, Se, B, C, H, N, or OR (R is alkyl or aryl).

The phosphate linker can also be modified by replacement of bridging oxygen, (i.e. oxygen that links the phosphate to the sugar of the monomer), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

In certain embodiments, the phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H)—C(═O)-5′)), hydroxylamino, siloxane (dialkylsiloxxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′-O—P(O)—O—SS—C5′, C3′-CH₂—NH—NH—C5′, 3′-NHP(O)(OCH₃)—O-5′ and 3′-NHP(O)(OCH₃)—O-5′ and nonionic linkages containing mixed N, O, S and CH₂ component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester intersugar linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phsophotriesters, aminoalkylphosphotrioesters, alkyl-phosphonaters (e.g., methyl-phosphonate), selenophosphates, phosphoramidates (e.g., N-alkylphosphoramidate), and boranophosphonates.

In some embodiments, the double-stranded iRNA agent of the invention comprises at least one (e.g., 1, 2, 3, 4, 5, 6, 7, 8, 9, 10, 11, 12, 13, 14, 15 or more and up to including all) modified or nonphosphodiester linkages. In some embodiments, the double-stranded iRNA agent of the invention comprises at least one (e.g., 1, 2, 3, 4, 5, 6, 7, 8, 9, 10, 11, 12, 13, 14, 15 or more and up to including all) phosphorothioate linkages.

The double-stranded iRNA agent of the inventions can also be constructed wherein the phosphate linker and the sugar are replaced by nuclease resistant nucleoside or nucleotide surrogates. While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone. Examples include the morpholino, cyclobutyl, pyrrolidine, peptide nucleic acid (PNA), aminoethylglycyl PNA (aegPNA) and backnone-extended pyrrolidine PNA (bepPNA) nucleoside surrogates. A preferred surrogate is a PNA surrogate.

The double-stranded iRNA agent of the inventions described herein can contain one or more asymmetric centers and thus give rise to enantiomers, diastereomers, and other stereoisomeric configurations that may be defined, in terms of absolute stereochemistry, as (R) or (S), such as for sugar anomers, or as (D) or (L) such as for amino acids et al. Included in the double-stranded iRNA agent of the inventions provided herein are all such possible isomers, as well as their racemic and optically pure forms.

In some embodiments, the double-stranded iRNA agent further comprises a phosphate or phosphate mimic at the 5′-end of the antisense strand. In one embodiment, the phosphate mimic is a 5′-vinyl phosphonate (VP).

In some embodiments, the 5′-end of the antisense strand of the double-stranded iRNA agent does not contain a 5′-vinyl phosphonate (VP).

Ends of the iRNA agent of the invention can be modified. Such modifications can be at one end or both ends. For example, the 3′ and/or 5′ ends of an iRNA can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs).

When a linker/phosphate-functional molecular entity-linker/phosphate array is interposed between two strands of a double stranded oligomeric compound, this array can substitute for a hairpin loop in a hairpin-type oligomeric compound.

Terminal modifications useful for modulating activity include modification of the 5′ end of iRNAs with phosphate or phosphate analogs. In certain embodiments, the 5′end of an iRNA is phosphorylated or includes a phosphoryl analog. Exemplary 5′-phosphate modifications include those which are compatible with RISC mediated gene silencing. Modifications at the 5′-terminal end can also be useful in stimulating or inhibiting the immune system of a subject. In some embodiments, the 5′-end of the oligomeric compound comprises the modification

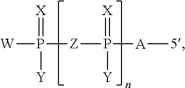

wherein W, X and Y are each independently selected from the group consisting of O, OR (R is hydrogen, alkyl, aryl), S, Se, BR₃ (R is hydrogen, alkyl, aryl), BH₃, C (i.e. an alkyl group, an aryl group, etc.,), H, NR₂ (R is hydrogen, alkyl, aryl), or OR (R is hydrogen, alkyl or aryl); A and Z are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and n is 0-2. In some embodiments, n is 1 or 2. It is understood that A is replacing the oxygen linked to 5′ carbon of sugar. When n is O, W and Y together with the P to which they are attached can form an optionally substituted 5-8 membered heterocyclic, wherein W an Y are each independently O, S, NR′ or alkylene. Preferably the heterocyclic is substituted with an aryl or heteroaryl. In some embodiments, one or both hydrogen on C5′ of the 5′-terminal nucleotides are replaced with a halogen, e.g., F.

Exemplary 5′-modifications include, but are not limited to, 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-monothiophosphate (phosphorothioate; (HO)2(S)P—O-5′); 5′-monodithiophosphate (phosphorodithioate; (HO)(HS)(S)P—O-5′), 5′-phosphorothiolate ((HO)2(O)P—S-5′); 5′-alpha-thiotriphosphate; 5′-beta-thiotriphosphate; 5′-gamma-thiotriphosphate; 5′-phosphoramidates ((HO)₂(O)P—NH-5′, (HO)(NH₂)(O)P—O-5′). Other 5′-modification include 5′-alkylphosphonates (R(OH)(O)P—O-5′, R=alkyl, e.g., methyl, ethyl, isopropyl, propyl, etc.,), 5-alkyletherphosphonates (R(OH)(O)P—O-5′, R=alkylether, e.g., methoxymethyl (CH₂OMe), ethoxymethyl, etc. . . . ). Other exemplary 5′-modifications include where Z is optionally substituted alkyl at least once, e.g., ((HO)₂(X)P—O[—(CH₂)ₐ—O—P(X)(OH)—O]_{b-}5′, ((HO)₂(X)P—O[—(CH₂)ₐ—P(X)(OH)—O]_{b}-5′, ((HO)2(X)P—[—(CH₂)ₐ—O—P(X)(OH)—O]_{b}-5′; dialkyl terminal phosphates and phosphate mimics: HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10. Other embodiments include replacement of oxygen and/or sulfur with BH₃, BH₃⁻ and/or Se.

Terminal modifications can also be useful for monitoring distribution, and in such cases the preferred groups to be added include fluorophores, e.g., fluorescein or an Alexa dye, e.g., Alexa 488. Terminal modifications can also be useful for enhancing uptake, useful modifications for this include targeting ligands. Terminal modifications can also be useful for cross-linking an oligonucleotide to another moiety; modifications useful for this include mitomycin C, psoralen, and derivatives thereof.

The compounds of the invention, such as iRNAs or dsRNA agents, can be optimized for RNA interference by increasing the propensity of the iRNA duplex to disassociate or melt (decreasing the free energy of duplex association) by introducing a thermally destabilizing modification in the sense strand at a site opposite to the seed region of the antisense strand (i.e., at positions 2-8 of the 5′-end of the antisense strand). This modification can increase the propensity of the duplex to disassociate or melt in the seed region of the antisense strand.

The thermally destabilizing modifications can include abasic modification; mismatch with the opposing nucleotide in the opposing strand; and sugar modification such as 2′-deoxy modification or acyclic nucleotide, e.g., unlocked nucleic acids (UNA) or glycerol nuceltic acid (GNA).

Exemplified abasic modifications are:

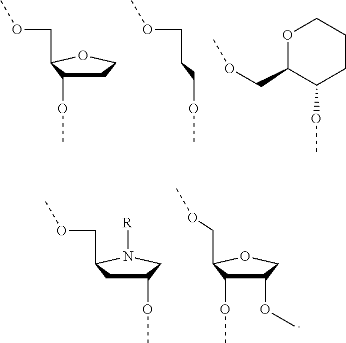

Exemplified sugar modifications are:

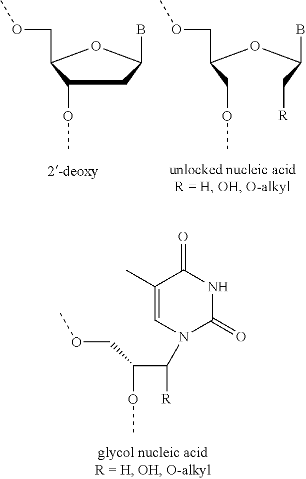

The term “acyclic nucleotide” refers to any nucleotide having an acyclic ribose sugar, for example, where any of bonds between the ribose carbons (e.g., C1′-C2′, C2′-C3′, C3′-C4′, C4′-O4′, or C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

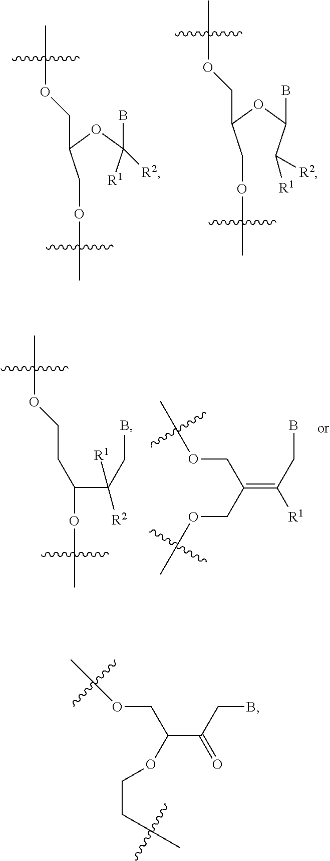

wherein B is a modified or unmodified nucleobase, R¹ and R² independently are H, halogen, OR₃, or alkyl; and R₃ is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

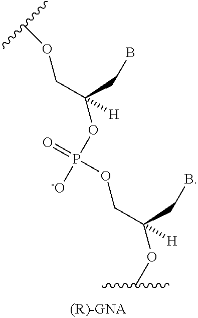

The thermally destabilizing modification can be mismatches (i.e., noncomplementary base pairs) between the thermally destabilizing nucleotide and the opposing nucleotide in the opposite strand within the dsRNA duplex. Exemplary mismatch basepairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the compounds of the invention, such as siRNA or iRNA agent, contains at least one nucleobase in the mismatch pairing that is a 2′-deoxy nucleobase; e.g., the 2′-deoxy nucleobase is in the sense strand.

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

Nucleobase modifications with impaired or completely abolished capability to form hydrogen bonds with bases in the opposite strand have been evaluated for destabilization of the central region of the dsRNA duplex as described in WO 2010/0011895, which is herein incorporated by reference in its entirety. Exemplary nucleobase modifications are:

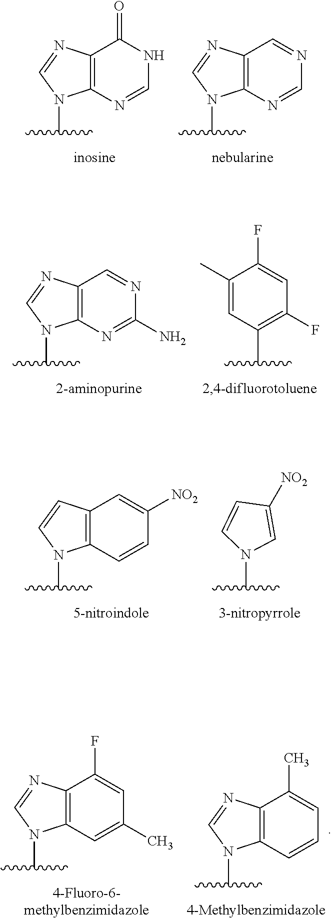

Exemplary phosphate modifications known to decrease the thermal stability of dsRNA duplexes compared to natural phosphodiester linkages are:

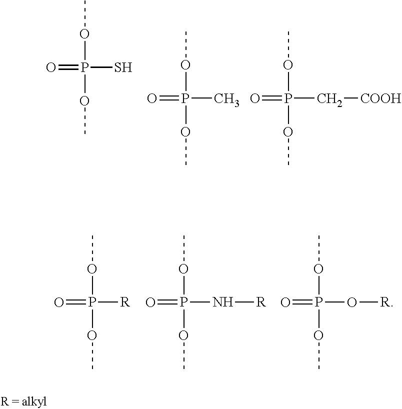

In some embodiments, compounds of the invention can comprise 2′-5′ linkages (with 2′-H, 2′-OH and 2′-OMe and with P═O or P═S). For example, the 2′-5′ linkages modifications can be used to promote nuclease resistance or to inhibit binding of the sense to the antisense strand, or can be used at the 5′ end of the sense strand to avoid sense strand activation by RISC.

In another embodiment, compounds of the invention can comprise L sugars (e.g., L ribose, L-arabinose with 2′-H, 2′-OH and 2′-OMe). For example, these L sugar modifications can be used to promote nuclease resistance or to inhibit binding of the sense to the antisense strand, or can be used at the 5′ end of the sense strand to avoid sense strand activation by RISC.

In one embodiment the iRNA agent of the invention is conjugated to a ligand via a carrier, wherein the carrier can be cyclic group or acyclic group; preferably, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and decalin; preferably, the acyclic group is selected from serinol backbone or diethanolamine backbone.

In some embodiments, at least one strand of the iRNA agent of the invention disclosed herein is 5′ phosphorylated or includes a phosphoryl analog at the 5′ prime terminus. 5′-phosphate modifications include those which are compatible with RISC mediated gene silencing. Suitable modifications include: 5′-monophosphate ((HO)2(O)P—O-5′); 5′-diphosphate ((HO)2(O)P—O—P(HO)(O)—O-5′); 5′-triphosphate ((HO)2(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)2(S)P—O-5′); 5′-monodithiophosphate (phosphorodithioate; (HO)(HS)(S)P—O-5′), 5′-phosphorothiolate ((HO)2(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)2(O)P—NH-5′, (HO)(NH2)(O)P—O-5′), 5′-alkylphosphonates (R=alkyl=methyl, ethyl, isopropyl, propyl, etc., e.g. RP(OH)(O)—O-5′-, 5′-alkenylphosphonates (i.e. vinyl, substituted vinyl), (OH)2(O)P-5′-CH₂—), 5′-alkyletherphosphonates (R=alkylether=methoxymethyl (MeOCH2-), ethoxymethyl, etc., e.g. RP(OH)(O)—O-5′-).

B. Modified iRNAs Comprising Motifs of the Invention

In certain aspects of the invention, the double stranded RNAi agents of the invention include agents with chemical modifications as disclosed, for example, in WO 2013/075035, filed on Nov. 16, 2012, the entire contents of which are incorporated herein by reference.

Accordingly, the invention provides double stranded RNAi agents capable of inhibiting the expression of a target gene (i.e., TTR) in an ocular cell in vivo. The RNAi agent comprises a sense strand and an antisense strand. Each strand of the RNAi agent may range from 12-30 nucleotides in length. For example, each strand may be between 14-30 nucleotides in length, 17-30 nucleotides in length, 25-30 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21 23 nucleotides in length.

The sense strand and antisense strand typically form a duplex double stranded RNA (“dsRNA”), also referred to herein as an “RNAi agent.” The duplex region of an RNAi agent may be 12-30 nucleotide pairs in length. For example, the duplex region can be between 14-30 nucleotide pairs in length, 17-30 nucleotide pairs in length, 27-30 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotides in length.

In one embodiment, the RNAi agent may contain one or more overhang regions and/or capping groups at the 3′-end, 5′-end, or both ends of one or both strands. The overhang can be 1-6 nucleotides in length, for instance 2 6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. The overhangs can be the result of one strand being longer than the other, or the result of two strands of the same length being staggered. The overhang can form a mismatch with the target mRNA or it can be complementary to the gene sequences being targeted or can be another sequence. The first and second strands can also be joined, e.g., by additional bases to form a hairpin, or by other non-base linkers.

In one embodiment, the nucleotides in the overhang region of the RNAi agent can each independently be a modified or unmodified nucleotide including, but no limited to 2′-sugar modified, such as, 2-F, 2′-Omethyl, thymidine (T), 2′-O-methoxyethyl-5-methyluridine (Teo), 2′-O-methoxyethyladenosine (Aeo), 2′-O-methoxyethyl-5-methylcytidine (m5Ceo), and any combinations thereof. For example, TT can be an overhang sequence for either end on either strand. The overhang can form a mismatch with the target mRNA or it can be complementary to the gene sequences being targeted or can be another sequence.

The 5′- or 3′-overhangs at the sense strand, antisense strand or both strands of the RNAi agent may be phosphorylated. In some embodiments, the overhang region(s) contains two nucleotides having a phosphorothioate between the two nucleotides, where the two nucleotides can be the same or different. In one embodiment, the overhang is present at the 3′-end of the sense strand, antisense strand, or both strands. In one embodiment, this 3′-overhang is present in the antisense strand. In one embodiment, this 3′-overhang is present in the sense strand.

The RNAi agent may contain only a single overhang, which can strengthen the interference activity of the RNAi, without affecting its overall stability. For example, the single-stranded overhang may be located at the 3-terminal end of the sense strand or, alternatively, at the 3-terminal end of the antisense strand. The RNAi may also have a blunt end, located at the 5′-end of the antisense strand (or the 3′-end of the sense strand) or vice versa. Generally, the antisense strand of the RNAi has a nucleotide overhang at the 3′-end, and the 5′-end is blunt. While not wishing to be bound by theory, the asymmetric blunt end at the 5′-end of the antisense strand and 3′-end overhang of the antisense strand favor the guide strand loading into RISC process.

In one embodiment, the RNAi agent is a double ended bluntmer of 19 nucleotides in length, wherein the sense strand contains at least one motif of three 2′-F modifications on three consecutive nucleotides at positions 7, 8, 9 from the 5′end. The antisense strand contains at least one motif of three 2′-O-methyl modifications on three consecutive nucleotides at positions 11, 12, 13 from the 5′end.

In another embodiment, the RNAi agent is a double ended bluntmer of 20 nucleotides in length, wherein the sense strand contains at least one motif of three 2′-F modifications on three consecutive nucleotides at positions 8, 9, 10 from the 5′end. The antisense strand contains at least one motif of three 2′-O-methyl modifications on three consecutive nucleotides at positions 11, 12, 13 from the 5′end.

In yet another embodiment, the RNAi agent is a double ended bluntmer of 21 nucleotides in length, wherein the sense strand contains at least one motif of three 2′-F modifications on three consecutive nucleotides at positions 9, 10, 11 from the 5′end. The antisense strand contains at least one motif of three 2′-O-methyl modifications on three consecutive nucleotides at positions 11, 12, 13 from the 5′end.

In one embodiment, the RNAi agent comprises a 21 nucleotide sense strand and a 23 nucleotide antisense strand, wherein the sense strand contains at least one motif of three 2′-F modifications on three consecutive nucleotides at positions 9, 10, 11 from the 5′end; the antisense strand contains at least one motif of three 2′-O-methyl modifications on three consecutive nucleotides at positions 11, 12, 13 from the 5′end, wherein one end of the RNAi agent is blunt, while the other end comprises a 2 nucleotide overhang. Preferably, the 2 nucleotide overhang is at the 3′-end of the antisense strand.

When the 2 nucleotide overhang is at the 3′-end of the antisense strand, there may be two phosphorothioate internucleotide linkages between the terminal three nucleotides, wherein two of the three nucleotides are the overhang nucleotides, and the third nucleotide is a paired nucleotide next to the overhang nucleotide. In one embodiment, the RNAi agent additionally has two phosphorothioate internucleotide linkages between the terminal three nucleotides at both the 5′-end of the sense strand and at the 5′-end of the antisense strand. In one embodiment, every nucleotide in the sense strand and the antisense strand of the RNAi agent, including the nucleotides that are part of the motifs are modified nucleotides. In one embodiment each residue is independently modified with a 2′-O-methyl or 3′-fluoro, e.g., in an alternating motif. Optionally, the RNAi agent further comprises a ligand (preferably GalNAc3).

In one embodiment, the RNAi agent comprises a sense and an antisense strand, wherein the sense strand is 25-30 nucleotide residues in length, wherein starting from the 5′ terminal nucleotide (position 1) positions 1 to 23 of the first strand comprise at least 8 ribonucleotides; the antisense strand is 36-66 nucleotide residues in length and, starting from the 3′ terminal nucleotide, comprises at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when the double stranded nucleic acid is introduced into a mammalian ocular cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In one embodiment, the RNAi agent comprises sense and antisense strands, wherein the RNAi agent comprises a first strand having a length which is at least 25 and at most 29 nucleotides and a second strand having a length which is at most 30 nucleotides with at least one motif of three 2′-O-methyl modifications on three consecutive nucleotides at position 11, 12, 13 from the 5′ end; wherein the 3′ end of the first strand and the 5′ end of the second strand form a blunt end and the second strand is 1-4 nucleotides longer at its 3′ end than the first strand, wherein the duplex region which is at least 25 nucleotides in length, and the second strand is sufficiently complementary to a target mRNA along at least 19 nucleotide of the second strand length to reduce target gene expression when the RNAi agent is introduced into a mammalian cell, and wherein dicer cleavage of the RNAi agent preferentially results in an siRNA comprising the 3′ end of the second strand, thereby reducing expression of the target gene in the mammal. Optionally, the RNAi agent further comprises a ligand.

In one embodiment, the sense strand of the RNAi agent contains at least one motif of three identical modifications on three consecutive nucleotides, where one of the motifs occurs at the cleavage site in the sense strand.

In one embodiment, the antisense strand of the RNAi agent can also contain at least one motif of three identical modifications on three consecutive nucleotides, where one of the motifs occurs at or near the cleavage site in the antisense strand.

For an RNAi agent having a duplex region of 17-23 nucleotide in length, the cleavage site of the antisense strand is typically around the 10, 11 and 12 positions from the 5′ end. Thus the motifs of three identical modifications may occur at the 9, 10, 11 positions; 10, 11, 12 positions; 11, 12, 13 positions; 12, 13, 14 positions; or 13, 14, 15 positions of the antisense strand, the count starting from the 1st nucleotide from the 5′ end of the antisense strand, or, the count starting from the 1st paired nucleotide within the duplex region from the 5′-end of the antisense strand. The cleavage site in the antisense strand may also change according to the length of the duplex region of the RNAi from the 5′-end.

The sense strand of the RNAi agent may contain at least one motif of three identical modifications on three consecutive nucleotides at the cleavage site of the strand; and the antisense strand may have at least one motif of three identical modifications on three consecutive nucleotides at or near the cleavage site of the strand. When the sense strand and the antisense strand form a dsRNA duplex, the sense strand and the antisense strand can be so aligned that one motif of the three nucleotides on the sense strand and one motif of the three nucleotides on the antisense strand have at least one nucleotide overlap, i.e., at least one of the three nucleotides of the motif in the sense strand forms a base pair with at least one of the three nucleotides of the motif in the antisense strand. Alternatively, at least two nucleotides may overlap, or all three nucleotides may overlap.

In one embodiment, the sense strand of the RNAi agent may contain more than one motif of three identical modifications on three consecutive nucleotides. The first motif may occur at or near the cleavage site of the strand and the other motifs may be a wing modification. The term “wing modification” herein refers to a motif occurring at another portion of the strand that is separated from the motif at or near the cleavage site of the same strand. The wing modification is either adjacent to the first motif or is separated by at least one or more nucleotides. When the motifs are immediately adjacent to each other then the chemistry of the motifs are distinct from each other and when the motifs are separated by one or more nucleotide than the chemistries can be the same or different. Two or more wing modifications may be present. For instance, when two wing modifications are present, each wing modification may occur at one end relative to the first motif which is at or near cleavage site or on either side of the lead motif.

Like the sense strand, the antisense strand of the RNAi agent may contain more than one motifs of three identical modifications on three consecutive nucleotides, with at least one of the motifs occurring at or near the cleavage site of the strand. This antisense strand may also contain one or more wing modifications in an alignment similar to the wing modifications that may be present on the sense strand.

In one embodiment, the wing modification on the sense strand or antisense strand of the RNAi agent typically does not include the first one or two terminal nucleotides at the 3′-end, 5′-end or both ends of the strand.

In another embodiment, the wing modification on the sense strand or antisense strand of the RNAi agent typically does not include the first one or two paired nucleotides within the duplex region at the 3′-end, 5′-end or both ends of the strand.

When the sense strand and the antisense strand of the RNAi agent each contain at least one wing modification, the wing modifications may fall on the same end of the duplex region, and have an overlap of one, two or three nucleotides.

When the sense strand and the antisense strand of the RNAi agent each contain at least two wing modifications, the sense strand and the antisense strand can be so aligned that two modifications each from one strand fall on one end of the duplex region, having an overlap of one, two or three nucleotides; two modifications each from one strand fall on the other end of the duplex region, having an overlap of one, two or three nucleotides; two modifications one strand fall on each side of the lead motif, having an overlap of one, two or three nucleotides in the duplex region.

In one embodiment, every nucleotide in the sense strand and antisense strand of the RNAi agent, including the nucleotides that are part of the motifs, may be modified. Each nucleotide may be modified with the same or different modification which can include one or more alteration of one or both of the non-linking phosphate oxygens and/or of one or more of the linking phosphate oxygens; alteration of a constituent of the ribose sugar, e.g., of the 21 hydroxyl on the ribose sugar; wholesale replacement of the phosphate moiety with “dephospho” linkers; modification or replacement of a naturally occurring base; and replacement or modification of the ribose-phosphate backbone.

As nucleic acids are polymers of subunits, many of the modifications occur at a position which is repeated within a nucleic acid, e.g., a modification of a base, or a phosphate moiety, or a non-linking O of a phosphate moiety. In some cases, the modification will occur at all of the subject positions in the nucleic acid but in many cases it will not. By way of example, a modification may only occur at a 3′ or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of a RNA or may only occur in a single strand region of a RNA. For example, a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′ end or ends can be phosphorylated.

It may be possible, e.g., to enhance stability, to include particular bases in overhangs, or to include modified nucleotides or nucleotide surrogates, in single strand overhangs, e.g., in a 5′ or 3′ overhang, or in both. For example, it can be desirable to include purine nucleotides in overhangs. In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In one embodiment, each residue of the sense strand and antisense strand is independently modified with LNA, CRN, cET, UNA, HNA, CeNA, 2′-methoxyethyl, 2′-O-methyl, 2′-O-allyl, 2′-C— allyl, 2′-deoxy, 2′-hydroxyl, or 2′-fluoro. The strands can contain more than one modification. In one embodiment, each residue of the sense strand and antisense strand is independently modified with 2′-O-methyl or 2′-fluoro.

At least two different modifications are typically present on the sense strand and antisense strand. Those two modifications may be the 2′-O-methyl or 2′-fluoro modifications, or others.

In one embodiment, the Na and/or Nb comprise modifications of an alternating pattern. The term “alternating motif” as used herein refers to a motif having one or more modifications, each modification occurring on alternating nucleotides of one strand. The alternating nucleotide may refer to one per every other nucleotide or one per every three nucleotides, or a similar pattern. For example, if A, B and C each represent one type of modification to the nucleotide, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “ACACAC . . . ” “BDBDBD . . . ” or “CDCDCD . . . ,” etc.

In one embodiment, the RNAi agent of the invention comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA duplex, the alternating motif in the sense strand may start with “ABABAB” from 5′ 3′ of the strand and the alternating motif in the antisense strand may start with “BABABA” from 5′-3′ of the strand within the duplex region. As another example, the alternating motif in the sense strand may start with “AABBAABB” from 5′ 3′ of the strand and the alternating motif in the antisense strand may start with “BBAABBAA” from 5′-3′ of the strand within the duplex region, so that there is a complete or partial shift of the modification patterns between the sense strand and the antisense strand.

In one embodiment, the RNAi agent comprises the pattern of the alternating motif of 2′-O-methyl modification and 2′-F modification on the sense strand initially has a shift relative to the pattern of the alternating motif of 2′-O-methyl modification and 2′-F modification on the antisense strand initially, i.e., the 2′-O-methyl modified nucleotide on the sense strand base pairs with a 2′-F modified nucleotide on the antisense strand and vice versa. The 1 position of the sense strand may start with the 2′-F modification, and the 1 position of the antisense strand may start with the 2′-O-methyl modification.

The introduction of one or more motifs of three identical modifications on three consecutive nucleotides to the sense strand and/or antisense strand interrupts the initial modification pattern present in the sense strand and/or antisense strand. This interruption of the modification pattern of the sense and/or antisense strand by introducing one or more motifs of three identical modifications on three consecutive nucleotides to the sense and/or antisense strand surprisingly enhances the gene silencing activity to the target gene.

In one embodiment, when the motif of three identical modifications on three consecutive nucleotides is introduced to any of the strands, the modification of the nucleotide next to the motif is a different modification than the modification of the motif. For example, the portion of the sequence containing the motif is “ . . . NaYYYNb . . . ,” where “Y” represents the modification of the motif of three identical modifications on three consecutive nucleotide, and “Na” and “Nb” represent a modification to the nucleotide next to the motif “YYY” that is different than the modification of Y, and where Na and Nb can be the same or different modifications. Alternatively, Na and/or Nb may be present or absent when there is a wing modification present.

The RNAi agent may further comprise at least one phosphorothioate or methylphosphonate internucleotide linkage. The phosphorothioate or methylphosphonate internucleotide linkage modification may occur on any nucleotide of the sense strand or antisense strand or both strands in any position of the strand. For instance, the internucleotide linkage modification may occur on every nucleotide on the sense strand and/or antisense strand; each internucleotide linkage modification may occur in an alternating pattern on the sense strand and/or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand. In one embodiment, a double-stranded RNAi agent comprises 6-8phosphorothioate internucleotide linkages. In one embodiment, the antisense strand comprises two phosphorothioate internucleotide linkages at the 5′-terminus and two phosphorothioate internucleotide linkages at the 3′-terminus, and the sense strand comprises at least two phosphorothioate internucleotide linkages at either the 5′-terminus or the 3′-terminus.

In one embodiment, the RNAi comprises a phosphorothioate or methylphosphonate internucleotide linkage modification in the overhang region. For example, the overhang region may contain two nucleotides having a phosphorothioate or methylphosphonate internucleotide linkage between the two nucleotides. Internucleotide linkage modifications also may be made to link the overhang nucleotides with the terminal paired nucleotides within the duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. These terminal three nucleotides may be at the 3′-end of the antisense strand, the 3′-end of the sense strand, the 5′-end of the antisense strand, and/or the 5′end of the antisense strand.

In one embodiment, the 2 nucleotide overhang is at the 3′-end of the antisense strand, and there are two phosphorothioate internucleotide linkages between the terminal three nucleotides, wherein two of the three nucleotides are the overhang nucleotides, and the third nucleotide is a paired nucleotide next to the overhang nucleotide. Optionally, the RNAi agent may additionally have two phosphorothioate internucleotide linkages between the terminal three nucleotides at both the 5′-end of the sense strand and at the 5′-end of the antisense strand.

In one embodiment, the RNAi agent comprises mismatch(es) with the target, within the duplex, or combinations thereof. The mismatch may occur in the overhang region or the duplex region. The base pair may be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In one embodiment, the RNAi agent comprises at least one of the first 1, 2, 3, 4, or 5 base pairs within the duplex regions from the 5′-end of the antisense strand independently selected from the group of: A:U, G:U, I:C, and mismatched pairs, e.g., non-canonical or other than canonical pairings or pairings which include a universal base, to promote the dissociation of the antisense strand at the 5′-end of the duplex.

In one embodiment, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

In another embodiment, the nucleotide at the 3′-end of the sense strand is deoxy-thymine (dT). In another embodiment, the nucleotide at the 3′-end of the antisense strand is deoxy-thymine (dT). In one embodiment, there is a short sequence of deoxy-thymine nucleotides, for example, two dT nucleotides on the 3′-end of the sense and/or antisense strand.

In one embodiment, the sense strand sequence may be represented by formula (I):

(I)
5′ nₚ-Nₐ-(X X X)ᵢ-N_{b}-Y Y Y-N_{b}-(Z Z Z)ⱼ-Nₐ-n_{q} 3′

wherein:
i and j are each independently 0 or 1;
p and q are each independently 0-6;
each Na independently represents an oligonucleotide sequence comprising 0-25 modified nucleotides, each sequence comprising at least two differently modified nucleotides;
each Nb independently represents an oligonucleotide sequence comprising 0-10 modified nucleotides;
each np and nq independently represent an overhang nucleotide;
wherein Nb and Y do not have the same modification; and
XXX, YYY and ZZZ each independently represent one motif of three identical modifications on three consecutive nucleotides. Preferably YYY is all 2′-F modified nucleotides.

In one embodiment, the Na and/or Nb comprise modifications of alternating pattern.

In one embodiment, the YYY motif occurs at or near the cleavage site of the sense strand. For example, when the RNAi agent has a duplex region of 17-23 nucleotides in length, the YYY motif can occur at or the vicinity of the cleavage site (e.g.: can occur at positions 6, 7, 8, 7, 8, 9, 8, 9, 10, 9, 10, 11, 10, 11, 12 or 11, 12, 13) of - the sense strand, the count starting from the 1st nucleotide, from the 5′ end; or optionally, the count starting at the 1st paired nucleotide within the duplex region, from the 5′-end.

In one embodiment, i is 1 and j is 0, or i is 0 and j is 1, or both i and j are 1. The sense strand can therefore be represented by the following formulas:

(Ib)
5′ np-Na-YYY-Nb-ZZZ-Na-nq 3′
(Ic)
5′ np-Na-XXX-Nb-YYY-Na-nq 3′
or
(Id)
5′ np-Na-XXX-Nb-YYY-Nb-ZZZ-Na-nq 3′.

When the sense strand is represented by formula (Ib), Nb represents an oligonucleotide sequence comprising 0-10, 0-7, 0-5, 0-4, 0-2 or 0 modified nucleotides. Each Na independently can represent an oligonucleotide sequence comprising 2-20, 2-15, or 2-10 modified nucleotides.

When the sense strand is represented as formula (Ic), Nb represents an oligonucleotide sequence comprising 0-10, 0-7, 0-10, 0-7, 0-5, 0-4, 0-2 or 0 modified nucleotides. Each Na can independently represent an oligonucleotide sequence comprising 2-20, 2-15, or 2-10 modified nucleotides.

When the sense strand is represented as formula (Id), each Nb independently represents an oligonucleotide sequence comprising 0-10, 0-7, 0-5, 0-4, 0-2 or 0 modified nucleotides. Preferably, Nb is 0, 1, 2, 3, 4, 5 or 6. Each Na can independently represent an oligonucleotide sequence comprising 2-20, 2-15, or 2-10 modified nucleotides.

Each of X, Y and Z may be the same or different from each other.

In other embodiments, i is 0 and j is 0, and the sense strand may be represented by the formula:

(Ia)
5′ np-Na-YYY-Na-nq 3′.

When the sense strand is represented by formula (Ia), each Na independently can represent an oligonucleotide sequence comprising 2-20, 2-15, or 2-10 modified nucleotides.

In one embodiment, the antisense strand sequence of the RNAi may be represented by formula (II):

(II)
5′ nq′-Na′-(Z′Z′Z′)k-Nb′-Y′Y′Y′-Nb′-(X′X′X′)l-N′a-
np′ 3′

wherein:
k and l are each independently 0 or 1;
p′ and q′ are each independently 0-6;
each Na′ independently represents an oligonucleotide sequence comprising 0-25 modified nucleotides, each sequence comprising at least two differently modified nucleotides;
each Nb′ independently represents an oligonucleotide sequence comprising 0-10 modified nucleotides;
each np′ and nq′ independently represent an overhang nucleotide;
wherein Nb′ and Y′ do not have the same modification; and X′X′X′, Y′Y′Y′ and Z′Z′Z′ each independently represent one motif of three identical modifications on three consecutive nucleotides.

In one embodiment, the Na′ and/or Nb′ comprise modifications of alternating pattern.

The Y′Y′Y′ motif occurs at or near the cleavage site of the antisense strand. For example, when the RNAi agent has a duplex region of 17-23 nucleotide in length, the Y′Y′Y′ motif can occur at positions 9, 10, 11; 10, 11, 12; 11, 12, 13; 12, 13, 14; or 13, 14, 15 of the antisense strand, with the count starting from the 1st nucleotide, from the 5′ end; or optionally, the count starting at the 1st paired nucleotide within the duplex region, from the 5′-end. Preferably, the Y′Y′Y′ motif occurs at positions 11, 12, 13.

In one embodiment, Y′Y′Y′ motif is all 2′-OMe modified nucleotides.

In one embodiment, k is 1 and l is 0, or k is 0 and l is 1, or both k and l are 1.

The antisense strand can therefore be represented by the following formulas:

(IIb)
5′ nq′-Na′-Z′Z′Z′-Nb′-Y′Y′Y′-Na′-np′ 3′;
(IIc)
5′ nq′-Na′-Y′Y′Y′-Nb′-X′X′X′-np′ 3′;
or
(IId)
5′ nq′-Na′- Z′Z′Z′-Nb′-Y′Y′Y′-Nb′- X′X′X′-Na′-
np′ 3′.

When the antisense strand is represented by formula (IIb), Nb′ represents an oligonucleotide sequence comprising 0-10, 0-7, 0-10, 0-7, 0-5, 0-4, 0-2 or 0 modified nucleotides. Each Na′ independently represents an oligonucleotide sequence comprising 2-20, 2-15, or 2-10 modified nucleotides.

When the antisense strand is represented as formula (IIc), Nb′ represents an oligonucleotide sequence comprising 0-10, 0-7, 0-10, 0-7, 0-5, 0-4, 0-2 or 0 modified nucleotides. Each Na′ independently represents an oligonucleotide sequence comprising 2-20, 2-15, or 2-10 modified nucleotides.

When the antisense strand is represented as formula (IId), each Nb′ independently represents an oligonucleotide sequence comprising 0-10, 0-7, 0-10, 0-7, 0-5, 0-4, 0-2 or 0 modified nucleotides. Each Na′ independently represents an oligonucleotide sequence comprising 2-20, 2-15, or 2-10 modified nucleotides. Preferably, Nb is 0, 1, 2, 3, 4, 5 or 6.

In other embodiments, k is 0 and l is 0 and the antisense strand may be represented by the formula:

(Ia)
5′ np′-Na′-Y′Y′Y′ - Na′-nq′ 3′.

When the antisense strand is represented as formula (IIa), each Na′ independently represents an oligonucleotide sequence comprising 2-20, 2-15, or 2-10 modified nucleotides.

Each of X′, Y′ and Z′ may be the same or different from each other.

Each nucleotide of the sense strand and antisense strand may be independently modified with LNA, CRN, UNA, cEt, HNA, CeNA, 2′-methoxyethyl, 2′-O-methyl, 2′-O-allyl, 2′-C— allyl, 2′-hydroxyl, or 2′-fluoro. For example, each nucleotide of the sense strand and antisense strand is independently modified with 2′-O-methyl or 2′-fluoro. Each X, Y, Z, X′, Y′ and Z′, in particular, may represent a 2′-O-methyl modification or a 2′-fluoro modification.

In one embodiment, the sense strand of the RNAi agent may contain YYY motif occurring at 9, 10 and 11 positions of the strand when the duplex region is 21 nt, the count starting from the 1st nucleotide from the 5′ end, or optionally, the count starting at the 1st paired nucleotide within the duplex region, from the 5′-end; and Y represents 2′-F modification. The sense strand may additionally contain XXX motif or ZZZ motifs as wing modifications at the opposite end of the duplex region; and XXX and ZZZ each independently represents a 2′-OMe modification or 2′-F modification.

In one embodiment the antisense strand may contain Y′Y′Y′ motif occurring at positions 11, 12, 13 of the strand, the count starting from the 1st nucleotide from the 5′ end, or optionally, the count starting at the 1st paired nucleotide within the duplex region, from the 5′-end; and Y′ represents 2′-O-methyl modification. The antisense strand may additionally contain X′X′X′ motif or Z′Z′Z′ motifs as wing modifications at the opposite end of the duplex region; and X′X′X′ and Z′Z′Z′ each independently represents a 2′-OMe modification or 2′-F modification.

The sense strand represented by any one of the above formulas (Ia), (Ib), (Ic), and (Id) forms a duplex with a antisense strand being represented by any one of formulas (IIa), (IIb), (IIc), and (IId), respectively.

Accordingly, the RNAi agents for use in the methods of the invention may comprise a sense strand and an antisense strand, each strand having 14 to 30 nucleotides, the RNAi duplex represented by formula (III):

(III)
sense:
5′ np -Na-(X X X)i -Nb- Y Y Y -Nb -(Z Z Z)j-Na-
nq 3′
antisense:
3′ np′-Na′-(X′X′X′)k-Nb′-Y′Y′Y′-Nb′-(Z′Z′Z′)1-Na′-
nq′ 5′

wherein:
i, j, k, and 1 are each independently 0 or 1;
p, p′, q, and q′ are each independently 0-6;
each Na and Na′ independently represents an oligonucleotide sequence comprising 0-25 modified nucleotides, each sequence comprising at least two differently modified nucleotides;
each Nb and Nb′ independently represents an oligonucleotide sequence comprising 0-10 modified nucleotides;
wherein each np′, np, nq′, and nq, each of which may or may not be present, independently represents an overhang nucleotide; and
XXX, YYY, ZZZ, X′X′X′, Y′Y′Y′, and Z′Z′Z′ each independently represent one motif of three identical modifications on three consecutive nucleotides.

In one embodiment, i is 0 and j is 0; or i is 1 and j is 0; or i is 0 and j is 1; or both i and j are 0; or both i and j are 1. In another embodiment, k is 0 and l is 0; or k is 1 and l is 0; k is 0 and l is 1; or both k and l are 0; or both k and l are 1.

Exemplary combinations of the sense strand and antisense strand forming a RNAi duplex include the formulas below:

(IIIa)
5′ np - Na - Y Y Y -Na-nq 3′
3′ np′-Na′-Y′Y′Y′ -Na′nq′ 5′
(IIIb)
5′ np -Na -Y Y Y -Nb -ZZZ -Na-nq 3′
3′ np′-Na′-Y′Y′Y′-Nb′-Z′Z′Z′-Na′nq′ 5′
(IIIc)
5′ np-Na- X X X -Nb - Y Y Y - Na-nq 3′
3′ np′-Na′-X′X′X′-Nb′-Y′Y′Y′-Na′-nq′ 5′
(IIId)
5′ np -Na -X X X -Nb-Y Y Y -Nb- ZZZ -Na-nq 3′
3′ np′-Na′-X′X′X′-Nb′-Y′Y′Y′-Nb′-Z′Z′Z′-Na-nq′ 5′

When the RNAi agent is represented by formula (IIIa), each Na independently represents an oligonucleotide sequence comprising 2-20, 2-15, or 2-10 modified nucleotides.

When the RNAi agent is represented by formula (IIIb), each Nb independently represents an oligonucleotide sequence comprising 1-10, 1-7, 1-5 or 1-4 modified nucleotides. Each Na independently represents an oligonucleotide sequence comprising 2-20, 2-15, or 2-10 modified nucleotides.

When the RNAi agent is represented as formula (IIIc), each Nb, Nb′ independently represents an oligonucleotide sequence comprising 0-10, 0-7, 0-10, 0-7, 0-5, 0-4, 0-2 or 0 modified nucleotides. Each Na independently represents an oligonucleotide sequence comprising 2-20, 2-15, or 2-10 modified nucleotides.

When the RNAi agent is represented as formula (IIId), each Nb, Nb′ independently represents an oligonucleotide sequence comprising 0-10, 0-7, 0-10, 0-7, 0-5, 0-4, 0-2 or 0 modified nucleotides. Each Na, Na′ independently represents an oligonucleotide sequence comprising 2-20, 2-15, or 2-10 modified nucleotides. Each of Na, Na′, Nb and Nb′ independently comprises modifications of alternating pattern.

Each of X, Y and Z in formulas (III), (IIIa), (IIIb), (IIIc), and (IIId) may be the same or different from each other.

When the RNAi agent is represented by formula (III), (IIIa), (IIIb), (IIIc), and (IIId), at least one of the Y nucleotides may form a base pair with one of the Y′ nucleotides. Alternatively, at least two of the Y nucleotides form base pairs with the corresponding Y′ nucleotides; or all three of the Y nucleotides all form base pairs with the corresponding Y′ nucleotides.

When the RNAi agent is represented by formula (IIIb) or (IIId), at least one of the Z nucleotides may form a base pair with one of the Z′ nucleotides. Alternatively, at least two of the Z nucleotides form base pairs with the corresponding Z′ nucleotides; or all three of the Z nucleotides all form base pairs with the corresponding Z′ nucleotides.

When the RNAi agent is represented as formula (IIIc) or (IIId), at least one of the X nucleotides may form a base pair with one of the X′ nucleotides. Alternatively, at least two of the X nucleotides form base pairs with the corresponding X′ nucleotides; or all three of the X nucleotides all form base pairs with the corresponding X′ nucleotides.

In one embodiment, the modification on the Y nucleotide is different than the modification on the Y′ nucleotide, the modification on the Z nucleotide is different than the modification on the Z′ nucleotide, and/or the modification on the X nucleotide is different than the modification on the X′ nucleotide.

In one embodiment, when the RNAi agent is represented by formula (IIId), the Na modifications are 2′-O-methyl or 2′-fluoro modifications. In another embodiment, when the RNAi agent is represented by formula (IIId), the Na modifications are 2-O-methyl or 2-fluoro modifications and np′>0 and at least one np′ is linked to a neighboring nucleotide a via phosphorothioate linkage. In yet another embodiment, when the RNAi agent is represented by formula (IIId), the Na modifications are 2-O-methyl or 2-fluoro modifications, np′>0 and at least one np′ is linked to a neighboring nucleotide via phosphorothioate linkage, and the sense strand is conjugated to one or more GalNAc derivatives attached through a bivalent or trivalent branched linker (described below). In another embodiment, when the RNAi agent is represented by formula (IIId), the Na modifications are 2-O-methyl or 2-fluoro modifications, np′>0 and at least one np′ is linked to a neighboring nucleotide via phosphorothioate linkage, the sense strand comprises at least one phosphorothioate linkage, and the sense strand is conjugated to one or more GalNAc derivatives attached through a bivalent or trivalent branched linker.

In one embodiment, when the RNAi agent is represented by formula (IIIa), the Na modifications are 2-O-methyl or 2-fluoro modifications, np′>0 and at least one np′ is linked to a neighboring nucleotide via phosphorothioate linkage, the sense strand comprises at least one phosphorothioate linkage, and the sense strand is conjugated to one or more GalNAc derivatives attached through a bivalent or trivalent branched linker.

In one embodiment, the RNAi agent is a multimer containing at least two duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In one embodiment, the RNAi agent is a multimer containing three, four, five, six or more duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In one embodiment, two RNAi agents represented by formula (III), (IIIa), (IIIb), (IIIc), and (IIId) are linked to each other at the 5′ end, and one or both of the 3′ ends and are optionally conjugated to a ligand. Each of the agents can target the same gene or two different genes; or each of the agents can target same gene at two different target sites.

In certain embodiments, an RNAi agent of the invention may contain a low number of nucleotides containing a 2′-fluoro modification, e.g., 10 or fewer nucleotides with 2′-fluoro modification. For example, the RNAi agent may contain 10, 9, 8, 7, 6, 5, 4, 3, 2, 1 or 0 nucleotides with a 2′-fluoro modification. In a specific embodiment, the RNAi agent of the invention contains 10 nucleotides with a 2′-fluoro modification, e.g., 4 nucleotides with a 2′-fluoro modification in the sense strand and 6 nucleotides with a 2′-fluoro modification in the antisense strand. In another specific embodiment, the RNAi agent of the invention contains 6 nucleotides with a 2′-fluoro modification, e.g., 4 nucleotides with a 2′-fluoro modification in the sense strand and 2 nucleotides with a 2′-fluoro modification in the antisense strand.

In other embodiments, an RNAi agent of the invention may contain an ultra low number of nucleotides containing a 2′-fluoro modification, e.g., 2 or fewer nucleotides containing a 2′-fluoro modification. For example, the RNAi agent may contain 2, 1 of 0 nucleotides with a 2′-fluoro modification. In a specific embodiment, the RNAi agent may contain 2 nucleotides with a 2′-fluoro modification, e.g., 0 nucleotides with a 2-fluoro modification in the sense strand and 2 nucleotides with a 2′-fluoro modification in the antisense strand.

Various publications describe multimeric RNAi agents that can be used in the methods of the invention. Such publications include WO2007/091269, U.S. Pat. No. 7,858,769, WO2010/141511, WO2007/117686, WO2009/014887 and WO2011/031520 the entire contents of each of which are hereby incorporated herein by reference.

As described in more detail below, the RNAi agent that contains conjugations of one or more carbohydrate moieties to an RNAi agent can optimize one or more properties of the RNAi agent. In many cases, the carbohydrate moiety will be attached to a modified subunit of the RNAi agent. For example, the ribose sugar of one or more ribonucleotide subunits of a dsRNA agent can be replaced with another moiety, e.g., a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS). A cyclic carrier may be a carbocyclic ring system, i.e., all ring atoms are carbon atoms, or a heterocyclic ring system, i.e., one or more ring atoms may be a heteroatom, e.g., nitrogen, oxygen, sulfur. The cyclic carrier may be a monocyclic ring system, or may contain two or more rings, e.g. fused rings. The cyclic carrier may be a fully saturated ring system, or it may contain one or more double bonds.

The ligand may be attached to the polynucleotide via a carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier into the backbone, e.g., the phosphate, or modified phosphate, e.g., sulfur containing, backbone, of a ribonucleic acid. A “tethering attachment point” (TAP) in some embodiments refers to a constituent ring atom of the cyclic carrier, e.g., a carbon atom or a heteroatom (distinct from an atom which provides a backbone attachment point), that connects a selected moiety. The moiety can be, e.g., a carbohydrate, e.g. monosaccharide, disaccharide, trisaccharide, tetrasaccharide, oligosaccharide and polysaccharide. Optionally, the selected moiety is connected by an intervening tether to the cyclic carrier. Thus, the cyclic carrier will often include a functional group, e.g., an amino group, or generally, provide a bond, that is suitable for incorporation or tethering of another chemical entity, e.g., a ligand to the constituent ring.

The RNAi agents may be conjugated to a ligand via a carrier, wherein the carrier can be cyclic group or acyclic group; preferably, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and decalin; preferably, the acyclic group is selected from serinol backbone or diethanolamine backbone.

In another aspect, the dsRNA agent comprises a sense strand and an antisense strand, each strand having 14 to 40 nucleotides. The dsRNA agent is represented by formula (I):

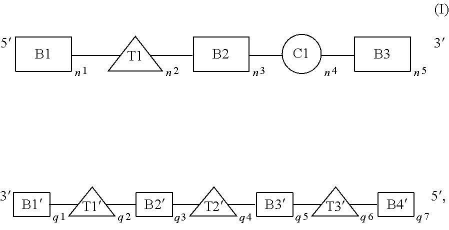

In formula (I), B1, B2, B3, B1′, B2′, B3′, and B4′ each are independently a nucleotide containing a modification selected from the group consisting of 2′-O-alkyl, 2′-substituted alkoxy, 2′-substituted alkyl, 2′-halo, ENA, and BNA/LNA. In one embodiment, B1, B2, B3, B1′, B2′, B3′, and B4′ each contain 2′-OMe modifications. In one embodiment, B1, B2, B3, B1′, B2′, B3′, and B4′ each contain 2′-OMe or 2′-F modifications. In one embodiment, at least one of B1, B2, B3, B1′, B2′, B3′, and B4′ contain 2′-O—N-methylacetamido (2′-O-NMA) modification.

C1 is a thermally destabilizing nucleotide placed at a site opposite to the seed region of the antisense strand (i.e., at positions 2-8 of the 5′-end of the antisense strand). For example, C1 is at a position of the sense strand that pairs with a nucleotide at positions 2-8 of the 5′-end of the antisense strand. In one example, C1 is at position 15 from the 5′-end of the sense strand. C1 nucleotide bears the thermally destabilizing modification which can include abasic modification; mismatch with the opposing nucleotide in the duplex; and sugar modification such as 2′-deoxy modification or acyclic nucleotide e.g., unlocked nucleic acids (UNA) or glycerol nucleic acid (GNA). In one embodiment, C1 has thermally destabilizing modification selected from the group consisting of: i) mismatch with the opposing nucleotide in the antisense strand; ii) abasic modification selected from the group consisting of:

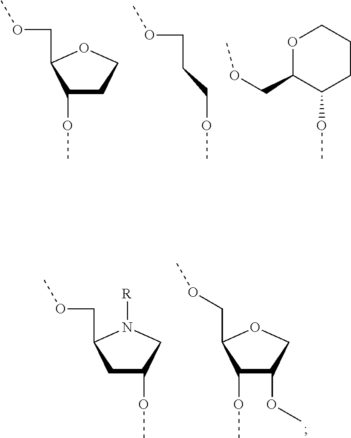

and iii) sugar modification selected from the group consisting of:

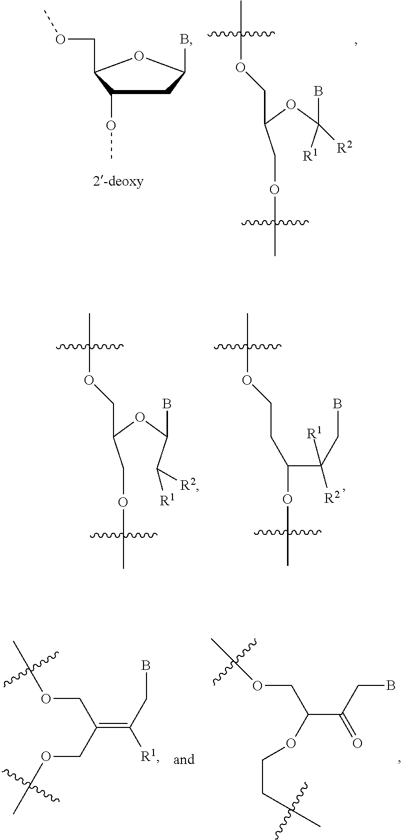

wherein B is a modified or unmodified nucleobase, R1 and R2 independently are H, halogen, OR3, or alkyl; and R3 is H, alkyl, cycloalkyl, aryl, aralkyl, heteroaryl or sugar. In one embodiment, the thermally destabilizing modification in C1 is a mismatch selected from the group consisting of G:G, G:A, G:U, G:T, A:A, A:C, C:C, C:U, C:T, U:U, T:T, and U:T; and optionally, at least one nucleobase in the mismatch pair is a 2′-deoxy nucleobase. In one example, the thermally destabilizing modification in C1 is GNA or

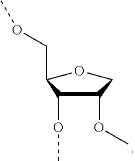

T1, T1′, T2′, and T3′ each independently represent a nucleotide comprising a modification providing the nucleotide a steric bulk that is less or equal to the steric bulk of a 2′-OMe modification.

A steric bulk refers to the sum of steric effects of a modification. Methods for determining steric effects of a modification of a nucleotide are known to one skilled in the art. The modification can be at the 2′ position of a ribose sugar of the nucleotide, or a modification to a non-ribose nucleotide, acyclic nucleotide, or the backbone of the nucleotide that is similar or equivalent to the 2′ position of the ribose sugar, and provides the nucleotide a steric bulk that is less than or equal to the steric bulk of a 2′-OMe modification. For example, T1, T1′, T2′, and T3′ are each independently selected from DNA, RNA, LNA, 2′-F, and 2′-F-5′-methyl. In one embodiment, T1 is DNA. In one embodiment, T1′ is DNA, RNA or LNA. In one embodiment, T2′ is DNA or RNA. In one embodiment, T3′ is DNA or RNA.
n1, n3, and q1 are independently 4 to 15 nucleotides in length.
n5, q3, and q7 are independently 1-6 nucleotide(s) in length.
n4, q2, and q6 are independently 1-3 nucleotide(s) in length; alternatively, n4 is 0.
q5 is independently 0-10 nucleotide(s) in length.
n2 and q4 are independently 0-3 nucleotide(s) in length.

Alternatively, n4 is 0-3 nucleotide(s) in length.

In one embodiment, n4 can be 0. In one example, n4 is 0, and q2 and q6 are 1. In another example, n4 is 0, and q2 and q6 are 1, with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, n4, q2, and q6 are each 1.

In one embodiment, n2, n4, q2, q4, and q6 are each 1.

In one embodiment, C1 is at position 14-17 of the 5′-end of the sense strand, when the sense strand is 19-22 nucleotides in length, and n4 is 1. In one embodiment, C1 is at position 15 of the 5′-end of the sense strand

In one embodiment, T3′ starts at position 2 from the 5′ end of the antisense strand. In one example, T3′ is at position 2 from the 5′ end of the antisense strand and q6 is equal to 1.

In one embodiment, T1′ starts at position 14 from the 5′ end of the antisense strand. In one example, T1′ is at position 14 from the 5′ end of the antisense strand and q2 is equal to 1.

In an exemplary embodiment, T3′ starts from position 2 from the 5′ end of the antisense strand and T1′ starts from position 14 from the 5′ end of the antisense strand. In one example, T3′ starts from position 2 from the 5′ end of the antisense strand and q6 is equal to 1 and T1′ starts from position 14 from the 5′ end of the antisense strand and q2 is equal to 1.

In one embodiment, T1′ and T3′ are separated by 11 nucleotides in length (i.e. not counting the T1′ and T3′ nucleotides).

In one embodiment, T1′ is at position 14 from the 5′ end of the antisense strand. In one example, T1′ is at position 14 from the 5′ end of the antisense strand and q2 is equal to 1, and the modification at the 2′ position or positions in a non-ribose, acyclic or backbone that provide less steric bulk than a 2′-OMe ribose.

In one embodiment, T3′ is at position 2 from the 5′ end of the antisense strand. In one example, T3′ is at position 2 from the 5′ end of the antisense strand and q6 is equal to 1, and the modification at the 2′ position or positions in a non-ribose, acyclic or backbone that provide less than or equal to steric bulk than a 2′-OMe ribose.

In one embodiment, T1 is at the cleavage site of the sense strand. In one example, T1 is at position 11 from the 5′ end of the sense strand, when the sense strand is 19-22 nucleotides in length, and n2 is 1. In an exemplary embodiment, T1 is at the cleavage site of the sense strand at position 11 from the 5′ end of the sense strand, when the sense strand is 19-22 nucleotides in length, and n2 is 1, In one embodiment, T2′ starts at position 6 from the 5′ end of the antisense strand. In one example, T2′ is at positions 6-10 from the 5′ end of the antisense strand, and q4 is 1.

In an exemplary embodiment, T1 is at the cleavage site of the sense strand, for instance, at position 11 from the 5′ end of the sense strand, when the sense strand is 19-22 nucleotides in length, and n2 is 1; T1′ is at position 14 from the 5′ end of the antisense strand, and q2 is equal to 1, and the modification to T1′ is at the 2′ position of a ribose sugar or at positions in a non-ribose, acyclic or backbone that provide less steric bulk than a 2′-OMe ribose; T2′ is at positions 6-10 from the 5′ end of the antisense strand, and q4 is 1; and T3′ is at position 2 from the 5′ end of the antisense strand, and q6 is equal to 1, and the modification to T3′ is at the 2′ position or at positions in a non-ribose, acyclic or backbone that provide less than or equal to steric bulk than a 2′-OMe ribose.

In one embodiment, T2′ starts at position 8 from the 5′ end of the antisense strand. In one example, T2′ starts at position 8 from the 5′ end of the antisense strand, and q4 is 2.

In one embodiment, T2′ starts at position 9 from the 5′ end of the antisense strand. In one example, T2′ is at position 9 from the 5′ end of the antisense strand, and q4 is 1.

In one embodiment, B1′ is 2′-OMe or 2′-F, q1 is 9, T1′ is 2′-F, q2 is 1, B2′ is 2′-OMe or 2′-F, q3 is 4, T2′ is 2′-F, q4 is 1, B3′ is 2′-OMe or 2′-F, q5 is 6, T3′ is 2′-F, q6 is 1, B4′ is 2′-OMe, and q7 is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 1, B3′ is 2′-OMe or 2′-F, q5 is 6, T3′ is 2′-F, q6 is 1, B4′ is 2′-OMe, and q7 is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n1 is 8, T1 is 2′F, n2 is 3, B2 is 2′-OMe, n3 is 7, n4 is 0, B3 is 2′OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-OMe, and q7 is 1.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-OMe, and q7 is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n1 is 6, T1 is 2′F, n2 is 3, B2 is 2′-OMe, n3 is 7, n4 is 0, B3 is 2′OMe, n5 is 3, B1′ is 2′-OMe or 2′-F, q1 is 7, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-OMe, and q7 is 1.

In one embodiment, B1 is 2′-OMe or 2′-F, n1 is 6, T1 is 2′F, n2 is 3, B2 is 2′-OMe, n3 is 7, n4 is 0, B3 is 2′-OMe, n5 is 3, B1′ is 2′-OMe or 2′-F, q1 is 7, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-OMe, and q7 is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n1 is 8, T1 is 2′F, n2 is 3, B2 is 2′-OMe, n3 is 7, n4 is 0, B3 is 2′OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 1, B3′ is 2′-OMe or 2′-F, q5 is 6, T3′ is 2′-F, q6 is 1, B4′ is 2′-OMe, and q7 is 1.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 1, B3′ is 2′-OMe or 2′-F, q5 is 6, T3′ is 2′-F, q6 is 1, B4′ is 2′-OMe, and q7 is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n1 is 8, T1 is 2′F, n2 is 3, B2 is 2′-OMe, n3 is 7, n4 is 0, B3 is 2′OMe, n5 is 3, B1′ is 2′-OMe or 2′-F, q1 is 9, T1′ is 2′-F, q2 is 1, B2′ is 2′-OMe or 2′-F, q3 is 5, T2′ is 2′-F, q4 is 1, B3′ is 2′-OMe or 2′-F, q5 is 5, T3′ is 2′-F, q6 is 1, B4′ is 2′-OMe, and q7 is 1; optionally with at least 2 additional TT at the 3′-end of the antisense strand.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 5, T2′ is 2′-F, q4 is 1, B3′ is 2′-OMe or 2′-F, q5 is 5, T3′ is 2′-F, q6 is 1, B4′ is 2′-OMe, and q7 is 1; optionally with at least 2 additional TT at the 3′-end of the antisense strand; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-OMe, and q7 is 1.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-OMe, and q7 is 1; with two phosphorothioate internucleotide linkage modifications within positions 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In one embodiment, B1 is 2′-OMe or 2′-F, n1 is 8, T1 is 2′F, n2 is 3, B2 is 2′-OMe, n3 is 7, n4 is 0, B3 is 2′OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-F, and q7 is 1.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-F, and q7 is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-F, and q7 is 1.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-F, and q7 is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

The dsRNA agent can comprise a phosphorus-containing group at the 5′-end of the sense strand or antisense strand. The 5′-end phosphorus-containing group can be 5′-end phosphate (5′-P), 5′-end phosphorothioate (5′-PS), 5′-end phosphorodithioate (5′-PS2), 5′-end vinylphosphonate (5′-VP), 5′-end methylphosphonate (MePhos), or 5′-deoxy-5′-C-malonyl

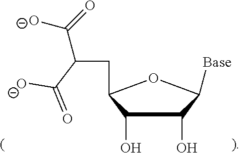

When the 5′-end phosphorus-containing group is 5′-end vinylphosphonate (5′-VP), the 5′-VP can be either 5′-E-VP isomer (i.e., trans-vinylphosphate,

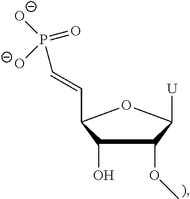

5′-Z—VP isomer (i.e., cis-vinylphosphate,

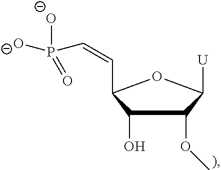

or mixtures thereof.

In one embodiment, the dsRNA agent comprises a phosphorus-containing group at the 5′-end of the sense strand. In one embodiment, the dsRNA agent comprises a phosphorus-containing group at the 5′-end of the antisense strand.

In one embodiment, the dsRNA agent comprises a 5′-P. In one embodiment, the dsRNA agent comprises a 5′-P in the antisense strand.

In one embodiment, the dsRNA agent comprises a 5′-PS. In one embodiment, the dsRNA agent comprises a 5′-PS in the antisense strand.

In one embodiment, the dsRNA agent comprises a 5′-VP. In one embodiment, the dsRNA agent comprises a 5′-VP in the antisense strand. In one embodiment, the dsRNA agent comprises a 5′-E-VP in the antisense strand. In one embodiment, the dsRNA agent comprises a 5′-Z—VP in the antisense strand.

In one embodiment, the dsRNA agent comprises a 5′-PS2. In one embodiment, the dsRNA agent comprises a 5′-PS2 in the antisense strand.

In one embodiment, the dsRNA agent comprises a 5′-PS2. In one embodiment, the dsRNA agent comprises a 5′-deoxy-5′-C-malonyl in the antisense strand.

In one embodiment, B1 is 2′-OMe or 2′-F, n1 is 8, T1 is 2′F, n2 is 3, B2 is 2′-OMe, n3 is 7, n4 is 0, B3 is 2′OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-OMe, and q7 is 1. The dsRNA agent also comprises a 5′-PS.

In one embodiment, B1 is 2′-OMe or 2′-F, n1 is 8, T1 is 2′F, n2 is 3, B2 is 2′-OMe, n3 is 7, n4 is 0, B3 is 2′OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-OMe, and q7 is 1. The dsRNA agent also comprises a 5′-P.

In one embodiment, B1 is 2′-OMe or 2′-F, n1 is 8, T1 is 2′F, n2 is 3, B2 is 2′-OMe, n3 is 7, n4 is 0, B3 is 2′OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-OMe, and q7 is 1. The dsRNA agent also comprises a 5′-VP. The 5′-VP may be 5′-E-VP, 5′-Z—VP, or combination thereof.

In one embodiment, B1 is 2′-OMe or 2′-F, n1 is 8, T1 is 2′F, n2 is 3, B2 is 2′-OMe, n3 is 7, n4 is 0, B3 is 2′OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-OMe, and q7 is 1. The dsRNA agent also comprises a 5′-PS2.

In one embodiment, B1 is 2′-OMe or 2′-F, n1 is 8, T1 is 2′F, n2 is 3, B2 is 2′-OMe, n3 is 7, n4 is 0, B3 is 2′OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-OMe, and q7 is 1. The dsRNA agent also comprises a 5′-deoxy-5′-C-malonyl.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-OMe, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-P.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-OMe, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-OMe, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-VP. The 5′-VP may be 5′-E-VP, 5′-Z—VP, or combination thereof.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-OMe, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS2.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-OMe, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-deoxy-5′-C-malonyl.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-OMe, and q7 is 1. The dsRNA agent also comprises a 5′-P.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-OMe, and q7 is 1. The dsRNA agent also comprises a 5′-PS.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-OMe, and q7 is 1. The dsRNA agent also comprises a 5′-VP. The 5′-VP may be 5′-E-VP, 5′-Z—VP, or combination thereof.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-OMe, and q7 is 1. The dsRNA agent also comprises a 5′-PS2.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-OMe, and q7 is 1. The dsRNA agent also comprises a 5′-deoxy-5′-C-malonyl.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-OMe, and q7 is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-P.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-OMe, and q7 is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-PS.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-OMe, and q7 is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-VP. The 5′-VP may be 5′-E-VP, 5′-Z—VP, or combination thereof.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-OMe, and q7 is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-PS2.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-OMe, and q7 is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-deoxy-5′-C-malonyl.

In one embodiment, B1 is 2′-OMe or 2′-F, n1 is 8, T1 is 2′F, n2 is 3, B2 is 2′-OMe, n3 is 7, n4 is 0, B3 is 2′OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-F, and q7 is 1. The dsRNA agent also comprises a 5′-P.

In one embodiment, B1 is 2′-OMe or 2′-F, n1 is 8, T1 is 2′F, n2 is 3, B2 is 2′-OMe, n3 is 7, n4 is 0, B3 is 2′OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-F, and q7 is 1. The dsRNA agent also comprises a 5′-PS.

In one embodiment, B1 is 2′-OMe or 2′-F, n1 is 8, T1 is 2′F, n2 is 3, B2 is 2′-OMe, n3 is 7, n4 is 0, B3 is 2′OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-F, and q7 is 1. The dsRNA agent also comprises a 5′-VP. The 5′-VP may be 5′-E-VP, 5′-Z—VP, or combination thereof.

In one embodiment, B1 is 2′-OMe or 2′-F, n1 is 8, T1 is 2′F, n2 is 3, B2 is 2′-OMe, n3 is 7, n4 is 0, B3 is 2′OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-F, and q7 is 1. The dsRNA agent also comprises a 5′-PS2.

In one embodiment, B1 is 2′-OMe or 2′-F, n1 is 8, T1 is 2′F, n2 is 3, B2 is 2′-OMe, n3 is 7, n4 is 0, B3 is 2′OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-F, and q7 is 1. The dsRNA agent also comprises a 5′-deoxy-5′-C-malonyl.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-F, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-P.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-F, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-F, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-VP. The 5′-VP may be 5′-E-VP, 5′-Z—VP, or combination thereof.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-F, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS2.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-F, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-deoxy-5′-C-malonyl.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-F, and q7 is 1. The dsRNA agent also comprises a 5′-P.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-F, and q7 is 1. The dsRNA agent also comprises a 5′-PS.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-F, and q7 is 1. The dsRNA agent also comprises a 5′-VP. The 5′-VP may be 5′-E-VP, 5′-Z—VP, or combination thereof.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-F, and q7 is 1. The dsRNA agent also comprises a 5′-PS2.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-F, and q7 is 1. The dsRNA agent also comprises a 5′-deoxy-5′-C-malonyl.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-F, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-P.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-F, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-F, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-VP. The 5′-VP may be 5′-E-VP, 5′-Z—VP, or combination thereof.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-F, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS2.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-F, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-deoxy-5′-C-malonyl.

In one embodiment, 100%, 95%, 90%, 85%, 80%, 75%, 70%, 65%, 60%, 55%, 50%, 45%, 40%, 35% or 30% of the dsRNA agent of the invention is modified. For example, when 50% of the dsRNA agent is modified, 50% of all nucleotides present in the dsRNA agent contain a modification as described herein.

In one embodiment, each of the sense and antisense strands of the dsRNA agent is independently modified with acyclic nucleotides, LNA, HNA, CeNA, 2′-methoxyethyl, 2′-O-methyl, 2′-O-allyl, 2′-C-allyl, 2′-deoxy, 2′-fluoro, 2′-O—N-methylacetamido (2′-O-NMA), a 2′-O-dimethylaminoethoxyethyl (2′-O-DMAEOE), 2′-O-aminopropyl (2′-O-AP), or 2′-ara-F.

In one embodiment, each of the sense and antisense strands of the dsRNA agent contains at least two different modifications.

In one embodiment, the dsRNA agent of Formula (I) further comprises 3′ and/or 5′ overhang(s) of 1-10 nucleotides in length. In one example, dsRNA agent of formula (I) comprises a 3′ overhang at the 3′-end of the antisense strand and a blunt end at the 5′-end of the antisense strand. In another example, the dsRNA agent has a 5′ overhang at the 5′-end of the sense strand.

In one embodiment, the dsRNA agent of the invention does not contain any 2′-F modification.

In one embodiment, the sense strand and/or antisense strand of the dsRNA agent comprises one or more blocks of phosphorothioate or methylphosphonate internucleotide linkages. In one example, the sense strand comprises one block of two phosphorothioate or methylphosphonate internucleotide linkages. In one example, the antisense strand comprises two blocks of two phosphorothioate or methylphosphonate internucleotide linkages. For example, the two blocks of phosphorothioate or methylphosphonate internucleotide linkages are separated by 16-18 phosphate internucleotide linkages.

In one embodiment, each of the sense and antisense strands of the dsRNA agent has 15-30 nucleotides. In one example, the sense strand has 19-22 nucleotides, and the antisense strand has 19-25 nucleotides. In another example, the sense strand has 21 nucleotides, and the antisense strand has 23 nucleotides.

In one embodiment, the nucleotide at position 1 of the 5′-end of the antisense strand in the duplex is selected from the group consisting of A, dA, dU, U, and dT. In one embodiment, at least one of the first, second, and third base pair from the 5′-end of the antisense strand is an AU base pair.

In one embodiment, the antisense strand of the dsRNA agent of the invention is 100% complementary to a target RNA to hybridize thereto and inhibits its expression through RNA interference. In another embodiment, the antisense strand of the dsRNA agent of the invention is at least 95%, at least 90%, at least 85%, at least 80%, at least 75%, at least 70%, at least 65%, at least 60%, at least 55%, or at least 50% complementary to a target RNA.

In one aspect, the invention relates to a dsRNA agent as defined herein capable of inhibiting the expression of a target gene. The dsRNA agent comprises a sense strand and an antisense strand, each strand having 14 to 40 nucleotides. The sense strand contains at least one thermally destabilizing nucleotide, wherein at least one of said thermally destabilizing nucleotide occurs at or near the site that is opposite to the seed region of the antisense strand (i.e. at position 2-8 of the 5′-end of the antisense strand). Each of the embodiments and aspects described in this specification relating to the dsRNA represented by formula (I) can also apply to the dsRNA containing the thermally destabilizing nucleotide.

The thermally destabilizing nucleotide can occur, for example, between positions 14-17 of the 5′-end of the sense strand when the sense strand is 21 nucleotides in length. The antisense strand contains at least two modified nucleic acids that are smaller than a sterically demanding 2′-OMe modification. Preferably, the two modified nucleic acids that are smaller than a sterically demanding 2′-OMe are separated by 11 nucleotides in length. For example, the two modified nucleic acids are at positions 2 and 14 of the 5′end of the antisense strand.

In one embodiment, the dsRNA agent further comprises at least one ASGPR ligand. For example, the ASGPR ligand is one or more GalNAc derivatives attached through a bivalent or trivalent branched linker, such as:

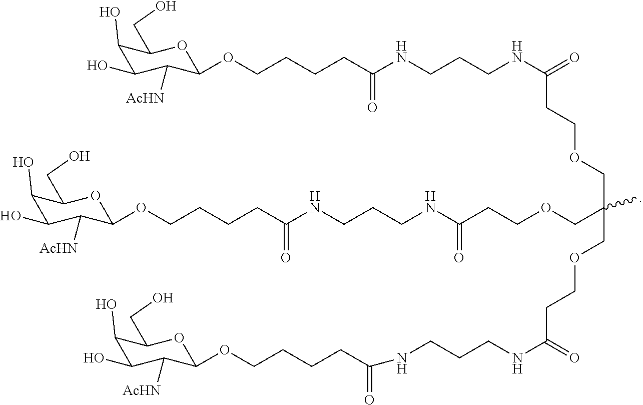 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In one example, the ASGPR ligand is attached to the 3′ end of the sense strand.

For example, the dsRNA agent as defined herein can comprise i) a phosphorus-containing group at the 5′-end of the sense strand or antisense strand; ii) with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand); and iii) a ligand, such as a ASGPR ligand (e.g., one or more GalNAc derivatives) at 5′-end or 3′-end of the sense strand or antisense strand. For instance, the ligand may be at the 3′-end of the sense strand.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-OMe, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-OMe, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-OMe, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-VP (e.g., a 5′-E-VP, 5′-Z—VP, or combination thereof), and a targeting ligand. In one embodiment, the 5′-VP is at the 5′-end of the antisense strand, and the targeting ligand is at the 3′-end of the sense strand.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-OMe, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS2 and a targeting ligand. In one embodiment, the 5′-PS2 is at the 5′-end of the antisense strand, and the targeting ligand is at the 3′-end of the sense strand.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-OMe, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-OMe, and q7 is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-OMe, and q7 is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-OMe, and q7 is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-VP (e.g., a 5′-E-VP, 5′-Z—VP, or combination thereof) and a targeting ligand. In one embodiment, the 5′-VP is at the 5′-end of the antisense strand, and the targeting ligand is at the 3′-end of the sense strand.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-OMe, and q7 is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-PS2 and a targeting ligand. In one embodiment, the 5′-PS2 is at the 5′-end of the antisense strand, and the targeting ligand is at the 3′-end of the sense strand.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-OMe, and q7 is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-F, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-F, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-F, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-VP (e.g., a 5′-E-VP, 5′-Z—VP, or combination thereof) and a targeting ligand. In one embodiment, the 5′-VP is at the 5′-end of the antisense strand, and the targeting ligand is at the 3′-end of the sense strand.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-F, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS2 and a targeting ligand. In one embodiment, the 5′-PS2 is at the 5′-end of the antisense strand, and the targeting ligand is at the 3′-end of the sense strand.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, T2′ is 2′-F, q4 is 2, B3′ is 2′-OMe or 2′-F, q5 is 5, T3′ is 2′-F, q6 is 1, B4′ is 2′-F, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-F, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-F, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-F, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-VP (e.g., a 5′-E-VP, 5′-Z—VP, or combination thereof) and a targeting ligand. In one embodiment, the 5′-VP is at the 5′-end of the antisense strand, and the targeting ligand is at the 3′-end of the sense strand.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-F, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS2 and a targeting ligand. In one embodiment, the 5′-PS2 is at the 5′-end of the antisense strand, and the targeting ligand is at the 3′-end of the sense strand.

In one embodiment, B1 is 2′-OMe or 2′-F, n1 is 8, T1 is 2′F, n2 is 3, B2 is 2′-OMe, n3 is 7, n4 is 0, B3 is 2′-OMe, n5 is 3, B1′ is 2′-OMe or 2′-F, q1 is 9, T1′ is 2′-F, q2 is 1, B2′ is 2′-OMe or 2′-F, q3 is 4, q4 is 0, B3′ is 2′-OMe or 2′-F, q5 is 7, T3′ is 2′-F, q6 is 1, B4′ is 2′-F, and q7 is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In a particular embodiment, the dsRNA agents of the present invention comprise:
a sense strand having:
a length of 21 nucleotides;
(ii) optionally an ASGPR ligand attached to the 3′-end, wherein said ASGPR ligand comprises three GalNAc derivatives attached through a trivalent branched linker; and
(iii) 2′-F modifications at positions 1, 3, 5, 7, 9 to 11, 13, 17, 19, and 21, and 2′-OMe modifications at positions 2, 4, 6, 8, 12, 14 to 16, 18, and 20 (counting from the 5′ end);
and
(b) an antisense strand having:
a length of 23 nucleotides;
(ii) 2′-OMe modifications at positions 1, 3, 5, 9, 11 to 13, 15, 17, 19, 21, and 23, and 2′F modifications at positions 2, 4, 6 to 8, 10, 14, 16, 18, 20, and 22 (counting from the 5′ end); and
(iii) phosphorothioate internucleotide linkages between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
a sense strand having:
a length of 21 nucleotides;
(ii) optionally an ASGPR ligand attached to the 3′-end, wherein said ASGPR ligand comprises three GalNAc derivatives attached through a trivalent branched linker;
(iii) 2′-F modifications at positions 1, 3, 5, 7, 9 to 11, 13, 15, 17, 19, and 21, and 2′-OMe modifications at positions 2, 4, 6, 8, 12, 14, 16, 18, and 20 (counting from the 5′ end); and
(iv) phosphorothioate internucleotide linkages between nucleotide positions 1 and 2, and between nucleotide positions 2 and 3 (counting from the 5′ end);
and
(b) an antisense strand having:
a length of 23 nucleotides;
(ii) 2′-OMe modifications at positions 1, 3, 5, 7, 9, 11 to 13, 15, 17, 19, and 21 to 23, and 2′F modifications at positions 2, 4, 6, 8, 10, 14, 16, 18, and 20 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
a sense strand having:
a length of 21 nucleotides;
(ii) optionally an ASGPR ligand attached to the 3′-end, wherein said ASGPR ligand comprises three GalNAc derivatives attached through a trivalent branched linker;
(iii) 2′-OMe modifications at positions 1 to 6, 8, 10, and 12 to 21, 2′-F modifications at positions 7, and 9, and a desoxy-nucleotide (e.g. dT) at position 11 (counting from the 5′ end); and
(iv) phosphorothioate internucleotide linkages between nucleotide positions 1 and 2, and between nucleotide positions 2 and 3 (counting from the 5′ end);
and
(b) an antisense strand having:
a length of 23 nucleotides;
(ii) 2′-OMe modifications at positions 1, 3, 7, 9, 11, 13, 15, 17, and 19 to 23, and 2′-F modifications at positions 2, 4 to 6, 8, 10, 12, 14, 16, and 18 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
a sense strand having:
a length of 21 nucleotides;
(ii) optionally an ASGPR ligand attached to the 3′-end, wherein said ASGPR ligand comprises three GalNAc derivatives attached through a trivalent branched linker;
(iii) 2′-OMe modifications at positions 1 to 6, 8, 10, 12, 14, and 16 to 21, and 2′-F modifications at positions 7, 9, 11, 13, and 15; and
(iv) phosphorothioate internucleotide linkages between nucleotide positions 1 and 2, and between nucleotide positions 2 and 3 (counting from the 5′ end);
and
(b) an antisense strand having:
a length of 23 nucleotides;
(ii) 2′-OMe modifications at positions 1, 5, 7, 9, 11, 13, 15, 17, 19, and 21 to 23, and 2′-F modifications at positions 2 to 4, 6, 8, 10, 12, 14, 16, 18, and 20 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
a sense strand having:
a length of 21 nucleotides;
(ii) optionally an ASGPR ligand attached to the 3′-end, wherein said ASGPR ligand comprises three GalNAc derivatives attached through a trivalent branched linker;
(iii) 2′-OMe modifications at positions 1 to 9, and 12 to 21, and 2′-F modifications at positions 10, and 11; and
(iv) phosphorothioate internucleotide linkages between nucleotide positions 1 and 2, and between nucleotide positions 2 and 3 (counting from the 5′ end);
and
(b) an antisense strand having:
a length of 23 nucleotides;
(ii) 2′-OMe modifications at positions 1, 3, 5, 7, 9, 11 to 13, 15, 17, 19, and 21 to 23, and 2′-F modifications at positions 2, 4, 6, 8, 10, 14, 16, 18, and 20 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
a sense strand having:
a length of 21 nucleotides;
(ii) optionally an ASGPR ligand attached to the 3′-end, wherein said ASGPR ligand comprises three GalNAc derivatives attached through a trivalent branched linker;
(iii) 2′-F modifications at positions 1, 3, 5, 7, 9 to 11, and 13, and 2′-OMe modifications at positions 2, 4, 6, 8, 12, and 14 to 21; and
(iv) phosphorothioate internucleotide linkages between nucleotide positions 1 and 2, and between nucleotide positions 2 and 3 (counting from the 5′ end);
and
(b) an antisense strand having:
a length of 23 nucleotides;
(ii) 2′-OMe modifications at positions 1, 3, 5 to 7, 9, 11 to 13, 15, 17 to 19, and 21 to 23, and 2′-F modifications at positions 2, 4, 8, 10, 14, 16, and 20 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
a sense strand having:
a length of 21 nucleotides;
(ii) optionally an ASGPR ligand attached to the 3′-end, wherein said ASGPR ligand comprises three GalNAc derivatives attached through a trivalent branched linker;
(iii) 2′-OMe modifications at positions 1, 2, 4, 6, 8, 12, 14, 15, 17, and 19 to 21, and 2′-F modifications at positions 3, 5, 7, 9 to 11, 13, 16, and 18; and
(iv) phosphorothioate internucleotide linkages between nucleotide positions 1 and 2, and between nucleotide positions 2 and 3 (counting from the 5′ end);
and
(b) an antisense strand having:
a length of 25 nucleotides;
(ii) 2′-OMe modifications at positions 1, 4, 6, 7, 9, 11 to 13, 15, 17, and 19 to 23, 2′-F modifications at positions 2, 3, 5, 8, 10, 14, 16, and 18, and desoxy-nucleotides (e.g. dT) at positions 24 and 25 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a four nucleotide overhang at the 3′-end of the antisense strand, and a blunt end at the 5′-end of the antisense strand.

In another particular embodiment, the dsRNA agents of the present invention comprise:
a sense strand having:
a length of 21 nucleotides;
(ii) optionally an ASGPR ligand attached to the 3′-end, wherein said ASGPR ligand comprises three GalNAc derivatives attached through a trivalent branched linker;
(iii) 2′-OMe modifications at positions 1 to 6, 8, and 12 to 21, and 2′-F modifications at positions 7, and 9 to 11; and
(iv) phosphorothioate internucleotide linkages between nucleotide positions 1 and 2, and between nucleotide positions 2 and 3 (counting from the 5′ end);
and
(b) an antisense strand having:
a length of 23 nucleotides;
(ii) 2′-OMe modifications at positions 1, 3 to 5, 7, 8, 10 to 13, 15, and 17 to 23, and 2′-F modifications at positions 2, 6, 9, 14, and 16 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
a sense strand having:
a length of 21 nucleotides;
(ii) optionally an ASGPR ligand attached to the 3′-end, wherein said ASGPR ligand comprises three GalNAc derivatives attached through a trivalent branched linker;
(iii) 2′-OMe modifications at positions 1 to 6, 8, and 12 to 21, and 2′-F modifications at positions 7, and 9 to 11; and
(iv) phosphorothioate internucleotide linkages between nucleotide positions 1 and 2, and between nucleotide positions 2 and 3 (counting from the 5′ end);
and
(b) an antisense strand having:
a length of 23 nucleotides;
(ii) 2′-OMe modifications at positions 1, 3 to 5, 7, 10 to 13, 15, and 17 to 23, and 2′-F modifications at positions 2, 6, 8, 9, 14, and 16 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
a sense strand having:
a length of 19 nucleotides;
(ii) optionally an ASGPR ligand attached to the 3′-end, wherein said ASGPR ligand comprises three GalNAc derivatives attached through a trivalent branched linker;
(iii) 2′-OMe modifications at positions 1 to 4, 6, and 10 to 19, and 2′-F modifications at positions 5, and 7 to 9; and
(iv) phosphorothioate internucleotide linkages between nucleotide positions 1 and 2, and between nucleotide positions 2 and 3 (counting from the 5′ end);
and
(b) an antisense strand having:
a length of 21 nucleotides;
(ii) 2′-OMe modifications at positions 1, 3 to 5, 7, 10 to 13, 15, and 17 to 21, and 2′-F modifications at positions 2, 6, 8, 9, 14, and 16 (counting from the 5′ end); and
(iii) phosphorothioate internucleotide linkages between nucleotide positions 1 and 2, between nucleotide positions 2 and 3, between nucleotide positions 19 and 20, and between nucleotide positions 20 and 21 (counting from the 5′ end);
wherein the dsRNA agents have a two nucleotide overhang at the 3′-end of the antisense strand, and a blunt end at the 5′-end of the antisense strand.

Various publications described multimeric siRNA and can all be used with the iRNA of the invention. Such publications include WO2007/091269, U.S. Pat. No. 7,858,769, WO2010/141511, WO2007/117686, WO2009/014887 and WO2011/031520, which are hereby incorporated by reference in their entirety.

In some embodiments, 100%, 99%, 98%, 97%, 96%, 95%, 94%, 93%, 02%, 91%, 90%, 85%, 80%, 75%, 70%, 65%, 60%, 55%, 50%, 45%, 40%, 35% or 30% of the iRNA agent of the invention is modified.

In some embodiments, each of the sense and antisense strands of the iRNA agent is independently modified with acyclic nucleotides, LNA, HNA, CeNA, 2′-methoxyethyl, 2′-O-methyl, 2′-O-allyl, 2′-C-allyl, 2′-deoxy, 2′-fluoro, 2′-O—N-methylacetamido (2′-O-NMA), a 2′-O-dimethylaminoethoxyethyl (2′-O-DMAEOE), 2′-O-aminopropyl (2′-O-AP), or 2′-ara-F.

In some embodiments, each of the sense and antisense strands of the iRNA agent contains at least two different modifications.

In some embodiments, the double-stranded iRNA agent of the invention of the invention does not contain any 2′-F modification.

In some embodiments, the double-stranded iRNA agent of the invention contains one, two, three, four, five, six, seven, eight, nine, ten, eleven or twelve 2′-F modification(s). In one example, double-stranded iRNA agent of the invention contains nine or ten 2′-F modifications.

The iRNA agent of the invention may further comprise at least one phosphorothioate or methylphosphonate internucleotide linkage. The phosphorothioate or methylphosphonate internucleotide linkage modification may occur on any nucleotide of the sense strand or antisense strand or both in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In one embodiment, the iRNA comprises the phosphorothioate or methylphosphonate internucleotide linkage modification in the overhang region. For example, the overhang region may contain two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a parried nucleotide next to the overhang nucleotide. Preferably, these terminal three nucleotides may be at the 3′-end of the antisense strand.

In some embodiments, the sense strand and/or antisense strand of the iRNA agent comprises one or more blocks of phosphorothioate or methylphosphonate internucleotide linkages. In one example, the sense strand comprises one block of two phosphorothioate or methylphosphonate internucleotide linkages. In one example, the antisense strand comprises two blocks of two phosphorothioate or methylphosphonate internucleotide linkages. For example, the two blocks of phosphorothioate or methylphosphonate internucleotide linkages are separated by 16-18 phosphate internucleotide linkages.

In some embodiments, the antisense strand of the iRNA agent of the invention is 100% complementary to a target RNA to hybridize thereto and inhibits its expression through RNA interference. In another embodiment, the antisense strand of the iRNA agent of the invention is at least 95%, at least 90%, at least 85%, at least 80%, at least 75%, at least 70%, at least 65%, at least 60%, at least 55%, or at least 50% complementary to a target RNA.

In one aspect, the invention relates to a iRNA agent capable of inhibiting the expression of a target gene. The iRNA agent comprises a sense strand and an antisense strand, each strand having 14 to 40 nucleotides. The sense strand contains at least one thermally destabilizing nucleotide, wherein at at least one said thermally destabilizing nucleotide occurs at or near the site that is opposite to the seed region of the antisense strand (i.e. at position 2-8 of the 5′-end of the antisense strand), For example, the thermally destabilizing nucleotide occurs between positions 14-17 of the 5′-end of the sense strand when the sense strand is 21 nucleotides in length. The antisense strand contains at least two modified nucleic acids that are smaller than a sterically demanding 2′-OMe modification. Preferably, the two modified nucleic acids that is smaller than a sterically demanding 2′-OMe are separated by 11 nucleotides in length. For example, the two modified nucleic acids are at positions 2 and 14 of the 5′end of the antisense strand.

### IV. iRNAs Conjugated to Ligands

In certain embodiments, the double-stranded iRNA agent of the invention is further modified by covalent attachment of one or more conjugate groups. In general, conjugate groups modify one or more properties of the attached double-stranded iRNA agent of the invention including but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to a parent compound such as an oligomeric compound. A preferred list of conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

In some embodiments, the double-stranded iRNA agent further comprises a targeting ligand that targets a receptor which mediates delivery to a specific CNS tissue. These targeting ligands can be conjugated in combination with the lipophilic moiety to enable specific intrathecal and systemic delivery.

Exemplary targeting ligands that targets the receptor mediated delivery to a CNS tissue are peptide ligands such as Angiopep-2, lipoprotein receptor related protein (LRP) ligand, bEnd.3 cell binding ligand; transferrin receptor (TfR) ligand (which can utilize iron transport system in brain and cargo transport into the brain parenchyma); manose receptor ligand (which targets olfactory ensheathing cells), glucose transporter protein, and LDL receptor ligand.

In some embodiments, the double-stranded iRNA agent further comprises a targeting ligand that targets a receptor which mediates delivery to a specific ocular tissue. These targeting ligands can be conjugated in combination with the lipophilic moiety to enable specific intravitreal and systemic delivery. Exemplary targeting ligands that targets the receptor mediated delivery to a ocular tissue are lipophilic ligands such as all-trans retinol (which targets the retinoic acid receptor); RGD peptide (which targets retinal pigment epithelial cells), such as H-Gly-Arg-Gly-Asp-Ser-Pro-Lys-Cys-OH (SEQ ID NO: 14) or Cyclo(-Arg-Gly-Asp-D-Phe-Cys) (SEQ ID NO:104); LDL receptor ligands; and carbohydrate based ligands (which targets endothelial cells in posterior eye).

Preferred conjugate groups amenable to the present invention include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996, 277, 923).

Generally, a wide variety of entities, e.g., ligands, can be coupled to the oligomeric compounds described herein. Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]₂, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g, steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a.helical cell-permeation agent).

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H2A peptides, Xenopus peptides, esculentinis-1, and caerins.

As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), Golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and branched polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to, AALEALAEALEALAEALEALAEAAAAGGC (GALA) (SEQ ID NO: 18); AALAEALAEALAEALAEALAEALAAAAGGC (EALA) (SEQ ID NO: 19); ALEALAEALEALAEA (SEQ ID NO: 20); GLFEAIEGFIENGWEGMIWDYG (INF-7) (SEQ ID NO: 21); GLFGAIAGFIENGWEGMIDGWYG (Inf HA-2) (SEQ ID NO: 22); GLFEAIEGFIENGWEGMIDGWYGCGLFEAIEGFIENGWEGMID GWYGC (diINF-7) (SEQ ID NO: 23); GLFEAIEGFIENGWEGMIDGGCGLFEAIEGFIENGWEGMIDGGC (diINF-3) (SEQ ID NO: 24); GLFGALAEALAEALAEHLAEALAEALEALAAGGSC (GLF) (SEQ ID NO: 25); GLFEAIEGFIENGWEGLAEALAEALEALAAGGSC (GALA-INF3) (SEQ ID NO: 26); GLF EAI EGFI ENGW EGnI DG K GLF EAI EGFI ENGW EGnI DG (INF-5, n is norleucine) (SEQ ID NO: 27); LFEALLELLESLWELLLEA (JTS-1) (SEQ ID NO: 28); GLFKALLKLLKSLWKLLLKA (ppTG1) (SEQ ID NO: 29); GLFRALLRLLRSLWRLLLRA (ppTG20) (SEQ ID NO: 30); WEAKLAKALAKALAKHLAKALAKALKACEA (KALA) (SEQ ID NO: 31); GLFFEAIAEFIEGGWEGLIEGC (HA) (SEQ ID NO: 32); GIGAVLKVLTTGLPALISWIKRKRQQ (Melittin) (SEQ ID NO: 33); HsWYG (SEQ ID NO: 34); and CHK₆HC (SEQ ID NO: 35).

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (also referred to as XTC herein).

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 20070036865; and 2004/0198687, contents of which are hereby incorporated by reference in their entirety.

Exemplary cell permeation peptides include, but are not limited to, RQIKIWFQNRRMKWKK (penetratin) (SEQ ID NO: 36); GRKKRRQRRRPPQC (Tat fragment 48-60) (SEQ ID NO: 37); GALFLGWLGAAGSTMGAWSQPKKKRKV (signal sequence based peptide) (SEQ ID NO: 38); LLIILRRRIRKQAHAHSK (PVEC) (SEQ ID NO: 39); GWTLNSAGYLLKINLKALAALAKKIL (transportan) (SEQ ID NO: 40); KLALKLALKALKAALKLA (amphiphilic model peptide) (SEQ ID NO: 41); RRRRRRRRR (Arg9) (SEQ ID NO: 42); KFFKFFKFFK (Bacterial cell wall permeating peptide) (SEQ ID NO: 43); LLGDFFRKSKEKIGKEFKRIVQRIKDFLRNLVPRTES (LL-37) (SEQ ID NO: 44); SWLSKTAKKLENSAKKRISEGIAIAIQGGPR (cecropin P1) (SEQ ID NO: 45); ACYCRIPACIAGERRYGTCIYQGRLWAFCC (α-defensin) (SEQ ID NO: 46); DHYNCVSSGGQCLYSACPIFTKIQGTCYRGKAKCCK (β-defensin) (SEQ ID NO: 47); RRRPRPPYLPRPRPPPFFPPRLPPRIPPGFPPRFPPRFPGKR-NH2 (PR-39) (SEQ ID NO: 48); ILPWKWPWWPWRR-NH2 (indolicidin) (SEQ ID NO: 49); AAVALLPAVLLALLAP (RFGF) (SEQ ID NO: 50); AALLPVLLAAP (RFGF analogue) (SEQ ID NO: 51); and RKCRIVVIRVCR (bactenecin) (SEQ ID NO: 52).

Exemplary cationic groups include, but are not limited to, protonated amino groups, derived from e.g., 0-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

Carbohydrate based targeting ligands include, but are not limited to, D-galactose, multivalent galactose, N-acetyl-D-galactosamine (GalNAc), multivalent GalNAc, e.g. GalNAc₂ and GalNAc₃ (GalNAc and multivalent GalNAc are collectively referred to herein as GalNAc conjugates); D-mannose, multivalent mannose, multivalent lactose, N-acetyl-gulucosamine, Glucose, multivalent Glucose, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as ligands are described in U.S. Pat. Nos. 2,816,110; 5,552,545; 6,335,434 and 7,128,893, contents of which are herein incorporated in their entireties by reference.

As used herein, the terms “PK modulating ligand” and “PK modulator” refers to molecules which can modulate the pharmacokinetics of the composition of the invention. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, PEGs, biotin, and transthyretia-binding ligands (e.g., tetraiidothyroacetic acid, 2, 4, 6-triiodophenol and flufenamic acid). Oligomeric compounds that comprise a number of phosphorothioate intersugar linkages are also known to bind to serum protein, thus short oligomeric compounds, e.g. oligonucleotides of comprising from about 5 to 30 nucleotides (e.g., 5 to 25 nucleotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleotide linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, scuh as those described in Oravcova, et al., Journal of Chromatography B (1996), 677: 1-27.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

The ligand or tethered ligand can be present on a monomer when said monomer is incorporated into a component of the double-stranded iRNA agent of the invention (e.g., double-stranded iRNA agent of the invention or linker). In some embodiments, the ligand can be incorporated via coupling to a “precursor” monomer after said “precursor” monomer has been incorporated into a component of the double-stranded iRNA agent of the invention (e.g., double-stranded iRNA agent of the invention or linker). For example, a monomer having, e.g., an amino-terminated tether (i.e., having no associated ligand), e.g., monomer-linker-NH₂ can be incorporated into a component of the compounds of the invention (e.g., an double-stranded iRNA agent of the invention or linker). In a subsequent operation, i.e., after incorporation of the precursor monomer into a component of the compounds of the invention (e.g., double-stranded iRNA agent of the invention or linker), a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether.

In another example, a monomer having a chemical group suitable for taking part in Click Chemistry reaction can be incorporated e.g., an azide or alkyne terminated tether/linker. In a subsequent operation, i.e., after incorporation of the precursor monomer into the strand, a ligand having complementary chemical group, e.g. an alkyne or azide can be attached to the precursor monomer by coupling the alkyne and the azide together.

In some embodiments, ligand can be conjugated to nucleobases, sugar moieties, or internucleosidic linkages of the double-stranded iRNA agent of the invention. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a ligand is conjugated to a nucleobase, the preferred position is one that does not interfere with hybridization, i.e., does not interfere with the hydrogen bonding interactions needed for base pairing.

Conjugation to sugar moieties of nucleosides can occur at any carbon atom. Example carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithiotate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

There are numerous methods for preparing conjugates of oligonuclotides. Generally, an oligonucleotide is attached to a conjugate moiety by contacting a reactive group (e.g., OH, SH, amine, carboxyl, aldehyde, and the like) on the oligonucleotide with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

The ligand can be attached to the double-stranded iRNA agent of the inventions via a linker or a carrier monomer, e.g., a ligand carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier monomer into the backbone, e.g., the phosphate, or modified phosphate, e.g., sulfur containing, backbone, of an oligonucleotide. A “tethering attachment point” (TAP) in refers to an atom of the carrier monomer, e.g., a carbon atom or a heteroatom (distinct from an atom which provides a backbone attachment point), that connects a selected moiety. The selected moiety can be, e.g., a carbohydrate, e.g. monosaccharide, disaccharide, trisaccharide, tetrasaccharide, oligosaccharide and polysaccharide. Optionally, the selected moiety is connected by an intervening tether to the carrier monomer. Thus, the carrier will often include a functional group, e.g., an amino group, or generally, provide a bond, that is suitable for incorporation or tethering of another chemical entity, e.g., a ligand to the constituent atom.

Representative U.S. patents that teach the preparation of conjugates of nucleic acids include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,149,782; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; 5,672,662; 5,688,941; 5,714,166; 6,153,737; 6,172,208; 6,300,319; 6,335,434; 6,335,437; 6,395,437; 6,444,806; 6,486,308; 6,525,031; 6,528,631; 6,559,279; contents of which are herein incorporated in their entireties by reference.

In some embodiments, the double-stranded iRNA agent further comprises a targeting ligand that targets a liver tissue. In some embodiments, the targeting ligand is a carbohydrate-based ligand. In one embodiment, the targeting ligand is a GalNAc conjugate.

In certain embodiments, the double-stranded iRNA agent of the invention further comprises a ligand having a structure shown below:

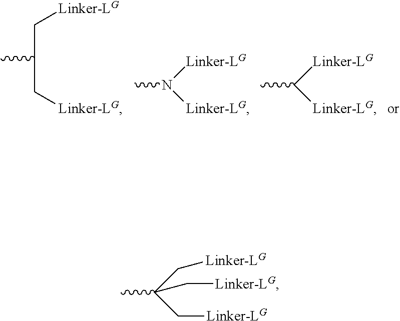

wherein:
L^{G} is independently for each occurrence a ligand, e.g., carbohydrate, e.g. monosaccharide, disaccharide, trisaccharide, tetrasaccharide, polysaccharide; and
Z′, Z″, Z′″ and Z″″ are each independently for each occurrence O or S.

In certain embodiments, the double-stranded iRNA agent of the invention comprises a ligand of Formula (II), (III), (IV) or (V):

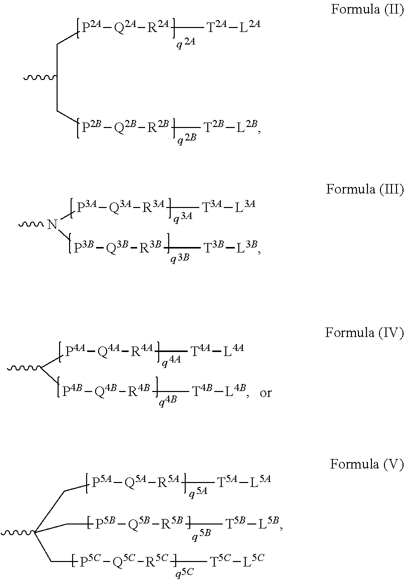

wherein:
q^{2A}, q^{2B}, q^{3A}, q^{3B}, q^{4A}, q^{4B}, q^{5A}, q^{5B} and q^{5C} represent independently for each occurrence 0-20 and wherein the repeating unit can be the same or different;
Q and Q′ are independently for each occurrence is absent, —(P⁷-Q⁷-R⁷)ₚ-T⁷- or -T⁷-Q⁷-T^{7′}-B-T^{8′}-Q⁸-T⁸;
P^{2A}, P^{2B}, P^{3A}, P^{3B}, P^{4A}, P^{4B}, P^{5A}, P^{5B}, P^{5C}, P⁷, T^{2A}, T^{2B}, T^{3A}, T^{3B}, T^{4A}, T^{4B}, T^{4A}, T^{5B}, T^{5C}, T⁷, T^{7′}, T⁸ and T^{8′} are each independently for each occurrence absent, CO, NH, O, S, OC(O), NHC(O), CH₂, CH₂NH or CH₂O;
B is —CH₂—N(B^{L})—CH₂—;
B^{L} is -T^{B}-Q^{B}-T^{B′}-R^{x;}
Q^{2A}, Q^{2B}, Q^{3A}, Q^{3B}, Q^{4A}, Q^{4B}, Q^{5A}, Q^{5B}, Q^{5C}, Q⁷, Q⁸ and Q^{B} are independently for each occurrence absent, alkylene, substituted alkylene and wherein one or more methylenes can be interrupted or terminated by one or more of O, S, S(O), SO₂, N(R^{N}), C(R′)═C(R′), C≡C or C(O);
T^{B} and T^{B′} are each independently for each occurrence absent, CO, NH, O, S, OC(O), OC(O)O, NHC(O), NHC(O)NH, NHC(O)O, CH₂, CH₂NH or CH₂O;
R^{x} is a lipophile (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), a vitamin (e.g., folate, vitamin A, vitamin E, biotin, pyridoxal), a peptide, a carbohydrate (e.g., monosaccharide, disaccharide, trisaccharide, tetrasaccharide, oligosaccharide, polysaccharide), an endosomolytic component, a steroid (e.g., uvaol, hecigenin, diosgenin), a terpene (e.g., triterpene, e.g., sarsasapogenin, Friedelin, epifriedelanol derivatized lithocholic acid), or a cationic lipid;
R¹, R², R^{2A}, R^{2B}, R^{3A}, R^{3B}, R^{4A}, R^{4B}, R^{5A}, R^{5B}, R^{5C}, R⁷ are each independently for each occurrence absent, NH, O, S, CH₂, C(O)O, C(O)NH, NHCH(R^{a})C(O), —C(O)—CH(R^{a})—NH—, CO,

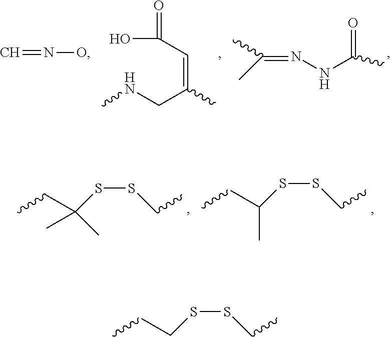

or heterocyclyl;
L¹, L^{2A}, L^{2B}, L^{3A}, L^{3B}, L^{4A}, L^{4B} L^{5A} L^{5B} and L^{5C} are each independently for each occurrence a carbohydrate, e.g., monosaccharide, disaccharide, trisaccharide, tetrasaccharide, oligosaccharide and polysaccharide;
R′ and R″ are each independently H, C₁-C₆ alkyl, OH, SH, or N(R^{N})₂;
R^{N} is independently for each occurrence H, methyl, ethyl, propyl, isopropyl, butyl or benzyl;
R^{a} is H or amino acid side chain;
Z′, Z″, Z′″ and Z″″ are each independently for each occurrence O or S;
p represents independently for each occurrence 0-20.

As discussed above, because the ligand can be conjugated to the iRNA agent via a linker or carrier, and because the linker or carrier can contain a branched linker, the iRNA agent can then contain multiple ligands via the same or different backbone attachment points to the carrier, or via the branched linker(s). For instance, the branchpoint of the branched linker may be a bivalent, trivalent, tetravalent, pentavalent, or hexavalent atom, or a group presenting such multiple valencies. In certain embodiments, the branchpoint is —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In other embodiment, the branchpoint is glycerol or glycerol derivative.

In certain embodiments, the double-stranded iRNA agent of the invention comprises a ligand of structure:

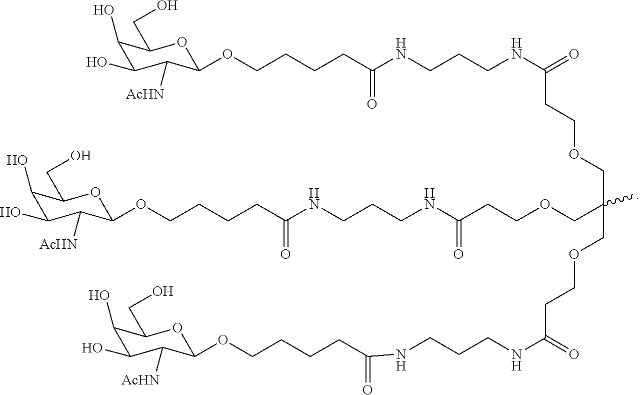 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In certain embodiments, the double-stranded iRNA agent of the invention comprises a ligand of structure:

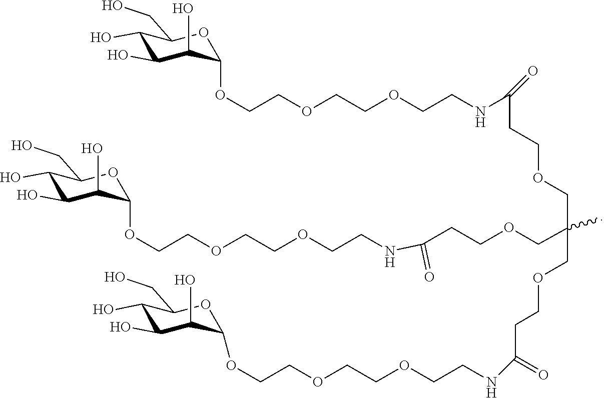

In certain embodiments, the double-stranded iRNA agent of the invention comprises a ligand of structure:

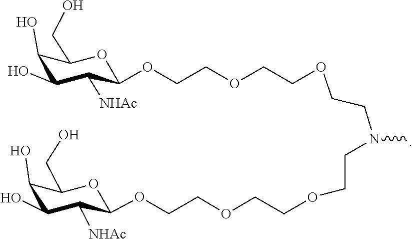

In certain embodiments, the double-stranded iRNA agent of the invention comprises a ligand of structure:

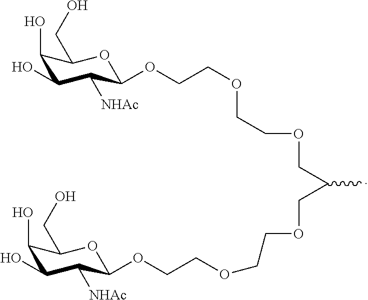

In certain embodiments, the double-stranded iRNA agent of the invention comprises a ligand of structure:

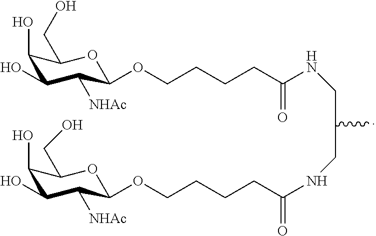

In certain embodiments, the double-stranded iRNA agent of the invention comprises a ligand of

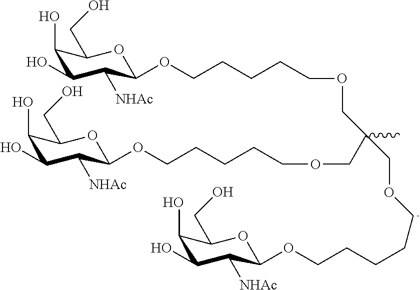

In certain embodiments, the double-stranded iRNA agent of the invention comprises a ligand of structure:

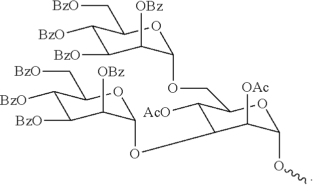

In certain embodiments, the double-stranded iRNA agent of the invention comprises a ligand of structure:

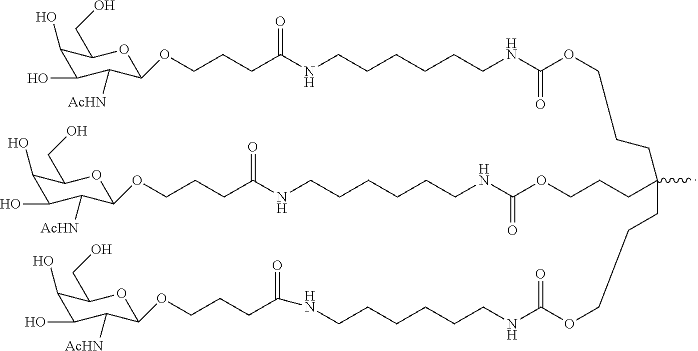 [SMILES: CC(=O)NC1C(OCCCC(=O)NCCCCCCNC(=O)OCCCC(C)(CCCOC(=O)NCCCCCCNC(=O)CCCOC2OC(CO)C(O)C(O)C2NC(C)=O)CCCOC(=O)NCCCCCCNC(=O)CCCOC2OC(CO)C(O)C(O)C2NC(C)=O)OC(CO)C(O)C1O]

In certain embodiments, the double-stranded iRNA agent of the invention comprises a ligand of structure:

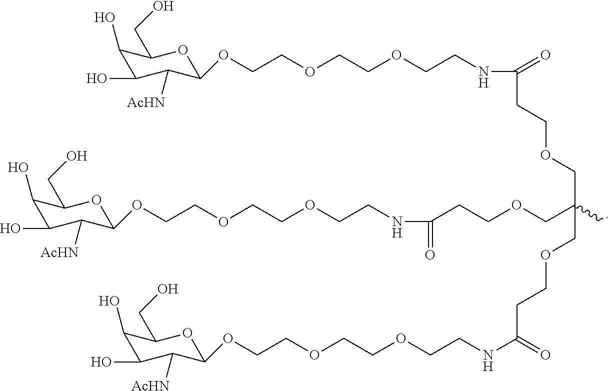 [SMILES: CC(=O)NC1C(OCCOCCOCCNC(=O)CCOCC(C)(COCCC(=O)NCCOCCOCCOC2OC(CO)C(O)C(O)C2NC(C)=O)COCCC(=O)NCCOCCOCCOC2OC(CO)C(O)C(O)C2NC(C)=O)OC(CO)C(O)C1O]

In certain embodiments, the double-stranded iRNA agent of the invention comprises a ligand of structure:

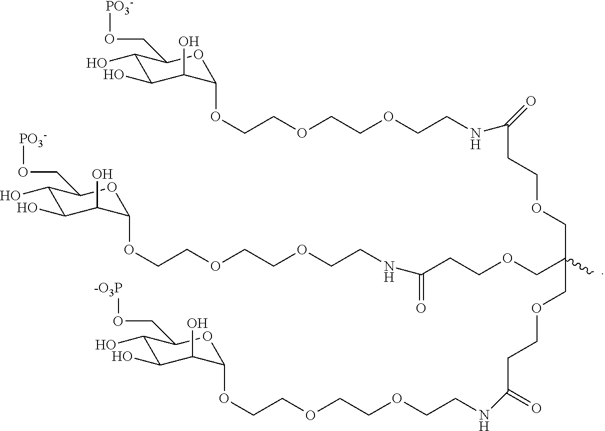

In certain embodiments, the double-stranded iRNA agent of the invention comprises a ligand of structure:

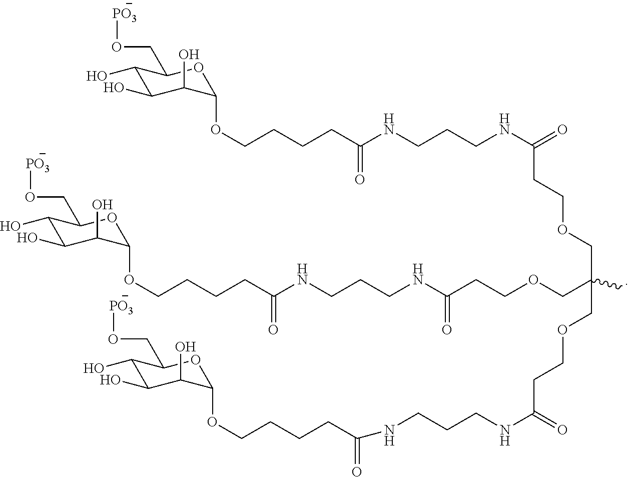

In certain embodiments, the double-stranded iRNA agent of the invention comprises a monomer of structure:

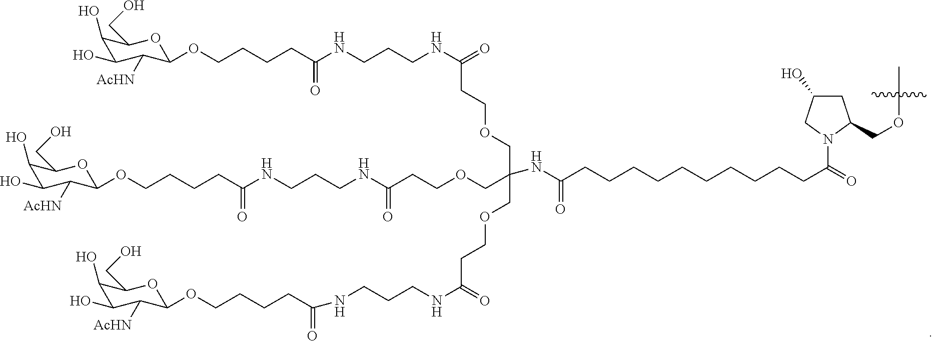 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In certain embodiments, the double-stranded iRNA agent of the invention comprises a ligand of structure:

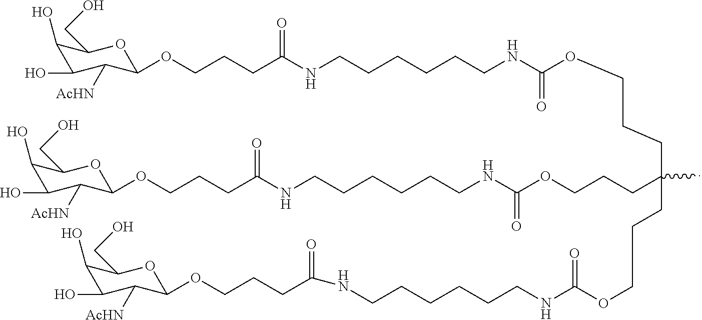 [SMILES: CC(=O)NC1C(OCCCC(=O)NCCCCCCNC(=O)OCCCC(C)(CCCOC(=O)NCCCCCCNC(=O)CCCOC2OC(CO)C(O)C(O)C2NC(C)=O)CCCOC(=O)NCCCCCCNC(=O)CCCOC2OC(CO)C(O)C(O)C2NC(C)=O)OC(CO)C(O)C1O]

In certain embodiments, the double-stranded iRNA agent of the invention comprises a monomer of structure:

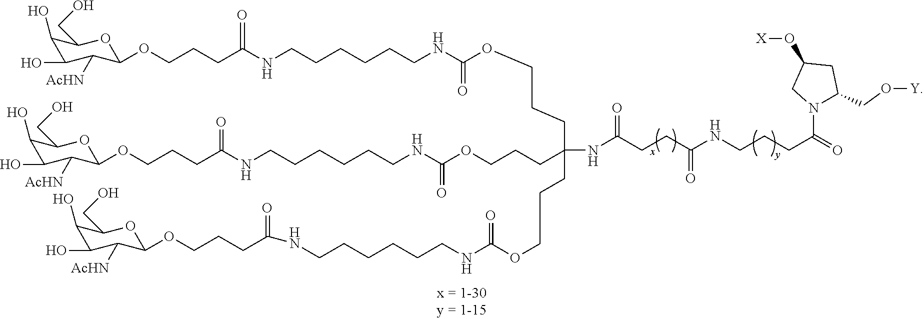

In certain embodiments, the double-stranded iRNA agent of the invention comprises a monomer of structure:

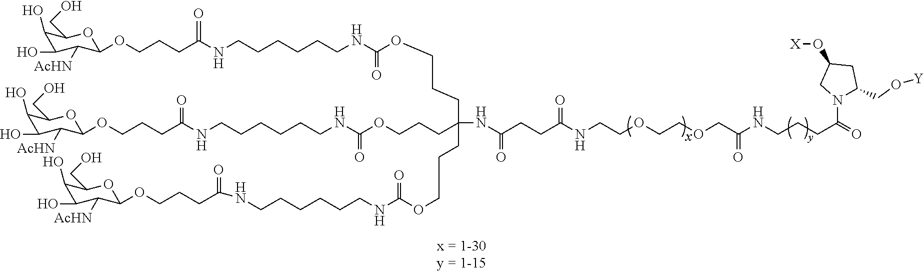

In certain embodiments, the double-stranded iRNA agent of the invention comprises a monomer of structure:

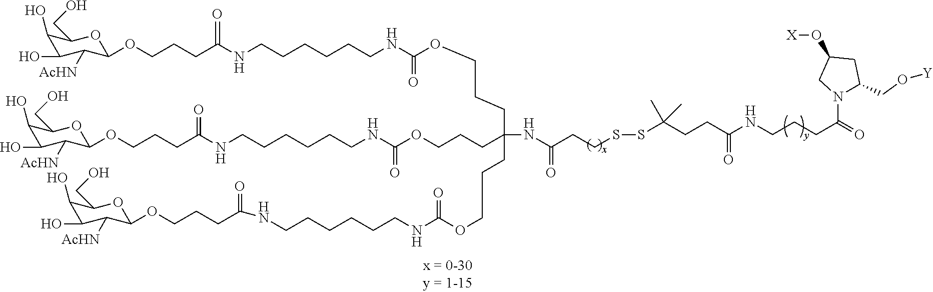

In certain embodiments, the double-stranded iRNA agent of the invention comprises a monomer of structure:

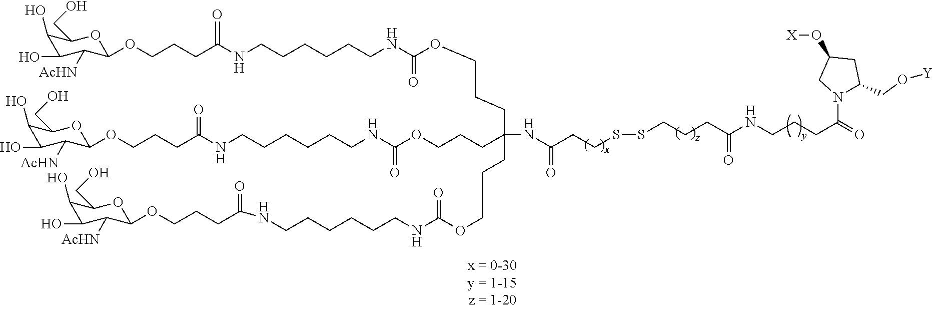

In certain embodiments, the double-stranded iRNA agent of the invention comprises a monomer of structure:

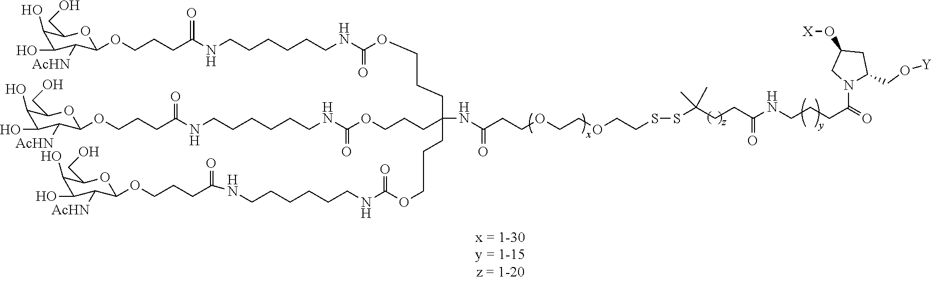

In certain embodiments, the double-stranded iRNA agent of the invention comprises a monomer of structure:

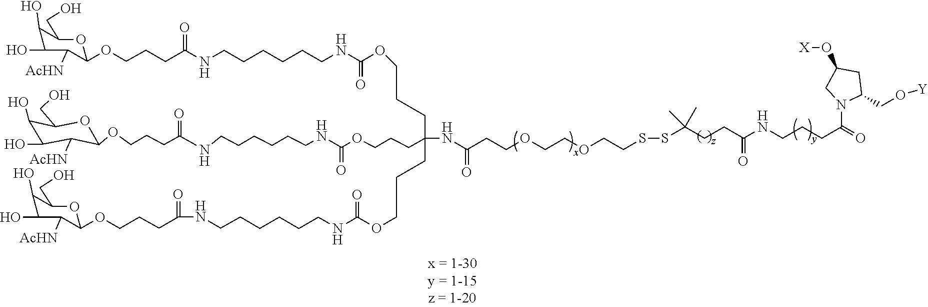

In certain embodiments, the double-stranded iRNA agent of the invention comprises a ligand of structure:

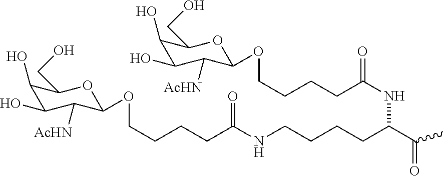

In certain embodiments, the double-stranded iRNA agent of the invention comprises a ligand of structure:

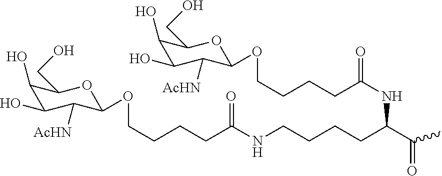

In certain embodiments, the double-stranded iRNA agent of the invention comprises a ligand of structure:

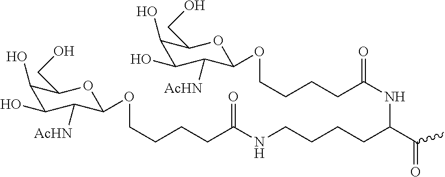

In certain embodiments, the double-stranded iRNA agent of the invention comprises a ligand of structure:

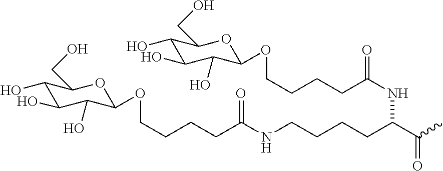

In certain embodiments, the double-stranded iRNA agent of the invention comprises a ligand of structure:

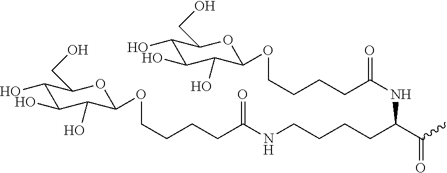

In certain embodiments, the double-stranded iRNA agent of the invention comprises a ligand of structure:

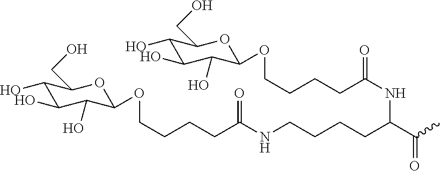

In certain embodiments, the double-stranded iRNA agent of the invention comprises a ligand of structure:

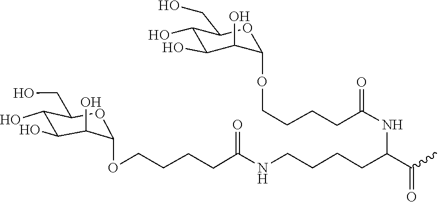

In certain embodiments, the double-stranded iRNA agent of the invention comprises a ligand of structure:

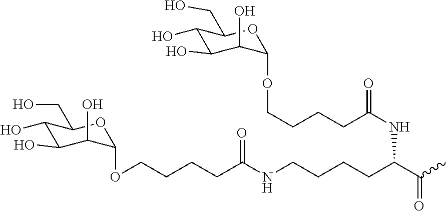

In certain embodiments, the double-stranded iRNA agent of the invention comprises a ligand of structure:

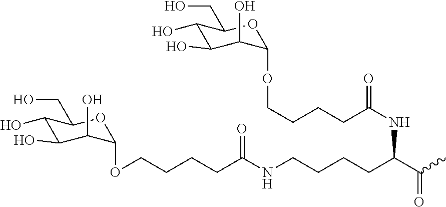

In certain embodiments, the double-stranded iRNA agent of the invention comprises a monomer of structure:

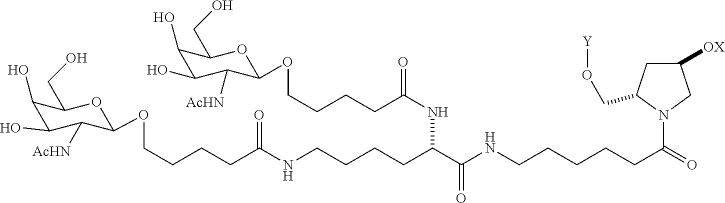

In certain embodiments, the double-stranded iRNA agent of the invention comprises a monomer of structure:

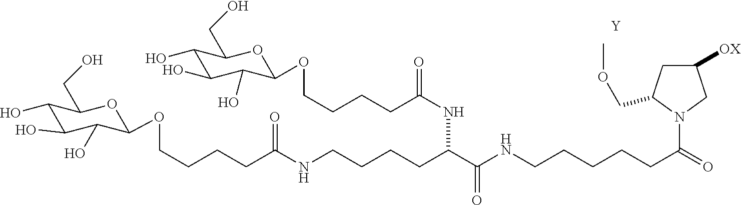

In certain embodiments, the double-stranded iRNA agent of the invention comprises a monomer of structure:

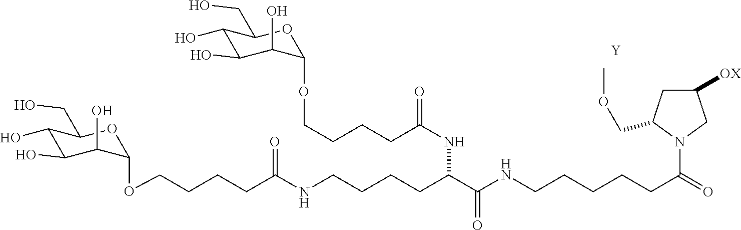

In certain embodiments, the double-stranded iRNA agent of the invention comprises a monomer of structure:

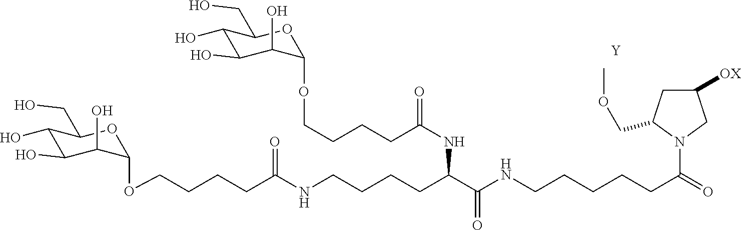

In certain embodiments, the double-stranded iRNA agent of the invention comprises a monomer of structure:

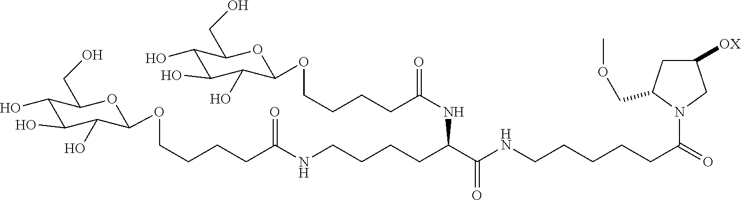

In certain embodiments, the double-stranded iRNA agent of the invention comprises a monomer of structure:

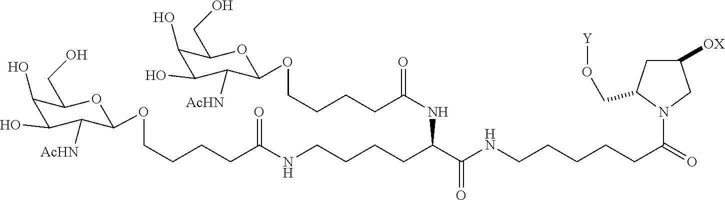

In some embodiments both L^{2A} and L^{2B} are different.

In some preferred embodiments both L^{3A} and L^{3B} are the same.

In some embodiments both L^{3A} and L^{3B} are different.

In some preferred embodiments both L^{4A} and L^{4B} are the same.

In some embodiments both L^{4A} and L^{4B} are different.

In some preferred embodiments all of L^{5A}, L^{5B} and L^{5C} are the same.

In some embodiments two of L^{5A}, L^{5B} and L^{5C} are the same

In some embodiments L^{5A} and L^{5B} are the same.

In some embodiments L^{5A} and L^{5C} are the same.

In some embodiments L^{5B} and L^{5C} are the same.

In certain embodiments, the double-stranded iRNA agent of the invention comprises a monomer of structure:

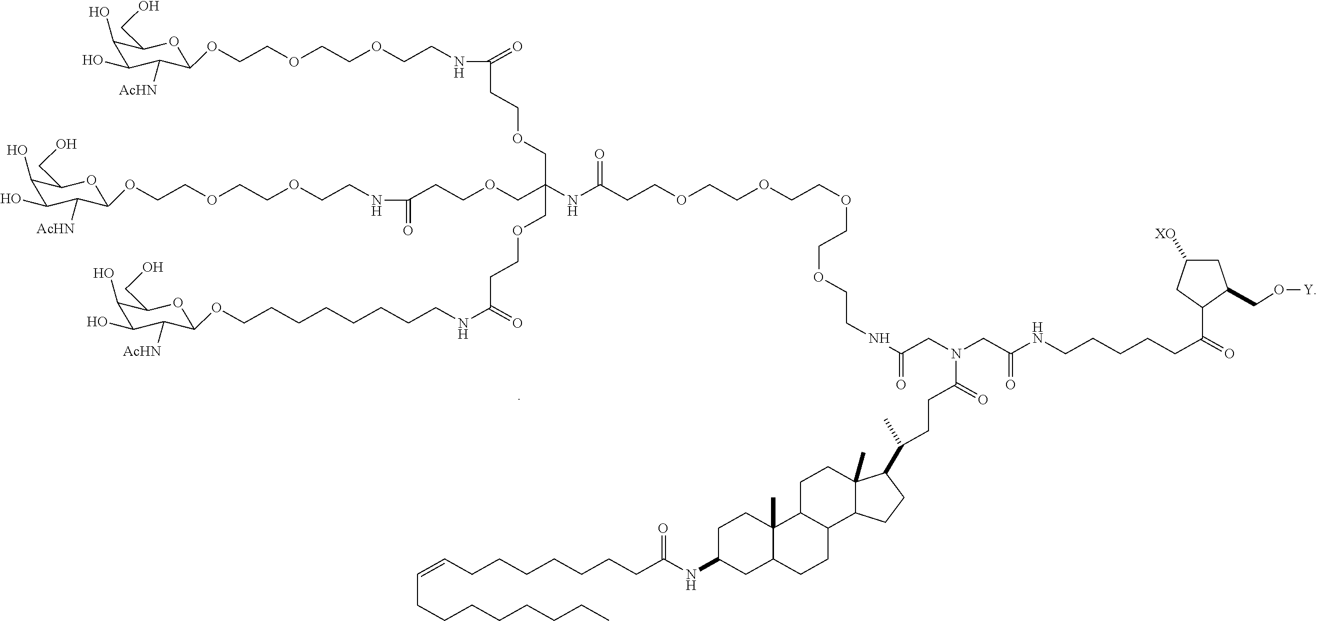

In certain embodiments, the double-stranded iRNA agent of the invention comprises a monomer of structure:

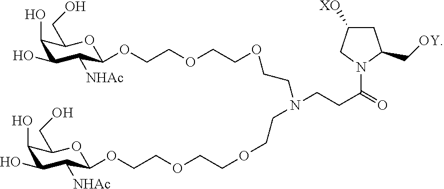

In certain embodiments, the double-stranded iRNA agent of the invention comprises a monomer of structure:

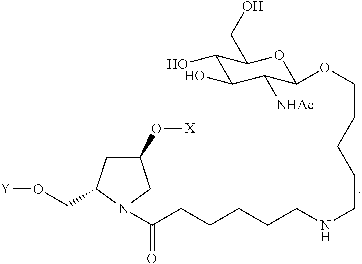

In certain embodiments, the double-stranded iRNA agent of the invention comprises a monomer of structure:

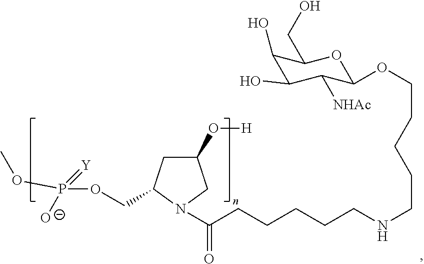

wherein Y is O or S, and n is 1-6.

In certain embodiments, the double-stranded iRNA agent of the invention comprises a monomer of structure:

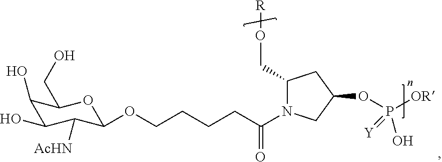

wherein Y is O or S, n is 1-6, R is hydrogen or nucleic acid, and R′ is nucleic acid.

In certain embodiments, the double-stranded iRNA agent of the invention comprises a monomer of structure:

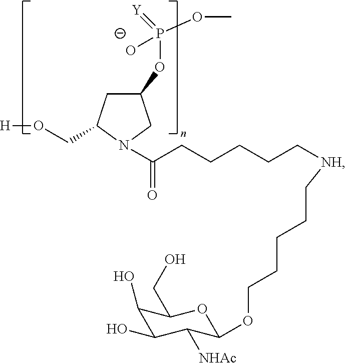

wherein Y is O or S, and n is 1-6.

In certain embodiments, the oligomeric compound described herein, including but not limited to double-stranded iRNA agent of the inventions, comprises a monomer of structure:

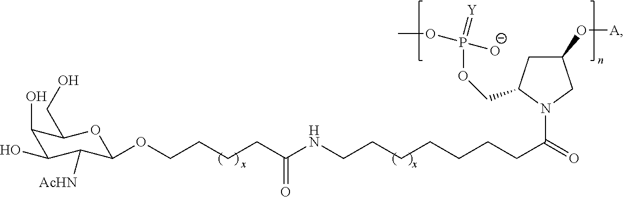

wherein Y is O or S, n is 2-6, x is 1-6, and A is H or a phosphate linkage.

In certain embodiments, the double-stranded iRNA agent of the invention comprises at least 1, 2, 3 or 4 monomer of structure:

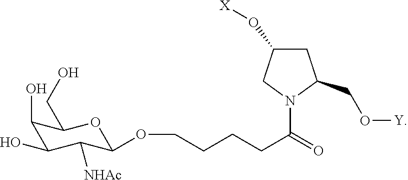

In certain embodiments, the double-stranded iRNA agent of the invention comprises a monomer of structure:

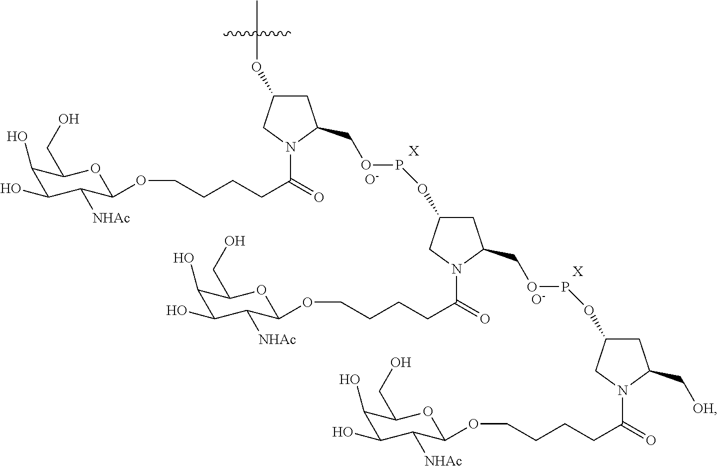

wherein X is O or S.

In certain embodiments, the oligomeric compound described herein, including but not limited to double-stranded iRNA agent of the inventions, comprises a monomer of structure:

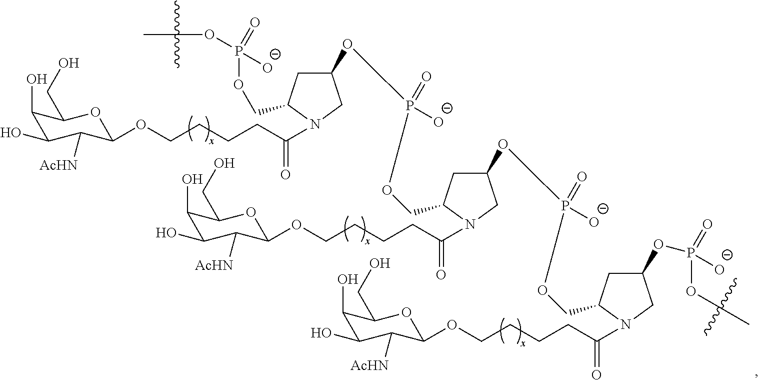

wherein x is 1-12.

In certain embodiments, the double-stranded iRNA agent of the invention comprises a monomer of structure:

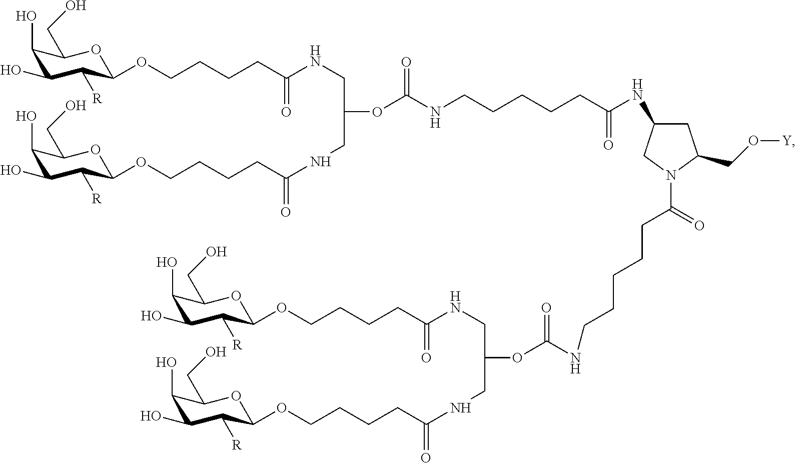

wherein R is OH or NHCOCH₃.

In certain embodiments, the double-stranded iRNA agent of the invention comprises a monomer of structure:

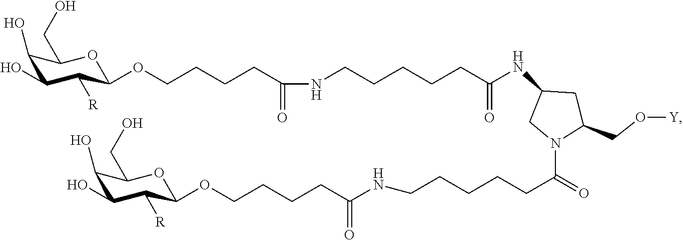

wherein R is OH or NHCOCH₃.

In certain embodiments, the double-stranded iRNA agent of the invention comprises a monomer of structure:

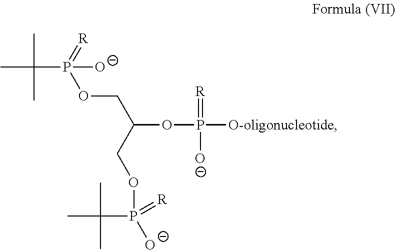

wherein R is O or S.

In certain embodiments, the double-stranded iRNA agent of the invention comprises a monomer of structure:

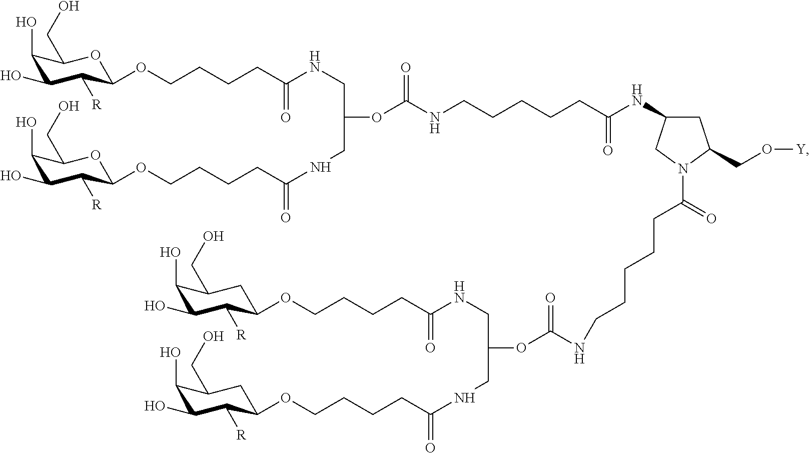

wherein R is OH or NHCOCH₃.

In certain embodiments, the double-stranded iRNA agent of the invention comprises a monomer of structure:

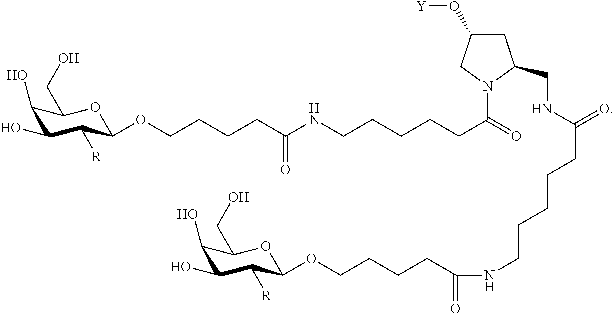

In certain embodiments, the double-stranded iRNA agent of the invention comprises a monomer of structure:

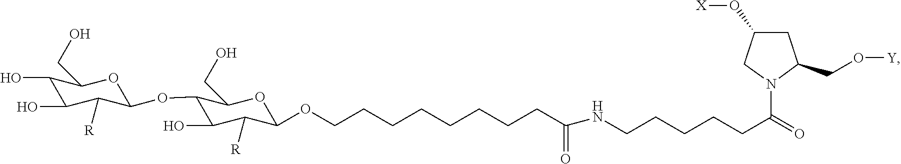

wherein R is OH or NHCOCH₃.

In certain embodiments, the double-stranded iRNA agent of the invention comprises a monomer of structure

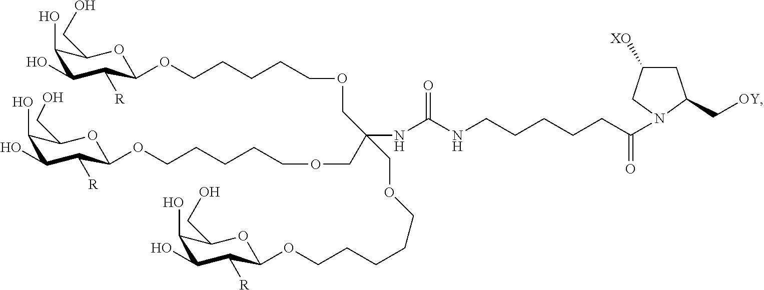

wherein R is OH or NHCOCH₃.

In certain embodiments, the double-stranded iRNA agent of the invention comprises a monomer of structure:

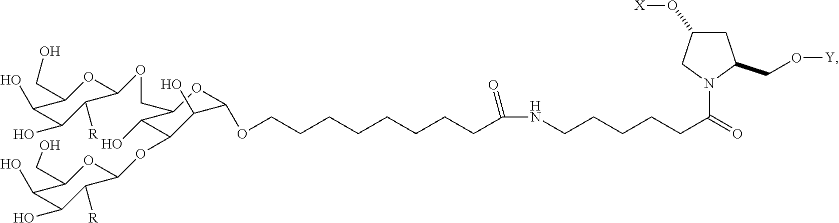

wherein R is OH or NHCOCH₃.

In certain embodiments, the double-stranded iRNA agent of the invention comprises a monomer of structure:

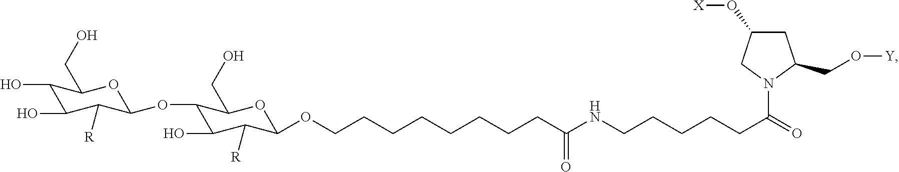

wherein R is OH or NHCOCH₃.

In certain embodiments, the double-stranded iRNA agent of the invention comprises a monomer of structure:

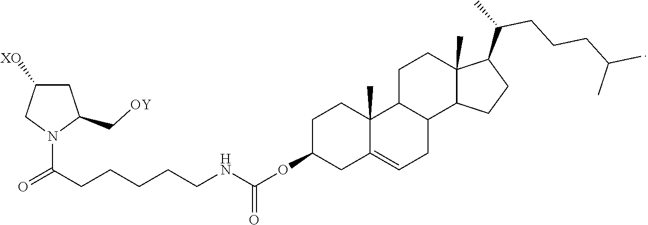

In the above described monomers, X and Y are each independently for each occurrence H, a protecting group, a phosphate group, a phosphodiester group, an activated phosphate group, an activated phosphite group, a phosphoramidite, a solid support, —P(Z′)(Z″)O-nucleoside, —P(Z′)(Z″)O-oligonucleotide, a lipid, a PEG, a steroid, a polymer, a nucleotide, a nucleoside, or an oligonucleotide; and Z′ and Z″ are each independently for each occurrence O or S.

In certain embodiments, the double-stranded iRNA agent of the invention is conjugated with a ligand of structure:

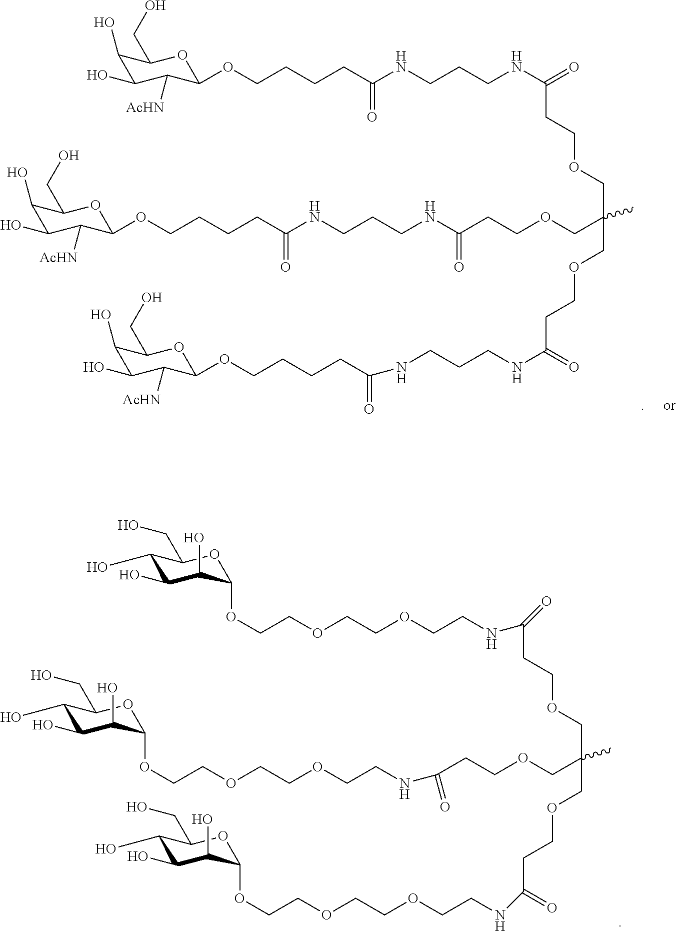

In certain embodiments, the double-stranded iRNA agent of the invention comprises a ligand of structure:

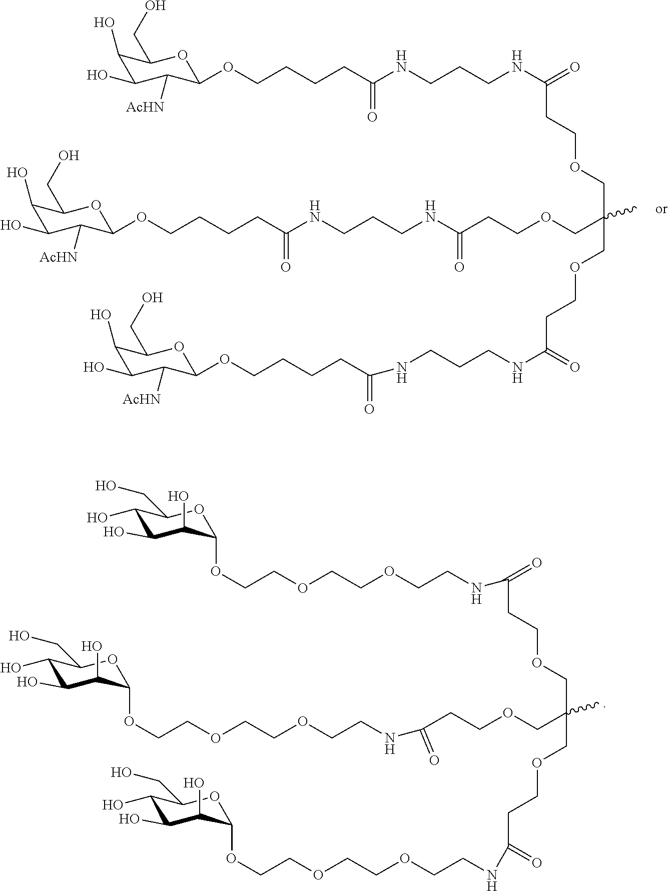

In certain embodiments, the double-stranded iRNA agent of the invention comprises a monomer of structure:

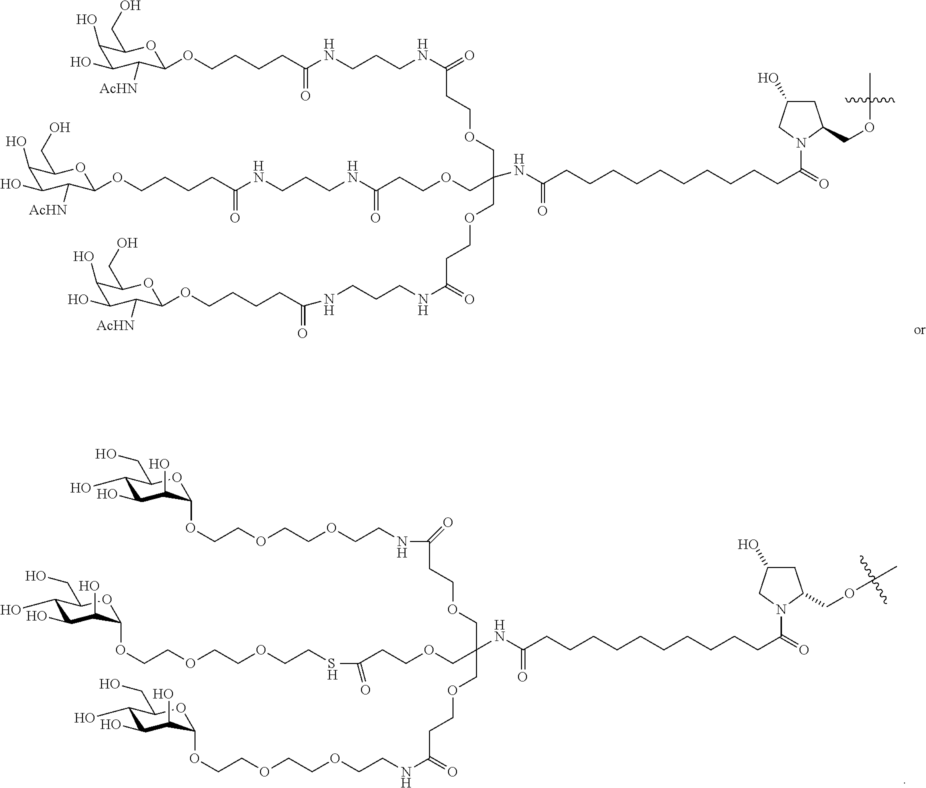

Synthesis of above described ligands and monomers is described, for example, in U.S. Pat. No. 8,106,022, the entire contents of which are incorporated herein by reference in its entirety.

### V. Pharmaceutical Compositions Suitable for Ocular Delivery

The present invention also includes pharmaceutical compositions and formulations which include the iRNAs of the invention. In one embodiment, provided herein are pharmaceutical compositions suitable for ocular delivery containing an iRNA, as described herein, and a pharmaceutically acceptable carrier. The pharmaceutical compositions containing the iRNA are useful for treating an ocular disease or disorder associated with the expression or activity of a TTR gene, e.g., expression of a TTR gene in the eye of a subject. The pharmaceutical compositions of the invention may be administered in dosages sufficient to inhibit expression of a TTR gene in an eye cell.

For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers.

For ocular administration, the siRNAs, double stranded RNA agents of the invention may be applied to the surface of the eye or nearby tissue, e.g., the inside of the eyelid. They can be applied topically, e.g., by spraying, in drops, as an eyewash, or an ointment. Administration can be provided by the subject or by another person, e.g., a health care provider. The medication can be provided in measured doses or in a dispenser which delivers a metered dose. The medication can also be administered to the interior of the eye, and can be introduced by a needle or other delivery device which can introduce it to a selected area or structure.

In one embodiment, the siRNAs, double stranded RNA agents of the invention, are administered to an ocular cell in a pharmaceutical composition by a topical route of administration.

In one embodiment, the pharmaceutical composition suitable for ocular delivery may include an siRNA compound mixed with a topical delivery agent. The topical delivery agent can be a plurality of microscopic vesicles. The microscopic vesicles can be liposomes. In some embodiments the liposomes are cationic liposomes.

In another embodiment, the dsRNA agent is admixed with a topical penetration enhancer. In one embodiment, the topical penetration enhancer is a fatty acid. The fatty acid can be arachidonic acid, oleic acid, lauric acid, caprylic acid, capric acid, myristic acid, palmitic acid, stearic acid, linoleic acid, linolenic acid, dicaprate, tricaprate, monolein, dilaurin, glyceryl 1-monocaprate, 1-dodecylazacycloheptan-2-one, an acylcarnitine, an acylcholine, or a C₁₋₁₀ alkyl ester, monoglyceride, diglyceride or pharmaceutically acceptable salt thereof.

In another embodiment, the topical penetration enhancer is a bile salt. The bile salt can be cholic acid, dehydrocholic acid, deoxycholic acid, glucholic acid, glycholic acid, glycodeoxycholic acid, taurocholic acid, taurodeoxycholic acid, chenodeoxycholic acid, ursodeoxycholic acid, sodium tauro-24,25-dihydro-fusidate, sodium glycodihydrofusidate, polyoxyethylene-9-lauryl ether or a pharmaceutically acceptable salt thereof.

In another embodiment, the penetration enhancer is a chelating agent. The chelating agent can be EDTA, citric acid, a salicylate, a N-acyl derivative of collagen, laureth-9, an N-amino acyl derivative of a beta-diketone or a mixture thereof.

In another embodiment, the penetration enhancer is a surfactant, e.g., an ionic or nonionic surfactant. The surfactant can be sodium lauryl sulfate, polyoxyethylene-9-lauryl ether, polyoxyethylene-20-cetyl ether, a perfluorochemical emulsion or mixture thereof.

In another embodiment, the penetration enhancer can be selected from a group consisting of unsaturated cyclic ureas, 1-alkyl-alkones, 1-alkenylazacyclo-alakanones, steroidal anti-inflammatory agents and mixtures thereof. In yet another embodiment the penetration enhancer can be a glycol, a pyrrol, an azone, or a terpenes.

In one aspect, the invention features a pharmaceutical composition suitable for ocular administration including an siRNA compound and a delivery vehicle. In one embodiment, the siRNA compound is (a) is 19-25 nucleotides long, for example, 21-23 nucleotides, (b) is complementary to an endogenous target RNA, and, optionally, (c) includes at least one 3′ overhang 1-5 nucleotides long.

In one embodiment, the delivery vehicle can deliver an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) to an ocular cell by a topical route of administration. The delivery vehicle can be microscopic vesicles. In one example the microscopic vesicles are liposomes. In some embodiments the liposomes are cationic liposomes. In another example the microscopic vesicles are micelles. In one aspect, the invention features a pharmaceutical composition including an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) in an injectable dosage form.

In one embodiment, the injectable dosage form of the pharmaceutical composition includes sterile aqueous solutions or dispersions and sterile powders. In some embodiments the sterile solution can include a diluent such as water; saline solution; fixed oils, polyethylene glycols, glycerin, or propylene glycol.

The iRNA molecules of the invention can be incorporated into pharmaceutical compositions suitable for ocular administration. Such compositions typically include one or more species of iRNA and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration to an ocular cell. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In certain embodiments, the double-stranded iRNA agents may be delivered directly to the eye by ocular tissue injection such as periocular, conjunctival, subtenon, intracameral, intravitreal, intraocular, anterior or posterior juxtascleral, subretinal, subconjunctival, retrobulbar, or intracanalicular injections; by direct application to the eye using a catheter or other placement device such as a retinal pellet, intraocular insert, suppository or an implant comprising a porous, non-porous, or gelatinous material; by topical ocular drops or ointments; or by a slow release device in the cul-de-sac or implanted adjacent to the sclera (transscleral) or in the sclera (intrascleral) or within the eye. Intracameral injection may be through the cornea into the anterior chamber to allow the agent to reach the trabecular meshwork. Intracanalicular injection may be into the venous collector channels draining Schlemm's canal or into Schlemm's canal.

In one embodiment, the double-stranded iRNA agents may be administered into the eye, for example the vitreous chamber of the eye, by intravitreal injection, such as with pre-filled syringes in ready-to-inject form for use by medical personnel.

For ophthalmic delivery, the double-stranded iRNA agents may be combined with ophthalmologically acceptable preservatives, co-solvents, surfactants, viscosity enhancers, penetration enhancers, buffers, sodium chloride, or water to form an aqueous, sterile ophthalmic suspension or solution. Solution formulations may be prepared by dissolving the conjugate in a physiologically acceptable isotonic aqueous buffer. Further, the solution may include an acceptable surfactant to assist in dissolving the double-stranded iRNA agents. Viscosity building agents, such as hydroxymethyl cellulose, hydroxyethyl cellulose, methylcellulose, polyvinylpyrrolidone, or the like may be added to the pharmaceutical compositions to improve the retention of the double-stranded iRNA agents.

To prepare a sterile ophthalmic ointment formulation, the double-stranded iRNA agents is combined with a preservative in an appropriate vehicle, such as mineral oil, liquid lanolin, or white petrolatum. Sterile ophthalmic gel formulations may be prepared by suspending the double-stranded iRNA agents in a hydrophilic base prepared from the combination of, for example, CARBOPOL®-940 (BF Goodrich, Charlotte, N.C.), or the like, according to methods known in the art.

The pharmaceutical composition can be administered once daily, or the iRNA can be administered as two, three, or more sub-doses at appropriate intervals throughout the day or delivery through a controlled release formulation. In that case, the iRNA contained in each sub-dose must be correspondingly smaller in order to achieve the total daily dosage. The dosage unit can also be compounded for delivery over several days, e.g., using a conventional sustained release formulation which provides sustained release of the iRNA over a several day period. Sustained release formulations are well known in the art and are particularly useful for delivery of agents at a particular site, such as could be used with the agents of the present invention. In this embodiment, the dosage unit contains a corresponding multiple of the daily dose.

In other embodiments, a single dose of the pharmaceutical compositions can be long lasting. In some embodiments of the invention, a single dose of the pharmaceutical compositions of the invention is administered bi-monthly. In other embodiments, a single dose of the pharmaceutical compositions of the invention is administered monthly. In still other embodiments, a single dose of the pharmaceutical compositions of the invention is administered quarterly. In still other embodiments, a single dose of the pharmaceutical compositions of the invention is administered bi-annually.

The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual iRNAs encompassed by the invention can be made using conventional methodologies or on the basis of in vivo testing using an appropriate animal model, as described elsewhere herein.

### VI. Methods for Inhibiting TTR Expression in an Ocular Cell

The present invention also provides methods of inhibiting expression of a transthyretin (TTR) in an ocular cell. The methods include contacting an ocular cell with an RNAi agent, e.g., double stranded RNAi agent, in an amount effective to inhibit expression of TTR in the ocular cell, thereby inhibiting expression of TTR in the ocular cell.

Contacting of an ocular cell with an RNAi agent, e.g., a double stranded RNAi agent, may be done in vitro or in vivo. Contacting an ocular cell in vivo with the RNAi agent includes contacting an ocular cell or group of ocular cells within a subject, e.g., a human subject, with the RNAi agent. Combinations of in vitro and in vivo methods of contacting an ocular cell or group of ocular cells are also possible. Contacting an ocular cell or a group of ocular cells may be direct or indirect, as discussed above. Furthermore, contacting an ocular cell or a group of ocular cells may be accomplished via one or more lipophilic moieties conjugated to one or more internal positions on at least one strand of a dsRNA agent, or conjugated to one or more positions on at least one strand of the double stranded region of a dsRNA agent, and/or via a targeting ligand, including any ligand described herein or known in the art. In one embodiment, the targeting ligand is a ligand that directs the RNAi agent to a site of interest, e.g., the ocular cells of a subject.

The term “inhibiting,” as used herein, is used interchangeably with “reducing,” “silencing,” “downregulating”, “suppressing”, and other similar terms, and includes any level of inhibition. Preferably inhibiting includes a statistically significant or clinically significant inhibition.

The phrase “inhibiting expression of a TTR” is intended to refer to inhibition of expression of any TTR gene (such as, e.g., a mouse TTR gene, a rat TTR gene, a monkey TTR gene, or a human TTR gene) as well as variants or mutants of a TTR gene. Thus, the TTR gene may be a wild-type TTR gene, a mutant TTR gene (such as a mutant TTR gene giving rise to amyloid deposition), or a transgenic TTR gene in the context of a genetically manipulated ocular cell, group of ocular cells, or organism.

“Inhibiting expression of a TTR gene” includes any level of inhibition of a TTR gene, e.g., at least partial suppression of the expression of a TTR gene. The expression of the TTR gene may be assessed based on the level, or the change in the level, of any variable associated with TTR gene expression, e.g., TTR mRNA level, TTR protein level, or the number or extent of amyloid deposits. This level may be assessed in an individual ocular cell or in a group of ocular cells, including, for example, a sample derived from a subject.

Inhibition may be assessed by a decrease in an absolute or relative level of one or more variables that are associated with TTR expression in the eye compared with a control level. The control level may be any type of control level that is utilized in the art, e.g., a pre-dose baseline level, or a level determined from a similar subject, ocular cell, or sample that is untreated or treated with a control (such as, e.g., buffer only control or inactive agent control).

In some embodiments of the methods of the invention, expression of a TTR gene in an ocular cell is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%%, or to below the level of detection of the assay. In some embodiments, the inhibition of expression of a TTR gene results in normalization of the level of the TTR gene such that the difference between the level before treatment and a normal control level is reduced by at least 30%, 35%, 40%, 45%, 50%, 55%, 60%, 65%, 70%, 75%, 80%, 85%, 90%, or 95%. In some embodiments, the inhibition is a clinically relevant inhibition.

Inhibition of the expression of a TTR gene may be manifested by a reduction of the amount of mRNA expressed by a first cell or group of ocular cells (such cells may be present, for example, in a sample derived from a subject) in which a TTR gene is transcribed and which has or have been treated (e.g., by contacting the ocular cell or ocular cells with an RNAi agent of the invention, or by administering an RNAi agent of the invention to a subject in which the cells are or were present) such that the expression of a TTR gene is inhibited, as compared to a second cell or group of ocular cells substantially identical to the first cell or group of ocular cells but which has not or have not been so treated (control cell(s)). In preferred embodiments, the inhibition is assessed by expressing the level of mRNA in treated cells as a percentage of the level of mRNA in control cells, using the following formula:

$$\frac{( m R N A in control cells ) - ( m R N A in treated cells )}{( m R N A in control cells )} 100 \%$$

Alternatively, inhibition of the expression of a TTR gene may be assessed in terms of a reduction of a parameter that is functionally linked to TTR gene expression, e.g., TTR protein expression, retinol binding protein level, vitamin A level, or presence of amyloid deposits comprising TTR. TTR gene silencing may be determined in any ocular cell expressing TTR, either constitutively or by genomic engineering, and by any assay known in the art.

Inhibition of the expression of a TTR protein may be manifested by a reduction in the level of the TTR protein that is expressed by an ocular cell or group of ocular cells (e.g., the level of protein expressed in a sample derived from a subject). As explained above for the assessment of mRNA suppression, the inhibition of protein expression levels in a treated ocular cell or group of ocular cells may similarly be expressed as a percentage of the level of protein in a control ocular cell or group of ocular cells.

A control ocular cell or group of ocular cells that may be used to assess the inhibition of the expression of a TTR gene includes an ocular cell or group of ocular cells that has not yet been contacted with an RNAi agent of the invention. For example, the control ocular cell or group of ocular cells may be derived from an individual subject (e.g., a human or animal subject) prior to treatment of the subject with an RNAi agent.

The level of TTR mRNA that is expressed by an ocular cell or group of ocular cells, or the level of circulating TTR mRNA, may be determined using any method known in the art for assessing mRNA expression. In one embodiment, the level of expression of TTR in a sample is determined by detecting a transcribed polynucleotide, or portion thereof, e.g., mRNA of the TTR gene. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilizing ribonucleic acid hybridization include nuclear run-on assays, RT-PCR, RNase protection assays (Melton et al., Nuc. Acids Res. 12:7035), Northern blotting, in situ hybridization, and microarray analysis. Circulating TTR mRNA may be detected using methods the described in PCT/US2012/043584, the entire contents of which are hereby incorporated herein by reference.

In one embodiment, the level of expression of TTR is determined using a nucleic acid probe. The term “probe”, as used herein, refers to any molecule that is capable of selectively binding to a specific TTR. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or Northern analyses, polymerase chain reaction (PCR) analyses and probe arrays. One method for the determination of mRNA levels involves contacting the isolated mRNA with a nucleic acid molecule (probe) that can hybridize to TTR mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array. A skilled artisan can readily adapt known mRNA detection methods for use in determining the level of TTR mRNA.

An alternative method for determining the level of expression of TTR in a sample involves the process of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, the level of expression of TTR is determined by quantitative fluorogenic RT-PCR (i.e., the TaqMan™ System).

The expression levels of TTR mRNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The determination of TTR expression level may also comprise using nucleic acid probes in solution.

In preferred embodiments, the level of mRNA expression is assessed using branched DNA (bDNA) assays or real time PCR (qPCR).

The level of TTR protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, Western blotting, radioimmunoassay (RIA), enzyme-linked immunosorbent assays (ELISAs), immunofluorescent assays, electrochemiluminescence assays, and the like.

In some embodiments, the efficacy of the methods of the invention can be monitored by detecting or monitoring a reduction in an amyloid TTR deposit. Reducing an amyloid TTR deposit, as used herein, includes any decrease in the size, number, or severity of TTR deposits, or to a prevention or reduction in the formation of TTR deposits, within the eye or area of an eye of a subject, as may be assessed in vitro or in vivo using any method known in the art. For example, some methods of assessing amyloid deposits are described in Gertz, M. A. & Rajukumar, S. V. (Editors) (2010), Amyloidosis: Diagnosis and Treatment, New York: Humana Press. Methods of assessing amyloid deposits may include biochemical analyses, as well as visual or computerized assessment of amyloid deposits, as made visible, e.g., using immunohistochemical staining, fluorescent labeling, light microscopy, electron microscopy, fluorescence microscopy, or other types of microscopy. Invasive or noninvasive imaging modalities, including, e.g., CT, PET, or NMR/MRI imaging may be employed to assess amyloid deposits.

The term “sample” as used herein refers to a collection of similar ocular fluids, ocular cells, or ocular tissues isolated from a subject, as well as ocular fluids, ocular cells, or ocular tissues present within a subject. Examples of biological fluids include ocular fluids, and the like. Tissue samples may include samples from tissues, organs or localized regions. For example, samples may be derived from particular organs, parts of organs, or fluids or cells within those organs. In certain embodiments, samples may be derived from the retina or parts of the retina (e.g., retinal pigment epithelium and/or ciliary epithelium). In preferred embodiments, a “sample derived from a subject” refers to retinal tissue derived from the subject.

In some embodiments of the methods of the invention, the RNAi agent is administered to a subject such that the RNAi agent is delivered to a specific site within the subject. The inhibition of expression of TTR may be assessed using measurements of the level or change in the level of TTR mRNA or TTR protein in a sample derived from fluid or tissue from the specific site within the subject. In one embodiment, the site is the retina. In another embodiment, the site is the liver. The site may also be a subsection or subgroup of cells from any one of the aforementioned sites (e.g., hepatocytes or retinal pigment epithelium). The site may also include cells that express a particular type of receptor (e.g., hepatocytes that express the asialogycloprotein receptor).

### VII. Methods for Treating or Preventing a TTR-Associated Ocular Disease

The present invention also provides methods for treating or preventing a TTR-associated ocular disease in a subject. The methods include intraocularly administering to the subject a therapeutically effective amount or prophylactically effective amount of an RNAi agent of the invention.

As used herein, a “subject” is an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, a horse, and a whale), or a bird (e.g., a duck or a goose). A subject may include a transgenic organism.

In an embodiment, the subject is a human, such as a human being treated or assessed for an ocular disease, disorder or condition that would benefit from reduction in TTR gene expression in an ocular cell; a human at risk for a disease, disorder or condition that would benefit from reduction in TTR gene expression in an ocular cell; a human having an ocular disease, disorder or condition that would benefit from reduction in TTR gene expression in an ocular cell; and/or human being treated for a disease, disorder or condition that would benefit from reduction in TTR gene expression in an ocular cell, as described herein.

In some embodiments, the subject is suffering from a TTR-associated ocular disease, e.g., a subject with a TTR mutation that has been treated or is being treated for other manifestations of the TTR mutation, e.g., a subject having a TTR-associated disease, such as, senile systemic amyloidosis (SSA); systemic familial amyloidosis; familial amyloidotic polyneuropathy (FAP); familial amyloidotic cardiomyopathy (FAC); and leptomeningeal amyloidosis, also known as leptomeningeal or meningocerebrovascular amyloidosis, central nervous system (CNS) amyloidosis, or amyloidosis VII form.

In one embodiment, the RNAi agents of the invention are administered to subjects suffering from familial amyloidotic cardiomyopathy (FAC). In another embodiment, the RNAi agents of the invention are administered to subjects suffering from FAC with a mixed phenotype, i.e., a subject having both cardiac and neurological impairments. In yet another embodiment, the RNAi agents of the invention are administered to subjects suffering from FAP with a mixed phenotype, i.e., a subject having both neurological and cardiac impairments. In one embodiment, the RNAi agents of the invention are administered to subjects suffering from FAP that has been treated with an orthotopic liver transplantation (OLT). In another embodiment, the RNAi agents of the invention are administered to subjects suffering from senile systemic amyloidosis (SSA). In other embodiments of the methods of the invention, RNAi agents of the invention are administered to subjects suffering from familial amyloidotic cardiomyopathy (FAC) and senile systemic amyloidosis (SSA). Normal-sequence TTR causes cardiac amyloidosis in people who are elderly and is termed senile systemic amyloidosis (SSA) (also called senile cardiac amyloidosis (SCA) or cardiac amyloidosis). SSA often is accompanied by microscopic deposits in many other organs. TTR mutations accelerate the process of TTR amyloid formation and are the most important risk factor for the development of clinically significant TTR amyloidosis (also called ATTR (amyloidosis-transthyretin type)). More than 85 amyloidogenic TTR variants are known to cause systemic familial amyloidosis.

In some embodiments of the methods of the invention, RNAi agents of the invention are administered to subjects suffering from transthyretin (TTR)-related familial amyloidotic polyneuropathy (FAP).

In other embodiments, the subject is a subject at risk for developing a TTR-associated ocular disease, e.g., a subject with a TTR gene mutation that is associated with the development of a TTR-associated ocular disease (e.g., before the onset of signs or symptoms suggesting the development of TTR ocular amyloidosis), a subject with a family history of TTR-associated ocular disease (e.g., before the onset of signs or symptoms suggesting the development of TTR ocular amyloidosis), or a subject who has signs or symptoms suggesting the development of TTR ocular amyloidosis.

A “TTR-associated ocular disease” includes any type of TTR amyloidosis (ATTR) wherein TTR plays a role in the formation of abnormal extracellular aggregates or amyloid deposits in the eye. TTR-associated ocular diseases or disorders include, but are not limited to, TTR-associated glaucoma, TTR-associated vitreous opacities, TTR-associated retinal abnormalities, TTR-associated retinal amyloid deposit, TTR-associated retinal angiopathy, TTR-associated iris amyloid deposit, TTR-associated scalloped iris, and TTR-associated amyloid deposits on lens.

In one aspect, the RNAi agents of the invention are intraocularly administered to subjects suffering from a TTR-associated ocular disease, such as TTR-associated glaucoma, TTR-associated vitreous opacities, TTR-associated retinal abnormalities, TTR-associated retinal amyloid deposit, TTR-associated retinal angiopathy, TTR-associated iris amyloid deposit, TTR-associated scalloped iris, and TTR-associated amyloid deposits on lens.

Intraocular administration may be via periocular, conjunctival, subtenon, intracameral, intravitreal, intraocular, anterior or posterior juxtascleral, subretinal, subconjunctival, retrobulbar, or intracanalicular injection.

In some embodiments, the RNAi agent is administered to a subject in an amount effective to inhibit TTR expression in an ocular cell, such as an RPE and/or CE cell within the subject. The amount effective to inhibit TTR expression in an ocular cell within a subject may be assessed using methods discussed above, including methods that involve assessment of the inhibition of TTR mRNA, TTR protein, or related variables, such as amyloid deposits.

In some embodiments, the RNAi agent is administered to a subject in a therapeutically or prophylactically effective amount.

“Therapeutically effective amount,” as used herein, is intended to include the amount of an RNAi agent that, when administered to a patient for treating a TTR-associated ocular disease, is sufficient to effect treatment of the disease (e.g., by diminishing, ameliorating or maintaining the existing disease or one or more symptoms of disease). The “therapeutically effective amount” may vary depending on the RNAi agent, how the agent is administered, the disease and its severity and the history, age, weight, family history, genetic makeup, stage of pathological processes mediated by TTR expression, the types of preceding or concomitant treatments, if any, and other individual characteristics of the patient to be treated.

“Prophylactically effective amount,” as used herein, is intended to include the amount of an RNAi agent that, when administered to a subject who does not yet experience or display symptoms of a TTR-associated disease, but who may be predisposed to the disease, is sufficient to prevent or ameliorate the disease or one or more symptoms of the disease. Symptoms that may be ameliorated include decreased visual acuity, decreased night vision, decreased peripheral vision, attenuation of the retinal vessels, tortuousness of retinal vessels, corneal sensitivity, retinal vein occlusion, and corneal lattice dystrophy, and other ophthalmoscopic symptoms or conditions associated with TTR-associated ocular disorders. In one embodiment, the RNAi agents are administered to subjects suffering from a vitreous amyloidosis. In one embodiment, the RNAi agents are administered to subjects suffering from an ocular amyloidosis in the ciliary epithelium (CE). In another embodiment, the RNAi agents are administered to subjects suffering from an ocular amyloidosis in the retinal pigment epithelium (RPE). Ameliorating the disease includes slowing the course of the disease or reducing the severity of later-developing disease. The “prophylactically effective amount” may vary depending on the RNAi agent, how the agent is administered, the degree of risk of disease, and the history, age, weight, family history, genetic makeup, the types of preceding or concomitant treatments, if any, and other individual characteristics of the patient to be treated.

A “therapeutically-effective amount” or “prophylactically effective amount” also includes an amount of an RNAi agent that produces some desired local or systemic effect at a reasonable benefit/risk ratio applicable to any treatment. RNAi agents employed in the methods of the present invention may be administered in a sufficient amount to produce a reasonable benefit/risk ratio applicable to such treatment.

As used herein, the phrases “therapeutically effective amount” and “prophylactically effective amount” also include an amount that provides a benefit in the treatment, prevention, or management of pathological processes or symptom(s) of pathological processes mediated by TTR expression. Symptoms of ocular TTR amyloidosis include decreased visual acuity, decreased night vision, decreased peripheral vision, attenuation of the retinal vessels, tortuousness of retinal vessels, corneal sensitivity, retinal vein occlusion, and corneal lattice dystrophy, and other ophthalmoscopic symptoms or conditions associated with TTR-associated ocular disorders.

The dose of an RNAi agent that is administered to a subject may be tailored to balance the risks and benefits of a particular dose, for example, to achieve a desired level of TTR gene suppression (as assessed, e.g., based on TTR mRNA suppression, TTR protein expression, or a reduction in an amyloid deposit, as defined above) or a desired therapeutic or prophylactic effect, while at the same time avoiding undesirable side effects.

In some embodiments, the agents are administered to the subject intravitreally. In some embodiments, a dose of the RNAi agent for subcutaneous administration is contained in a volume of less than or equal to one ml of, e.g., a pharmaceutically acceptable carrier.

In some embodiments, the administration is via a depot injection. A depot injection may release the RNAi agent in a consistent way over a prolonged time period. Thus, a depot injection may reduce the frequency of dosing needed to obtain a desired effect, e.g., a desired inhibition of TTR, or a therapeutic or prophylactic effect.

In some embodiments, the administration is via a pump. The pump may be an external pump or a surgically implanted pump.

In some embodiments, the RNAi agent is administered to a subject in an amount effective to inhibit TTR expression in an ocular cell within the subject. The amount effective to inhibit TTR expression in an ocular cell within a subject may be assessed using methods discussed above, including methods that involve assessment of the inhibition of TTR mRNA, TTR protein, or related variables, such as amyloid deposits.

The methods of the present invention may also improve the prognosis of the subject being treated. For example, the methods of the invention may provide to the subject a reduction in probability of a clinical worsening event during the treatment period.

The dose of an RNAi agent that is administered to a subject may be tailored to balance the risks and benefits of a particular dose, for example, to achieve a desired level of TTR gene suppression (as assessed, e.g., based on TTR mRNA suppression, TTR protein expression, or a reduction in an amyloid deposit, as defined above) or a desired therapeutic or prophylactic effect, while at the same time avoiding undesirable side effects.

In one embodiment, an iRNA agent of the invention is administered to a subject as a weight-based dose. A “weight-based dose” (e.g., a dose in mg/kg) is a dose of the iRNA agent that will change depending on the subject's weight. In another embodiment, an iRNA agent is administered to a subject as a fixed dose. A “fixed dose” (e.g., a dose in mg) means that one dose of an iRNA agent is used for all subjects regardless of any specific subject-related factors, such as weight. In one particular embodiment, a fixed dose of an iRNA agent of the invention is based on a predetermined weight or age.

Subjects can be administered a therapeutic amount of iRNA, such as about 0.01 mg/kg to about 50 mg/kg dsRNA. Values and ranges intermediate to the recited values are also intended to be part of this invention.

In some embodiments, the RNAi agent is administered as a fixed dose of between about 0.01 mg to about 1 mg. In certain embodiments, the subject is administered a fixed dose of about 0.001 mg to about 1 mg of the double stranded RNAi agent. In certain embodiments, the subject is administered a fixed dose of about 0.001 mg to about 0.1 mg of the double stranded RNAi agent. In certain embodiments, the agent is delivered about once per month. In certain embodiments, the agent is administered once per quarter (i.e., about once every three months). In certain embodiments, the agent is administered semi-annually (i.e., about once every six months).

In certain embodiments, the RNAi agent is administered to a subject as a fixed dose of about 0.001, 0.003, 0.01, 0.02, 0.03, 0.04, 0.05, 0.06, 0.07, 0.08, 0.09, 0.1, 0.1, 0.2, 0.3, 0.4, 0.5, 0.6, 0.7, 0.8, 0.9, or about 1 mg once every month, once every two months, once every three months (i.e., once a quarter), once every four months, once every five months, once every six month (i.e., bi-annually), or once a year.

In some embodiments, the RNAi agent is administered in two or more doses. If desired to facilitate repeated or frequent infusions, implantation of a reservoir may be advisable. In some embodiments, the number or amount of subsequent doses is dependent on the achievement of a desired effect, e.g., the suppression of a TTR gene, or the achievement of a therapeutic or prophylactic effect, e.g., reducing an amyloid deposit or reducing a symptom of a TTR-associated ocular disease.

In some embodiments, the RNAi agent is administered with other therapeutic agents or other therapeutic regimens. For example, other agents or other therapeutic regimens suitable for treating a TTR-associated disease may include a liver transplant, which can reduce mutant TTR levels in the body; Patisiran (ONPATTRO™); Tafamidis (Vyndagel), which kinetically stabilizes the TTR tetramer preventing tetramer dissociation required for TTR amyloidogenesis; diuretics, which may be employed, for example, to reduce edema in TTR amyloidosis with cardiac involvement.

In one embodiment, a subject is administered an initial dose and one or more maintenance doses of an RNAi agent. The maintenance dose or doses can be the same or lower than the initial dose, e.g., one-half of the initial dose. Further, the treatment regimen may last for a period of time which will vary depending upon the nature of the particular disease, its severity and the overall condition of the patient. Following treatment, the patient can be monitored for changes in his/her condition. The dosage of the RNAi agent may either be increased in the event the patient does not respond significantly to current dosage levels, or the dose may be decreased if an alleviation of the symptoms of the disease state is observed, if the disease state has been ablated, or if undesired side-effects are observed.

### VIII. Kits of the Invention

The present invention also provides kits for performing any of the methods of the invention. Such kits include one or more RNAi agent(s) and instructions for use, e.g., instructions for inhibiting expression of a TTR in an ocular cell by contacting the ocular cell with the RNAi agent(s) in an amount effective to inhibit expression of the TTR in the ocular cell. The kits may optionally further comprise means for contacting the ocular cell with the RNAi agent (e.g., an injection device or an infusion pump), or means for measuring the inhibition of TTR (e.g., means for measuring the inhibition of TTR mRNA or TTR protein). Such means for measuring the inhibition of TTR may comprise a means for obtaining a sample from a subject. The kits of the invention may optionally further comprise means for administering the RNAi agent(s) to a subject or means for determining the therapeutically effective or prophylactically effective amount.

The RNAi agent may be provided in any convenient form, such as a solution in sterile water for injection.

The invention is further illustrated by the following examples, which should not be construed as further limiting. The contents of all references, pending patent applications and published patents, cited throughout this application are hereby expressly incorporated by reference.

### EXAMPLES

The following experiments demonstrated the beneficial effects of conjugating one or more lipophilic moieties to one or more internal positions, or within the double stranded region, on at least one strand of a double stranded RNAi agent, on the silencing activity of RNAi agents that target TTR in an ocular cell.

### Example 1. Inhibition of Ocular TTR Expression in Rats

DsRNA agents optimized for ocular cell deliver, e.g., a dsRNA agent comprising a ligand that mediates delivery to an ocular cell (OC conjugated; AD-67175), or partially modified, e.g., not all of the nucleotides of the sense strand and antisense strand comprise a nucleotide modification (AD-23043), or optimized for hepatic delivery, e.g., a dsRNA agent comprising a ligand that targets delivery to the dsRNA agent to a hepatic cell (ESC; AD-65808), were intravitreally administered to rats in order to determine the efficacy of ocular TTR inhibition by these agents.

The modified sense and antisense strand nucleotide sequences of these agents are provided in the Table below.

| | | | | | SEQ |
|---|---|---|---|---|---|
| | | | | | ID |
| Duplex | Oligo | Target | Strand | Modified Sequence (5′ to 3′) | NO: |
| AD- | A-51593.1 | TTR_rodent | sense | cAGuGuucuuGcucuAuAAdTdTsL10 | 53 |
| 23043.1 | A-32756.1 | TTR | antissense | UuAuAGAGcAAGAAcACUGdTdT | 54 |
| AD- | A-130279.1 | mTTRsc | sense | AfsasCfaGfuGfuUfCfUfuGfcUfcUfaUfasAfdTdTL | 55 |
| 65808.1 | | | | 10 | |
| | A-117800.10 | m/r TTR | antissense | usUfsaUfaGfaGfcAfagaAfcAfcUfgUfususu | 56 |
| AD- | A-130279.1 | mTTRsc | sense | AfsasCfaGfuGfuUfCfUfuGfcUfcUfaUfasAfdTdTL | 55 |
| 67175.1 | | | | 10 | |
| | A-130284.1 | mTTRsc | antissense | VPusUfsaUfaGfaGfcAfagaAfcAfcUfgUfususu | 57 |

One eye of each rat was administered a single 50 μg dose of a dsRNA agent that was optimized for ocular cell deliver (OC conjugated), or a single 50 μg dose of a dsRNA agent that was partially modified, or a single 50 μg dose of a dsRNA agent that was optimized for hepatic delivery (ESC), or PBS (as a control) via intravitreal injection.

Efficacy of treatment was evaluated by measurement of TTR mRNA levels in the eye at 14 days post-dose. Briefly, the eyes were harvested and the vitreous fluid was removed. Tissue lysates were prepared using a protocol similar to the protocol described in Foster D. J., et al. (2018) Mol. Ther. 26:708. Ocular mRNA levels were assayed using a quantitative bDNA assay (Panomics). The mRNA level was calculated for each group and normalized to untreated tissue sample to give relative TTR mRNA as a % message remaining compared to the untreated tissue.

As shown in FIG. 1, the OC conjugated agent significantly reduced the mRNA level of TTR in rat ocular tissues as compared to either the agent that was partially modified or that agent having ESC modifications.

It has previously been shown that TTR protein is primarily produced in the eye in retinal pigmented epithelia cells (RPEs) and ciliary epithelial cells (CEs) (see, e.g., Hara et al. (2010) Arch Ophthalmal 128: 206, and Kawaji et al. Exp Eye Res, 81, 2005, 306).

Accordingly, to demonstrate that the OC conjugated agent specifically inhibited TTR expression in RPEs and CEs, posterior tissues (retina, retinal pigment epithelium, choroid, and sclera) and anterior tissues (ciliary epithelium, cornea, lens, iris, and aqueous humor) of rat eyes that were administered a single 50 g intravitreal dose of the OC conjugated dsRNA agent (AD-67175) or an unconjugated dsRNA agent (unconjugated; AD-77745) were isolated and TTR mRNA levels in these tissues were determined as described above.

The modified sense and antisense strand nucleotide sequences of these agents are provided in the Table below.

| | | | | | SEQ |
|---|---|---|---|---|---|
| | | | | | ID |
| Duplex | Oligo | Target | Strand | Modified Sequence (5′ to 3′) | NO: |
| AD- | A-130279.1 | mTTRsc | sense | AfsasCfaGfuGfuUfCfUfuGfcUfcUfaUfasAfdT | 55 |
| 67175.1 | | | | dTL10 | |
| | A-130284.1 | mTTRsc | antissense | VPusUfsaUfaGfaGfcAfagaAfcAfcUfgUfususu | 57 |
| AD- | A-147399.1 | m/rTTR | sense | AfsasCfaGfuGfuUfCfUfuGfcUfcUfaUfsasAf | 58 |
| 77745.1 | A-130284.2 | mTTRsc | antissense | VPusUfsaUfaGfaGfcAfagaAfcAfcUfgUfususu | 57 |

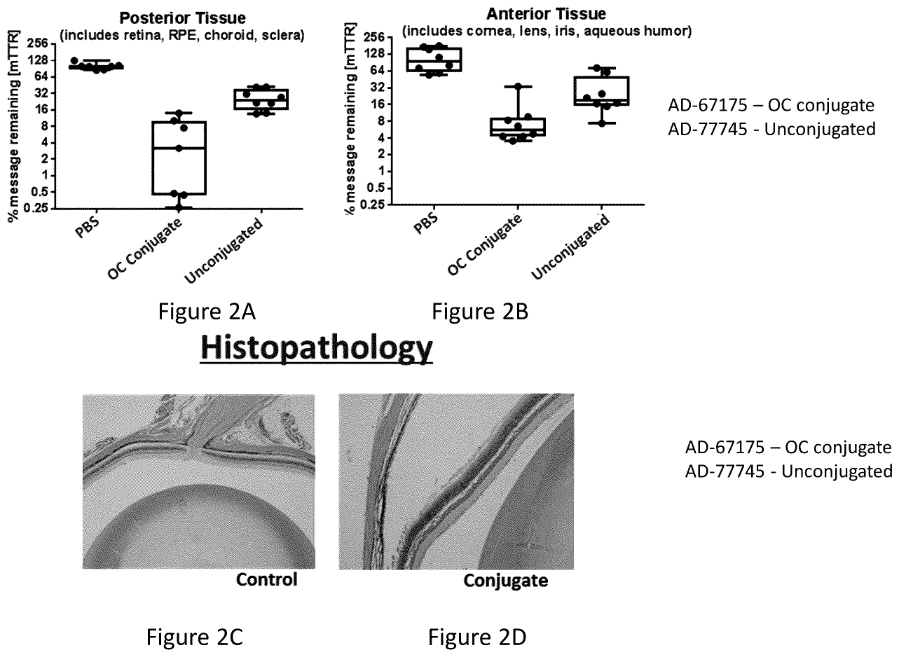

As shown in FIGS. 2A and 2B, the OC conjugated agent significantly reduced the expression of TTR in both the posterior tissues and the anterior tissues.

Furthermore, and as illustrated in FIG. 2D, histopathological analysis of the posterior and anterior eye tissues demonstrated that intravitreal administration of the OC conjugated agent was not associated with any treatment related pathological micro-findings.

### Example 2. Inhibition of Human Ocular TTR Expression in Transgenic Mice

To assess the specificity of RNAi agents optimized for ocular delivery to inhibit human TTR expression, an OC conjugated RNAi agent for ocular delivery, AD-70191, was administered to transgenic mice that express human TTR with the V30M mutation (see Santos, S D., Fernaandes, R., and Saraiva, M J. (2010) Neurobiology of Aging, 31, 280-289). The V30M mutation is known to cause familial amyloid polyneuropathy type I in humans. See, e.g., Lobato, L. (2003) J Nephrol., 16(3):438-42.

A single 2.5 μg or 7.5 μg dose of AD-70191 was administered intravitreally to transgenic mice at Day 0. At Day 7, ocular tissues were harvested and mRNA levels of TTR were determined as described above. For comparison, mRNA levels of mouse TTR, mouse cone-rod homeobox and, mouse rhodopsin were also determined.

The modified sense and antisense strand nucleotide sequences of AD-70191 are provided in the Table below.

| | | | | | SEQ |
|---|---|---|---|---|---|
| | | | | | ID |
| Duplex | Oligo | Target | Strand | Modified Sequence (5′ to 3′) | NO: |
| AD-70191.1 | A-139575.1 | hTTRsc02 | sense | usgsggauUfuCfAfUfguaaccaagsadTdTL10 | 59 |
| | A-131902.1 | h/c TTR | antissense | VPusCfsuugGfuuAfcaugAfaAfucccasusc | 17 |

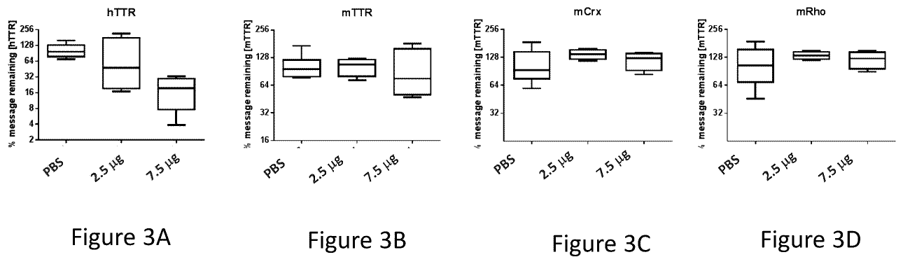

As illustrated in FIG. 3A, a single 2.5 μg or 7.5 μg dose of AD-70191 significantly reduced the expression of hTTR in the transgenic mice. The 7.5 μg dose was more efficacious than the 2.5 μg dose. In contrast, as illustrated in FIG. 3B-3D, the mRNA levels of mouse TTR, cone-rod homeobox and rhodopsin were not decreased, indicating that AD-AD-70191 was specifically targeting human TTR.

### Example 3. Inhibition of Ocular TTR Expression in Non-Human Primates

The efficacy of dsRNA agents variously modified for ocular delivery, AD-291845, AD-70500, AD-290674, AD-290676, and AD-290675, was also assessed in the eyes of non-human primates. Male Cynomolgous monkeys (n=3) were intravitreally administered a single 1 mg or 3 mg dose of AD-291845, AD-70500, AD-290674, AD-290676, or AD-290675 on Day 0. Eyes were collected 31 days post administration. Tissues (RPE and CE) were dissected and lysates were prepared from the tissues. TTR mRNA levels were determined as described above.

The modified and unmodified sense and antisense strand nucleotide sequences of these agents are provided in the Table below.

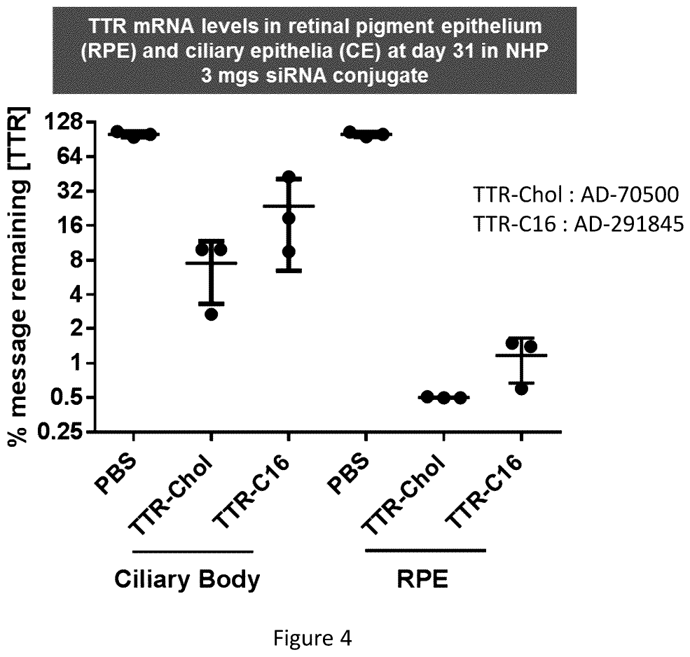

As illustrated in FIG. 4, a single 3 mg dose of AD-291845 or AD-70500 significantly reduced the mRNA levels of TTR in both ciliary epithelium (CE) and retinal pigment epithelium (RPE).

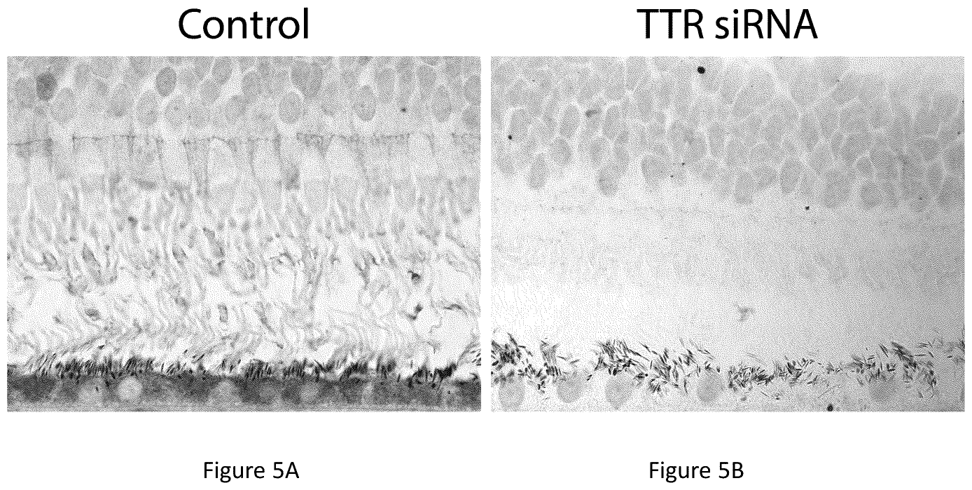

Furthermore, as illustrated in FIG. 5B, immunohistochemical (IHC) analysis showed that the single 3 mg dose administration of the AD-29185 significantly reduced TTR protein at Day 31.

Ophthalmoscopic examination of the injected eyes on Days −7, 3, 8, and 30 (Table 1) and histopathological examination on Day 31 (Table 2) revealed no significant treatment related pathological findings associated with intravitreal administration of AD-29185.

These data demonstrate that AD-291845 specifically and efficaciously reduces TTR mRNA and protein in ocular tissues.

| | | | | | SEQ | |
|---|---|---|---|---|---|---|
| | | | | | ID | |
| Duplex | Oligo | Target | Strand | Modified Sequence (5′ to 3′) | NO: | Ligand |
| AD- | A-140611 | hTTRsc02 | sense | usgsggauUfuCfAfUfguaaccaagaL10 | 60 | 3′-cholesterol |
| 70500.1 | A-131902 | h/c TTR | antissense | VPusCfsuugGfuuAfcaugAfaAfuccca | 17 | |
| | | | | susc | | |
| AD- | A-515644 | h/c TTR | sense | usgsggauUfuCfAfUfguaaccaagaL57 | 61 | 3′-C18 |
| 290674.1 | A-131902 | h/c TTR | antissense | VPusCfsuugGfuuAfcaugAfaAfuccca | 17 | |
| | | | | susc | | |
| AD- | A-515644 | h/c TTR | sense | usgsggauUfuCfAfUfguaaccaagaL57 | 61 | 3′-C18 |
| 290675.1 | A-265470 | TTR | antissense | VPuCfuugGfuuAfcaugAfaAfucccasu | 62 | |
| | | | | sc | | |
| AD- | | | | | 60 | 3′-cholesterol |
| 290676.1 | A-140611 | hTTRsc02 | sense | usgsggauUfuCfAfUfguaaccaagaL10 | | |
| | A-265470 | TTR | antissense | VPuCfuugGfuuAfcaugAfaAfucccasu | 62 | |
| | | | | sc | | |
| AD- | A-555719 | TTR | sense | usgsgga(Uhd)UfuCfAfUfguaaccaasg | 15 | C16 |
| 291845.1 | | | | sa | | |
| | A-131902 | h/c TTR | antissense | VPusCfsuugGfuuAfcaugAfaAfuccca | 17 | |
| | | | | susc | | |

TABLE 1
Ophthalmoscopic Examination Summary of non-human primates
on Days −7, 3, 8, and 30 post administration.

| PBS (Individual | TTR siRNA -AD-291845 (Individual |
|---|---|
| Animals, N = 3) | Animals, N = 3) |
| Normal | Normal |
| Blepharitis Eye/Right | Normal |
| Normal | Normal |

TABLE 2
Histopathology Summary (Day 31) of non-human primates

| | PBS | TTR siRNA -AD-291845 |
|---|---|---|
| Eye right | (N = 3) | (N = 3) |
| Mono nuclear infiltrate | Normal | Normal |
| (ciliary body, uvea, sclera) | | |
| Degeneration; lens | Normal | Normal |
| Degeneration retina | Normal | Normal |
| Degeneration optic nerve | Normal | Normal |

The Tables below summarize the unmodified and modified sense and antisense strand nucleotide sequences used in Examples 1-3.

| | | | SEQ | | | SEQ | |
|---|---|---|---|---|---|---|---|
| | | | ID | | | ID | |
| Duplex | Oligo | Modified Sequence (5′ to 3′) | NO: | Strand | Unmodified sequence | NO: | Target |
| AD-23043 | A-51593 | cAGuGuucuuGcucuAuAAdTdTsL10 | 53 | sense | CAGUGUUCUUGCUCUAUAA | 63 | TTR_ |
| | | | | | | | rodent |
| | A-32756 | UuAuAGAGcAAGAAcACUGdTdT | 54 | antis | UUAUAGAGCAAGAACACUG | 64 | TTR |
| AD-65808 | A-130279 | AfsasCfaGfuGfuUfCfUfuGfcUfcUfaUfas | 55 | sense | AACAGUGUUCUUGCUCUAUAA | 65 | mTTRsc |
| | | AfdTdTL10 | | | | | |
| | A-117800 | usUfsaUfaGfaGfcAfagaAfcAfcUfgUfusu | 56 | antis | UUAUAGAGCAAGAACACUGUU | 66 | m/r TTR |
| | | su | | | UU | | |
| AD-67175 | A-130279 | AfsasCfaGfuGfuUfCfUfuGfcUfcUfaUfas | 55 | sense | AACAGUGUUCUUGCUCUAUAA | 65 | mTTRsc |
| | | AfdTdTL10 | | | | | |
| | A-130284 | VPusUfsaUfaGfaGfcAfagaAfcAfcUfgUfu | 57 | antis | UUAUAGAGCAAGAACACUGUU | 66 | mTTRsc |
| | | susu | | | UU | | |
| AD-70191 | A-139575 | usgsggauUfuCfAfUfguaaccaagsadTdTL10 | 59 | sense | UGGGAUUUCAUGUAACCAAGA | 12 | hTTRsc02 |
| | A-131902 | VPusCfsuugGfuuAfcaugAfaAfucccasusc | 17 | antis | UCUUGGUUACAUGAAAUCCCA | 13 | h/c TTR |
| | | | | | UC | | |
| AD-70500 | A-140611 | usgsggauUfuCfAfUfguaaccaagaL10 | 60 | sense | UGGGAUUUCAUGUAACCAAGA | 12 | hTTRsc02 |
| | A-131902 | VPusCfsuugGfuuAfcaugAfaAfucccasusc | 17 | antis | UCUUGGUUACAUGAAAUCCCA | 13 | h/c TTR |
| | | | | | UC | | |
| AD-77745 | A-147399 | AfsasCfaGfuGfuUfCfUfuGfcUfcUfaUfsa | 58 | sense | AACAGUGUUCUUGCUCUAUAA | 65 | m/rTTR |
| | | sAf | | | | | |
| | A-130284 | VPusUfsaUfaGfaGfcAfagaAfcAfcUfgUfu | 57 | antis | UUAUAGAGCAAGAACACUGUU | 66 | mTTRsc |
| | | susu | | | UU | | |
| AD-291845 | A-555719 | usgsgga(Uhd)UfuCfAfUfguaaccaasgsa | 15 | sense | UGGGAUUUCAUGUAACCAAGA | 12 | TTR |
| | A-131902 | VPusCfsuugGfuuAfcaugAfaAfucccasusc | 17 | antis | UCUUGGUUACAUGAAAUCCCA | 13 | h/c TTR |
| | | | | | UC | | |
| AD-70500 | A-140611 | usgsggauUfuCfAfUfguaaccaagaL10 | 60 | sense | UGGGAUUUCAUGUAACCAAGA | 12 | hTTRsc02 |
| | A-131902 | VPusCfsuugGfuuAfcaugAfaAfucccasusc | 17 | antis | UCUUGGUUACAUGAAAUCCCA | 13 | h/c TTR |
| | | | | | UC | | |
| AD-290674 | A-515644 | usgsggauUfuCfAfUfguaaccaagaL57 | 61 | sense | UGGGAUUUCAUGUAACCAAGA | 12 | h/c TTR |
| | A-131902 | VPusCfsuugGfuuAfcaugAfaAfucccasusc | 17 | antis | UCUUGGUUACAUGAAAUCCCA | 13 | h/c TTR |
| | | | | | UC | | |
| AD-290675 | A-515644 | usgsggauUfuCfAfUfguaaccaagaL57 | 61 | sense | UGGGAUUUCAUGUAACCAAGA | 12 | h/c TTR |
| | A-265470 | VPuCfuugGfuuAfcaugAfaAfucccasusc | 62 | antis | UCUUGGUUACAUGAAAUCCCA | 13 | TTR |
| | | | | | UC | | |
| AD-290676 | A-140611 | usgsggauUfuCfAfUfguaaccaagaL10 | 60 | sense | UGGGAUUUCAUGUAACCAAGA | 12 | hTTRsc02 |
| | A-265470 | VPuCfuugGfuuAfcaugAfaAfucccasusc | 62 | antis | UCUUGGUUACAUGAAAUCCCA | 13 | TTR |
| | | | | | UC | | |
| AD-291845 | A-555719 | usgsgga(Uhd)UfuCfAfUfguaaccaasgsa | 15 | sense | UGGGAUUUCAUGUAACCAAGA | 12 | TTR |
| | A-131902 | VPusCfsuugGfuuAfcaugAfaAfucccasusc | 17 | antis | UCUUGGUUACAUGAAAUCCCA | 13 | h/c TTR |
| | | | | | UC | | |

TABLE 3
Abbreviations of nucleotide monomers used in nucleic
acid sequence representation. It will be understood
that these monomers, when present in an oligonucleotide,
are mutually linked by 5′-3′-phosphodiester bonds.

| Abbreviation | Nucleotide(s) |
|---|---|
| A | Adenosine-3′-phosphate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| N | any nucleotide (G, A, C, T or U) |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methylthymine-3′-phosphate |
| ts | 2′-O-methyl-5-methylthymine-3′-phosphorothioate |
| u | 2′-O-methyluridine-3′-phosphate |
| us | 2′-O-methyluridine-3′-phosphorothioate |
| s | phosphorothioate linkage |
| L96 | N-[tris(GalNAc-alkyl)-amidodecanoyl)]-4-hydroxyprolinol |
| | Hyp-(GalNAc-alkyl)3 |
| dA | deoxy-adenosine |
| dC | deoxy-cytodine |
| dG | deoxy-guanosine |
| (dT) | 2′-deoxythymidine-3′-phosphate |
| Y34 | 2-hydroxymethyl-tetrahydrofurane-4-methoxy-3-phosphate |
| | (abasic 2′-OMe furanose) |
| Y44 | 2-hydroxymethyl-tetrahydrofurane-5-phosphate |
| P | Phosphate |
| VP | Vinyl-phosphonate |
| (Ahd) | 2′-O-hexadecyl-adenosine-3′-phosphate |
| (Ahds) | 2′-O-hexadecyl-adenosine-3′-phosphorothioate |
| (Chd) | 2′-O-hexadecyl-cytidine-3′-phosphate |
| (Chds) | 2′-O-hexadecyl-cytidine-3′-phosphorothioate |
| (Ghd) | 2′-O-hexadecyl-guanosine-3′-phosphate |
| Ghds) | 2′-O-hexadecyl-guanosine-3′-phosphorothioate |
| (Uhd) | 2′-O-hexadecyl-uridine-3′-phosphate |
| (Uhds) | 2′-O-hexadecyl-uridine-3′-phosphorothioate |
| L10 | N-(cholesterylcarboxamidocaproyl)-4-hydroxyprolinol |
| | (Hyp-C6-Chol) |
| L57 | N-(stearylcarboxamidocaproyl)-4-hydroxyprolinol |
| | (Hyp-C6-C18) |

TABLE 4
Exemplary Unmodified Sense and Antisense Strand Sequences of TTR dsRNAs

| | | SEQ | Antisense Start Site | | SEQ |
|---|---|---|---|---|---|
| | | ID | Relative to | | ID |
| Duplex ID | Sense sequence (5′ to 3′) | NO: | NM_000371.2 | Antisense sequence (5′ to 3′) | NO: |
| AD-68322.2 | AUGGGAUUUCAUGUAACCAAA | 67 | 504 | UUUGGUUACAUGAAAUCCCAUCC | 77 |
| AD-60668.3 | AUGGGAUUUCAUGUAACCAAA | 67 | 504 | UUUGGUUACAUGAAAUCCCAUCC | 77 |
| AD-68330.1 | AUGGGAUUUCAUGUAACCAAA | 67 | 504 | UUUGGUUACAUGAAAUCCCAUCC | 77 |
| AD-64474.4 | UGGGAUUUCAUGUAACCAAGA | 12 | 505 | UCUUGGUUACAUGAAAUCCCAUC | 13 |
| AD-65468.2 | UGGGAUUUCAUGUAACCAAGA | 12 | 505 | UCUUGGUUACAUGAAAUCCCAUC | 13 |
| AD-65492.1 | UGGGAUUUCAUGUAACCAAGA | 12 | 505 | UCUUGGUUACAUGAAAUCCCAUC | 13 |
| AD-65480.2 | UGGGAUUUCAUGUAACCAAGA | 12 | 505 | UCUUGGUUACAUGAAAUCCCAUC | 13 |
| AD-60636.3 | UUUCAUGUAACCAAGAGUAUU | 68 | 510 | AAUACUCUUGGUUACAUGAAAUC | 78 |
| AD-68320.2 | UUUCAUGUAACCAAGAGUAUU | 68 | 510 | AAUACUCUUGGUUACAUGAAAUC | 78 |
| AD-68326.1 | UUUCAUGUAACCAAGAGUAUU | 68 | 510 | AAUACUCUUGGUUACAUGAAAUC | 78 |
| AD-60611.4 | UGUAACCAAGAGUAUUCCAUU | 69 | 515 | AAUGGAAUACUCUUGGUUACAUG | 79 |
| AD-68331.1 | UGUAACCAAGAGUAUUCCAUU | 69 | 515 | AAUGGAAUACUCUUGGUUACAUG | 79 |
| AD-68315.2 | UGUAACCAAGAGUAUUCCAUU | 69 | 515 | AAUGGAAUACUCUUGGUUACAUG | 79 |
| AD-68319.2 | AACCAAGAGUAUUCCAUUUUU | 70 | 518 | AAAAAUGGAAUACUCUUGGUUAC | 80 |
| AD-60612.5 | AACCAAGAGUAUUCCAUUUUU | 70 | 518 | AAAAAUGGAAUACUCUUGGUUAC | 80 |
| AD-68316.2 | AACCAAGAGUAUUCCAUUUUU | 70 | 518 | AAAAAUGGAAUACUCUUGGUUAC | 80 |
| AD-60664.3 | UUUUUACUAAAGCAGUGUUUU | 71 | 534 | AAAACACUGCUUUAGUAAAAAUG | 81 |
| AD-68321.2 | UUUUUACUAAAGCAGUGUUUU | 71 | 534 | AAAACACUGCUUUAGUAAAAAUG | 81 |
| AD-68318.2 | UUUUUACUAAAGCAGUGUUUU | 71 | 534 | AAAACACUGCUUUAGUAAAAAUG | 81 |
| AD-60665.5 | UUACUAAAGCAGUGUUUUCAA | 72 | 537 | UUGAAAACACUGCUUUAGUAAAA | 82 |
| AD-60642.4 | CUAAAGCAGUGUUUUCACCUA | 73 | 540 | UAGGUGAAAACACUGCUUUAGUA | 83 |
| AD-68329.1 | CUAAAGCAGUGUUUUCACCUA | 73 | 540 | UAGGUGAAAACACUGCUUUAGUA | 83 |
| AD-68334.1 | CUAAAGCAGUGUUUUCACCUA | 73 | 540 | UAGGUGAAAACACUGCUUUAGUA | 83 |
| AD-68328.1 | GGCAGAGACAAUAAAACAUUA | 74 | 582 | UAAUGUUUUAUUGUCUCUGCCUG | 84 |
| AD-68333.1 | GGCAGAGACAAUAAAACAUUA | 74 | 582 | UAAUGUUUUAUUGUCUCUGCCUG | 84 |
| AD-60639.3 | GGCAGAGACAAUAAAACAUUA | 74 | 582 | UAAUGUUUUAUUGUCUCUGCCUG | 84 |
| AD-60643.4 | CAGAGACAAUAAAACAUUCCU | 75 | 584 | AGGAAUGUUUUAUUGUCUCUGCC | 85 |
| AD-68317.2 | CAGAGACAAUAAAACAUUCCU | 75 | 584 | AGGAAUGUUUUAUUGUCUCUGCC | 85 |
| AD-68335.1 | CAGAGACAAUAAAACAUUCCU | 75 | 584 | AGGAAUGUUUUAUUGUCUCUGCC | 85 |
| AD-68327.1 | CAAUAAAACAUUCCUGUGAAA | 76 | 590 | UUUCACAGGAAUGUUUUAUUGUC | 86 |
| AD-68332.1 | CAAUAAAACAUUCCUGUGAAA | 76 | 590 | UUUCACAGGAAUGUUUUAUUGUC | 86 |
| AD-60637.2 | CAAUAAAACAUUCCUGUGAAA | 76 | 590 | UUUCACAGGAAUGUUUUAUUGUC | 86 |

### Example 4. Dose-Response Inhibition of Ocular TTR Expression in Non-Human Primates

The efficacy of the dsRNA agent AD-291845 to knockdown ocular TTR expression was assessed in the eyes of non-human primates in a dose-response study. Male Cynomolgous monkeys (n=2 per group) were intravitreally administered a single 0.1 mg, 0.3 mg, 1 mg, or 3 mg dose of AD-291845 in a total volume of 50 μl on Day 0. Vitreous humor and aqueous humor were collected from the eyes. Ocular tissues (RPE and CE) were dissected. Lysates were prepared from the ocular tissues, liver, and kidney. TTR mRNA levels are determined as described above. TTR protein levels were determined by ELISA and immunohistochemistry (IHC).

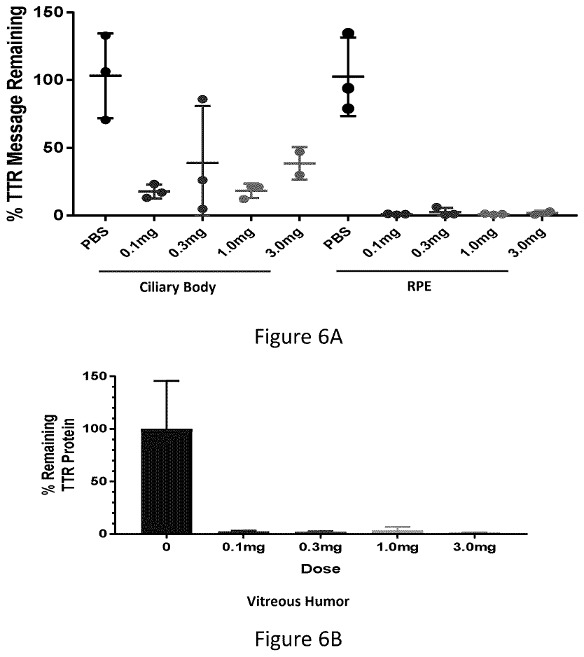

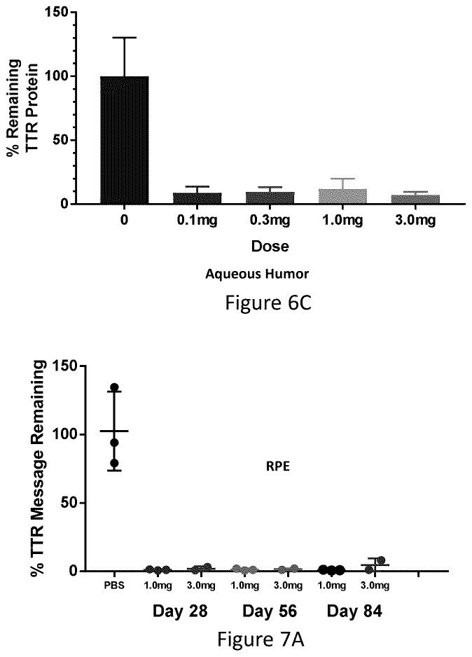

As illustrated in FIG. 6A, a single 0.1 mg, 0.3 mg, 1 mg, or 3 mg dose of AD-291845 significantly reduced the mRNA levels of TTR in both ciliary epithelium and retinal pigment epithelium at Day 28. The results were confirmed by IHC. Administration of even the lowest dose of AD-291854 resulted in near complete reduction of TTR protein in vitreous humor (FIG. 6B) and aqueous humor (FIG. 6C) at Day 28 as determined by ELISA.

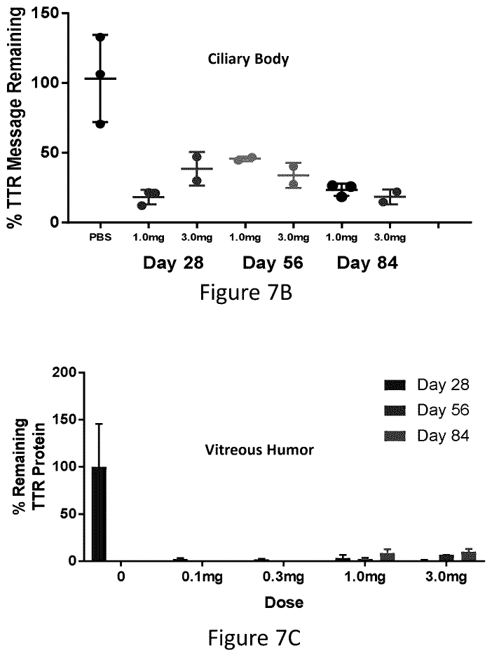

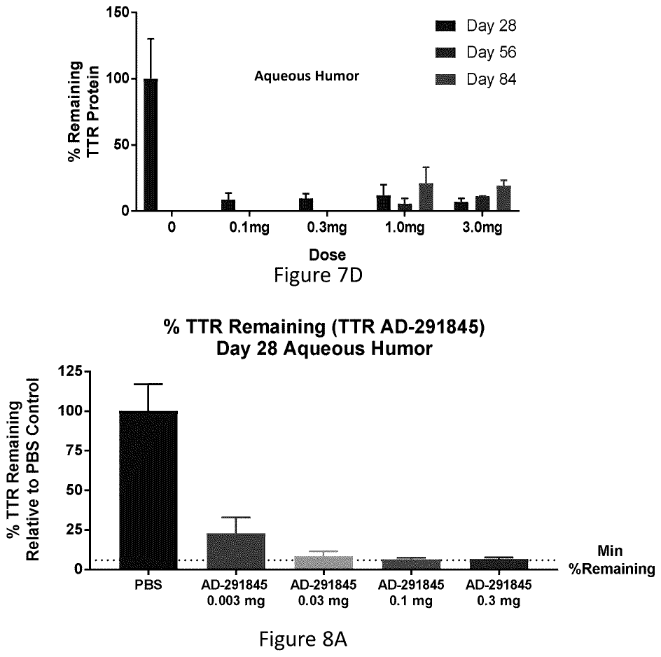

Further, robust knockdown of TTR was observed at all time points tested. A single 1 mg or 3 mg dose of AD-291845 significantly reduced the mRNA levels of TTR in both ciliary epithelium (FIG. 7A) and retinal pigment epithelium (FIG. 7B) at Days 28, 56, and 84. A single 1 mg or 3 mg dose of AD-291845 resulted in near complete reduction of TTR protein in vitreous humor (FIG. 7C) and aqueous humor (FIG. 7D) at a single 0.1 mg or 0.3 mg dose on Day 28, or a single 1 mg or 3 mg dose on Days 28, 56, and 84 as determined by ELISA.

These data demonstrate that AD-291845 robustly and durably reduces TTR mRNA and protein in ocular tissues.

### Example 5. Low Dose Inhibition of Ocular TTR Expression in Non-Human Primates

Having demonstrated robust and durable knockdown of both TTR mRNA and protein by intravitreal administration of AD-291845 at doses as low as 0.1 mg/eye, lower doses of AD-291845 were tested in a separate study in non-human primates.

| | | | | | SEQ ID |
|---|---|---|---|---|---|
| Duplex | Oligo | Target | Strand | Modified Sequence (5′ to 3′) | NO: |
| AD-291845 | A-555719 | TTR | sense | usgsgga(Uhd)UfuCfAfUfguaaccaasgsa | 15 |
| | A-131902 | h/c TTR | antis | VPusCfsuugGfuuAfcaugAfaAfucccasusc | 17 |

Female Cynomolgous monkeys (n=2 per group) were intravitreally administered a single 0.003 mg, 0.03 mg, 0.1 mg, or 0.3 mg dose of AD-291845 in a total volume of 50 μl on Day 0. Aqueous humor was collected on Day 28. Eyes, livers, and kidneys were collected on Day 168 post administration. Vitreous humor and aqueous humor were collected from eyes. Ocular tissues (RPE and CE) were dissected. Lysates were prepared from the ocular tissues, liver, and kidney. TTR mRNA levels were determined as described above. TTR protein levels were determined by ELISA and immunohistochemistry (IHC).

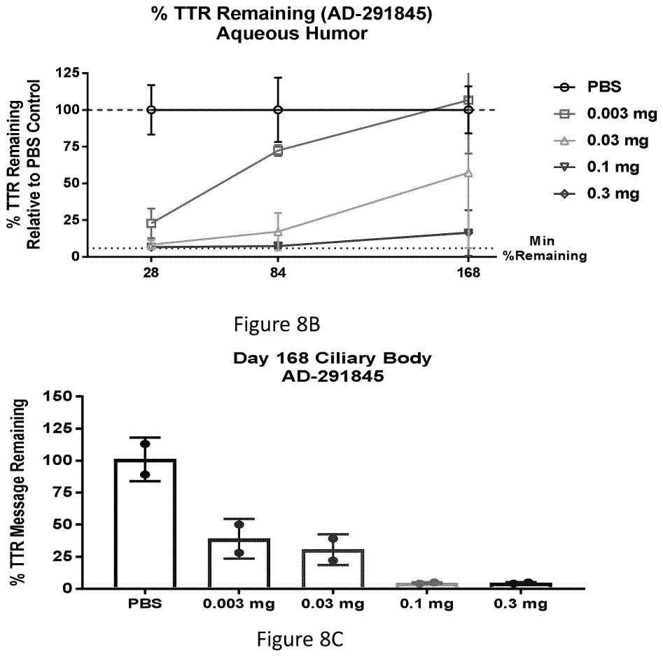

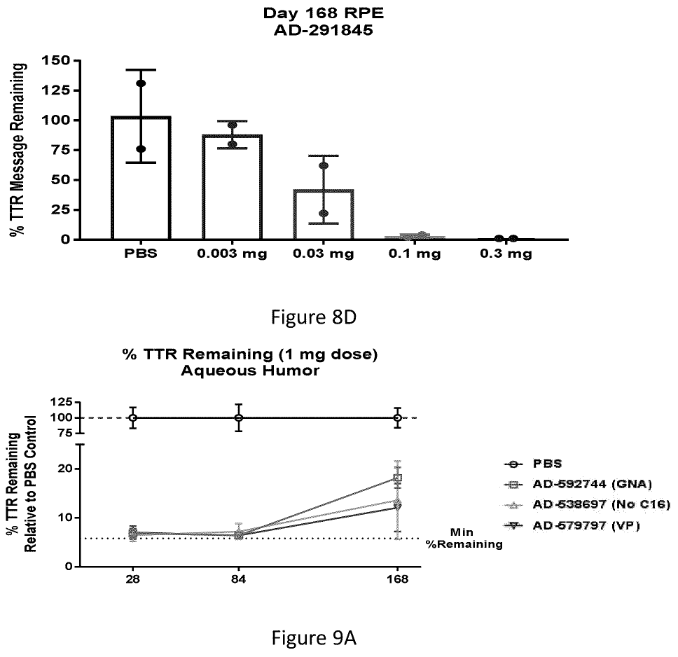

As shown in FIG. 8A, a single 0.003 mg, 0.03 mg, 0.1 mg, or 0.3 mg dose of AD-291845 resulted in near complete reduction of TTR protein in aqueous humor at Day 28 as determined by ELISA as compared to PBS treated control. A graph showing percent TTR protein remaining in aqueous humor as compared to PBS control on Days 28, 84, and 168 post injection at each of the four doses is provided in FIG. 8B. The results show a dose response with higher doses of AD-291845 providing greater levels and more sustained knockdown of TTR in aqueous humor.

At the terminal time point of 168 days, eyes were harvested and the ciliary body and retinal pigment epithelium (RPE) were isolated and the level of TTR message remaining as compared to PBS treated control was determined. Results are shown in FIGS. 8C and 8D, respectively. Again, the results show a dose response with higher doses of AD-291845 providing greater levels of TTR mRNA knockdown in both the ciliary body and RPE.

These data demonstrate that AD-291845 robustly and durably reduces TTR mRNA and protein expression in ocular tissues even at low doses.

### Example 6. Inhibition of Ocular TTR Expression in Mice

Further dsRNA agents with the same nucleotide sequence as AD-65808 and AD-67175 provided above with a C16 modification at various locations were designed. The agents are shown in Table 5.

TABLE 5
Modified sense and antisense strand sequences of mouse specific TTR dsRNA

| | | SEQ | | SEQ |
|---|---|---|---|---|
| Duplex | | ID | | ID |
| Name | Sense sequence (5′ to 3′) | NO: | Antisense sequence (5′ to 3′) | NO: |
| AD-307566 | (Ahds)ascaguGfuUfCfUfugcucuausasa | 87 | VPuUfauaGfagcaagaAfcAfcuguususu | 98 |
| AD-307567 | as(Ahds)caguGfuUfCfUfugcucuausasa | 88 | VPuUfauaGfagcaagaAfcAfcuguususu | 98 |
| AD-307570 | asasca(Ghd)uGfuUfCfUfugcucuausasa | 89 | VPuUfauaGfagcaagaAfcAfcuguususu | 98 |
| AD-307571 | asascag(Uhd)GfuUfCfUfugcucuausasa | 90 | VPuUfauaGfagcaagaAfcAfcuguususu | 98 |
| AD-307572 | asascagu(Ghd)uUfCfUfugcucuausasa | 91 | VPuUfauaGfagcaagaAfcAfcuguususu | 98 |
| AD-307575 | asascaguGfuUf(Chd)Ufugcucuausasa | 92 | VPuUfauaGfagcaagaAfcAfcuguususu | 98 |
| AD-307576 | asascaguGfuUfCf(Uhd)ugcucuausasa | 93 | VPuUfauaGfagcaagaAfcAfcuguususu | 98 |
| AD-307580 | asascaguGfuUfCfUfugc(Uhd)cuausasa | 94 | VPuUfauaGfagcaagaAfcAfcuguususu | 98 |
| AD-307585 | asascaguGfuUfCfUfugcucuaus(Ahds)a | 95 | VPuUfauaGfagcaagaAfcAfcuguususu | 98 |
| AD-307586 | asascaguGfuUfCfUfugcucuausas(Ahd) | 96 | VPuUfauaGfagcaagaAfcAfcuguususu | 98 |
| AD-307590 | asascaguGfuUfCfUfugcucuausasa | 97 | VPuUfau(Ahd)GfagcaagaAfcAfcuguususu | 99 |
| AD-307600 | asascaguGfuUfCfUfugcucuausasa | 97 | VPuUfauaGfagcaagaAf(Chd)Afcuguususu | 100 |
| AD-307601 | asascaguGfuUfCfUfugcucuausasa | 97 | VPuUfauaGfagcaagaAfc(Ahd)cuguususu | 101 |

Female C57BL/6 mice (n=3 mice, 6 eyes per group) were intravitreally administered a single 7.5 μg/eye of one of the duplexes listed in Table 5 or PBS control on Day 0. At Day 13, eyes were harvested and the percent of mouse TTR mRNA remaining as compared to PBS control. The results are shown in Table 6.

TABLE 6
mTTR Single 7.5 μg Dose Screen in Mouse Eye

| Duplex | Average | Standard Deviation |
|---|---|---|
| PBS | 100.00 | 11.19 |
| naïve | 114.18 | 24.76 |
| AD-307566 | 10.65 | 10.52 |
| AD-307567 | 18.34 | 11.53 |
| AD-307570 | 51.94 | 15.25 |
| AD-307571 | 18.13 | 3.02 |
| AD-307572 | 13.91 | 2.87 |
| AD-307575 | 84.73 | 21.65 |
| AD-307576 | 42.42 | 9.26 |
| AD-307580 | 8.27 | 2.65 |
| AD-307585 | 6.67 | 3.86 |
| AD-307586 | 5.82 | 5.72 |
| AD-307590 | 30.77 | 9.60 |
| AD-307600 | 34.37 | 16.23 |
| AD-307601 | 7.97 | 3.86 |

These data demonstrate that the position of the C16 modification can alter the level of mTTR silencing in mouse eye.

### Example 7. Inhibition of Ocular TTR Expression in Non-Human Primates

Further dsRNA agents were tested for inhibition of TTR expression in non-human primates. Female Cynomolgous monkeys (n=2 per group) were intravitreally administered a single 1 mg dose of AD-592744, AD-538697, or AD-597979 in a total volume of 50 μl on Day 0. Aqueous humor was collected on Days 28, 84, and 168 post administration. Eyes were collected on 168 post administration. Ocular tissues (RPE and CE) were dissected. Lysates were prepared from the ocular tissues. TTR mRNA levels were determined as described above. TTR protein levels were determined by ELISA and immunohistochemistry (IHC).

TABLE 7
TTR Single 1 mg Dose Screen in NHP Eye

| | | | | | SEQ |
|---|---|---|---|---|---|
| | | | | | ID |
| Duplex | Oligo | Target | Strand | Modified Sequence (5′ to 3′) | NO: |
| AD-592744 | A-555719 | TTR | sense | usgsgga(Uhd)UfuCfAfUfguaaccaasgsa | 15 |
| | A-1104003 | TTR | antissense | VPusCfsuugGf(Tgn)uAfcaugAfaAfucccasusc | 102 |
| AD-538697 | A-801811 | h/cTTR | sense | usgsggauUfuCfAfUfguaaccaasgsa | 103 |
| | A-131902 | h/c TTR | antissense | VPusCfsuugGfuuAfcaugAfaAfucccasusc | 17 |
| AD-579797 | A-555719 | TTR | sense | usgsgga(Uhd)UfuCfAfUfguaaccaasgsa | 15 |
| | A-131359 | TTR | antissense | usCfsuugGfuuAfcaugAfaAfucccasusc | 16 |

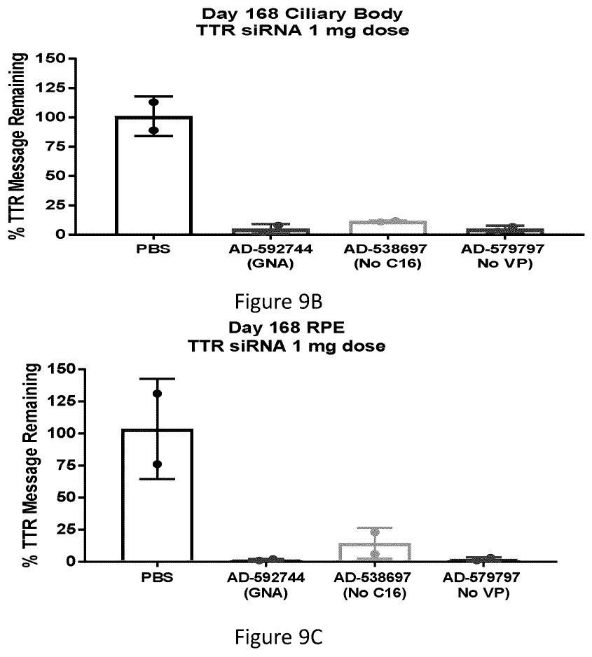

The results are shown in FIGS. 9A-9C. A graph showing percent TTR protein remaining in aqueous humor as compared to PBS control on Days 28, 84, and 168 post injection with each of the three dsRNA agents is provided in FIG. 9A.

At the terminal time point of 168 days, eyes were harvested and the ciliary body and retinal pigment epithelium (RPE) were isolated and the level of TTR message remaining as compared to PBS treated control was determined. Results are shown in FIGS. 9B and 9C, respectively. The results show effective mRNA knockdown in both the ciliary body and RPE by all three dsRNA agents.

These data demonstrate that AD-592744, AD-538697, and AD-597979 all robustly and durably reduce TTR mRNA and protein expression in ocular tissues.

### Example 8. Inhibition of Ocular TTR Expression in Non-Human Primates

Further dsRNA agents were tested for inhibition of TTR expression in non-human primates. Female Cynomolgous monkeys (n=2 per group) were intravitreally administered a single dose of a dsRNA agent in a total volume of 50 μl on Day 0 as shown in the table below.

TABLE 8
TTR Single Dose Screen in NHP Eye

| | | | | SEQ | |
|---|---|---|---|---|---|
| | | | | ID | Dose |
| Duplex | Oligo | Strand | Modified Sequence (5′ to 3′) | NO: | (mg) |
| AD-291845 | A-555719 | sense | usgsgga(Uhd)UfuCfAfUfguaaccaasgsa | 15 | 0.01; |
| | A-131902 | antissense | VPusCfsuugGfuuAfcaugAfaAfucccasusc | 17 | 0.003 |
| AD-291846 | A-555719 | sense | usgsgga(Uhd)UfuCfAfUfguaaccaasgsa | 15 | 0.003 |
| | A-265470 | antissense | VPuCfuugGfuuAfcaugAfaAfucccasusc | 62 | |
| AD-592744 | A-555719 | sense | usgsgga(Uhd)UfuCfAfUfguaaccaasgsa | 15 | 0.03; |
| | A-1104003 | antissense | VPusCfsuugGf(Tgn)uAfcaugAfaAfucccasusc | 102 | 0.01; |
| | | | | | 0.003 |
| AD-538697 | A-801811 | sense | usgsggauUfuCfAfUfguaaccaasgsa | 103 | 0.03; |
| | A-131902 | antissense | VPusCfsuugGfuuAfcaugAfaAfucccasusc | 17 | 0.01 |
| AD-579797 | A-555719 | sense | usgsgga(Uhd)UfuCfAfUfguaaccaasgsa | 15 | 0.01 |
| | A-131359 | antissense | usCfsuugGfuuAfcaugAfaAfucccasusc | 16 | |

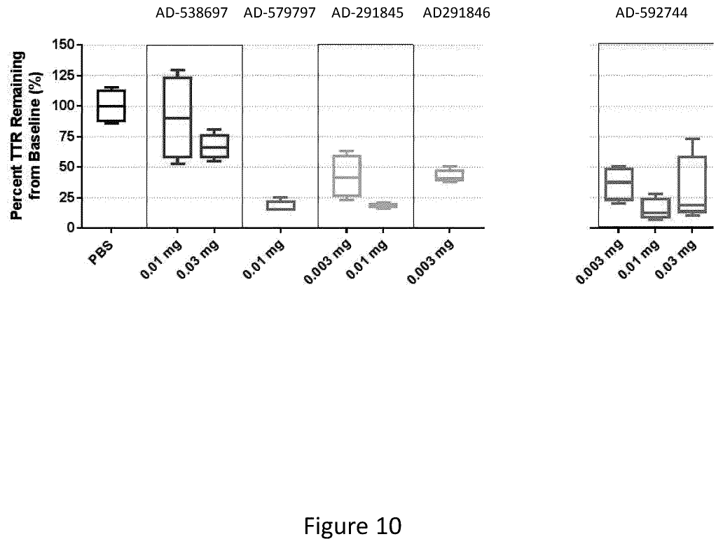

Aqueous humor was collected on Days 28 post administration. Aqueous humor is also collected on Days 56 and 85 post administration. Eyes are collected on Day 85 post administration to assess TTR knockdown and histology. A graph showing percent TTR protein remaining in aqueous humor as compared to PBS control on Day 28 post injection at with each of the three dsRNA agents is provided in FIG. 10.

These data demonstrate efficient knockdown of TTR expression by the dsRNA agents in eyes of NHP even at low doses, especially by dsRNA conjugates containing a C16 lipophilic moiety.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

1-3. (canceled)

4. A double stranded RNAi agent, or a salt thereof, for inhibiting expression of TTR in a cell, wherein the double stranded RNAi agent comprises a sense strand and an antisense strand forming a double stranded region;
wherein the sense strand comprises the nucleotide sequence 5′-UGGGAUUUCAUGUAACCAAGA-3′ of SEQ ID NO:12 and the antisense strand comprises the nucleotide sequence 5′-UCUUGGUUACAUGAAAUCCCAUC-3′ of SEQ ID NO: 13;
wherein substantially all of the nucleotides of the sense strand and substantially all of the nucleotides of the antisense strand comprise a nucleotide modification; and
wherein one or more lipophilic moieties are conjugated to one or more internal positions on at least one strand, or one or more positions on at least one strand within the double stranded region.

5. The double stranded RNAi agent, or a salt thereof, of claim 4, wherein the sense strand comprises a nucleotide sequence differing by no more than 4 modified nucleotides from the nucleotide sequence 5′-usgsggauUfuCfAfUfguaaccaaga-3′ of SEQ ID NO: 10 and the antisense strand comprises a nucleotide sequence differing by no more than 4 modified nucleotides from the nucleotide sequence 5′-usCfsuugGfuuAfcaugAfaAfucccasusc-3′ of SEQ ID NO: 7,
wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively; and s is a phosphorothioate linkage.

6. (canceled)

7. The double stranded RNAi agent, or a salt thereof, of claim 4, wherein the one or more lipophilic moieties are conjugated to the one or more internal positions on at least one strand, or the one or more positions on at least one strand within the double stranded region, via a linker or carrier.

8-14. (canceled)

15. The double stranded RNAi agent, or a salt thereof, of claim 4, wherein the one or more internal positions include all positions except the terminal two positions from each end of the at least one strand.

16-21. (canceled)

22. The double stranded RNAi agent, or a salt thereof, of claim 15, wherein the internal positions include all positions except the terminal two positions from each end of the at least one strand and positions 11-13 on the sense strand, counting from the 3′-end, and positions 12-14 on the antisense strand, counting from the 5′-end.

23. The double stranded RNAi agent, or a salt thereof, of claim 4, wherein the one or more lipophilic moieties are conjugated to the one or more of the internal positions selected from the group consisting of positions 4-8 and 13-18 on the sense strand, and positions 6-10 and 15-18 on the antisense strand, counting from the 5′ end of each strand.

24. The double stranded RNAi agent, or a salt thereof, of claim 23, wherein the one or more lipophilic moieties are conjugated to the one or more of the internal positions selected from the group consisting of positions 5, 6, 7, 15, and 17 on the sense strand, and positions 15 and 17 on the antisense strand, counting from the 5′-end of each strand.

25. (canceled)

26. The double stranded RNAi agent of claim 4,
(a) wherein the lipophilic moiety is an aliphatic, alicyclic, or polyalicyclic compound;
(b) wherein the lipophilic moiety is selected from the group consisting of lipid, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine;
(c) wherein the lipophilic moiety contains a saturated or unsaturated C4-C30 hydrocarbon chain, and an optional functional group selected from the group consisting of hydroxyl, amine, carboxylic acid, sulfonate, phosphate, thiol, azide, and alkyne;
(d) wherein the lipophilic moiety contains a saturated or unsaturated C6-C18 hydrocarbon chain; and/or
(e) wherein the lipophilic moiety contains a saturated or unsaturated C16 hydrocarbon chain.

27-57. (canceled)

58. The double stranded RNAi agent, or a salt thereof, of claim 4, further comprising at least one phosphorothioate or methylphosphonate internucleotide linkage.

59-73. (canceled)

74. The double stranded RNAi agent, or a salt thereof, of claim 4, further comprising a phosphate or phosphate mimic at the 5′-end of the antisense strand.

75. The double stranded RNAi agent, or a salt thereof, of claim 74, wherein the phosphate mimic is a 5′-vinyl phosphonate (VP).

76-78. (canceled)

79. The double stranded RNAi agent, or a salt thereof, of claim 4, wherein the sense strand and the antisense strand comprise sense and antisense strand nucleotide sequences selected from the group consisting of
5′-usgsgga(Uhd)UfuCfAfUfguaaccaasgsa-3′ of SEQ ID NO: 15 and
5′-VPusCfsuugGfuuAfcaugAfaAfucccasusc-3′ of SEQ ID NO: 17;
5′-usgsgga(Uhd)UfuCfAfUfguaaccaasgsa-3′ of SEQ ID NO: 15 and
5′-VPuCfuugGfuuAfcaugAfaAfucccasusc-3′ of SEQ ID NO: 62;
5′-usgsgga(Uhd)UfuCfAfUfguaaccaasgsa-3′ of SEQ ID NO: 15 and
5′-VPusCfsuugGf(Tgn)uAfcaugAfaAfucccasusc-3′ of SEQ ID NO: 102;
and
5′-usgsgga(Uhd)UfuCfAfUfguaaccaasgsa-3′ of SEQ ID NO: 15 and
5′-usCfsuugGfuuAfcaugAfaAfucccasusc-3′ of SEQ ID NO: 16,
wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively; (Uhd) is 2′-O-hexadecyl-uridine-3′-phosphate; (Tgn) is thymidine-glycol nucleic acid (GNA)S-Isomer; s is a phosphorothioate linkage; and VP is a vinyl phosphonate.

80. A double stranded ribonucleic acid (RNAi) agent, or a salt thereof, that inhibits expression of transthyretin (TTR) in a cell, comprising a sense strand and an antisense strand forming a double stranded region,
wherein each of the sense strand and the antisense strand independently comprise nucleotide sequences differing by no more than 4 modified nucleotides from the sense and antisense strand nucleotide sequences of a duplex selected from the group consisting of
5′-usgsgga(Uhd)UfuCfAfUfguaaccaasgsa-3′ of SEQ ID NO: 15 and
5′-VPusCfsuugGfuuAfcaugAfaAfucccasusc-3′ of SEQ ID NO: 17;
5′-usgsgga(Uhd)UfuCfAfUfguaaccaasgsa-3′ of SEQ ID NO: 15 and
5′-VPuCfuugGfuuAfcaugAfaAfucccasusc-3′ of SEQ ID NO: 62;
5′-usgsgga(Uhd)UfuCfAfUfguaaccaasgsa-3′ of SEQ ID NO: 15 and
5′-VPusCfsuugGf(Tgn)uAfcaugAfaAfucccasusc-3′ of SEQ ID NO: 102;
and
5′-usgsgga(Uhd)UfuCfAfUfguaaccaasgsa-3′ of SEQ ID NO: 15 and
5′-usCfsuugGfuuAfcaugAfaAfucccasusc-3′ of SEQ ID NO: 16,
wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively;
(Uhd) is 2′-O-hexadecyl-uridine-3′-phosphate; (Tgn) is thymidine-glycol nucleic acid (GNA)S-Isomer; s is a phosphorothioate linkage; and VP is a vinyl phosphonate.

81-83. (canceled)

84. The double stranded RNAi agent, or a salt thereof, of claim 80, wherein the sense strand comprises the nucleotide sequence 5′-usgsgga(Uhd)UfuCfAfUfguaaccaasgsa-3′ of SEQ ID NO: 15 and the antisense strand comprises the nucleotide sequence 5′-usCfsuugGfuuAfcaugAfaAfucccasusc-3′ of SEQ ID NO: 16.

85. The double stranded RNAi agent, or a salt thereof, of claim 80, wherein the sense strand consists of the nucleotide sequence 5′-usgsgga(Uhd)UfuCfAfUfguaaccaasgsa-3′ of SEQ ID NO: 15 and the antisense strand comprises the nucleotide sequence 5′-usCfsuugGfuuAfcaugAfaAfucccasusc-3′ of SEQ ID NO: 16.

86. The double stranded RNAi agent, or a salt thereof, of claim 80, further comprising a phosphate or phosphate mimic at the 5′-end of the antisense strand.

87. (canceled)

88. A double stranded RNAi agent, or a salt thereof, for inhibiting expression of transthyretin (TTR) in a cell, wherein the RNAi agent comprises a sense strand and an antisense strand forming a double stranded region, wherein the sense strand comprises the nucleotide sequence 5′-usgsgga(Uhd)UfuCfAfUfguaaccaasgsa-3′ of SEQ ID NO: 15 and the antisense strand comprises the nucleotide sequence 5′-VPusCfsuugGfuuAfcaugAfaAfucccasusc-3′ of SEQ ID NO: 17,
wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively; s is a phosphorothioate linkage; (Uhd) is 2′-O-hexadecyl-uridine-3′-phosphate; and VP is a vinyl phosphonate.

89. A pharmaceutical composition comprising the double stranded RNAi agent, or a salt thereof, of claim 4.

90. A method of inhibiting transthyretin (TTR) expression in an ocular cell, the method comprising:
contacting the cell with the double stranded RNAi agent, or a salt thereof, of claim 4, thereby inhibiting expression of the TTR gene in the ocular cell.

91. The method of claim 90, wherein the cell is within a subject, or wherein the subject is a human.

92. (canceled)

93. (canceled)

94. A method of treating a subject suffering from a TTR-associated ocular disease, comprising administering to the subject a therapeutically effective amount of the double stranded RNAi agent, or a salt thereof, of claim 4, thereby treating the subject.

95. The method of claim 94, wherein the TTR-associated ocular disease or disorder is selected from the group consisting of TTR-associated glaucoma, TTR-associated vitreous opacities, TTR-associated retinal abnormalities, TTR-associated retinal amyloid deposit, TTR-associated retinal angiopathy, TTR-associated iris amyloid deposit, TTR-associated scalloped iris, and TTR-associated amyloid deposits on lens.

96-104. (canceled)
